(19) United States
(12) Patent Application Publication
(10) Pub. No.: US 2014/0294765 A1
(43) Pub. Date: Oct. 2, 2014

(54) LSR ANTIBODIES, AND USES THEREOF FOR TREATMENT OF CANCER

(71) Applicant: Compugen Ltd., Tel Aviv-Yafo (IL)

(72) Inventors: Gad S. Cojocaru, Ramat HaSharon (IL); Liat Dassa, Yehud (IL); Galit Rotman, Herzliyya (IL); Ofer Levi, Moshav Mesisraelat Zion (IL); Andrew Pow, San Francisco, CA (US); Shirley Sameach-Greenwald, Kfar Saba (IL); Zurit Levine, Herzliyya (IL)

(73) Assignee: COMPUGEN LTD., Tel Aviv-Yafo (IL)

(21) Appl. No.: 14/361,571

(22) PCT Filed: Jun. 19, 2013
(86) PCT No.: PCT/IL2013/050527

§ 371 Date: May 29, 2014

Related U.S. Application Data
(60) Provisional application No. 61/662,470, filed on Jun. 21, 2012.

(51) Int. Cl. C07K 16/28; G01N 33/574; A61K 31/675; A61K 45/06; A61K 39/395

(52) U.S. Cl. CPC C07K 16/28; A61K 45/06; A61K 39/3955; A61K 31/675; G01N 33/57492
USPC 424/85.2; 424/139.1; 424/85.7; 424/85.6; 424/85.5; 424/133.1; 424/136.1; 435/7.23

(57) ABSTRACT

This invention relates to antibodies and antigen binding fragments and conjugates containing same, and/or alternative scaffolds, specific for LSR molecules, which are suitable drugs for immunotherapy and treatment of specific cancer.

### FIELD OF THE INVENTION

This invention relates to LSR (lipolysis stimulated lipoprotein receptor)-specific antibodies, antibody fragments, conjugates and compositions comprising same, for treatment of cancer.

### BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection. Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimmulatory factors in the B7-CD28 and TNF families, as well as by attracting regulatory T cells, which inhibit anti-tumor T cell responses (see Wang (2006) Immune Suppression by Tumor Specific CD4+ Regulatory T cells in Cancer. Semin Cancer. Biol. 16:73-79; Greenwald, et al. (2005) The B7 Family Revisited. Ann. Rev. Immunol. 23:515-48; Watts (2005) TNF/TNFR Family Members in Co-stimulation of T Cell Responses Ann. Rev. Immunol. 23:23-68; Sadum, et al. (2007) Immune Signatures of Murine and Human Cancers Reveal Unique Mechanisms of Tumor Escape and New Targets for Cancer Immunotherapy. Clin. Cane. Res. 13(13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases. As engineered cancer vaccines continue to improve, it is becoming clear that such immunologic checkpoints are a major barrier to the vaccines' ability to induce therapeutic anti-tumor responses. In that regard, costimulatory molecules can serve as adjuvants for active (vaccination) and passive (antibody-mediated) cancer immunotherapy, providing strategies to thwart immune tolerance and stimulate the immune system.

In addition, such agents could be of use in other types of cancer immunotherapy, such as adoptive immunotherapy, in which tumor-specific T cell populations are expanded and directed to attack and kill tumor cells. Agents capable of augmenting such anti-tumor response have great therapeutic potential and may be of value in the attempt to overcome the obstacles to tumor immunotherapy. Recently, novel agents that modulate several costimulatory pathways were indeed introduced to the clinic as cancer immunotherapy.

Regulating costimulation using agonists and/or antagonists to various costimulatory proteins has been extensively studied as a strategy for treating autoimmune diseases, graft rejection, allergy and cancer. This field has been clinically pioneered by CTLA4-Ig (Abatacept, Orencia®) which is approved for treatment of RA, mutated CTLA4-Ig (Belatacept, Nulojix®) for prevention of acute kidney transplant rejection and by the anti-CTLA4 antibody (Ipilimumab, Yervoy CD), recently approved for the treatment of melanoma. Other costimulation regulators are currently in advanced stages of clinical development including anti-PD-1 antibody (BMS-936558) which is in development for treatment of Non Small Cell Lung cancer and other cancers. Furthermore, such agents are also in clinical development for viral infections, for example the anti PD-1 Ab, MDX-1106, which is being tested for treatment of hepatitis C, and the anti-CTLA-4 Ab CP-675,206 (tremelimumab) which is in a clinical trial in hepatitis C virus-infected patients with hepatocellular carcinoma.

Accumulations of inducible regulatory T cells (iTregs) are commonly seen in many tumors, and form the major subset of immune suppressor cells in the tumor tissue. Tregs create an immunosuppressive environment and regulate anti-tumor immunity, and thus represent a major tumor resistance mechanism from immune surveillance. iTregs are therefore viewed as important cellular targets for cancer therapy.

In addition to their function in dampening effector T cell responses, multiple immune-checkpoint receptors, such as CTLA4 and PD-1, and others like TIM3 and LAG3, are expressed at high levels on the surface of iTregs and directly promote Treg cell-mediated suppression of effector immune responses. Many of the immune-checkpoint antibodies in clinical testing most likely block the immunosuppressive activity of iTregs as a mechanism of enhancing anti-tumor immunity. Indeed, two important factors in the mode of action of CTLA4 blockade by ipilimumab are the enhancement of effector T cell activity, and inhibition of Treg immunosuppressive activity.

Several strategies, used alone or in combination with conventional treatments or immunotherapies, are in development in order to disarm iTregs and restore antitumor functions of effector T cells.

B cells play a critical role in recognition of foreign antigens and they produce the antibodies necessary to provide protection against various type of infectious agents. T cell help to B cells is a pivotal process of adaptive immune responses. Follicular helper T (Tfh) cells are a subset of CD4+T cells specialized in B cell help (reviewed by Crotty, Annu. Rev. Immunol. 29: 621-663, 2011). Tfh cells express the B cell homing chemokine receptor, CXCR5, which drives Tfh cell migration into B cell follicles within lymph nodes in a CXCL13-dependent manner. The requirement of Tfh cells for B cell help and T cell-dependent antibody responses, indicates that this cell type is of great importance for protective immunity against various types of infectious agents, as well as for rational vaccine design.

### BRIEF SUMMARY OF THE INVENTION

Despite recent progress in the understanding of cancer biology and cancer treatment, the success rate for cancer therapy remains low. Therefore, there is an unmet need for new therapies which can successfully treat cancer, such as for example, specific blocking antibodies, which have a therapeutic application in stimulating the immune system against tumors.

By “blocking antibody” it is meant any antibody that binds to a particular protein or epitope on a protein, and then optionally blocks interactions of that protein with one or more other binding partners.

According to at least some embodiments there is provided an immune molecule, comprising an antigen-binding region having an amino acid sequence selected from the group consisting of SEQ ID NOs 229, 235, 242-243, 245-249, 258, 274-278, 264-272, 251-256, 280, 287, 284, 285, 290-292, 287, 288, 259, 295, 297-302, 306-307, wherein the antigen-binding regions are adapted to specifically bind to a protein having the amino acid sequence of SEQ ID NO:10.

Optionally the immune molecule comprises at least one of SEQ ID NOs 227, 228 or 229 and at least one of SEQ ID NOs 233, 234 or 235; or alternatively at least one of SEQ ID NOs 245, 246, 247, 248, or 249, and at least one of SEQ ID NOs: 239, 240, 241, 252, 256, 287, 288, 242 or 243; or alternatively at least one of SEQ ID NOs 303, 304, 261, 306, or 262, and at least one of SEQ ID NOs: 251, 252, 253, 254, 255, 256, 257, 258 or 259; or alternatively at least one of SEQ ID NOs 274, 275, 276, 277, or 278, and at least one of SEQ ID NOs: 264, 265, 266, 267, 268, 269, 270, 271 or 272; or alternatively at least one of SEQ ID NOs 274, 275, 276, 277, or 282, and at least one of SEQ ID NOs: 264, 265, 266, 267, 268, 269, 280, 271, or 272; or alternatively at least one of SEQ ID NOs 303, 304, 305, 306, or 307, and at least one of SEQ ID NOs: 239, 240, 241, 252, 256, 287, 288, 284, or 285; or alternatively at least one of SEQ ID NOs 290, 291, 305, 306, or 292, and at least one of SEQ ID NOs: 251, 252, 253, 254, 256, 287, 288, 258 or 259; or alternatively at least one of SEQ ID NOs 303, 304, 305, 306, or 307, and at least one of SEQ ID NOs: 294, 295, 296, 297, 298, 299, 300, 301 or 302. Optionally the immune molecule further comprises at least one of SEQ ID NOs: 227-229, 239-243, 251-299, 264-272, 280, 284-285, 287-288, 293-301 or 302 and at least one of SEQ ID NOs 233-235, 245-249, 261-262, 274-278, 282, 290-292, 303-306 or 307.

Optionally the immune molecule is in the form of an antibody fragment.

Optionally the immune molecule comprises at least two antigen-binding regions having amino acid sequences selected from the group consisting of SEQ ID NOs 239-243, 245-249, 251-259, 261-262, 264-272, 274-278, 280-281, 282, 284-285, 287-288, 290-292, 294-307.

Optionally the immune molecule comprises at least one antigen-binding region having an amino acid sequence selected from the group consisting of SEQ ID NOs 227, 228, 229, 245, 246, 247, 248, 249, 261, 262, 274, 275, 276, 277, 278, 282, 303, 304, 305, 306, 307, 290, 291, and 292.

Optionally the immune molecule comprises at least one antigen-binding region having an amino acid sequence selected from the group consisting of SEQ ID NOs 233, 234, 235, 239, 240, 241, 287, 288, 242, 243, 251, 252, 253, 254, 255, 256, 257, 258, 259, 264, 265, 266, 267, 268, 269, 270, 271, 272, 280, 294, 295, 296, 297, 298, 299, 300, 301, 302, 284, 285, 287, 288, 258, and 259.

Optionally the immune molecule comprises a protein having the amino acid sequence of any of SEQ ID NOs: 220, 244, 260, 273, 281, 289, 308, or a sequence 95% homologous thereto.

Optionally the immune molecule comprises a protein having the amino acid sequence of any of SEQ ID NOs: 218, 238, 250, 263, 279, 283, 286, 293, or a sequence 95% homologous thereto.

Optionally the immune molecule comprises a protein having the amino acid sequence of SEQ ID NO: 220 and of SEQ ID NO:218 or a sequence 95% homologous thereto;

or alternatively comprising a protein having the amino acid sequence of SEQ ID NO: 244 and of SEQ ID NO:238 or a sequence 95% homologous thereto;

or alternatively comprising a protein having the amino acid sequence of SEQ ID NO: 260 and of SEQ ID NO:259 or a sequence 95% homologous thereto;

or alternatively comprising a protein having the amino acid sequence of SEQ ID NO: 273 and of SEQ ID NO:263 or a sequence 95% homologous thereto;

or alternatively comprising a protein having the amino acid sequence of SEQ ID NO: 281 and of SEQ ID NO:279 or a sequence 95% homologous thereto;

or alternatively comprising a protein having the amino acid sequence of SEQ ID NO: 308 and of SEQ ID NO:283 or a sequence 95% homologous thereto;

or alternatively comprising a protein having the amino acid sequence of SEQ ID NO: 289 and of SEQ ID NO:286 or a sequence 95% homologous thereto;

or alternatively comprising a protein having the amino acid sequence of SEQ ID NO: 308 and of SEQ ID NO:293 or a sequence 95% homologous thereto;

or alternatively comprising a protein having the following amino acid sequences: one of SEQ ID NOs 245 or 246; one of SEQ ID NOs 247 or 248; SEQ ID NO: 249; one of SEQ ID NOs 239, 240, 241, or 252; one of SEQ ID NOs 256, 287, 288; and one of SEQ ID NOs 242 or 243;

or alternatively comprising a protein having the following amino acid sequences: one of SEQ ID NOs 303 or 304; one of SEQ ID NOs 261 or 306; SEQ ID NO:262; one of SEQ ID NOs 251, 252, 253, or 254; one of SEQ ID NOs 255, 256 or 257; and one of SEQ ID NOs 258 or 259;

or alternatively comprising a protein having the following amino acid sequences: one of SEQ ID NOs 274 or 275; one of SEQ ID NOs:276 or 277; SEQ ID NO:278; one of SEQ ID NOs:264, 265, 266, or 267; one of SEQ ID NOs:268, 269, or 270; and one of SEQ ID NOs:271 or 272;

or alternatively comprising a protein having the following amino acid sequences: one of SEQ ID NOs 274 or 275; one of SEQ ID NOs:276 or 277; SEQ ID NO:282 one of SEQ ID NOs:264, 265, 266, 267; one of SEQ ID NOs:268, 269, or 280; and one of SEQ ID NOs:271 or 272;

or alternatively comprising a protein having the following amino acid sequences: one of SEQ ID NOs: 303 or 304; one of SEQ ID NOs: 305 or 306; SEQ ID NO:307; one of SEQ ID NOs:239, 240, 241 or 252; one of SEQ ID NOs:256, 287 or 288; and one of SEQ ID NOs:284 or 285;

or alternatively comprising a protein having the following amino acid sequences: one of SEQ ID NOs: 290 or 291; one of SEQ ID NOs:305 or 306; SEQ ID NO:292; one of SEQ ID NOs:251, 252, 253, or 254; one of SEQ ID NOs:256, 287, or 288; and one of SEQ ID NOs:258 or 259;

or alternatively comprising a protein having the following amino acid sequences: one of SEQ ID NOs: 303 or 304; one of SEQ ID NOs:305 or 306; SEQ ID NO:307; one of SEQ ID NOs:294, 295, 296, or 297; one of SEQ ID NOs:298, 299 or 300; and one of SEQ ID NOs:301 or 302.

According to at least some embodiments there is provided a polynucleotide encoding for the immune molecule as described herein.

Optionally the polynucleotide comprises a nucleic acid sequence selected from the group consisting of SEQ NOs. 224, 225, 226, 230, 231, 232, 217 and 219, and degenerate variants thereof.

According to at least some embodiments there is provided a vector comprising the polynucleotide as described herein.

According to at least some embodiments there is provided a recombinant cell comprising the vector described herein, capable of expressing the immune molecule as described herein.

According to at least some embodiments there is provided a method for producing the immune molecule as described herein, comprising introducing the vector into a cell to form a recombinant cell; and producing the immune molecule by the recombinant cell.

According to at least some embodiments there is provided an antibody or an antigen binding fragment thereof, said antibody having an antigen-binding region that binds specifically to amino acids 30-110 of SEQ ID NO 10 and that does not specifically bind to any other portion of SEQ ID NO 10, wherein said other portion of SEQ ID NO:10 comprises amino acids 1-29 or amino acids 111 to 234 of SEQ ID NO: 10.

Optionally said antibody has an antigen-binding region that binds specifically to SEQ ID NO 215 or to SEQ ID NO 216 and that does not specifically bind to any other portion of SEQ ID NO 10, wherein said other portion of SEQ ID NO:10 comprises amino acids 1-80 or amino acids 99 to 234 of SEQ ID NO: 10 for SEQ ID NO 215, or wherein said other portion of SEQ ID NO:10 comprises amino acids 1-117 or amino acids 136 to 234 of SEQ ID NO: 10 for SEQ ID NO 216.

Optionally the antibody is a fully human antibody, chimeric antibody, humanized or primatized antibody.

Optionally the antibody is selected from the group consisting of Fab, Fab′, F(ab′)2, F(ab′), F(ab), Fv or scFv fragment and minimal recognition unit.

Optionally the antibody is coupled to a therapeutic agent selected from a drug, a radionuclide, a fluorophore, an enzyme, a toxin, a therapeutic agent, or a chemotherapeutic agent; and wherein the detectable marker is a radioisotope, a metal chelator, an enzyme, a fluorescent compound, a bioluminescent compound or a chemiluminescent compound.

According to at least some embodiments there is provided a pharmaceutical composition comprising the immune molecule, antibody or the antigen binding fragment as described herein.

According to at least some embodiments there is provided use of the immune molecule, antibody, antibody binding fragment or pharmaceutical composition as described herein for treating a disease selected from the group consisting of cancer, immune condition or an infectious disease, in a subject in need thereof.

Optionally said antibody or fragment modulates immune cell activity, increases T cell activation, alleviates T-cell suppression, decreases immunosuppressive cytokine secretion, increases pro-inflammatory cytokine secretion, increases IL-2 secretion; increases interferon-gamma production by T-cells, promotes cancer epitope spreading, increases T cell response in a mammal, decreases or eliminates M2 macrophages, reduces M2 macrophage pro-tumorigenic activity, enhances antigen-specific memory responses, enhances apoptosis of cancer cells, enhances cytotoxic or cytostatic effect on cancer cells, enhances direct killing of cancer cells, induces complement dependent cytotoxicity and/or antibody dependent cell-mediated cytotoxicity.

Optionally said antibody or fragment increases immune response against the cancer.

Optionally the treatment is combined with another therapeutic agent or therapy useful for treating cancer.

Optionally the therapy comprises one or more of radiotherapy, cryotherapy, antibody therapy, chemotherapy, photodynamic therapy, surgery, hormonal deprivation or combination therapy with conventional drugs.

Optionally the therapeutic agent is selected from the group consisting of cytotoxic drugs, tumor vaccines, antibodies, peptides, pepti-bodies, small molecules, chemotherapeutic agents, cytotoxic and cytostatic agents, immunological modifiers, interferons, interleukins, immunostimulatory growth hormones, cytokines, vitamins, minerals, aromatase inhibitors, RNAi, Histone Deacetylase Inhibitors, and proteasome inhibitors.

Optionally the immune molecule, antibody, antibody binding fragment, composition as described herein is administered to a subject simultaneously or sequentially in combination with one or more potentiating agents to obtain a therapeutic effect, wherein said one or more potentiating agents is selected from the group consisting of radiotherapy, conventional/classical anti-cancer therapy potentiating anti-tumor immune responses, Targeted therapy potentiating anti-tumor immune responses, Therapeutic agents targeting Tregs and/or MDSCs, Immunostimulatory antibodies, Cytokine therapy, Therapeutic cancer vaccines, Adoptive cell transfer.

Optionally the conventional/classical anti-cancer agent is selected from platinum based compounds, antibiotics with anti-cancer activity, Anthracyclines, Anthracenediones, alkylating agents, antimetabolites, Antimitotic agents, Taxanes, Taxoids, microtubule inhibitors, Vinca alkaloids, Folate antagonists, Topoisomerase inhibitors, Antiestrogens, Antiandrogens, Aromatase inhibitors, GnRh analogs, inhibitors of 5α-reductase, biphosphonates.

Optionally the Targeted therapy agent is selected from the group consisting of histone deacetylase (HDAC) inhibitors, proteasome inhibitors, mTOR pathway inhibitors, JAK2 inhibitors, tyrosine kinase inhibitors (TKIs), PI3K inhibitors, Protein kinase inhibitors, Inhibitors of serine/threonine kinases, inhibitors of intracellular signaling, inhibitors of Ras/Raf signaling, MEK inhibitors, AKT inhibitors, inhibitors of survival signaling proteins, cyclin dependent kinase inhibitors, therapeutic monoclonal antibodies, TRAIL pathway agonists, anti-angiogenic agents, metalloproteinase inhibitors, cathepsin inhibitors, inhibitors of urokinase plasminogen activator receptor function, immunoconjugates, antibody drug conjugates, antibody fragments, bispecfic antibodies, bispecific T cell engagers (BiTEs).

Optionally the antibody therapy is selected from cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept.

Optionally the Therapeutic agent targeting immunosuppressive cells Tregs and/or MDSCs is selected from antimitotic drugs, cyclophosphamide, gemcitabine, mitoxantrone, fludarabine, thalidomide, thalidomide derivatives, COX-2 inhibitors, depleting or killing antibodies that directly target Tregs through recognition of Treg cell surface receptors, anti-CD25 daclizumab, basiliximab, ligand-directed toxins, denileukin diftitox (Ontak)—a fusion protein of human IL-2 and diphtheria toxin, or LMB-2—a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin.

Optionally the Immunostimulatory antibody is selected from antagonistic antibodies targeting one or more of CTLA4, PD-1, PDL-1, LAG-3, TIM-3, BTLA, B7-H4, B7-H3, VISTA, and/or Agonistic antibodies targeting one or more of CD40, CD137, OX40, GITR, CD27, CD28 or ICOS.

Optionally the Therapeutic cancer vaccine is selected from exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cells, dendritic cell-based vaccines, whole tumor cell vaccines, gene modified tumor cells expressing GM-CSF, ICOS and/or Flt3-ligand, oncolytic virus vaccines.

Optionally the Cytokine therapy is selected from one or more of the following cytokines such as IL-2, IL-7, IL-12, IL-15, IL-17, IL-18 and IL-21, IL23, IL-27, GM-CSF, IFNα (interferon alpha), IFNα-2b, IFNβ, IFNγ, and their different strategies for delivery.

Optionally the adoptive cell transfer therapy is carried out following ex vivo treatment selected from expansion of the patient autologous naturally occurring tumor specific T cells or genetic modification of T cells to confer specificity for tumor antigens.

According to at least some embodiments there is provided a use of an antibody or immune molecule or a pharmaceutical composition as described herein to perform one or more of the following in a subject to treat a disease: (a) upregulating cytokines, (b) increases T-cell proliferation and/or expansion, (c) increases interferon-gamma production by T-cells (d) increases IL-2 secretion (e) stimulates antibody responses; (f) inhibits cancer cell growth, (g) promoting antigenic specific T cell immunity, (g) promoting CD4+ and/or CD8+T cell activation, (i) alleviating T-cell suppression, (j) alleviating apoptosis or lysis of cancer cells, (k) cytotoxic or cytostatic effect on cancer cells.

According to at least some embodiments there is provided a diagnostic method for diagnosing a disease in a subject, wherein the disease is selected from the group consisting of cancer or an autoimmune disease, wherein the diagnostic method is performed ex vivo, comprising contacting a tissue sample from the subject with the immune molecule or antibody as described herein ex vivo and detecting specific binding thereto.

According to at least some embodiments there is provided a diagnostic method for diagnosing a disease in a subject, wherein the disease is selected from the group consisting of cancer, autoimmune disease, or an infectious disease wherein the diagnostic method is performed in vivo, comprising administering the immune molecule or antibody as described herein to the subject and detecting specific binding of the immune molecule or antibody as described herein to a tissue of the subject.

Optionally the diagnostic method is performed before administering the immune molecule or antibody or pharmaceutical composition to the subject.

Optionally the use or method further comprises determining an LSR level in a tissue of the subject before administering the immune molecule or antibody or pharmaceutical composition to the subject.

Optionally said administering the immune molecule or antibody or pharmaceutical composition to the subject only if said LSR level is sufficient.

Optionally the use or method further comprises determining said LSR level according to expression level of said LSR.

Optionally said determining said expression level comprises applying an IHC (immunohistochemistry) assay or a gene expression assay to a tissue of the subject.

Optionally said applying said IHC assay comprises determining if a level of expression is at least 1 on a scale of 0 to 3.

Optionally said tissue comprises cancer cells or immune infiltrate.

Optionally said determining said LSR level in said tissue comprises contacting the tissue with the antibody or immune molecule as described herein and detecting specific binding thereto.

According to at least some embodiments there is provided an assay for diagnosing a disease in a tissue sample taken from a subject, comprising the immune molecule or antibody as described herein and at least one reagent for diagnosing a disease selected from the group consisting of cancer, autoimmune disease, or infectious disease.

According to at least some embodiments there is provided use as described herein for screening for a disease, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations.

Optionally said cancer, said immune cells infiltrating the tumor or both express LSR at a sufficient level and wherein said cancer is selected from the group consisting of breast cancer, cervical cancer, ovary cancer, endometrial cancer, melanoma, bladder cancer, lung cancer, pancreatic cancer, colon cancer, prostate cancer, leukemia, acute lymphocytic leukemia, chronic lymphocytic leukemia, B-cell lymphoma, Burkitt's lymphoma, multiple myeloma, Non-Hodgkin's lymphoma, myeloid leukemia, acute myelogenous leukemia (AML), chronic myelogenous leukemia, thyroid cancer, thyroid follicular cancer, myelodysplastic syndrome (MDS), fibrosarcomas and rhabdomyosarcomas, melanoma, uveal melanoma, teratocarcinoma, neuroblastoma, glioma, glioblastoma, benign tumor of the skin, keratoacanthomas, renal cancer, anaplastic large-cell lymphoma, esophageal squamous cells carcinoma, hepatocellular carcinoma, follicular dendritic cell carcinoma, intestinal cancer, muscle-invasive cancer, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, prostatic hypertrophy, myelodysplasia, Waldenstrom's macroglobinaemia, nasopharyngeal, neuroendocrine cancer, myelodysplastic syndrome, mesothelioma, angiosarcoma, Kaposi's sarcoma, carcinoid, oesophagogastric, fallopian tube cancer, peritoneal cancer, papillary serous mullerian cancer, malignant ascites, gastrointestinal stromal tumor (GIST), Li-Fraumeni syndrome and Von Hippel-Lindau syndrome (VHL); with the proviso that if the cancer is ovarian cancer, it is not Granulosa cell tumor of the ovary and with the proviso that if the cancer is brain cancer, it is not Astrocytoma grade 2.

Optionally said cancer is selected from the group consisting of ductal-adenocarcinoma, infiltrating ductal carcinoma, Lobular carcinoma of breast, mucinous adenocarcinoma of the breast, Intra duct and invasive ductal carcinoma, Moderate to Poorly Differentiated Adenocarcinoma of the cecum, Well to Poorly Differentiated Adenocarcinoma of the colon, Grade 2 Tubular adenocarcinoma of the ascending colon, colon adenocarcinoma Duke's stage C1, invasive adenocarcinoma of the colon, Adenocarcinoma of the rectum, Grade 3 Adenocarcinoma of the rectum, Moderately Differentiated Adenocarcinoma of the rectum, Moderately Differentiated Mucinous adenocarcinoma of the rectum, Well to Poorly differentiated Non-small cell carcinoma, Moderately to poorly differentiated squamous carcinoma of the lung, Moderately well differentiated keratinising squamous cell carcinoma of the lung, large cell adenocarcinoma of the lung, prostate Adenocarcinoma Gleason Grade 7 to 9, prostate Infiltrating adenocarcinoma, Moderately differentiated adenocarcinoma of the prostate, serous papillary cystic carcinoma of the ovary, Serous cystadenocarcinoma of the ovary, grade 4 Astrocytoma, Glioblastoma multiforme, Clear cell renal cell carcinoma, Hepatocellular carcinoma, and Low Grade hepatocellular carcinoma; with the proviso that if the cancer is ovarian cancer, it is not Granulosa cell tumor of the ovary and with the proviso that if the cancer is brain cancer, it is not Astrocytoma grade 2.

Optionally said breast cancer is selected from the group consisting of ductal-adenocarcinoma, infiltrating ductal carcinoma, lobular carcinoma, mucinous adenocarcinoma, intra duct and invasive ductal carcinoma.

Optionally said breast cancer is Scirrhous adenocarcinoma.

Optionally said colon cancer is selected from the group consisting of Moderate to Poorly Differentiated Adenocarcinoma of the cecum, Well, Moderate and Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, and Moderately Differentiated Mucinous adenocarcinoma of the rectum.

Optionally said lung cancer is selected from the group consisting of Well to Poorly differentiated Non-small cell carcinoma, Squamous Cell Carcinoma, preferably Moderately Differentiated Squamous Cell Carcinoma, Moderately to poorly differentiated squamous carcinoma, Moderately well differentiated keratinising squamous cell carcinoma, large cell adenocarcinoma and Small cell lung cancer.

Optionally said prostate cancer is selected from the group consisting of Adenocarcinoma Gleason Grade 5 to 9, Infiltrating adenocarcinoma, High grade prostatic intraepithelial neoplasia, and undifferentiated carcinoma.

Optionally said stomach cancer is moderately differentiated gastric adenocarcinoma.

Optionally said ovarian cancer is selected from the group consisting of serous papillary cystic carcinoma, Serous cystadenocarcinoma and Invasive serous papillary carcinoma.

Optionally said brain cancer is selected from the group consisting of Glioblastoma multiforme and Astrocytoma other than Astrocytoma grade 2.

Optionally said astrocytoma is grade 4 Astrocytoma.

Optionally said kidney cancer is Clear cell renal cell carcinoma.

Optionally liver cancer is Hepatocellular carcinoma.

Optionally said Hepatocellular carcinoma is Low Grade hepatocellular carcinoma or Fibrolamellar Hepatocellular Carcinoma.

Optionally said hematological cancer is selected from the group consisting of large cell lymphoma, and High and low grade Non-Hodgkin's Lymphoma.

Optionally said disease is immune condition and wherein said immune condition is selected from the group consisting of autoimmune disease, transplant rejection, and graft versus host disease.

Optionally said autoimmune disease is selected from the group consisting of wherein the autoimmune disease is selected from a group consisting of multiple sclerosis, psoriasis; rheumatoid arthritis; psoriatic arthritis, systemic lupus erythematosus (SLE); ulcerative colitis; Crohn's disease; benign lymphocytic angiitis, thrombocytopenic purpura, idiopathic thrombocytopenia, idiopathic autoimmune hemolytic anemia, pure red cell aplasia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, cryoglobulinemic vasculitis, ANCA-associated vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, autoimmune atrophic gastritis, pernicious anaemia, pemphigus, pemphigus vulgarus, cirrhosis, primary biliary cirrhosis, dermatomyositis, polymyositis, fibromyositis, myogelosis, celiac disease, immunoglobulin A nephropathy, Henoch-Schonlein purpura, Evans syndrome, atopic dermatitis, psoriasis, psoriasis arthropathica, Graves' disease, Graves' ophthalmopathy, scleroderma, systemic scleroderma, progressive systemic scleroderma, asthma, allergy, primary biliary cirrhosis, Hashimoto's thyroiditis, primary myxedema, sympathetic ophthalmia, autoimmune uveitis, hepatitis, chronic action hepatitis, collagen diseases, ankylosing spondylitis, periarthritis humeroscapularis, panarteritis nodosa, chondrocalcinosis, Wegener's granulomatosis, microscopic polyangiitis, chronic urticaria, bullous skin disorders, pemphigoid, atopic eczema, Devic's disease, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, myocarditis, vasculitis, gastritis, gout, gouty arthritis, and inflammatory skin disorders, normocomplementemic urticarial vasculitis, pericarditis, myositis, anti-synthetase syndrome, scleritis, macrophage activation syndrome, Bechet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, autoimmune retinopathy, age-related macular degeneration, atherosclerosis, chronic prostatitis and TNF receptor-associated periodic syndrome (TRAPS).

Optionally the treatment is combined with another moiety useful for treating immune related condition.

Optionally said disease is infectious disease and wherein said infectious disease is selected from the disease caused by bacterial infection, viral infection, fungal infection and/or other parasite infection.

Optionally the infectious disease is selected from hepatitis B, hepatitis C, infectious mononucleosis, EBV, cytomegalovirus, AIDS, HIV-1, HIV-2, tuberculosis, malaria and schistosomiasis.

Optionally the treatment is combined with another moiety useful for treating infectious disease.

According to at least some embodiments there is provided use of an antibody or a fragment specifically binding to SEQ ID NO: 10 to treat or diagnose a subject suffering from a disease selected from the group consisting of ductal-adenocarcinoma, infiltrating ductal carcinoma, lobular carcinoma, mucinous adenocarcinoma, intra duct and invasive ductal carcinoma, Scirrhous adenocarcinoma, Moderate to Poorly Differentiated Adenocarcinoma of the cecum, Well, Moderate and Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum, Well to Poorly differentiated Non-small cell carcinoma, Squamous Cell Carcinoma, preferably Moderately Differentiated Squamous Cell Carcinoma, Moderately to poorly differentiated squamous carcinoma, Moderately well differentiated keratinising squamous cell carcinoma, large cell adenocarcinoma, Small cell lung cancer, Adenocarcinoma Gleason Grade 5 to 9, Infiltrating adenocarcinoma, High grade prostatic intraepithelial neoplasia, undifferentiated carcinoma, moderately differentiated gastric adenocarcinoma, serous papillary cystic carcinoma, Serous cystadenocarcinoma, Invasive serous papillary carcinoma, Glioblastoma multiforme, Astrocytoma, Astrocytoma grade 4, Clear cell renal cell carcinoma, Hepatocellular carcinoma, Low Grade hepatocellular carcinoma, Fibrolamellar Hepatocellular Carcinoma, large cell lymphoma, and High and low grade Non-Hodgkin's Lymphoma; with the proviso that if the cancer is ovarian cancer, it is not Granulosa cell tumor of the ovary and with the proviso that if the cancer is brain cancer, it is not Astrocytoma grade 2.

According to at least some embodiments there is provided a pharmaceutical composition comprising an antibody or a fragment specifically binding to SEQ ID NO: 10 to treat a subject suffering from a disease selected from the group consisting of ductal-adenocarcinoma, infiltrating ductal carcinoma, lobular carcinoma, mucinous adenocarcinoma, intra duct and invasive ductal carcinoma, Scirrhous adenocarcinoma, Moderate to Poorly Differentiated Adenocarcinoma of the cecum, Well, Moderate and Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum, Well to Poorly differentiated Non-small cell carcinoma, Squamous Cell Carcinoma, preferably Moderately Differentiated Squamous Cell Carcinoma, Moderately to poorly differentiated squamous carcinoma, Moderately well differentiated keratinising squamous cell carcinoma, large cell adenocarcinoma, Small cell lung cancer, Adenocarcinoma Gleason Grade 5 to 9, Infiltrating adenocarcinoma, High grade prostatic intraepithelial neoplasia, undifferentiated carcinoma, moderately differentiated gastric adenocarcinoma, serous papillary cystic carcinoma, Serous cystadenocarcinoma, Invasive serous papillary carcinoma, Glioblastoma multiforme, Astrocytoma, Astrocytoma grade 4, Clear cell renal cell carcinoma, Hepatocellular carcinoma, Low Grade hepatocellular carcinoma, Fibrolamellar Hepatocellular Carcinoma, large cell lymphoma, and High and low grade Non-Hodgkin's Lymphoma; with the proviso that if the cancer is ovarian cancer, it is not Granulosa cell tumor of the ovary and with the proviso that if the cancer is brain cancer, it is not Astrocytoma grade 2.

Optionally the cancer is selected from the group consisting of ductal-adenocarcinoma, infiltrating ductal carcinoma, Lobular carcinoma of breast, mucinous adenocarcinoma of the breast, Intra duct and invasive ductal carcinoma, Moderate to Poorly Differentiated Adenocarcinoma of the cecum, Well to Poorly Differentiated Adenocarcinoma of the colon, Grade 2 Tubular adenocarcinoma of the ascending colon, colon adenocarcinoma Duke's stage C1, invasive adenocarcinoma, Adenocarcinoma of the rectum, Grade 3 Adenocarcinoma of the rectum, Moderately Differentiated Adenocarcinoma of the rectum, Moderately Differentiated Mucinous adenocarcinoma of the rectum, Well to Poorly differentiated Non-small cell carcinoma, Squamous Cell Carcinoma: Moderately Differentiated, Moderately to poorly differentiated squamous carcinoma, Moderately well differentiated keratinising squamous cell carcinoma, large cell adenocarcinoma, Adenocarcinoma Gleason Grade 7 to 9, Infiltrating adenocarcinoma, Moderately differentiated adenocarcinoma, serous papillary cystic carcinoma, Serous cystadenocarcinoma, grade 4 Astrocytoma, Glioblastoma multiforme, Clear cell renal cell carcinoma, Hepatocellular carcinoma, and Low Grade hepatocellular carcinoma; with the proviso that if the cancer is ovarian cancer, it is not Granulosa cell tumor of the ovary and with the proviso that if the cancer is brain cancer, it is not Astrocytoma grade 2.

Optionally the treatment is combined with another therapeutic agent or therapy useful for treating cancer.

Optionally the therapy comprises one or more of radiotherapy, cryotherapy, antibody therapy, chemotherapy, photodynamic therapy, surgery, hormonal deprivation or combination therapy with conventional drugs.

Optionally the therapeutic agent is selected from the group consisting of cytotoxic drugs, tumor vaccines, antibodies, peptides, pepti-bodies, small molecules, chemotherapeutic agents, cytotoxic and cytostatic agents, immunological modifiers, interferons, interleukins, immunostimulatory growth hormones, cytokines, vitamins, minerals, aromatase inhibitors, RNAi, Histone Deacetylase Inhibitors, and proteasome inhibitors.

Optionally the antibody or composition is administered to a subject simultaneously or sequentially in combination with one or more potentiating agents to obtain a therapeutic effect, wherein said one or more potentiating agents is selected from the group consisting of radiotherapy, conventional/classical anti-cancer therapy potentiating anti-tumor immune responses, Targeted therapy potentiating anti-tumor immune responses, Therapeutic agents targeting Tregs and/or MDSCs, Immunostimulatory antibodies, Cytokine therapy, Adoptive cell transfer.

Optionally the conventional/classical anti-cancer agent is selected from platinum based compounds, antibiotics with anti-cancer activity, Anthracyclines, Anthracenediones, alkylating agents, antimetabolites, Antimitotic agents, Taxanes, Taxoids, microtubule inhibitors, Vinca alkaloids, Folate antagonists, Topoisomerase inhibitors, Antiestrogens, Antiandrogens, Aromatase inhibitors, GnRh analogs, inhibitors of 5α-reductase, biphosphonates.

Optionally the Targeted therapy agent is selected from the group consisting of histone deacetylase (HDAC) inhibitors, proteasome inhibitors, mTOR pathway inhibitors, JAK2 inhibitors, tyrosine kinase inhibitors (TKIs), PI3K inhibitors, Protein kinase inhibitors, Inhibitors of serine/threonine kinases, inhibitors of intracellular signaling, inhibitors of Ras/Raf signaling, MEK inhibitors, AKT inhibitors, inhibitors of survival signaling proteins, cyclin dependent kinase inhibitors, therapeutic monoclonal antibodies, TRAIL pathway agonists, anti-angiogenic agents, metalloproteinase inhibitors, cathepsin inhibitors, inhibitors of urokinase plasminogen activator receptor function, immunoconjugates, antibody drug conjugates, antibody fragments, bispecfic antibodies, bispecific T cell engagers (BiTEs).

Optionally the antibody is selected from cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept.

Optionally the Therapeutic agent targeting immunosuppressive cells Tregs and/or MDSCs is selected from antimitotic drugs, cyclophosphamide, gemcitabine, mitoxantrone, fludarabine, thalidomide, thalidomide derivatives, COX-2 inhibitors, depleting or killing antibodies that directly target Tregs through recognition of Treg cell surface receptors, anti-CD25 daclizumab, basiliximab, ligand-directed toxins, denileukin diftitox (Ontak)—a fusion protein of human IL-2 and diphtheria toxin, or LMB-2—a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin.

Optionally the Immunostimulatory antibody is selected from antagonistic antibodies targeting one or more of CTLA4, PD-1, PDL-1, LAG-3, TIM-3, BTLA, B7-H4, B7-H3, VISTA, and/or Agonistic antibodies targeting one or more of CD40, CD137, OX40, GITR, CD27, CD28 or ICOS.

Optionally the Therapeutic cancer vaccine is selected from exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cell-based vaccines, whole tumor cell vaccines, gene modified tumor cells expressing GM-CSF, ICOS and/or Flt3-ligand, oncolytic virus vaccines.

Optionally the Cytokine therapy is selected from one or more of the following cytokines such as IL-2, IL-7, IL-12, IL-15, IL-17, IL-18 and IL-21, IL23, IL-27, GM-CSF, IFNα (interferon alpha), IFNα-2b, IFNβ, IFNγ, and their different strategies for delivery.

Optionally the adoptive cell transfer therapy is carried out following ex vivo treatment selected from expansion of the patient autologous naturally occurring tumor specific T cells or genetic modification of T cells to confer specificity for tumor antigens.

According to at least some embodiments there is provided a diagnostic method for determining whether to perform the use or to administer the composition as described herein, comprising performing the diagnostic method as described herein.

Optionally the cancer is non-metastatic.

Optionally the cancer is invasive.

Optionally the cancer is metastatic.

### BRIEF DESCRIPTION OF THE FIGURES

FIG. 1 demonstrates Western Blot analysis of the expression of LSR_P5a_Flag_m protein (SEQ ID: 144) in stably-transfected recombinant HEK293T cells, as detected with anti Flag (Sigma cat#A8592) (FIG. 1A) and anti LSR antibodies as follow: Abnova, cat#H00051599-B01P (FIG. 1B) Abcam, cat ab59646 (FIG. 1C) and Sigma cat# HPA007270 (FIG. 1D). Lane 1: HEK293T_pIRESpuro3; lane 2: HEK293T_pIRESpuro3_LSR_P5a_Flag.

FIG. 2 presents detection of human LSR_WT with 3 different commercial Abs against LSR: 2A refers to SIGMA Ab, whereas 2B to Abcam Ab and 2C to Abnova Ab (as detailed in Table 1). Lane #1 represents HEK293T_pIRESpuro3 empty vector transfected cells which were used as a negative control, whereas lane #2 represents HEK293T_pIRESpuro3_human WT LSR_Flag transfected cells.

FIG. 3 presents detection of the human LSR_skip4 protein using Abcam Ab. Lane #1 represents HEK293T_pIRESpuro3 empty vector transfected cells which were used as a negative control, whereas lane #2 represents HEK293T_pIRESpuro3_human LSR skip4_Flag transfected cells.

FIG. 4 presents detection of the cyno LSR_WT in HEK293T_pIRESpuro3_cyno WT LSR_Flag transfected cells (lane 2) and LSR_skip4 in HEK293T_pIRESpuro3_cyno skip4 LSR_Flag transfected cells (lane 3) proteins, both compared to the empty vector HEK293T_pIRESpuro3 transfected cells lysate (lane 1). The detection was done using Anti LSR antibody (Abcam 1:4000).

FIG. 5 presents detection by anti-flag Abs of mouse LSR_WT protein in CHO-K1 cells transfected with pcDNA3.1_mouse WT LSR_Flag (lane 2) and in HEK293 cells transfected with pcDNA3.1_mouse WT LSR_Flag (lane 4) compared to the corresponding empty vector transfected cells lysate (lanes 1 and 3, respectively).

FIG. 6 demonstrates the endogenous expression of LSR in various cell lines. A band at ˜72 kDa corresponding to LSR was detected with anti LSR antibody in cell extracts of (1) Caov3, (2) ES2, (3) OV-90, (4) OVCAR₃, (5) SK-OV3, (6) TOV112D, (7) CaCo2, (8) HeLa, (9) Hep G2, (10) MCF-7, (11) SkBR₃ and (12) 293T_LSR_P5a_Flag (FIG. 3A). Anti GAPDH (Abcam cat# ab9484) served as a loading control (FIG. 3B).

FIG. 7 shows a specific knock down of the ectopic expression of the Human WT LSR-flag protein demonstraeted by dramatic decrease in the signal intensity of the ˜70 kDa band, corresponding to the Human WT LSR-flag protein, following transfection of the HEK293T cells stably transfected with pIRESpuro3_human WT LSR_Flag, with the LSR specific siRNA (Thermo Scientific Cat#M-009672-00-0005) (lane 2), as compared to the scrambled siRNA (Thermo Scientific Cat#D-001810-01-05) (lane 1).

FIG. 8 demonstrates the subcellular localization of LSR_P5a_Flag_m. LSR_P5a_Flag_m (SEQ ID NO: 144) is localized mainly to the cell cytoplasm, but can also be detected on the cell surface as detected with anti Flag (Sigma cat# A9594) (FIG. 8A) and anti LSR antibodies as follows: Abcam, cat ab59646 (FIG. 8B) Abnova, cat#H00051599-B01P (FIG. 8C) and Sigma cat# HPA007270 (FIG. 8D).

FIG. 9 presents confocal microscopy image demonstrating the subcellular localization of human LSR_WT protein (SEQ ID NOs:11) in human LSR WT HEK293T transfected cells.

FIG. 10 presents confocal microscopy image demonstrating the subcellular localization of cyno LSR_WT protein (SEQ ID NO: 211) in cyno LSR_WT HEK293T transfected cells.

FIG. 11 presents confocal microscopy image demonstrating the subcellular localization of mouse LSR_WT protein (SEQ ID NO: 31) in mouse LSR_WT transfected HEK293 cells.

FIG. 12 presents confocal microscopy image demonstrating the subcellular localization of cyno LSR skip4 protein (SEQ ID NO:212) in HEK293T transfected cells.

FIG. 13 presents confocal microscopy image demonstrating the subcellular localization of mouse LSR_WT protein (SEQ ID NOs: 31) in mouse LSR_WT transfected CHO-K1 cells.

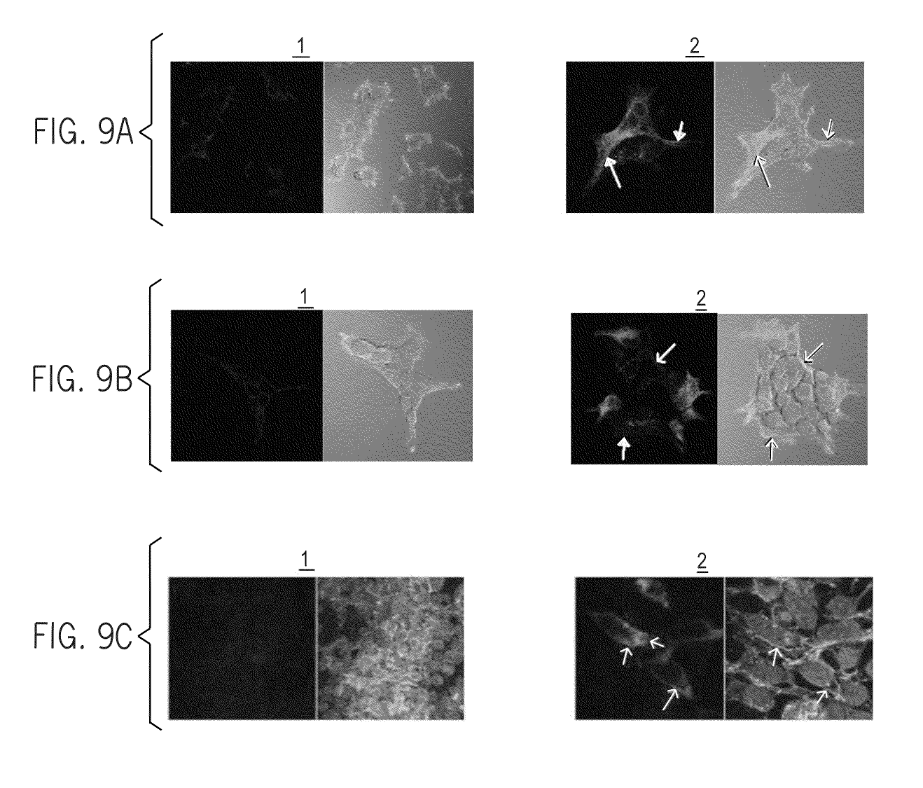

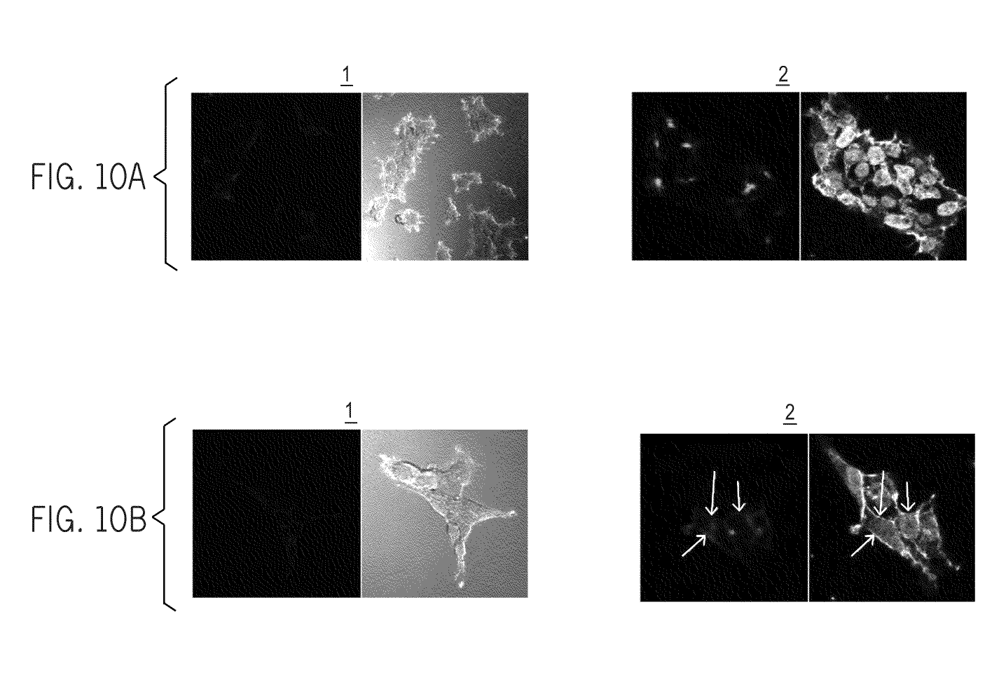

FIGS. 9A, 10A, 11A, 12A and 13A present LSR protein localization with anti Flag antibody (Sigma cat# A9594). LSR protein localization with anti LSR antibodies is presented in FIG. 9C (Abcam, cat ab59646), and FIGS. 9B, 10B, 11B, 12B and 13B (Sigma cat# HPA007270). Figures A-1, B-1 and C-1 in FIGS. 9-13 represent the results of empty vector transfected cells which were used as a negative control. Arrows indicate membrane staining. Figures A-2, B-2 and C-2 in FIGS. 9-13 represent the results of the subcellular localization of human, cyno and mouse LSR_WT protein in recombinant cells expressing LSR proteins.

FIG. 14 presents the results of binding of different concentrations (1 ug/ml, 2.5 ug/ml, 5 ug/ml and 10 ug/ml) of 8C8 mAb to cells stably expressing human WT LSR protein (SEQ ID NO:11), cyno WT LSR protein (SEQ ID NO:211), mouse WT LSR protein (SEQ ID NO:31), human Skip4 LSR variant (SEQ ID NO:13) and cyno Skip4 LSR variant (SEQ ID NO: 212).

FIG. 15 demonstrates endogenous expression of LSR proteins (corresponding to ˜70 kDa band) in various cell lines, as detailed below. Lanes 1 and 14 represent the positive control cells, corresponding to HEK293T cells stably transfected with pIRESpuro3_human WT LSR_Flag. Lane 2 corresponds to SKBR3; lane 3 corresponds to MCF7, lane 4 corresponds to HepG2, lane 5 corresponds to Hela, lane 6 corresponds to CaCO2, lane 7 corresponds to TOV112D, lane 8 corresponds to SKOV3, lane 9 corresponds to OvCar3, lane 10 corresponds to OV-90, lane 11 corresponds to ES2, lane 12 corresponds to CaOV3, lane 13 corresponds to HEK293T_pIRESpuro3 negative control, lane 15 corresponds to HepG2, lane 16 corresponds to HepG2C3A, lane 17 corresponds to Hep3B, lane 18 corresponds to SNU182, lane 19 corresponds to SkHep1, lane 20 corresponds to PLC/PRF5, lane 21 corresponds to Chang Liver, lane 22 corresponds to HT29 cell line.

FIG. 16 presents a westen blot results demonstrating a specific knockdown of endogenous LSR (SEQ ID NO:11) protein expression.

FIG. 17 demonstrates FACS analysis results of HT29 (FIG. 17A) and HepG2/C3A (FIG. 17B) cells following transient transfection with the LSR-specific siRNA (Thermo Scientific Cat#M-009672-00-0005).

FIG. 18: shows the DNA sequence and the amino acid sequence of the heavy (FIG. 18A) and light (FIG. 18B) chains of the 8C8 antibody. The leader sequence is shown in Italic font; the sequences of CDR1, CDR2, CDR3 are shown in bold. The constant regions FRE FR2, FR3 and FR4 are shown in a regular font.

FIG. 19 presents sections of LSR positive cells following pH9.0 antigen retrival (X60). Panel A shows the slides incubated with LSR antibodies applied at 2 μg/ml. Panel B shows the adjacent ‘no primary’ incubated sections. Positive LSR immunoreactivity is seen in Panel A, demonstrating valid antibody working conditions. The adjacent no primary section (Panel B) does not show apparent immunoreactivity.

FIG. 20 demonstrates binding assay results of mouse LSR ECD fused to mouse IgG2a Fc (SEQ ID NO:221) (at 2 μg/5 μl/well) to anti CD3/CD28 or Con A activated murine T cells. SEQ ID NO: 223 corresponds to protein used as positive control.

FIG. 21 shows binding of 2 ug, 3 ug, 4 ug or 5 ug of biotin labeled mouse LSR-ECD fused to mouse IgG2a Fc (SEQ ID NO:221) or of a 5 μg biotin-labeled control mIgG2a to KARPAS-299 cells. FIG. 23A presents histograms showing Median Fluorescence Intensity (MFI). FIG. 23B presents the dose-dependent binding of LSR-ECD fused to mouse IgG2a Fc (SEQ ID NO:221) to Karpas-299 cells. The Y axis represents MFI ratio of mouse LSR-ECD fused to mouse IgG2a Fc (SEQ ID NO:221) vs. control-mIgG2a at various protein concentrations.

FIG. 22 presents mRNA levels of INOS, which is a prototypic M1 marker, evaluated under the different stimulation conditions in three independent experimental repetitions (EXP1, EXP2 and EXP3). Axis Y represents the absolute value of mRNA expression. Axis X represents the different stimulating conditions as detailed in Example 12, (IFNg, LPS, IFNg+LPS, IL4, TGFb, PGE2, TSNT (tumor supernatant). NS stands for non-stimulated.

FIG. 23 presents mRNA levels of inducible ALOX15, which is a prototypic M2 marker, evaluated under the different stimulation conditions in three independent experimental repetitions (EXP1, EXP2 and EXP3) Axis Y represents the absolute value of mRNA expression. Axis X represents the different stimulating conditions as detailed in Example 12 (IFNg, LPS, IFNg+LPS, IL4, TGFb, PGE2, TSNT (tumor supernatant). NS stands for non-stimulated.

FIG. 24 presents mRNA levels of LSR evaluated under the different stimulation conditions in three independent experimental repetitions (EXP1, EXP2 and EXP3) Axis Y represents the absolute value of mRNA expression. Axis X represents the different stimulating conditions as detailed in Example 12. (IFNg, LPS, IFNg+LPS, IL4, TGFb, PGE2, TSNT (tumor supernatant). NS stands for non-stimulated. The mRNA levels of LSR were detected using mouse LSR Taqman probe (Applied Biosystems; cat#: Mm00660290_ml).

FIG. 25 shows that anti-LSR human IgG1 antibodies demonstrate CDC activity against HEK293 expressing LSR. HEK293 cell lines expressing cyno LSR (Cyno LSR Hek) or GFP (GFP MT Hek) were incubated with antibodies against LSR or isotype controls hIgG1 (ET901) or mIgG2a (Mopc173), in the presence of complement, and viability measured after 1 hr to assess % CDC activity.

FIG. 26 shows FACS staining of anti-LSR antibodies on HEK293 cells ectopically expressing LSR.

FIG. 26A represents binding of the anti-LSR mouse IgG2a monoclonal antibody 8C8, FIG. 26B, C, D, E, F, G, H represent binding of the anti-LSR human IgG1 monoclonal antibodies S32-03.G11, S11-01.E02, S11-01.F08, S11-04.C11, S11-04.D11, S11-04.H07, S11-04.H09, respectively. The graphs show MFI values of the binding of the various antibodies to HEK293 cells expressing cyno LSR (“cyno”, circles) or HEK293 cells transfected with empty vector (“MT”, squares). Binding of the Ig isotype control (huIgG1) to both types of cells is shown in triangles.

FIG. 27 shows that LSR expressed on HEK-293T cells inhibits Jurkat cells activation.

FIG. 28 shows that HEK-293T cells expressing LSR inhibit Jurkat cells activated with anti-CD3, as opposed to HEK-293T cells expressing CD20.

### DETAILED DESCRIPTION OF THE INVENTION

The present invention, in at least some embodiments, relates to polyclonal and monoclonal antibodies and fragments and/or conjugates thereof, and/or pharmaceutical composition comprising same, and/or diagnostic composition comprising same, wherein these antibodies specifically bind LSR proteins, and wherein said antibodies are adapted to be used as therapeutic and/or diagnostic agents, particularly for treatment and/or diagnosis of specific cancer as described herein, particularly human, humanized or chimeric monoclonal antibodies, including those that promote or inhibit activities elicited by LSR.

Without wishing to be limited by a closed list or by a single hypothesis, an antibody according to various embodiments of the present invention may optionally have one or more of the following properties. Such neutralizing antibody may optionally promote Th2 to Th1 shift, thereby potentially reverting the shift towards a Th2/M2 environment induced in the tumor micro-environment that reducesthe immune response towards the tumor. The antibody may therefore optionally promote the immune system component which acts against the tumor (Th1), while inhibiting the component which promotes the cancer (Th2). The antibody may promote or inhibit activities elicited by LSR, including those relating to modulation of immune costimulation, e.g. B7 related costimulation, increases T cell activation and cytokine secretion, or/and induce direct killing of cancer cells.

According to at least some embodiments of the present invention, such an antibody may optionally inhibit iTregs accumulation and immunosuppressive function, and/or enhance effector T cell activity.

The term “cancer” as used herein should be understood to encompass any neoplastic disease (whether invasive or metastatic) which is characterized by abnormal and uncontrolled cell division causing malignant growth or tumor, non-limiting examples of which are described herein.

According to at least some embodiments of the present invention, the antibodies are derived from particular heavy and light chain germline sequences and/or comprise particular structural features such as at least one CDR regions comprising particular amino acid sequences. According to at least some embodiments, the present invention provides isolated antibodies, methods of making such antibodies, immunoconjugates and bispecific molecules comprising such antibodies and pharmaceutical and diagnostic compositions containing the antibodies, immunoconjugates, alternative scaffolds or bispecific molecules according to at least some embodiments of the present invention.

According to at least some embodiments the present invention relates to in vitro and in vivo methods of using the antibodies and fragments thereof, to detect any one of LSR proteins.

According to at least some embodiments the present invention further relates to methods of using the foregoing antibodies and fragments and/or conjugates thereof and/or pharmaceutical and/or diagnostic composition comprising same, to treat and/or to diagnose cancer, as described herein.

LSR is a multimeric protein complex in the liver that undergoes conformational changes upon binding of free fatty acids, thereby revealing a binding site (s) that recognizes both apoB and apoE. Complete inactivation of the LSR gene is embryonic lethal in mice. Removal of a single LSR allele (LSR−/+) caused statistically significant increases in both plasma triglyceride and cholesterol levels. The biology of LSR has been thoroughly investigated in humans, rats and mice. LSR is mainly expressed in liver membranes and most elucidated function is to mediate clearance of chylomicrons after activations of free fatty acids. However, other functions may exist, since LSR is also expressed in other tissues. LSR expression has been shown in tumor tissue like ovarian cancer, and bladder cancer where LSR was one of thirty genes identified as a potential tumor marker. Upon LSR knockdown in epithelial cells, Tight Junction formation was affected and the epithelial barrier function was diminished. (BMC Med. Genomics. 2008; 1: 31. Diabetes. 2009 May; 58(5): 1040-1049, J Cell Sci, 15 Feb. 2011 124, 548-555).

LSR protein is disclosed in PCT Application No: PCT/IB2012/051868, owned in common with the present application, which is hereby incorporated by reference, as if fully set forth herein. This application demonstrates that the ECD sequence of mouse LSR molecule fused to mouse IgG2a inhibits mouse T-cell activation, induced by anti CD3 and anti-CD28, cytokine secretion. The fusion protein ameliorates disease symptoms in mice model of multiple sclerosis (EAE model) demonstrating that LSR has an important role in immune modulation. PCT/IB2012/051868 describes LSR antibodies that are potentially useful as therapeutic and/or diagnostic agents (both in vitro and in vivo diagnostic methods). Included in particular are antibodies and fragments that are immune activating or immune suppressing such as antibodies or fragments that target cells via ADCC (antibody dependent cellular cytotoxicity) or CDC (complement dependent cytotoxicity) activities, particularly for treating conditions wherein the LSR antigen is expressed, including cancers, and/or in infectious disorders, and/or immune related disorders.

As used herein, the term LSR refers to any one of the proteins set forth in anyone of SEQ ID NOs: 10-18, 21, 22, 31, 32, 47-50, 62-69, 143, 211-212, 237, and/or amino acid sequences corresponding to LSR IGV domains selected from the group consisting of any one of SEQ ID NOs: 95, 102, and/or the amino acid sequences corresponding to the unique edges of SEQ ID NO: 18, and/or variants thereof, and/or orthologs and/or fragments thereof, and/or nucleic acid sequences encoding for same, that are differentially expressed in cancer, on the cancer cells or in the immune cells infiltrating the tumor.

LSR protein is listed among many other proteins in WO2005019258; WO2005016962; WO2010105298, for the diagnosis and treatment of immune related diseases.

LSR protein is listed in US patent application No: US20070054268, among many other proteins proposed for diagnosing ovarian cancer and/or a likelihood for survival, or recurrence of disease.

LSR protein is listed in US patent application No: US20070154889, among many other proteins for identifying a melanoma, useful for distinguishing a malignant from benign melanocyte.

LSR protein is listed in PCT application No: WO06133923, among many other proteins for diagnosis, prognosis, and prediction of breast cancer.

LSR protein is listed in PCT application No: WO06138275, among many other proteins within stem cell gene signatures for use in the diagnosis and management of cancer.

US patent Nos: U.S. Pat. No. 7,919,091, U.S. Pat. No. 6,635,431 and U.S. Pat. No. 7,291,709, and other related patent family members disclose LSR proteins and LSR specific antibodies, particularly for treating obesity and other metabolic disorders.

US patent application No: 20120064100 disclose LSR protein among several other proteins for treating a condition associated with regulatory T (Treg) cell-mediated suppression of a immune system or for modulating an immune response.

However, the above referenced patents and/or patent applications do not teach or suggest or provide any incentive that would direct a skilled artisan to use antibodies comprising one or more of the specific CDRs as described herein, specifically binding to one or more polypeptides having amino acid sequences selected from the group consisting of SEQ ID NOs 215 and 216, and that does not specifically bind to any other portion of SEQ ID NO 10, wherein said other portion of SEQ ID NO:10 comprises amino acids 1-80 or amino acids 99 to 234 of SEQ ID NO: 10 for SEQ ID NO 215, or wherein said other portion of SEQ ID NO:10 comprises amino acids 1-117 or amino acids 136 to 234 of SEQ ID NO: 10 for SEQ ID NO 216, or amino acids 30-110 of SEQ ID NO 10 and that does not specifically bind to any other portion of SEQ ID NO 10, wherein said other portion of SEQ ID NO:10 comprises amino acids 1-29 or amino acids 111 to 234 of SEQ ID NO: 10.

Furthermore, the above referenced patents and/or patent applications do not teach or suggest or provide any incentive that would direct a skilled artisan to use antibodies specific to the LSR ECD for treatment and/or diagnosis of cancer as described herein. Examples of the utility of such antibodies for treatment and/or diagnosis of cancer are given below.

Furthermore, the above referenced patents and/or patent applications do not teach or suggest or provide any incentive that would direct a skilled artisan to use antibodies specific to the LSR ECD for treatment and/or diagnosis of immune related disorders as described herein. Without wishing to be limited in any way, it is expected that these antibodies have cytotoxic activity, including antibody-dependent or complement dependent cytotoxic activity, on immune cells, resulting in their depletion, leading to amelioration of the immune disease. Furthermore, still without wishing to be limited in any way, it is expected that these antibodies may enhance the inhibitory effect of LSR on T-cell activation, resulting in a dampening of immune cell response and amelioration of the immune disease.

Furthermore, the above referenced patents and/or patent applications do not teach or suggest or provide any incentive that would direct a skilled artisan to use antibodies specific to the LSR ECD for treatment and/or diagnosis of infectious disease as described herein. Without wishing to be limited in any way, it is expected that as an “infection” comprises a disorder, disease and/or condition caused by the persistence of foreign antigen, leading to diminished immune responses against the foreign antigen, the antibodies would be effective in activating the immune system to attack the infectious agent. Such diminished immune responses are characterized by impaired functionality which can be manifested as T cell exhausting, reduced cell proliferation and cytokine production, and can be reversed by blocking inhibitory pathways using antibodies as described herein.

As used herein, the term “antibody” may optionally refer to any of the following (and also optionally combinations of the following): monoclonal and/or polyclonal antibodies and antigen binding fragments and/or alternative scaffolds and/or conjugates and/or immunoconjugates.

According to at least some embodiments, the present invention provides antibodies and fragments as described herein, optionally and preferably wherein the antibody binds to human LSR with a KD of 1×10⁻⁸ M or less, and wherein the antibody exhibits at least one of the following properties: modulates B7 related costimulation, increases T cell activation, alleviates T-cell suppression, increases cytokine secretion, increases IL-2 secretion; increases interferon-gamma production by T-cells, increases Th1 response, decreases Th2 response, decreases or eliminates M2 macrophages, reduces M2 macrophage pro-tumorigenic activity, promotes cancer epitope spreading, reduces inhibition of T cell activation, increases T cell response in a mammal, stimulates antigen-specific memory responses, elicits apoptosis or lysis of cancer cells, stimulates cytotoxic or cytostatic effect on cancer cells, induces direct killing of cancer cells, induces complement dependent cytotoxicity and/or antibody dependent cell-mediated cytotoxicity.

Optionally said antibody or fragment increases immune response against the cancer.

Optionally said antibody or fragment reduces activity of regulatory T lymphocytes (T-regs).

Optionally said antibody or fragment inhibits iTreg differentiation.

According to at least some embodiments, the present invention provides the foregoing antibodies and fragments thereof, wherein the antibody is a chimeric, humanized, fully human antibody and/or is an antibody or antibody fragment having CDC or ADCC activities on target cells.

Included in particular are antibodies and fragments that are immune activating or immune suppressing such as antibodies or fragments that target cells via ADCC (antibody dependent cellular cytotoxicity) or CDC (complement dependent cytotoxicity) activities.

According to at least some embodiments, the present invention provides blocking antibody that specifically binds any one of LSR proteins, selected from the group consisting of any one of SEQ ID NOs: 10-18, 21, 22, 31, 32, 47-50, 62-69, 143, 211-212, and/or amino acid sequences corresponding to extracellular domains thereof, selected from the group consisting of any one of SEQ ID NOs: 12, 14, 47-50, and/or fragments, and/or epitopes thereof, may optionally and preferably be specifically applied to cancer immunotherapy, alone or in combination with a potentiating agent(s), which increase an endogenous anti-tumor responses.

Furthermore, surprisingly, it has been found that an antibody that specifically binds any one of LSR proteins, selected from the group consisting of any one of SEQ ID NOs: 10-18, 21, 22, 31, 32, 47-50, 62-69, 95, 102, 143, 211-212, and/or their corresponding extracellular domains, selected from the group consisting of any one of SEQ ID NOs:12, 14, 47-50, and/or fragments, and/or epitopes thereof, may optionally and preferably be specifically applied to treatment of certain cancers, against which such an antibody demonstrates particular efficacy. Pharmaceutical compositions comprising such an antibody, in conjunction with a pharmaceutically acceptable carrier, are also provided herein.

Furthermore, surprisingly, it has been found that said antibody demonstrates particular efficacy in specific cancers, including cancers in which LSR is expressed on malignant cells, immune cells infiltrating into the tumor (such as T-cells, B-cell, macrophages, myeloid derive suppressor cells, mast cells) and/or stromal tumor cells. LSR expression on any of the cells listed above could be either present prior to treatment by standared of care agents or induced post treatment.

Furthermore, surprisingly, it has been found that improved outcome can be achieved using the above LSR antibodies for treatment of any one or more of:
Breast cancer, preferably any of ductal-adenocarcinoma, infiltrating ductal carcinoma, lobular carcinoma, mucinous adenocarcinoma, intra duct and invasive ductal carcinoma, preferably Scirrhous adenocarcinoma;
Colorectal cancer, preferably any of Moderate to Poorly Differentiated Adenocarcinoma of the cecum, Well, Moderate and Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum;
Lung cancer, preferably any of Well to Poorly differentiated Non-small cell carcinoma, Squamous Cell Carcinoma, preferably Moderately Differentiated Squamous Cell Carcinoma, Moderately to poorly differentiated squamous carcinoma, Moderately well differentiated keratinising squamous cell carcinoma, large cell adenocarcinoma, Small cell lung cancer;
Prostate cancer, preferably any of Adenocarcinoma Gleason Grade 5 to 9, Infiltrating adenocarcinoma, High grade prostatic intraepithelial neoplasia, undifferentiated carcinoma;
Stomach cancer, preferably moderately differentiated gastric adenocarcinoma;
Ovary cancer, preferably any of serous papillary cystic carcinoma, Serous cystadenocarcinoma, Invasive serous papillary carcinoma;
Brain cancer, preferably any of Astrocytoma, preferably grade 4 Astrocytoma, Glioblastoma multiforme;
Kidney cancer, preferably Clear cell renal cell carcinoma;
Liver cancer, preferably any of Hepatocellular carcinoma, preferably Low Grade hepatocellular carcinoma, Fibrolamellar Hepatocellular Carcinoma;
Hematological cancer, preferably any of large cell lymphoma, High and low grade Non-Hodgkin's Lymphoma.

It should be noted that surprisingly and contrary to the art of record, the following cancer subtypes, Hodgkin's Lymphoma, Granulosa cell tumor of the ovary, and Astrocytoma grade 2, were found to frequently, if not typically, fail to express LSR at a sufficient level, and therefore patients suffering from these subtypes are unlikely to benefit from treatment with anti-LSR antibodies.

Even more unexpectedly, it was found that ductal-adenocarcinoma, infiltrating ductal carcinoma, Lobular carcinoma of breast, mucinous adenocarcinoma of the breast, Intra duct and invasive ductal carcinoma, Moderate to Poorly Differentiated Adenocarcinoma of the cecum, Well to Poorly Differentiated Adenocarcinoma of the colon, Grade 2 Tubular adenocarcinoma of the ascending colon, colon adenocarcinoma Duke's stage C1, invasive adenocarcinoma, Adenocarcinoma of the rectum, Grade 3 Adenocarcinoma of the rectum, Moderately Differentiated Adenocarcinoma of the rectum, Moderately Differentiated Mucinous adenocarcinoma of the rectum, Well to Poorly differentiated Non-small cell carcinoma, Squamous Cell Carcinoma: Moderately Differentiated, Moderately to poorly differentiated squamous carcinoma, Moderately well differentiated keratinising squamous cell carcinoma, large cell adenocarcinoma, Adenocarcinoma Gleason Grade 7 to 9, Infiltrating adenocarcinoma, Moderately differentiated adenocarcinoma, serous papillary cystic carcinoma, Serous cystadenocarcinoma, grade 4 Astrocytoma, Glioblastoma multiforme, Clear cell renal cell carcinoma, Hepatocellular carcinoma, and Low Grade hepatocellular carcinoma, are especially susceptible to treatment with anti-LSR antibodies because of the significantly high levels of LSR expression found on these cancer cells.

According to at least some embodiments, for any of the above described cancers, optionally each of the above described cancer type or subtype may optionally form a separate embodiment and/or may optionally be combined as embodiments or subembodiments.

According to at least some embodiments, for any of the above described cancers, methods of treatment and also uses of the antibodies and pharmaceutical compositions described herein are provided wherein the cancer expresses LSR polypeptides comprised in SEQ ID NOs: 10-18, 21, 22, 31, 32, 47-50, 62-69, 95, 102, 143, 211-212, and/or their corresponding extracellular domains, selected from the group consisting of any one of SEQ ID NOs: 12, 14, 47-50, and/or fragments, such as for example SEQ ID NO:237, and/or epitopes thereof, on the cancer cells or in the immune cells infiltrating the tumor.

As used herein, when the term “epitopes thereof” appears, it may optionally and without limitation refer to epitopes as embodied in SEQ ID NOs: 215, 216 and/or SEQ ID NO 237.

Optionally the antibodies may be used for treatment of cancer as described herein. Optionally, said cancer, said immune infiltrate or both express LSR at a sufficient level and said cancer is as described herein. By immune infiltrate it is meant immune cells infiltrating to the tumor or to the area of the cancerous cells. By “expressing LSR at a sufficient level” it is meant that such cells express LSR protein at a high enough level according to an assay. For example, if the assay is IHC (immunohistochemistry), and expression is measured on a scale of 0 to 3 (0—no expression, 1—faint staining, 2—moderate and 3—strong expression), then a sufficient level of LSR expression would optionally be at least 1, preferably be at least 2 and more preferably be at least 3. Optionally the antibodies or immune molecules as described herein may be used for such an assay.

More preferably, the antibody binds to corresponding human LSR antigen with a KD of 3×10-8 M or less, or with a KD of 1×10-9 M or less, or with a KD of 0.1×10-9 M or less, or with a KD Of 0.05×10-9 M or less or with a KD of between 1×10-9 and 1×10-11 M.

In addition, preferably these antibodies and/or conjugates thereof are effective in eliciting selective killing of such cancer cells and for modulating immune responses involved in autoimmunity and cancer.

Standard assays to evaluate the binding ability of the antibodies toward LSR are known in the art, including for example, ELISAs, Western blots and RIAs. Suitable assays are described in detail in the Examples. The binding kinetics (e.g., binding affinity) of the antibodies also can be assessed by standard assays known in the art, such as by Biacore analysis.

Upon production of anti-LSR antibody sequences from antibodies can bind to LSR the VH and VL sequences can be “mixed and matched” to create other anti-LSR, binding molecules according to at least some embodiments of the invention. LSR binding of such “mixed and matched” antibodies can be tested using the binding assays described above. e.g., ELISAs). Preferably, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence. For example, the VH and VL sequences of homologous antibodies are particularly amenable for mixing and matching.

Optionally, the antibody comprises CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more conservative amino acid substitutions except for the Serine residue in heavy chain CDR3 at position 100A (Kabat numbering system); (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein.

Optionally, for any antibody or fragment described herein, the antibody may be bispecific, meaning that one arm of the Ig molecule is specific for binding to the target protein or epitope as described herein, and the other arm of the Ig molecule has a different specificity that can enhance or redirect the biological activity of the antibody or fragment. In this regard, a multi-specific antibody is also considered to be at least bispecific. The antibody or fragment also can be multi-specific in the sense of being multi-valent.

According to at least some embodiments the invention relates to protein scaffolds with specificities and affinities in a range similar to specific antibodies. According to at least some embodiments the present invention relates to an antigen-binding construct comprising a protein scaffold which is linked to one or more epitope-binding domains. Such engineered protein scaffolds are usually obtained by designing a random library with mutagenesis focused at a loop region or at an otherwise permissible surface area and by selection of variants against a given target via phage display or related techniques. According to at least some embodiments the invention relates to alternative scaffolds including, but not limited to, anticalins, DARPins, Armadillo repeat proteins, protein A, lipocalins, fibronectin domain, ankyrin consensus repeat domain, thioredoxin, chemically constrained peptides and the like. According to at least some embodiments the invention relates to alternative scaffolds that are used as therapeutic agents for treatment of cancer as recited herein, as well as for in vivo diagnostics.

According to at least some embodiments, there is provided a method of performing one or more of the following in a subject:

(a) upregulating cytokines, (b) increases T-cell proliferation and/or expansion, (c) increases interferon-gamma production by T-cells (d) increases IL-2 secretion (e) stimulates antibody responses; (f) inhibits cancer cell growth, (g) promoting antigenic specific T cell immunity, (g) promoting CD4+ and/or CD8+T cell activation, (i) alleviating T-cell suppression, (j) alleviating apoptosis or lysis of cancer cells, (k) cytotoxic or cytostatic effect on cancer cells,

comprising administering an antibody or immune molecule as described herein or a pharmaceutical composition as described herein to the subject.

In order that the present invention in various embodiments may be more readily understood, certain terms are first defined. Additional definitions are set forth throughout the detailed description.

As used herein the term “isolated” refers to a compound of interest (for example a polynucleotide or a polypeptide) that is in an environment different from that in which the compound naturally occurs e.g. separated from its natural milieu such as by concentrating a peptide to a concentration at which it is not found in nature. “Isolated” includes compounds that are within samples that are substantially enriched for the compound of interest and/or in which the compound of interest is partially or substantially purified.

An “immune cell” refers to any cell from the hemopoietic origin including but not limited to T cells, B cells, monocytes, dendritic cells, and macrophages.

As used herein, the term “polypeptide” refers to a chain of amino acids of any length, regardless of modification (e.g., phosphorylation or glycosylation).

As used herein, a “costimulatory polypeptide” or “costimulatory molecule” is a polypeptide that, upon interaction with a cell-surface molecule on T cells, modulates T cell responses.

As used herein, “costimulatory signaling” is the signaling activity resulting from the interaction between costimulatory polypeptides on antigen presenting cells and their receptors on T cells during antigen-specific T cell responses. Without wishing to be limited by a single hypothesis, the antigen-specific T cell response is believed to be mediated by two signals: 1) engagement of the T cell Receptor (TCR) with antigenic peptide presented in the context of MHC (signal 1), and 2) a second antigen-independent signal delivered by contact between different costimulatory receptor/ligand pairs (signal 2). Without wishing to be limited by a single hypothesis, this “second signal” is critical in determining the type of T cell response (activation vs inhibition) as well as the strength and duration of that response, and is regulated by both positive and negative signals from costimulatory molecules, such as the B7 family of proteins.

As used herein, the term “B7” polypeptide means a member of the B7 family of proteins that costimulate T cells including, but not limited to B7-1, B7-2, B7-DC, B7-H5, B7-H1, B7-H2, B7-H3, B7-H4, B7-H6, B7-S3 and biologically active fragments and/or variants thereof. Representative biologically active fragments include the extracellular domain or fragments of the extracellular domain that costimulate T cells.

As used herein, a “variant” polypeptide contains at least one amino acid sequence alteration as compared to the amino acid sequence of the corresponding wild-type polypeptide.

As used herein, “conservative” amino acid substitutions are substitutions wherein the substituted amino acid has similar structural or chemical properties. As used herein, the term “host cell” refers to prokaryotic and eukaryotic cells into which a recombinant vector can be introduced.

As used herein, the term “an edge portion” or “a new junction” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

In some embodiments, a bridge between a tail or a head or a unique insertion, and a “known protein” portion of a variant, comprises at least about 10 amino acids, or in some embodiments at least about 20 amino acids, or in some embodiments at least about 30 amino acids, or in some embodiments at least about 40 amino acids, in which at least one amino acid is from the tail/head/insertion and at least one amino acid is from the “known protein” portion of a variant. In some embodiments, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of the protein, comprising a polypeptide having a length “n”, wherein n is at least about 10 amino acids in length, optionally at least about 20 amino acids, at least about 30 amino acids, at least about 40 amino acids, or at least about 50 amino acids in length, wherein at least two amino acids comprise XX (2 amino acids in the center of the bridge, one from each end of the edge), having a structure as follows (numbering according to the sequence of the protein): a sequence starting from any of amino acid numbers 49-x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49-x (for example) is not less than 1, nor 50+((n−2)−x) (for example) greater than the total sequence length.

As used herein, the term “vaccine” refers to a biological preparation that improves immunity to a particular disease, wherein the vaccine includes cancer antigen, against which immune responses are elicited. A vaccine typically includes an adjuvant as immune potentiator to stimulate the immune system. As used herein, the term “therapeutic vaccine” and/or “therapeutic vaccination” refers to a vaccine used to treat cancer.

As used herein, the term “adjuvant” refers to an agent used to stimulate the immune system and increase the response to a vaccine, without having any specific antigenic effect in itself.

As used herein, the terms “immunologic”, “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. Without wishing to be limited by a single hypothesis, a cellular immune response is elicited by the presentation of polypeptide epitopes in association with Class II or Class I MHC molecules to activate antigen-specific CD4+T helper cells and/or CD8+ cytotoxic T cells, respectively. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4+T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

An “immunogenic agent” or “immunogen” is capable of inducing an immunological response against itself on administration to a mammal, optionally in conjunction with an adjuvant.

The term “antibody” as referred to herein includes whole polyclonal and monoclonal antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of at least one heavy chain variable region (abbreviated herein as VH) and a heavy chain constant region. The heavy chain constant region is comprised of three domains, CH1, CH2 and CH3. Each light chain is comprised of at least one light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., LSR molecules, and/or a fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V Light, V Heavy, Constant light (CL) and CH1 domains; (ii) a F(ab′).2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds LSR proteins and/or fragments thereof, and is substantially free of antibodies that specifically bind antigens other than LSR, respectively. An isolated antibody that specifically binds LSR proteins may, however, have cross-reactivity to other antigens, such as LSR molecules from other species, respectively. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies according to at least some embodiments of the present invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human LSR proteins” is intended to refer to an antibody that binds to LSR proteins, preferably one with a KD of 5×10⁻⁸ M or less, more preferably 3×10⁻⁸ M or less, even more preferably 1×10⁻⁹ M or less, even more preferably 1×10⁻¹° M, even more preferably 1×10⁻¹¹ M and even more preferably 1×10⁻¹² M or less.

The term “K-assoc” or “Ka”, as used herein, is intended to refer to the association rate of a particular antibody-antigen interaction, whereas the term “Kdiss” or “Kd,” as used herein, is intended to refer to the dissociation rate of a particular antibody-antigen interaction. The term “KD”, as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface Plasmon resonance, preferably using a biosensor system such as a Biacore® system.

As used herein, the term “high affinity” for an IgG antibody refers to an antibody having a KD of 10⁻⁸ M or less, more preferably 10⁻⁹ M or less and even more preferably 10⁻¹⁰ M or less for a target antigen. However, “high affinity” binding can vary for other antibody isotypes. For example, “high affinity” binding for an IgM isotype refers to an antibody having a KD of 10⁻⁷ M or less, more preferably 10⁻⁸ M or less.

As used herein, the term “subject” or “patient” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

Antibodies Having Particular Germline Sequences

In certain embodiments, an antibody of the invention comprises a heavy chain variable region from a particular germline heavy chain immunoglobulin gene and/or a light chain variable region from a particular germline light chain immunoglobulin gene.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95, 96, 97, 98 or 99%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Homologous Antibodies

In yet another embodiment, an antibody of the invention comprises heavy and light chain variable regions comprising amino acid sequences that are homologous to isolated anti-LSR amino acid sequences of preferred anti-LSR antibodies, respectively, wherein the antibodies retain the desired functional properties of the parent anti-LSR antibodies.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on preferred anti-anti-LSR antibodies isolated and produced using methods herein, or conservative modifications thereof, and wherein the antibodies retain the desired functional properties of anti-LSR antibodies according to at least some embodiments of the invention, respectively.

In various embodiments, the anti-LSR antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody according to at least some embodiments of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody according to at least some embodiments of the invention can be replaced with other amino acid residues from the same side chain family and the altered antibody can be tested for retained function (i.e., the functions set forth in (c) through (j) above) using the functional assays described herein.

Antibodies that Bind to the Same Epitope as Anti-LSR According to at Least Some Embodiments of the Invention.

In another embodiment, the invention provides antibodies that bind to preferred epitopes on human LSR which possess desired functional properties such as modulation of co-stimulation and related functions. Other antibodies with desired epitope specificity may be selected and will have the ability to cross-compete for binding to LSR antigen with the desired antibodies.

Engineered and Modified Antibodies

An antibody according to at least some embodiments of the invention further can be prepared using an antibody having one or more of the VH and/or VL sequences derived from an anti-LSR antibody starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant regions, for example to alter the effector functions of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Suitable framework sequences can be obtained from public DNA databases or published references that include germline antibody gene sequences. For example, germline DNA sequences for human heavy and light chain variable region genes can be found in the “VBase” human germline sequence database (available on the Internet), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline VH Sequences Reveals about Fifty Groups of VH Segments with Different Hypervariable Loops” J. Mol. Biol. 227:776-798; and Cox, J. P. L. et al. (1994) “A Directory of Human Germ-line VH Segments Reveals a Strong Bias in their Usage” Eur. J. Immunol. 24:827-836; the contents of each of which are expressly incorporated herein by reference.

Another type of variable region modification is to mutate amino acid residues within the VH and/or VL CDR 1, CDR2 and/or CDR3 regions to thereby improve one or more binding properties (e.g., affinity) of the antibody of interest. Site-directed mutagenesis or PCR-mediated mutagenesis can be performed to introduce the mutations and the effect on antibody binding, or other functional property of interest, can be evaluated in appropriate in vitro or in vivo assays. Preferably conservative modifications (as discussed above) are introduced. The mutations may be amino acid substitutions, additions or deletions, but are preferably substitutions. Moreover, typically no more than one, two, three, four or five residues within a CDR region are altered.

Engineered antibodies according to at least some embodiments of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

In addition or alternative to modifications made within the framework or CDR regions, antibodies according to at least some embodiments of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcy receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for Fc gamma RI, Fc gamma RII, Fc gammaRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve Fcgamma.RIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan CA and Carter PJ (2010) Nature Rev Immunol 10:301-316).

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8.−/− cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)—N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

Methods of Engineering Antibodies

As discussed above, anti-LSR antibodies having VH and VK sequences disclosed herein can be used to create new anti-LSR antibodies, respectively, by modifying the VH and/or VL sequences, or the constant regions attached thereto. Thus, in another aspect according to at least some embodiments of the invention, the structural features of an anti-LSR antibody according to at least some embodiments of the invention, are used to create structurally related anti-LSR antibodies that retain at least one functional property of the antibodies according to at least some embodiments of the invention, such as binding to human LSR, respectively. For example, one or more CDR regions of one LSR antibody or mutations thereof, can be combined recombinantly with known framework regions and/or other CDRs to create additional, recombinantly-engineered, anti-LSR antibodies according to at least some embodiments of the invention, as discussed above. Other types of modifications include those described in the previous section. The starting material for the engineering method is one or more of the VH and/or VK sequences provided herein, or one or more CDR regions thereof. To create the engineered antibody, it is not necessary to actually prepare (i.e., express as a protein) an antibody having one or more of the VH and/or VK sequences provided herein, or one or more CDR regions thereof. Rather, the information contained in the sequences is used as the starting material to create a “second generation” sequences derived from the original sequences and then the “second generation” sequences is prepared and expressed as a protein.

Standard molecular biology techniques can be used to prepare and express altered antibody sequence.

Preferably, the antibody encoded by the altered antibody sequences is one that retains one, some or all of the functional properties of the anti-LSR antibodies, respectively, produced by methods and with sequences provided herein, which functional properties include binding to LSR antigen with a specific KD level or less and/or modulating B7 costimulation and/or selectively binding to desired target cells such as for example, that express LSR antigen.

The functional properties of the altered antibodies can be assessed using standard assays available in the art and/or described herein.

In certain embodiments of the methods of engineering antibodies according to at least some embodiments of the invention, mutations can be introduced randomly or selectively along all or part of an anti-LSR antibody coding sequence and the resulting modified anti-LSR antibodies can be screened for binding activity and/or other desired functional properties.

Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

Nucleic Acid Molecules Encoding Antibodies

Another aspect of the invention pertains to nucleic acid molecules that encode the antibodies according to at least some embodiments of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid according to at least some embodiments of the invention can be, for example, DNA or RNA and may or may not contain intronic sequences. In a preferred embodiment, the nucleic acid is a cDNA molecule.

Nucleic acids according to at least some embodiments of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker.

The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH— and VL-encoding DNA fragments are operatively linked to another fragment encoding a flexible linker, e.g., encoding the amino acid sequence (Gly-4-Ser)3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Production of Anti-LSR Monoclonal Antibodies

Monoclonal antibodies (mAbs) of the present invention can be produced by a variety of techniques, including conventional monoclonal antibody methodology e.g., the standard somatic cell hybridization technique of Kohler and Milstein (1975) Nature 256:495. Although somatic cell hybridization procedures are preferred, in principle, other techniques for producing monoclonal antibody can be employed e.g., viral or oncogenic transformation of B lymphocytes.

A preferred animal system for preparing hybridomas is the murine system. Hybridoma production in the mouse is a very well-established procedure Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

According to at least some embodiments of the invention, the antibodies are human monoclonal antibodies. Such human monoclonal antibodies directed against LSR can be generated using transgenic or transchromosomic mice carrying parts of the human immune system rather than the mouse system. These transgenic and transchromosomic mice include mice referred to herein as the HuMAb Mouse RTM and KM Mouse RTM, respectively, and are collectively referred to herein as “human Ig mice.” The HuMAb Mouse TM. (Medarex. Inc.) contains human immunoglobulin gene miniloci that encode unrearranged human heavy (.mu. and.gamma.) and.kappa. light chain immunoglobulin sequences, together with targeted mutations that inactivate the endogenous.mu. and.kappa. chain loci (see e.g., Lonberg, et al. (1994) Nature 368(6474): 856-859). Accordingly, the mice exhibit reduced expression of mouse IgM or.kappa., and in response to immunization, the introduced human heavy and light chain transgenes undergo class switching and somatic mutation to generate high affinity human IgGkappa. monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann N.Y. Acad. Sci. 764:536-546). The preparation and use of the HuMab Mouse RTM., and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6:579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies according to at least some embodiments of the invention can be raised using a mouse that carries human immunoglobulin sequences on transgenes and transchomosomes, such as a mouse that carries a human heavy chain transgene and a human light chain transchromosome. Such mice, referred to herein as “KM mice TM.”, are described in detail in PCT Publication WO 02/43478 to Ishida et al.

Still further, alternative transgenic animal systems expressing human immunoglobulin genes are available in the art and can be used to raise anti-LSR antibodies according to at least some embodiments of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-LSR antibodies according to at least some embodiments of the invention. For example, mice carrying both a human heavy chain transchromosome and a human light chain transchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-LSR antibodies according to at least some embodiments of the invention.

Human monoclonal antibodies according to at least some embodiments of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies according to at least some embodiments of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Immunization of Human Ig Mice

When human Ig mice are used to raise human antibodies according to at least some embodiments of the invention, such mice can be immunized with a purified or enriched preparation of LSR antigen and/or recombinant LSR, or LSR fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50.mu.g) of LSR antigen can be used to immunize the human Ig mice intraperitoneally.

Prior experience with various antigens by others has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-LSR human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo 12). Alternatively or additionally, the KM Mouse. RTM. strain can be used.

Generation of Hybridomas Producing Human Monoclonal Antibodies

To generate hybridomas producing human monoclonal antibodies according to at least some embodiments of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×10-5 in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-Sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80 degrees C.

Generation of Transfectomas Producing Monoclonal Antibodies

Antibodies according to at least some embodiments according to at least some embodiments of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segments within the vector and the VK segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors according to at least some embodiments of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or.beta.-globin promoter. Still further, regulatory elements composed of sequences from different sources, such as the SR alpha. promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors according to at least some embodiments of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vectors encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies according to at least some embodiments of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies according to at least some embodiments of the invention include Chinese Hamster Ovary (CHO cells) (including dhfr-CHO cells, described in Urlaub and ChasM, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Characterization of Antibody Binding to Antigen

Antibodies according to at least some embodiments of the invention can be tested for binding to LSR by, for example, standard ELISA. Briefly, microtiter plates are coated with purified L LSR at 0.25.mu.g/ml in PBS, and then blocked with 5% bovine serum albumin in PBS. Dilutions of antibody (e.g., dilutions of plasma from -immunized mice) are added to each well and incubated for 1-2 hours at 37 degrees C. The plates are washed with PBS/Tween and then incubated with secondary reagent (e.g., for human antibodies, a goat-anti-human IgG Fc-specific polyclonal reagent) conjugated to alkaline phosphatase for 1 hour at 37 degrees C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with LSR immunogen. Hybridomas that bind with high avidity to LSR are subcloned and further characterized. One clone from each hybridoma, which retains the reactivity of the parent cells (by ELISA), can be chosen for making a 5-10 vial cell bank stored at −140 degrees C., and for antibody purification.

To purify anti-LSR antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80 degrees C.

To determine if the selected anti-LSR monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using LSR coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1.mu.g/ml of anti-human immunoglobulin overnight at 4 degrees C. After blocking with 1% BSA, the plates are reacted with 1 mug/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-LSR human IgGs can be further tested for reactivity with LSR antigen, respectively, by Western blotting. Briefly, LSR antigen can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Alternative Scaffolds

According to at least some embodiments the invention relates to protein scaffolds with specificities and affinities in a range similar to specific antibodies. According to at least some embodiments the present invention relates to an antigen-binding construct comprising a protein scaffold which is linked to one or more epitope-binding domains. Such engineered protein scaffolds are usually obtained by designing a random library with mutagenesis focused at a loop region or at an otherwise permissible surface area and by selection of variants against a given target via phage display or related techniques. According to at least some embodiments the invention relates to alternative scaffolds including, but not limited to, anticalins, DARPins, Armadillo repeat proteins, protein A, lipocalins, fibronectin domain, ankyrin consensus repeat domain, thioredoxin, chemically constrained peptides and the like. According to at least some embodiments the invention relates to alternative scaffolds that are used as therapeutic agents for treatment of cancer, autoimmune and infectious diseases as well as for in vivo diagnostics.

According to at least some embodiments the invention further provides a pharmaceutical composition comprising an antigen binding construct as described herein a pharmaceutically acceptable carrier.

The term ‘Protein Scaffold’ as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. Such protein scaffolds may comprise antigen-binding sites in addition to the one or more constant regions, for example where the protein scaffold comprises a full IgG. Such protein scaffolds will be capable of being linked to other protein domains, for example protein domains which have antigen-binding sites, for example epitope-binding domains or ScFv domains.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The phrase “immunoglobulin single variable domain” refers to an antibody variable domain (VH, V-HH, V-L) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid V-HH dAbs. Camelid V-HH are immunoglobulin single variable domain polypeptides that are derived from species including camel, llama, alpaca, dromedary, and guanaco, which produce heavy chain antibodies naturally devoid of light chains. Such V-HH domains may be humanised according to standard techniques available in the art, and such domains are still considered to be “domain antibodies” according to the invention. As used herein “VH includes camelid V-HH domains. NARV are another type of immunoglobulin single variable domain which were identified in cartilaginous fish including the nurse shark. These domains are also known as Novel Antigen Receptor variable region (commonly abbreviated to V(NAR) or NARV). For further details see MoI. Immunol. 44, 656-665 (2006) and US20050043519A.

The term “epitope-binding domain” refers to a domain that specifically binds an antigen or epitope independently of a different V region or domain, this may be a domain antibody (dAb), for example a human, camelid or shark immunoglobulin single variable domain or it may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human &#947; -crystallin and human ubiquitin (affilins); PDZ domains; scorpion toxinkunitz type domains of human protease inhibitors; Armadillo repeat proteins, thioredoxin, and fibronectin (adnectin); which has been subjected to protein engineering in order to obtain binding to a ligand other than the natural ligand.

Loops corresponding to CDRs of antibodies can be substituted with heterologous sequence to confer different binding properties i.e. Evibodies. For further details see Journal of Immunological Methods 248 (1-2), 31-45 (2001) Lipocalins are a family of extracellular proteins which transport small hydrophobic molecules such as steroids, bilins, retinoids and lipids. They have a rigid secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. SeI. 17, 455-462 (2004) and EP1641818A1 Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007) A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem. 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha helices; -beta turn. They can be engineered to bind different target antigens by randomising residues in the first alpha-helix and a beta-turn of each repeat. Their binding interface can be increased by increasing the number of modules (a method of affinity maturation). For further details see J. MoI. Biol. 332, 489-503 (2003), PNAS100(4), 1700-1705 (2003) and J. MoI. Biol. 369, 1015-1028 (2007) and US20040132028A1.

Fibronectin is a scaffold which can be engineered to bind to antigen. Adnectins consists of a backbone of the natural amino acid sequence of the 10th domain of the 15 repeating units of human fibronectin type 111 (FN3). Three loops at one end of the beta; -sandwich can be engineered to enable an Adnectin to specifically recognize a therapeutic target of interest. For further details see Protein Eng. Des. SeI. 18, 435-444 (2005), US200801 39791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5. 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include upto 25 amino acids without affecting the overall fold of the microprotein. For further details of engineered knottin domains, see WO2008098796.

Other epitope binding domains include proteins which have been used as a scaffold to engineer different target antigen binding properties include human beta-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7-Non-Antibody Scaffolds from Handbook of Therapeutic Antibodies (2007, edited by Stefan Dubel) and Protein Science 15:14-27 (2006). Epitope binding domains of the present invention could be derived from any of these alternative protein domains.

Conjugates or Immunoconjugates

The present invention encompasses conjugates for use in immune therapy comprising the LSR antigen and soluble portions thereof including the ectodomain or portions or variants thereof. For example the invention encompasses conjugates wherein the ECD of the LSR antigen is attached to an immunoglobulin or fragment thereof. The invention contemplates the use thereof for promoting or inhibiting LSR antigen activities such as immune costimulation and the use thereof in treating transplant, autoimmune, and cancer indications described herein.

In another aspect, the present invention features antibody-drug conjugates (ADCs), used for example for treatment of cancer, consisting of an antibody (or antibody fragment such as a single-chain variable fragment (scFv) linked to a payload drug (often cytotoxic). The antibody causes the ADC to bind to the target cancer cells. Often the ADC is then internalized by the cell and the drug is released into the cell. Because of the targeting, the side effects are lower and give a wider therapeutic window. Hydrophilic linkers (e.g., PEG4Ma1) help prevent the drug being pumped out of resistant cancer cells through MDR (multiple drug resistance) transporters.

In another aspect, the present invention features immunoconjugates comprising an anti-LSR antibody, or a fragment thereof, conjugated to a therapeutic agent, such as a cytotoxin, a drug (e.g., an immunosuppressant) or a radiotoxin. Such conjugates are referred to herein as “immunoconjugates” Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of therapeutic cytotoxins that can be conjugated to an antibody according to at least some embodiments of the invention include duocarmycins, calicheamicins, maytansines and auristatins, and derivatives thereof. An example of a calicheamicin antibody conjugate is commercially available (Mylotarg™; Wyeth).

Cytotoxins can be conjugated to antibodies according to at least some embodiments of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine 131, indium 111, yttrium 90 and lutetium 177. Methods for preparing radioimmunconjugates are established in the art. Examples of radioimmunoconjugates are commercially available, including Zevalin (IDEC Pharmaceuticals) and Bexxar. (Corixa Pharmaceuticals), and similar methods can be used to prepare radioimmunoconjugates using the antibodies according to at least some embodiments of the invention.

The antibody conjugates according to at least some embodiments of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Bispecific Molecules

According to at least some embodiments the invention encompasses also a multispecific antibody. Multispecific antibodies are monoclonal antibodies that have binding specificities for at least two different sites. In another aspect, the present invention features bispecific molecules comprising an anti-LSR antibody, or a fragment thereof, according to at least some embodiments of the invention. An antibody according to at least some embodiments of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody according to at least some embodiments of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule according to at least some embodiments of the invention, an antibody can be functionally linked (e.g., by chemical coupling, genetic fusion, noncovalent association or otherwise) to one or more other binding molecules, such as another antibody, antibody fragment, peptide or binding mimetic, such that a bispecific molecule results. In certain embodiments, one of the binding specificities of the bispecific antibodies is for LSR and the other is for any other antigen. In certain embodiments, bispecific antibodies may bind to two different epitopes of LSR. Bispecific antibodies may also be used to localize cytotoxic agents to cells which express LSR. Bispecific antibodies can be prepared as full length antibodies or antibody fragments.

A bispecific antibody according to at least some embodiments of the invention, is an antibody which can bind simultaneously to two targets which are of different structure. Bispecific antibodies (bsAb) and bispecific antibody fragments (bsFab) according to at least some embodiments of the invention, have at least one arm that specifically binds to a B-cell antigen or epitope and at least one other arm that specifically binds a targetable conjugate.

According to at least some embodiments the invention encompasses also a fusion antibody protein, which is a recombinantly produced antigen-binding molecule in which two or more different single-chain antibody or antibody fragment segments with the same or different specificities are linked. A variety of bispecific fusion antibody proteins can be produced using molecular engineering. In one form, the bispecific fusion antibody protein is monovalent, consisting of, for example, a sent with a single binding site for one antigen and a Fab fragment with a single binding site for a second antigen. In another form, the bispecific fusion antibody protein is divalent, consisting of, for example, an IgG with two binding sites for one antigen and two scFv with two binding sites for a second antigen.

According to at least some embodiments the invention encompasses also engineered antibodies with three or more functional antigen binding sites, including “Octopus antibodies” (see, e.g. US 2006/0025576A1).

According to at least some embodiments the invention encompasses also a “Dual Acting FAb” or “DAF” comprising an antigen binding site that binds to LSR as well as another, different antigen (see e.g. US 2008/0069820).

Accordingly, the present invention includes bispecific molecules comprising at least one first binding specificity for LSR and a second binding specificity for a second target epitope. According to at least some embodiments of the invention, the second target epitope is an Fc receptor, e.g., human Fc gamma R1 (CD64) or a human Fc alpha receptor (CD89). Therefore, the invention includes bispecific molecules capable of binding both to Fc gamma. R, Fc alpha R or Fc epsilon R expressing effector cells (e.g., monocytes, macrophages or polymorphonuclear cells (PMNs)), and to target cells expressing LSR, respectively. These bispecific molecules target LSR expressing cells to effector cell and trigger Fc receptor-mediated effector cell activities, such as phagocytosis of an LSR expressing cells, antibody dependent cell-mediated cytotoxicity (ADCC), cytokine release, or generation of superoxide anion.

According to at least some embodiments of the invention in which the bispecific molecule is multispecific, the molecule can further include a third binding specificity, in addition to an anti-Fc binding specificity. In one embodiment, the third binding specificity is an anti-enhancement factor (EF) portion, e.g., a molecule which binds to a surface protein involved in cytotoxic activity and thereby increases the immune response against the target cell.

The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g., via CD2, CD3, CD8, CD28, CD4, CD40, ICAM-1 or other immune cell that results in an increased immune response against the target cell).

According to at least some embodiments of the invention, the bispecific molecules comprise as a binding specificity at least one antibody, or an antibody fragment thereof, including, e.g., an Fab, Fab′, F(ab′).sub.2, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In one embodiment, the binding specificity for an Fc gamma receptor is provided by a monoclonal antibody, the binding of which is not blocked by human immunoglobulin G (IgG). As used herein, the term “IgG receptor” refers to any of the eight.gamma.-chain genes located on chromosome 1. These genes encode a total of twelve transmembrane or soluble receptor isoforms which are grouped into three Fc.gamma. receptor classes: Fc gamma R1 (CD64), Fc gamma RII (CD32), and Fc gamma.RIII (CD 16). In one preferred embodiment, the Fc gamma. receptor a human high affinity Fc.gamma RI. The human Fc gammaRl is a 72 kDa molecule, which shows high affinity for monomeric IgG (10 8-10-9 M.-1).

The production and characterization of certain preferred anti-Fc gamma. monoclonal antibodies are described by Fanger et al. in PCT Publication WO 88/00052 and in U.S. Pat. No. 4,954,617, the teachings of which are fully incorporated by reference herein. These antibodies bind to an epitope of Fc.gamma.R1, FcγRII or FcγRIII at a site which is distinct from the Fc.gamma. binding site of the receptor and, thus, their binding is not blocked substantially by physiological levels of IgG. Specific anti-Fc.gamma.RI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcy receptor antibody is a humanized form of monoclonal antibody 22 (H22). The production and characterization of the H22 antibody is described in Graziano, R. F. et al. (1995) J. Immunol. 155 (10): 4996-5002 and PCT Publication WO 94/10332. The H22 antibody producing cell line is deposited at the American Type Culture Collection under the designation HAO22CLI and has the accession no. CRL 11177.

In still other preferred embodiments, the binding specificity for an Fc receptor is provided by an antibody that binds to a human IgA receptor, e.g., an Fc-alpha receptor (Fc alpha.R1(CD89)), the binding of which is preferably not blocked by human immunoglobulin A (IgA). The term “IgA receptor” is intended to include the gene product of one alpha.-gene (Fc alpha.R1) located on chromosome 19. This gene is known to encode several alternatively spliced transmembrane isoforms of 55 to 10 kDa.

Fc.alpha.RI (CD89) is constitutively expressed on monocytes/macrophages, eosinophilic and neutrophilic granulocytes, but not on non-effector cell populations. Fc alpha R1 has medium affinity (Approximately 5×10⁻⁷ M-1) for both IgA1 and IgA2, which is increased upon exposure to cytokines such as G-CSF or GM-CSF (Morton, H. C. et al. (1996) Critical Reviews in Immunology 16:423-440). Four FcaRI-specific monoclonal antibodies, identified as A3, A59, A62 and A77, which bind Fc.alpha.RI outside the IgA ligand binding domain, have been described (Monteiro, R. C. et al. (1992) J. Immunol. 148:1764).

Fc. alpha. RI and Fc gamma. RI are preferred trigger receptors for use in the bispecific molecules according to at least some embodiments of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules according to at least some embodiments of the invention are murine, chimeric and humanized monoclonal antibodies.

The bispecific molecules of the present invention can be prepared by conjugating the constituent binding specificities, e.g., the anti-FcR and anti-LSR binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyld-ithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAbXmAb, mAbXFab, FabXF(ab′)2 or ligandXFab fusion protein. A bispecific molecule according to at least some embodiments of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); controlled Fab-arm exchange (see Labrijn et al., PNAS110(13):5145-50 (2013)); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma. counter or a scintillation counter or by autoradiography.

Uses of Antibodies and Pharmaceutical Compositions Thereof—Cancer

“Treatment” refers to both therapeutic treatment and prophylactic or preventative measures, which in this Example relates to treatment of cancer; however, also as described below, uses of antibodies and pharmaceutical compositions are also provided for treatment of infectious disease and/or autoimmune conditions. Those in need of treatment include those already with cancer as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it is meant reducing the severity of the disease, reducing the frequency of episodes of the disease, reducing the duration of such episodes, reducing the severity of such episodes, slowing/reducing cancer cell growth or proliferation, slowing progression of at least one symptom, ameliorization of at least one measurable physical parameter and the like.

“Mammal” for purposes of treatment refers to any animal classified as a mammal, including humans, domestic and farm animals, and zoo, sports, or pet animals, such as dogs, horses, cats, cows, etc. Preferably, the mammal is human. Preferably the mammal is a human which is diagnosed with one of the disease, disorder or conditions described hereinabove, or alternatively is predisposed to at least one type of cancer.

The term “therapeutically effective amount” refers to an amount of agent according to the present invention that is effective to treat a disease or disorder in a mammal.

The therapeutic agents of the present invention can be provided to the subject alone, or as part of a pharmaceutical composition where they are mixed with a pharmaceutically acceptable carrier.

Anti LSR antibody, a fragment, a conjugate thereof and/or a pharmaceutical composition comprising same, according to at least some embodiments of the present invention also can be administered in combination therapy, i.e., combined with other potentiating agents and/or other therapies. According to at least some embodiments, the anti LSR antibody could be used in combination with any of the known in the art standart of care cancer treatment (as can be found, for example, in http://www.cancer.govic ancertopics).

For example, the combination therapy can include an anti LSR antibody, a fragment, a conjugate thereof and/or a pharmaceutical composition comprising same, combined with at least one other therapeutic or immune modulatory agent, other compounds or immunotherapies, or immunostimulatory strategy, including, but not limited to, tumor vaccines, adoptive T cell therapy, Treg depletion, antibodies (e.g. bevacizumab, erbitux, Ipilimumab), peptides, pepti-bodies, small molecules, chemotherapeutic agents such as cytotoxic and cytostatic agents (e.g. paclitaxel, cisplatin, vinorelbine, docetaxel, gemcitabine, temozolomide, irinotecan, 5FU, carboplatin), immunological modifiers such as interferons and interleukins, immunostimulatory antibodies, growth hormones or other cytokines, folic acid, vitamins, minerals, aromatase inhibitors, RNAi, Histone Deacetylase Inhibitors, proteasome inhibitors, and so forth. In another example, the combination therapy can include an anti-LSR antibody or LSR modulating agent according to at least some embodiments of the present invention, such as a soluble polypeptide conjugate containing the ectodomain of the LSR antigen or a small molecule such as a peptide, ribozyme, aptamer, siRNA, or other drug that binds LSR, combined with at least one other therapeutic or immune modulatory agent.

According to at least some embodiments of the present invention, therapeutic agents that can be used in combination with anti-LSR antibodies, are potentiating agents that enhance anti-tumor responses.

Various strategies are available for combining an anti-LSR blocking antibody with potentiating agents for cancer immunotherapy. According to at least some embodiments of the present invention, anti-LSR antibody for cancer immunotherapy is used in combination with potentiating agents that are primarily geared to increase endogenous anti-tumor responses, such as Radiotherapy, Cryotherapy, Conventional/classical chemotherapy potentiating anti-tumor immune responses, Targeted therapy potentiating anti-tumor immune responses, Anti-angiogenic therapy, Therapeutic agents targeting immunosuppressive cells such as Tregs and MDSCs, Immunostimulatory antibodies, Cytokine therapy, Therapeutic cancer vaccines, Adoptive cell transfer.

The scientific rationale behind the combined use with some chemotherapy or anti-cancer conventional drugs is that cancer cell death, a consequence of the cytotoxic action of most chemotherapeutic compounds, may result in increased levels of tumor antigen leading to enhanced antigen presentation and stimulation of anti-tumor immune responses (i e immunogenic cell death), resulting in potentiating effects with the anti LSR antibody (Zitvogel et al 2008, Galluzzi et al 2012). Other combination therapies that may potentiate anti-tumor responses through tumor cell death are radiotherapy, Cryotherapy, surgery, and hormone deprivation. Each of these cancer therapies creates a source of tumor antigen in the host.

According to at least some embodiments of the invention, classical chemotherapies and conventional anti-cancer therapies as agents potentiating anti-tumor immune responses for combination with anti LSR antibodies are selected from the group consistin of but not limited to: Platinum based compounds such as oxaliplatin, cisplatin, carboplatin; Antibiotics with anti-cancer activity, such as dactinomycin, bleomycin, mitomycin-C, mithramycin and Anthracyclines, such as doxorubicin, daunorubicin, epirubicin, idarubicin; Anthracenediones, such as mitoxantrone; Alkylating agents, such as dacarbazine, melphalan, cyclophosphamide, temozolomide, chlorambucil, busulphan, nitrogen mustard, nitrosoureas; Antimetabolites, such as fluorouracil, raltitrexed, gemcitabine, cytosine arabinoside, hydroxyurea and Folate antagonists, such as methotrexate, trimethoprim, pyrimethamine, pemetrexed; Antimitotic agents such as polokinase inhibitors and Microtubule inhibitors, such as Taxanes and Taxoids, such as paclitaxel, docetaxel; Vinca alkaloids such as vincristine, vinblastine, vindesine, vinorelbine; Topoisomerase inhibitors, such as etoposide, teniposide, amsacrine, topotecan, irinotecan, camptothecin; Cytostatic agents including Antioestrogens such as tamoxifen, fulvestrant, toremifene, raloxifene, droloxifene, iodoxyfene, Antiandrogens such as bicalutamide, flutamide, nilutamide and cyproterone acetate, Progestogens such as megestrol acetate, Aromatase inhibitors such as anastrozole, letrozole, vorazole, exemestane; GnRH analogs, such as leuprorelin, goserelin, buserelin, degarelix; inhibitors of 5α-reductase such as finasteride.

According to at least some embodiments of the present invention, anti-LSR antibody for cancer immunotherapy is used in combination with Bisphosphonates, especially amino-bisphosphonates (ABP), which have shown to have anti-cancer activity. Some of the activities associated with ABPs are on human γδT cells that straddle the interface of innate and adaptive immunity and have potent anti-tumour activity. Targeted therapies can also stimulate tumor-specific immune response by inducing the immunogenic death of tumor cells or by engaging immune effector mechanisms (Galluzzi et al 2012, Vanneman and Dranoff 2012). In addition, according to at least some embodiments of the present invention, anti-LSR antibody for cancer immunotherapy is used in combination with any of the following: certain therapeutic monoclonal antibodies, trastuzumab, that favor the generation of tumor-specific cytotoxic CD8 T cells, and NK cells infiltration to the tumor and NK cell mediated cytotoxicity; certain tyrosine kinase inhibitors (TKIs) that promote cancer-directed immune responses by increasing MHC class II expression, decreased levels of tumor infiltrating immunosuppressive cells—Tregs and MDScs, reducing the expression of the immunosuppressive enzyme IDO by tumor cells, and/or inhibition of DC functions; Histone deacetylase (HDAC) inhibitors which were found increase the expression of NK-activating receptor ligands on the surface of cancer cells, thereby facilitating tumor cell recognition by NK cells, while proteasome inhibitors were found to sensitize tumor cells to CTL-mediated or NK-mediated cell lysis. According to at least some embodiments of the invention, Targeted therapies used as agents for combination with anti LSR antibodies for treatment of cancer are selected from the group consisting of but not limited to: histone deacetylase (HDAC) inhibitors, such as vorinostat, romidepsin, panobinostat, belinostat, mocetinostat, abexinostat, entinostat, resminostat, givinostat, quisinostat, sodium butyrate; Proteasome inhibitors, such as bortezomib, carfilzomib, disulfuram; mTOR pathway inhibitors, such as temsirolimus, rapamycin, everolimus; PI3K inhibitors, such as perifosine, CAL101, PX-866, IPI-145, BAY 80-6946; B-raf inhibitors such as vemurafenib, sorafenib; JAK2 inhibitors, such as lestaurtinib, pacritinib; Tyrosine kinase inhibitors (TKIs), such as erlotinib, imatinib, sunitinib, lapatinib, gefitinib, sorafenib, nilotinib, toceranib, bosutinib, neratinib, vatalanib, regorafenib, cabozantinib; other Protein kinase inhibitors, such as crizotinib; Inhibitors of serine/threonine kinases for example Ras/Raf signalling inhibitors such as farnesyl transferase inhibitors; Inhibitors of serine proteases for example matriptase, hepsin, urokinase; Inhibitors of intracellular signaling such as tipifarnib, perifosine; Inhibitors of cell signalling through MEK and/or AKT kinases; aurora kinase inhibitors such as AZD1152, PH739358, VX-680, MLN8054, R763, MP235, MP529, VX-528, AX39459; Cyclin dependent kinase inhibitors such as CDK2 and/or CDK4 inhibitors; Inhibitors of survival signaling proteins including Bcl-2, Bcl-XL, such as ABT-737; HSP90 inhibitors; Therapeutic monoclonal antibodies, such as anti-EGFR mAbs cetuximab, panitumumab, nimotuzumab, anti-ERBB2 mAbs trastuzumab, pertuzumab, anti-CD20 mAbs such as rituximab, ofatumumab, veltuzumab and mAbs targeting other tumor antigens such as alemtuzumab, labetuzumab, adecatumumab, oregovomab, onartuzumab; TRAIL pathway agonists, such as dulanermin (soluble rhTRAIL), apomab, mapatumumab, lexatumumab, conatumumab, tigatuzumab; Antibody fragments, bi-specific antibodies and bi-specific T-cell engagers (BiTEs), such as catumaxomab, blinatumomab; Antibody drug conjugates (ADC) and other immunoconjugates, such as ibritumomab triuxetan, tositumomab, brentuximab vedotin, gemtuzumab ozogamicin, clivatuzumab tetraxetan, pemtumomab, trastuzumab emtansine; Anti-angiogenic therapy such as bevacizumab, etaracizumab, volociximab, ramucirumab, aflibercept, sorafenib, sunitinib, regorafenib, axitinib, nintedanib, motesanib, pazopanib, cediranib; Metalloproteinase inhibitors such as marimastat; Inhibitors of urokinase plasminogen activator receptor function; Inhibitors of cathepsin activity.

Other cancer immunotherapies that also increase endogenous anti-tumor responses could also potentiate the effect of the anti LSR antibody by enhancing immune effector mechanisms, such as Adoptive T cell therapy, Therapeutic cancer vaccines, reduced immune suppressive cells and their function, Cytokine therapy, or Immunostimulatory antibodies.

According to at least some embodiments of the present invention, anti-LSR antibody for cancer immunotherapy is used in combination with Therapeutic agents targeting regulatory immunosuppressive cells such as regulatory T cells (Tregs) and myeloid derived suppressor cells (MDSCs). A number of commonly used chemotherapeutics exert non-specific targeting of Tregs and reduce the number or the immunosuppressive capacity of Tregs or MDSCs (Facciabene et al 2012; Byrne et al 2011; Gabrilovich and Nagaraj 2009). In this regard, metronomic therapy with some chemotherapy drugs results in immunostimulatory rather than immunosuppressive effects, via modulation of regulatory cells. Thus, according to at least some embodiments of the present invention, anti-LSR antibody for cancer immunotherapy is used in combination with drugs selected from but not limited to cyclophosphamide, gemcitabine, mitoxantrone, fludarabine, fludarabine, docetaxel, paclitaxel, thalidomide and thalidomide derivatives.

In addition, according to at least some embodiments of the present invention, anti-LSR antibody for cancer immunotherapy is used in combination with novel Treg-specific targeting agents including: 1) depleting or killing antibodies that directly target Tregs through recognition of Treg cell surface receptors such as anti-CD25 mAbs daclizumab, basiliximab or 2) ligand-directed toxins such as denileukin diftitox (Ontak)—a fusion protein of human IL-2 and diphtheria toxin, or LMB-2—a fusion between an scFv against CD25 and the pseudomonas exotoxin. 3) antibodies targeting Treg cell surface receptors such as CTLA4, PD-1, OX40 and GITR.

According to at least some embodiments of the present invention, anti-LSR antibody for cancer immunotherapy is used in combination with any of the options described below for disrupting Treg induction and/or function, including TLR (toll like receptors) agonists; agents that interfere with the adenosinergic pathway, such as ectonucleotidase inhibitors, or inhibitors of the A2A adenosine receptor; TGF-β inhibitors, such as fresolimumab, lerdelimumab, metelimumab, trabedersen, LY2157299, LY210976; blockade of Tregs recruitment to tumor tissues including chemokine receptor inhibitors, such as the CCR4/CCL2/CCL22 pathway.

According to at least some embodiments of the present invention, anti-LSR antibody for cancer immunotherapy is used in combination with any of the options described below for inhibiting the immunosuppressive tumor microenvironment, including inhibitors of cytokines and enzymes which exert immunosuppressive activities, such as IDO (indoleamine-2,3-dioxygenase) inhibitors; inhibitors of anti-inflammatory cytokines which promote an immunosuppressive microenvironment, such as IL-10, IL-35, IL-4 and IL-13; Bevacizumab which reduces Tregs and favors the differentiation of DCs.

According to at least some embodiments of the present invention, anti-LSR antibody for cancer immunotherapy is used in combination with any of the options described below for targeting MDSCs, including promoting their differentiation into mature myeloid cells that do not have suppressive functions By Vitamin D3, or VitaminA metabolites, such as retinoic acid, all-trans retinoic acid (ATRA); inhibition of MDSCs suppressive activity by COX2 inhibitors, phosphodiesterase 5 inhibitors like sildenafil, ROS inhibitors such as nitroaspirin.

According to at least some embodiments of the present invention, anti-LSR antibody for cancer immunotherapy is used in combination with Immunostimulatory antibodies as agents potentiating anti-tumor immune responses (Pardoll 2012):

Immunostimulatory antibodies promote anti-tumor immunity by directly modulating immune functions, i.e. blocking other inhibitory targets or enhancing costimulatory proteins. According to at least some embodiments of the present invention, anti-LSR antibody for cancer immunotherapy is used in combination with antagonistic antibodies targeting immune checkpoints including Anti-CTLA4 mAbs, such as ipilimumab, tremelimumab; Anti-PD-1 such as nivolumab BMS-936558/MDX-1106/ONO-4538, AMP224, CT-011, MK-3475; Anti-PDL-1 antagonists such as BMS-936559/MDX-1105, MEDI4736, RG-7446/MPDL3280A; Anti-LAG-3 such as IMP-321), Anti-TIM-3, Anti-BTLA, Anti-B7-H4, Anti-B7-H3, Anti-VISTA; Agonistic antibodies targeting immunostimulatory proteins, including Anti-CD40 mAbs such as CP-870,893, lucatumumab, dacetuzumab; Anti-CD137 mAbs such as BMS-663513 urelumab, PF-05082566; Anti-OX40 mAbs, such as Anti-OX40; Anti-GITR mAbs such as TRX518; Anti-CD27 mAbs, such as CDX-1127; Anti-ICOS mAbs.

Cytokines are molecular messengers that allow the cells of the immune system to communicate with one another to generate a coordinated, robust, but self-limited response to a target antigen. Cytokine-based therapies embody a direct attempt to stimulate the patient's own immune system to reject cancer. The growing interest over the past two decades in harnessing the immune system to eradicate cancer has been accompanied by heightened efforts to characterize cytokines and exploit their vast signaling networks to develop cancer treatments. Cytokines directly stimulate immune effector cells and stromal cells at the tumor site and enhance tumor cell recognition by cytotoxic effector cells. Numerous animal tumor model studies have demonstrated that cytokines have broad anti-tumor activity and this has been translated into a number of cytokine-based approaches for cancer therapy (Lee and Margolin 2011). A number of cytokines are in preclinical or clinical development as as agents potentiating anti-tumor immune responses for cancer immunotherapy, including among others: IL-2, IL-7, IL-12, IL-15, IL-17, IL-18 and IL-21, IL23, IL-27, GM-CSF, IFNα (interferon alpha), IFNβ, IFNγ.

Several cytokines have been approved for therapy of cancer and many more are under development. However, therapeutic efficacy is often hampered by severe side effects and poor pharmacokinetic properties. Thus, in addition to systemic administration of cytokines, a variety of strategies can be employed for the delivery of therapeutic cytokines and their localization to the tumor site, in order to improve their pharmacokinetics, as well as their efficacy and/or toxicity, including antibody-cytokine fusion molecules (immunocytokines), chemical conjugation to polyethylene glycol (PEGylation), transgenic expression of cytokines in autologous whole tumor cells, incorporation of cytokine genes into DNA vaccines, recombinant viral vectors to deliver cytokine genes, etc. In the case of immunocytokines, fusion of cytokines to tumor-specific antibodies or antibody fragments allows for targeted delivery and therefore improved efficacy and pharmacokinetics, and reduced side effects.

According to at least some embodiments of the present invention, anti-LSR antibody for cancer immunotherapy is used in combination with Cytokine therapy, involving the use of cytokines as agents potentiating anti-tumor immune responses, including cytokines such as IL-2, IL-7, IL-12, IL-15, IL-17, IL-18 and IL-21, IL23, IL-27, GM-CSF, IFNα (interferon alpha), IFNα-2b, IFNβ, IFNγ, and their different strategies for delivery, as described above.

Cancer vaccines are used to treat existing cancer (therapeutic) or prevent the development of cancer in certain high-risk individuals (prophylactic). Therapeutic cancer vaccines allow for improved priming of T cells and improved antigen presentation, and can be used as therapeutic agents for potentiating anti-tumor immune responses (Mellman et al 2011; Schlom 2012).

Several types of therapeutic cancer vaccines are in preclinical and clinical development. These include for example:

1) Whole tumor cell vaccines, in which cancer cells removed during surgery are treated to enhance their immunogenicity, and injected into the patient to induce immune responses against antigens in the tumor cells. The tumor cell vaccine can be autologous, i.e. a patient's own tumor, or allogeneic which typically contain two or three established and characterized human tumor cell lines of a given tumor type, such as the GVAX vaccine platforms.

2) Tumor antigen vaccines, in which a tumor antigen (or a combination of a few tumor antigens), usually proteins or peptides, are administered to boost the immune system (possibly with an adjuvant and/or with immune modulators or attractants of dendritic cells such as GM-CSF). The tumor antigens may be specific for a certain type of cancer, but they are not made for a specific patient.

3) Vector-based tumor antigen vaccines and DNA vaccines can be used as a way to provide a steady supply of antigens to stimulate an anti-tumor immune response. Vectors encoding for tumor antigens are injected into the patient (possibly with proinflammatory or other attractants such as GM-CSF), taken up by cells in vivo to make the specific antigens, which would then provoke the desired immune response. Vectors may be used to deliver more than one tumor antigen at a time, to increase the immune response. In addition, recombinant virus, bacteria or yeast vectors should trigger their own immune responses, which may also enhance the overall immune response.

4) Oncolytic virus vaccines, such as OncoVex/T-VEC, which involves the intratumoral injection of replication-conditional herpes simplex virus which preferentially infects cancer cells. The virus, which is also engineered to express GM-CSF, is able to replicate inside a cancer cell causing its lysis, releasing new viruses and an array of tumor antigens, and secreting GM-CSF in the process. Thus, such oncolytic virus vaccines enhance DCs function in the tumor microenvironment to stimulate anti-tumor immune responses.

5) Dendritic cell vaccines (Palucka and Banchereau 2012): Dendritic cells (DCs) phagocytose tumor cells and present tumor antigens to tumor specific T cells. In this approach, DCs are isolated from the cancer patient and primed for presenting tumor-specific T cells. To this end several methods can be used: DCs are loaded with tumor cells or lysates; DCs are loaded with fusion proteins or peptides of tumor antigens; coupling of tumor antigens to DC-targeting mAbs. The DCs are treated in the presence of a stimulating factor (such as GM-CSF), activated and matured ex vivo, and then re-infused back into the patient in order provoke an immune response to the cancer cells. Dendritic cells can also be primed in vivo by injection of patients with irradiated whole tumor cells engineered to secrete stimulating cytokines (such as GM-CSF). Similar approaches can be carried out with monocytes. Sipuleucel-T (Provenge), a therapeutic cancer vaccine which has been approved for treatment of advanced prostate cancer, is an example of a dendritic cell vaccine.

Thus, according to at least some embodiments of the present invention, anti-LSR antibody for cancer immunotherapy is used in combination with Therapeutic cancer vaccines. Non limiting examples of such therapeutic cancer vaccines include Whole tumor cell vaccines, Tumor antigen vaccines, Vector-based vaccines, Oncolytic virus vaccines, Dendritic-cell vaccines, as described above.

One approach to cancer immunotherapy is based on adoptive T cell therapy or adoptive cell transfer (ACT), which involves the ex vivo identification and expansion of autologous naturally occurring tumor specific T cells, which are then adoptively transferred back into the cancer patient (Restifo et al 2012). Cells that are infused back into a patient after ex vivo expansion can traffic to the tumor and mediate its destruction. Prior to this adoptive transfer, hosts can be immunodepleted by irradiation and/or chemotherapy. The combination of lymphodepletion, adoptive cell transfer, and a T cell growth factor (such as IL-2), can lead to prolonged tumor eradication in tumor patients. A more novel approach involves the ex vivo genetic modification of normal peripheral blood T cells to confer specificity for tumor-associated antigens. For example, clones of TCRs of T cells with particularly good anti-tumor responses can be inserted into viral expression vectors and used to infect autologous T cells from the patient to be treated. Another option is the use of chimeric antigen receptors (CARs) which are essentially a chimeric immunoglobulin-TCR molecule, also known as a T-body. CARs have antibody-like specificities and recognize MHC-nonrestricted structures on the surface of target cells (the extracellular target-binding module), grafted onto the TCR intracellular domains capable of activating T cells (Restifo et al 2012, Shi et al 2013).

According to at least some embodiments of the present invention, anti-LSR antibody for cancer immunotherapy is used in combination with Adoptive cell transfer to potentiate anti-tumor immune responses, including genetically modified T cells, as described above.

The LSR specific antibodies, and/or alternative scaffolds and/or multispecific and bispecific molecules and immunoconjugates, compositions comprising same according to at least some embodiments of the present invention can be co-administered together with one or more other therapeutic agents, which acts in conjunction with or synergistically with the composition according to at least some embodiments of the present invention to treat or prevent the cancer. The LSR related therapeutic agents and the one or more other therapeutic agents can be administered in either order or simultaneously. The other therapeutic agents are for example, a cytotoxic agent, a radiotoxic agent or an immunosuppressive agent. The composition can be linked to the agent (as an immunocomplex) or can be administered separately from the agent. In the latter case (separate administration), the composition can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the human anti-LSR antibodies, or antigen binding fragments and/or alternative scaffolds thereof, according to at least some embodiments of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody. In other embodiments, the subject can be additionally treated with an agent that modulates, e.g., enhances or inhibits, the expression or activity of Fcy or Fcy receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-gamma (IFN-gamma), and tumor necrosis factor (TNF).

Target-specific effector cells, e.g., effector cells linked to compositions (e.g., human antibodies, multispecific and bispecific molecules) according to at least some embodiments of the present invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10-8 to 10-9 but will vary depending on the therapeutic purpose. In general, the amount will be sufficient to obtain localization at the target cell, e.g., a tumor cell expressing LSR proteins, and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) according to at least some embodiments of the present invention and/or effector cells armed with these compositions can be used in conjunction with chemotherapy. Additionally, combination immunotherapy may be used to direct two distinct cytotoxic effector populations toward tumor cell rejection. For example, anti-LSR antibodies linked to anti-Fc-gamma R1 or anti-CD3 may be used in conjunction with IgG- or IgA-receptor specific binding agents.

Bispecific and multispecific molecules according to at least some embodiments of the present invention can also be used to modulate FcgammaR or FcgammaR levels on effector cells, such as by capping and elimination of receptors on the cell surface. Mixtures of anti-Fc receptors can also be used for this purpose.

The therapeutic compositions (e.g., human antibodies, alternative scaffolds multispecific and bispecific molecules and immunoconjugates) according to at least some embodiments of the present invention which have complement binding sites, such as portions from IgG1, -2, or -3 or IgM which bind complement, can also be used in the presence of complement. In one embodiment, ex vivo treatment of a population of cells comprising target cells with a binding agent according to at least some embodiments of the present invention and appropriate effector cells can be supplemented by the addition of complement or serum containing complement. Phagocytosis of target cells coated with a binding agent according to at least some embodiments of the present invention can be improved by binding of complement proteins. In another embodiment target cells coated with the compositions (e.g., human antibodies, multispecific and bispecific molecules) according to at least some embodiments of the present invention can also be lysed by complement. In yet another embodiment, the compositions according to at least some embodiments of the present invention do not activate complement.

The therapeutic compositions (e.g., human antibodies, alternative scaffolds multispecific and bispecific molecules and immunoconjugates) according to at least some embodiments of the present invention can also be administered together with complement. Thus, according to at least some embodiments of the present invention there are compositions, comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules according to at least some embodiments of the present invention and the complement or serum can be administered separately.

A “therapeutically effective dosage” of an anti-LSR antibody according to at least some embodiments of the present invention preferably results in a decrease in severity of disease symptoms, an increase in frequency and duration of disease symptom-free periods, an increase in lifepan, disease remission, or a prevention or reduction of impairment or disability due to the disease affliction. For example, for the treatment of LSR positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

The anti-LSR antibodies, according to at least some embodiments of the present invention, can be used as neutralizing antibodies. A Neutralizing antibody (Nabs), is an antibody that is capable of binding and neutralizing or inhibiting a specific antigen thereby inhibiting its biological effect, for example by blocking the receptors on the cell or the virus, inhibiting the binding of the virus to the host cell. NAbs will partially or completely abrogate the biological action of an agent by either blocking an important surface molecule needed for its activity or by interfering with the binding of the agent to its receptor on a target cell.

As used herein “therapeutic agent” is any one of the monoclonal and/or polyclonal antibodies, and/or antigen binding fragments, and/or conjugates containing same, and/or alternative scaffolds, thereof comprising an antigen binding site that binds specifically to any one of the LSR polypeptides or an epitope thereof, adopted for treatment of cancer, as recited herein.

According to an additional aspect of the present invention the therapeutic agents can be used to prevent pathologic inhibition of T cell activity, such as that directed against cancer cells.

According to an additional aspect of the present invention the therapeutic agents can be used to inhibit T cell activation, as can be manifested for example by T cell proliferation and cytokine secretion.

Thus, according to an additional aspect of the present invention there is provided a method of treating cancer as recited herein, and/or for promoting immune stimulation mediated by the LSR polypeptide in a subject by administering to a subject in need thereof an effective amount of any one of the therapeutic agents and/or a pharmaceutical composition comprising any of the therapeutic agents and further comprising a pharmaceutically acceptable diluent or carrier.

A therapeutic agent or pharmaceutical composition according to at least some embodiments of the present invention may also be administered in conjunction with other compounds or immunotherapies. For example, the combination therapy can include a compound of the present invention combined with at least one other therapeutic or immune modulatory agent, or immunostimulatory strategy, including, but not limited to, tumor vaccines, adoptive T cell therapy, Treg depletion, antibodies (e.g. bevacizumab, erbitux), peptides, pepti-bodies, small molecules, chemotherapeutic agents such as cytotoxic and cytostatic agents (e.g. paclitaxel, cisplatin, vinorelbine, docetaxel, gemcitabine, temozolomide, irinotecan, 5FU, carboplatin), immunological modifiers such as interferons and interleukins, immunostimulatory antibodies, growth hormones or other cytokines, folic acid, vitamins, minerals, aromatase inhibitors, RNAi, Histone Deacetylase Inhibitors, proteasome inhibitors, and so forth.

According to at least some embodiments, immune cells, preferably T cells, can be contacted in vivo or ex vivo with the therapeutic agents to modulate immune responses. The T cells contacted with the therapeutic agents can be any cell which expresses the T cell receptor, including α/β and γ/δ T cell receptors. T-cells include all cells which express CD3, including T-cell subsets which also express CD4 and CDS. T-cells include both naive and memory cells and effector cells such as CTL. T-cells also include cells such as Th1, Tc1, Th2, Tc2, Th3, Th17, Th22, Treg, and Tr1 cells. T-cells also include NKT-cells and similar unique classes of the T-cell lineage.

LSR blockade may also be combined with standard cancer treatments. LSR blockade may be effectively combined with chemotherapeutic regimes. In these instances, it may be possible to reduce the dose of chemotherapeutic reagent administered. An example of such a combination is an anti-LSR antibody in combination with Temsirolimus for the treatment of late stage renal cell cancer. Another example of such a combination is an anti-LSR antibody in combination with interleukin-2 (IL-2) for the treatment of late stage renal cell cancer.as well as combination with Ipilimumab or BMS-936558. The scientific rationale behind the combined use of LSR blockade and chemotherapy is that cell death, that is a consequence of the cytotoxic action of most chemotherapeutic compounds, should result in increased levels of tumor antigen in the antigen presentation pathway. Other combination therapies that may result in synergy with LSR blockade through cell death are radiotherapy, cryotherapy, surgery, and hormone deprivation. Other additional combination therapies with additional immunomodulatory molecules will synergistically contribute to the stimulation of the immune system to eradicate the cancer. Each of these protocols creates a source of tumor antigen in the host. Angiogenesis inhibitors may also be combined with LSR blockade. Inhibition of angiogenesis leads to tumor cell death which may feed tumor antigen into host antigen presentation pathways.

LSR blocking antibodies can also be used in combination with bispecific antibodies that target Fc alpha or Fc γ receptor-expressing effectors cells to tumor cells (see, e.g., U.S. Pat. Nos. 5,922,845 and 5,837,243). Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. The T cell arm of these responses would by augmented by the use of LSR blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-beta (Kehrl, J. et al. (1986) J. Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). Antibodies to each of these entities may be used in combination with anti-LSR to counteract the effects of the immunosuppressive agent and favor tumor immune responses by the host.

Other antibodies which may be used to activate host immune responsiveness can be used in combination with anti-LSR. These include molecules on the surface of dendritic cells which activate DC function and antigen presentation. Anti-CD40 antibodies are able to substitute effectively for T cell helper activity (Ridge, J. et al. (1998) Nature 393: 474-478) and can be used in conjunction with LSR antibodies (Ito, N. et al. (2000) Immunobiology 201 (5) 527-40). Activating antibodies to T cell costimulatory molecules such as OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al. (1999) Nature 397: 262-266) as well as antibodies which block the activity of negative costimulatory molecules such as CTLA-4 (e.g., U.S. Pat. No. 5,811,097, implimumab) or BTLA (Watanabe, N. et al. (2003) Nat Immunol 4:670-9), B7-H4 (Sica, G L et al. (2003) Immunity 18:849-61) PD-1 (may also provide for increased levels of T cell activation. Bone marrow transplantation is currently being used to treat a variety of tumors of hematopoietic origin. While graft versus host disease is a consequence of this treatment, therapeutic benefit may be obtained from graft vs. tumor responses. LSR blockade can be used to increase the effectiveness of the donor engrafted tumor specific T cells.

There are also several experimental treatment protocols that involve ex vivo activation and expansion of antigen specific T cells and adoptive transfer of these cells into recipients in order to antigen-specific T cells against tumor (Greenberg, R. & Riddell, S. (1999) Science 285: 546-51). These methods may also be used to activate T cell responses to infectious agents such as CMV. Ex vivo activation in the presence of anti-LSR antibodies may be expected to increase the frequency and activity of the adoptively transferred T cells.

Optionally, antibodies to LSR can be combined with an immunogenic agent, such as cancerous cells, purified tumor antigens (including recombinant proteins, peptides, and carbohydrate molecules), cells, and cells transfected with genes encoding immune stimulating cytokines (He et al (2004) J. Immunol. 173:4919-28). Non-limiting examples of tumor vaccines that can be used include peptides of MUC1 for treatment of colon cancer, peptides of MUC-1/CEA/TRICOM for the treatment of ovary cance, or tumor cells transfected to express the cytokine GM-CSF (discussed further below).

In humans, some tumors have been shown to be immunogenic such as RCC. It is anticipated that by raising the threshold of T cell activation by LSR blockade, we may expect to activate tumor responses in the host.

LSR blockade is likely to be most effective when combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci. U.S.A. 90: 3539-43).

The study of gene expression and large scale gene expression patterns in various tumors has led to the definition of so-called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. LSR blockade may be used in conjunction with a collection of recombinant proteins and/or peptides expressed in a tumor in order to generate an immune response to these proteins. These proteins are normally viewed by the immune system as self antigens and are therefore tolerant to them. The tumor antigen may also include the protein telomerase, which is required for the synthesis of telomeres of chromosomes and which is expressed in more than 85% of human cancers and in only a limited number of somatic tissues (Kim, N et al. (1994) Science 266: 2011-2013). (These somatic tissues may be protected from immune attack by various means). Tumor antigen may also be “neo-antigens” expressed in cancer cells because of somatic mutations that alter protein sequence or create fusion proteins between two unrelated sequences (i.e. bcr-abl in the Philadelphia chromosome), or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with LSR blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot, R & Srivastava, P (1995) Science 269:1585-1588; Tamura, Y. et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle, F. et al. (1998) Nature Medicine 4: 328-332). DCs may also be transduced by genetic means to express these tumor antigens as well. DCs have also been fused directly to tumor cells for the purposes of immunization (Kugler, A. et al. (2000) Nature Medicine 6:332-336). As a method of vaccination, DC immunization may be effectively combined with LSR blockade to activate more potent anti-tumor responses.

### Use of the Therapeutic Agents According to at Least Some Embodiments of the Invention as Adjuvant for Cancer Vaccination:

Immunization against tumor-associated antigens (TAAs) is a promising approach for cancer therapy and prevention, but it faces several challenges and limitations, such as tolerance mechanisms associated with self-antigens expressed by the tumor cells. Costimulatory molecules such as B7.1 (CD80) and B7.2 (CD86) have improved the efficacy of gene-based and cell-based vaccines in animal models and are under investigation as adjuvant in clinical trials. This adjuvant activity can be achieved either by enhancing the costimulatory signal or by blocking inhibitory signal that is transmitted by negative costimulators expressed by tumor cells (Neighbors et al., 2008 J Immunother.; 31(7):644-55).

According to at least some embodiments of the invention, any one of polyclonal or monoclonal antibody and/or antigen binding fragments and/or conjugates containing same, and/or alternative scaffolds, specific to any one of LSR proteins, can be used as adjuvant for cancer vaccination. According to at least some embodiments, the invention provides methods for improving immunization against TAAs, comprising administering to a patient an effective amount of any one of polyclonal or monoclonal antibody and/or antigen binding fragments and/or conjugates containing same, and/or alternative scaffolds, specific to any one of LSR proteins.

### Use of the Therapeutic Agents According to at Least Some Embodiments of the Invention for Immunoenhancement

### 1. Treatment of Cancer

The therapeutic agents provided herein are generally useful in vivo and ex vivo as immune response-stimulating therapeutics. In general, the disclosed therapeutic agent compositions are useful for treating a subject having or being predisposed to any disease or disorder to which the subject's immune system mounts an immune response. The ability of therapeutic agents to modulate LSR immune signals enable a more robust immune response to be possible. The therapeutic agents according to at least some embodiments of the invention are useful to stimulate or enhance immune responses involving immune cells, such as T cells.

The therapeutic agents according to at least some embodiments of the invention are useful for stimulating or enhancing an immune response in host for treating cancer by administering to a subject an amount of a therapeutic agent effective to stimulate T cells in the subject.

### 2. Use of the Therapeutic Agents in Vaccines

The therapeutic agents according to at least some embodiments of the invention, are administered alone or in combination with any other suitable treatment. In one embodiment the therapeutic agents can be administered in conjunction with, or as a component of a vaccine composition as described above. The therapeutic agents according to at least some embodiments of the invention can be administered prior to, concurrently with, or after the administration of a vaccine. In one embodiment the therapeutic agents is administered at the same time as administration of a vaccine.

### Use of Antibodies and Pharmaceutical Compositions for Treatment of Autoimmune Disease

According to at least some embodiments, antibodies and pharmaceutical compositions as described herein may optionally be used for treating an immune system related disease.

Optionally, the immune system related condition comprises an immune related condition, autoimmune diseases as recited herein, transplant rejection and graft versus host disease and/or for blocking or promoting immune costimulation mediated by LSR, immune related diseases as recited herein and/or for immunotherapy (promoting or inhibiting immune costimulation).

Optionally the immune condition is selected from autoimmune disease, transplant rejection, or graft versus host disease.

Optionally the treatment is combined with another moiety useful for treating immune related condition.

Optionally the moiety is selected from the group consisting of immunosuppressants such as corticosteroids, cyclosporin, cyclophosphamide, prednisone, azathioprine, methotrexate, rapamycin, tacrolimus, leflunomide or an analog thereof; mizoribine; mycophenolic acid; mycophenolate mofetil; 15-deoxyspergualine or an analog thereof; biological agents such as TNF-alpha blockers or antagonists, or any other biological agent targeting any inflammatory cytokine, nonsteroidal antiinflammatory drugs/Cox-2 inhibitors, hydroxychloroquine, sulphasalazopryine, gold salts, etanercept, infliximab, mycophenolate mofetil, basiliximab, atacicept, rituximab, cytoxan, interferon beta-1a, interferon beta-1b, glatiramer acetate, mitoxantrone hydrochloride, anakinra and/or other biologics and/or intravenous immunoglobulin (IVIG), interferons such as IFN-beta-1a (REBIF®. AVONEX® and CINNOVEX®) and IFN-beta-1b (BETASERON®); EXTAVIA®, BETAFERON®, ZIFERON®); glatiramer acetate (COPAXONE®), a polypeptide; natalizumab (TYSABRI®), mitoxantrone (NOVANTRONE®), a cytotoxic agent, a calcineurin inhibitor, e.g. cyclosporin A or FK506; an immunosuppressive macrolide, e.g. rapamycine or a derivative thereof; e.g. 40-O-(2-hydroxy)ethyl-rapamycin, a lymphocyte homing agent, e.g. FTY720 or an analog thereof, corticosteroids; cyclophosphamide; azathioprene; methotrexate; leflunomide or an analog thereof; mizoribine; mycophenolic acid; mycophenolate mofetil; 15-deoxyspergualine or an analog thereof; immunosuppressive monoclonal antibodies, e.g., monoclonal antibodies to leukocyte receptors, e.g., MHC, CD2, CD3, CD4, CD11a/CD18, CD7, CD25, CD27, B7, CD40, CD45, CD58, CD137, ICOS, CD150 (SLAM), OX40, 4-1BB or their ligands; or other immunomodulatory compounds, e.g. CTLA4-Ig (abatacept, ORENCIA®, belatacept), CD28-Ig, B7-H4-Ig, or other costimulatory agents, or adhesion molecule inhibitors, e.g. mAbs or low molecular weight inhibitors including LFA-1 antagonists, Selectin antagonists and VLA-4 antagonists, or another immunomodulatory agent.

Thus, treatment of multiple sclerosis using the agents according to at least some embodiments of the present invention may be combined with, for example, any known therapeutic agent or method for treating multiple sclerosis. Non-limiting examples of such known therapeutic agent or method for treating multiple sclerosis include interferon class, IFN-beta-1a (REBIF®. AVONEX® and CINNOVEX®) and IFN-beta-1b (BETASERON®, EXTAVIA®, BETAFERON®, ZIFERON®); glatiramer acetate (COPAXONE®), a polypeptide; natalizumab (TYSABRI®); and mitoxantrone (NOVANTRONE®), a cytotoxic agent, Fampridine (AMPYRA®). Other drugs include corticosteroids, methotrexate, cyclophosphamide, azathioprine, and intravenous immunoglobulin (IVIG), inosine, Ocrelizumab (R1594), Mylinax (Caldribine), alemtuzumab (Campath), daclizumab (Zenapax), Panaclar/dimethyl fumarate (BG-12), Teriflunomide (HMR1726), fingolimod (FTY720), laquinimod (ABR216062), as well as Haematopoietic stem cell transplantation, Neurovax, Rituximab (Rituxan) BCG vaccine, low dose naltrexone, helminthic therapy, angioplasty, venous stents, and alternative therapy, such as vitamin D, polyunsaturated fats, medical marijuana.

Thus, treatment of rheumatoid arthritis, using the agents according to at least some embodiments of the present invention may be combined with, for example, any known therapeutic agent or method for treating rheumatoid arthritis. Non-limiting examples of such known therapeutic agents or methods for treating rheumatoid arthritis include glucocorticoids, nonsteroidal anti-inflammatory drug (NSAID) such as salicylates, or cyclooxygenase-2 inhibitors, ibuprofen and naproxen, diclofenac, indomethacin, etodolac Disease-modifying antirheumatic drugs (DMARDs)-Oral DMARDs: Auranofin (Ridaura), Azathioprine (Imuran), Cyclosporine (Sandimmune, Gengraf, Neoral, generic), D-Penicillamine (Cuprimine), Hydroxychloroquine (Plaquenil), IM gold Gold sodium thiomalate (Myochrysine) Aurothioglucose (Solganal), Leflunomide (Arava), Methotrexate (Rheumatrex), Minocycline (Minocin), Staphylococcal protein A immunoadsorption (Prosorba column), Sulfasalazine (Azulfidine). Biologic DMARDs: TNF-α blockers including Adalimumab (Humira), Etanercept (Enbrel), Infliximab (Remicade), golimumab (Simponi), certolizumab pegol (Cimzia), and other Biological DMARDs, such as Anakinra (Kineret), Rituximab (Rituxan), Tocilizumab (Actemra), CD28 inhibitor including Abatacept (Orencia) and B elatacept.

Thus, treatment of IBD, using the agents according to at least some embodiments of the present invention may be combined with, for example, any known therapeutic agent or method for treating IBD. Non-limiting examples of such known therapeutic agents or methods for treating IBD include immunosuppression to control the symptom, such as prednisone, Mesalazine (including Asacol, Pentasa, Lialda, Aspiro),azathioprine (Imuran), methotrexate, or 6-mercaptopurine, steroids, Ondansetron, TNF-α blockers (including infliximab, adalimumab golimumab, certolizumab pegol), Orencia (abatacept), ustekinumab (Stelara®), Briakinumab (ABT-874), Certolizumab pegol (Cimzia®), ITF2357 (givinostat), Natalizumab (Tysabri), Firategrast (SB-683699), Remicade (infliximab), vedolizumab (MLN0002), other drugs including GSK1605786 CCX282-B (Traficet-EN), AJM300, Stelara (ustekinumab), Semapimod (CNI-1493) tasocitinib (CP-690550), LMW Heparin MMX, Budesonide MMX, Simponi (golimumab), MultiStem®, Gardasil HPV vaccine, Epaxal Berna (virosomal hepatitis A vaccine), surgery, such as bowel resection, strictureplasty or a temporary or permanent colostomy or ileostomy; antifungal drugs such as nystatin (a broad spectrum gut antifungal) and either itraconazole (Sporanox) or fluconazole (Diflucan); alternative medicine, prebiotics and probiotics, cannabis, Helminthic therapy or ova of the Trichuris suis helminth.

Thus, treatment of psoriasis, using the agents according to at least some embodiments of the present invention may be combined with, for example, any known therapeutic agent or method for treating psoriasis. Non-limiting examples of such known therapeutics for treating psoriasis include topical agents, typically used for mild disease, phototherapy for moderate disease, and systemic agents for severe disease. Non-limiting examples of topical agents: bath solutions and moisturizers, mineral oil, and petroleum jelly; ointment and creams containing coal tar, dithranol (anthralin), corticosteroids like desoximetasone (Topicort), Betamethasone, fluocinonide, vitamin D3 analogues (for example, calcipotriol), and retinoids. Non-limiting examples of phototherapy: sunlight; wavelengths of 311-313 nm, psoralen and ultraviolet A phototherapy (PUVA). Non-limiting examples of systemic agents: Biologics, such as interleukin antagonists, TNF-α blockers including antibodies such as infliximab (Remicade), adalimumab (Humira), golimumab, certolizumab pegol, and recombinant TNF-α decoy receptor, etanercept (Enbrel); drugs that target T cells, such as efalizumab (Xannelim/Raptiva), alefacept (Ameviv), dendritic cells such Efalizumab; monoclonal antibodies (MAbs) targeting cytokines, including anti-IL-12/IL-23 (ustekinumab (brand name Stelara)) and anti-Interleukin-17; Briakinumab (ABT-874); small molecules, including but not limited to ISA247; Immunosuppressants, such as methotrexate, cyclosporine; vitamin A and retinoids (synthetic forms of vitamin A); and alternative therapy, such as changes in diet and lifestyle, fasting periods, low energy diets and vegetarian diets, diets supplemented with fish oil rich in Vitamin A and Vitamin D (such as cod liver oil), Fish oils rich in the two omega-3 fatty acids eicosapentaenoic acid (EPA) and docosahexaenoic acid (DHA) and contain Vitamin E. Ichthyotherapy, Hypnotherapy, cannabis.

Thus, treatment of type 1 diabetes, using the agents according to at least some embodiments of the present invention may be combined with, for example, any known therapeutic agent or method for treating type ldiabetes. Non-limiting examples of such known therapeutics for treating type 1 diabetes include insulin, insulin analogs, islet transplantation, stem cell therapy including PROCHYMAL®, non-insulin therapies such as il-1beta inhibitors including Anakinra (Kineret®), Abatacept (Orencia®), Diamyd, alefacept (Ameviv®), Otelixizumab, DiaPep277 (Hsp60 derived peptide), Alpha 1-Antitrypsin, Prednisone, azathioprine, Ciclosporin, E1-INT (an injectable islet neogenesis therapy comprising an epidermal growth factor analog and a gastrin analog), statins including Zocor®, Simlup®, Simcard®, Simvacor®, Sitagliptin (dipeptidyl peptidase (DPP-4) inhibitor), Anti-CD3 mAb (e.g., Teplizumab); CTLA4-Ig (abatacept), Anti IL-1B eta (Canakinumab), Anti-CD20 mAb (e.g, rituximab).

Thus, treatment of uveitis, using the agents according to at least some embodiments of the present invention may be combined with, for example, any known therapeutic agent or method for treating uveitis. Non-limiting examples of such known therapeutics for treating uveitis include corticosteroids, topical cycloplegics, such as atropine or homatropine, or injection of PSTTA (posterior subtenon triamcinolone acetate), antimetabolite medications, such as methotrexate, TNF-α blockers (including infliximab, adalimumab, etanercept, golimumab, certolizumab pegol).

Thus, treatment for Sjogren's syndrome, using the agents according to at least some embodiments of the present invention may be combined with, for example, any known therapeutic agent or method for treating for Sjogren's syndrome. Non-limiting examples of such known therapeutics for treating for Sjogren's syndrome include Cyclosporine, pilocarpine (Salagen) and cevimeline (Evoxac), Hydroxychloroquine (Plaquenil), cortisone (prednisone and others) and/or azathioprine (Imuran) or cyclophosphamide (Cytoxan), Dexamethasone, Thalidomide, Dehydroepiandrosterone, NGX267, Rebamipide, FID 114657, Etanercept, Raptiv a, Belimumab, MabThera (rituximab); Anakinra, intravenous immune globulin (IVIG), Allogeneic Mesenchymal Stem Cells (AlloMSC), Automatic neuro-electrostimulation by “Saliwell Crown”.

Thus, treatment for systemic lupus erythematosus, using the agents according to at least some embodiments of the present invention may be combined with, for example, any known therapeutic agent or method for treating for systemic lupus erythematosus. Non-limiting examples of such known therapeutics for treating for systemic lupus erythematosus include corticosteroids and Disease-modifying antirheumatic drugs (DMARDs), commonly anti-malarial drugs such as plaquenil and immunosuppressants (e.g. methotrexate and azathioprine) Hydroxychloroquine, cytotoxic drugs (e.g., cyclophosphamide and mycophenolate), Hydroxychloroquine (HCQ), Benlysta (belimumab), nonsteroidal anti-inflammatory drugs, Prednisone, Cellcept, Prograf, Atacicept, Lupuzor, Intravenous Immunoglobulins (IVIGs), CellCept (mycophenolate mofetil), Orencia, CTLA4-IgG4m (RG2077), rituximab, Ocrelizumab, Epratuzumab, CNTO 136, Sifalimumab (MEDI-545), A-623 (formerly AMG 623), AMG 557, Rontalizumab, paquinimod (ABR-215757), LY2127399, CEP-33457, Dehydroepiandrosterone, Levothyroxine, abetimus sodium (LJP 394), Memantine, Opiates, Rapamycin, Renal transplantation, stem cell transplantation.

The therapeutic agents and/or a pharmaceutical composition comprising same, as recited herein, according to at least some embodiments of the invention, may be administered as the sole active ingredient or together with other drugs in immunomodulating regimens or other anti-inflammatory agents e.g. for the treatment or prevention of alto- or xenograft acute or chronic rejection or inflammatory or autoimmune disorders, or to induce tolerance.

The term “autoimmune disease” as used herein should be understood to encompass any autoimmune disease and chronic inflammatory conditions. According to at least some embodiments of the invention, the autoimmune diseases should be understood to encompass any disease disorder or condition selected from the group including but not limited to multiple sclerosis, including relapsing-remiting multiple sclerosis, primary progressive multiple sclerosis, and secondary progressive multiple sclerosis; psoriasis; rheumatoid arthritis; psoriatic arthritis, systemic lupus erythematosus (SLE); ulcerative colitis; Crohn's disease; benign lymphocytic angiitis, thrombocytopenic purpura, idiopathic thrombocytopenia, idiopathic autoimmune hemolytic anemia, pure red cell aplasia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, cryoglobulinemic vasculitis, ANCA-associated vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, autoimmune atrophic gastritis, pernicious anaemia, pemphigus, pemphigus vulgarus, cirrhosis, primary biliary cirrhosis, dermatomyositis, polymyositis, fibromyositis, myogelosis, celiac disease, immunoglobulin A nephropathy, Henoch-Schonlein purpura, Evans syndrome, atopic dermatitis, psoriasis, psoriasis arthropathica, Graves' disease, Graves' ophthalmopathy, scleroderma, systemic scleroderma, progressive systemic scleroderma, asthma, allergy, primary biliary cirrhosis, Hashimoto's thyroiditis, primary myxedema, sympathetic ophthalmia, autoimmune uveitis, hepatitis, chronic action hepatitis, collagen diseases, ankylosing spondylitis, periarthritis humeroscapularis, panarteritis nodosa, chondrocalcinosis, Wegener's granulomatosis, microscopic polyangiitis, chronic urticaria, bullous skin disorders, pemphigoid, atopic eczema, Devic's disease, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne, normocomplementemic urticarial vasculitis, pericarditis, myositis, anti-synthetase syndrome, scleritis, macrophage activation syndrome, Bechet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, autoimmune retinopathy, age-related macular degeneration, atherosclerosis, chronic prostatitis and TNF receptor-associated periodic syndrome (TRAPS).

Optionally and preferably, the autoimmune disease includes but is not limited to any of the types and subtypes of any of multiple sclerosis, rheumatoid arthritis, type I diabetes, psoriasis, systemic lupus erythematosus, inflammatory bowel disease, uveitis, or Sjogren's syndrome.

As used herein, “multiple sclerosis” comprises one or more of multiple sclerosis, benign multiple sclerosis, relapsing remitting multiple sclerosis, secondary progressive multiple sclerosis, primary progressive multiple sclerosis, progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, and acute multiple sclerosis. Optionally, “conditions relating to multiple sclerosis” include, e.g., Devic's disease, also known as Neuromyelitis Optica; acute disseminated encephalomyelitis, acute demyelinating optic neuritis, demyelinative transverse myelitis, Miller-Fisher syndrome, encephalomyelradiculoneuropathy, acute demyelinative polyneuropathy, tumefactive multiple sclerosis and Balo's concentric sclerosis.

As used herein, “rheumatoid arthritis” comprises one or more of rheumatoid arthritis, gout and pseudo-gout, juvenile idiopathic arthritis, juvenile rheumatoid arthritis, Still's disease, ankylosing spondylitis, rheumatoid vasculitis. Optionally, conditions relating to rheumatoid arthritis include, e.g., osteoarthritis, sarcoidosis, Henoch-Schönlein purpura, Psoriatic arthritis, Reactive arthritis, Spondyloarthropathy, septic arthritis, Haemochromatosis, Hepatitis, vasculitis, Wegener's granulomatosis, Lyme disease, Familial Mediterranean fever, Hyperimmunoglobulinemia D with recurrent fever, TNF receptor associated periodic syndrome, and Enteropathic arthritis associated with inflammatory bowel disease.

As used herein, “Uveitis” comprises one or more of uveitis, anterior uveitis (or iridocyclitis), intermediate uveitis (pars planitis), posterior uveitis (or chorioretinitis) and the panuveitic form.

As used herein, “inflammatory bowel disease” comprises one or more of inflammatory bowel disease Crohn's disease, ulcerative colitis (UC), Collagenous colitis, Lymphocytic colitis, Ischaemic colitis, Diversion colitis, Behget's disease, Indeterminate colitis.

As used herein, “psoriasis” comprises one or more of psoriasis, Nonpustular Psoriasis including Psoriasis vulgaris and Psoriatic erythroderma (erythrodermic psoriasis), Pustular psoriasis including Generalized pustular psoriasis (pustular psoriasis of von Zumbusch), Pustulosis palmaris et plantaris (persistent palmoplantar pustulosis, pustular psoriasis of the Barber type, pustular psoriasis of the extremities), Annular pustular psoriasis, Acrodermatitis continua, Impetigo herpetiformis. Optionally, conditions relating to psoriasis include, e.g., drug-induced psoriasis, Inverse psoriasis, Napkin psoriasis, Seborrheic-like psoriasis, Guttate psoriasis, Nail psoriasis, Psoriatic arthritis.

As used herein, “type 1 diabetes” comprises one or more of type 1 diabetes, insulin-dependent diabetes mellitus, idiopathic diabetes, juvenile type ldiabetes, maturity onset diabetes of the young, latent autoimmune diabetes in adults, gestational diabetes. Conditions relating to type 1 diabetes include, neuropathy including polyneuropathy, mononeuropathy, peripheral neuropathy and autonomicneuropathy; eye complications: glaucoma, cataracts, retinopathy.

As used herein, “Sjogren's syndrome” comprises one or more of Sjogren's syndrome, Primary Sjogren's syndrome and Secondary Sjogren's syndrome, as well as conditions relating to Sjogren's syndrome including connective tissue disease, such as rheumatoid arthritis, systemic lupus erythematosus, or scleroderma. Other complications include pneumonia, pulmonary fibrosis, interstitial nephritis, inflammation of the tissue around the kidney's filters, glomerulonephritis, renal tubular acidosis, carpal tunnel syndrome, peripheral neuropathy, cranial neuropathy, primary biliary cirrhosis (PBC), cirrhosis, Inflammation in the esophagus, stomach, pancreas, and liver (including hepatitis), Polymyositis, Raynaud's phenomenon, Vasculitis, Autoimmune thyroid problems, lymphoma.

As used herein, “systemic lupus erythematosus”, comprises one or more of systemic lupus erythematosus, discoid lupus, lupus arthritis, lupus pneumonitis, lupus nephritis. Conditions relating to systemic lupus erythematosus include osteoarticular tuberculosis, antiphospholipid antibody syndrome, inflammation of various parts of the heart, such as pericarditis, myocarditis, and endocarditis, Lung and pleura inflammation, pleuritis, pleural effusion, chronic diffuse interstitial lung disease, pulmonary hypertension, pulmonary emboli, pulmonary hemorrhage, and shrinking lung syndrome, lupus headache, Guillain-Barré syndrome, aseptic meningitis, demyelinating syndrome, mononeuropathy, mononeuritis multiplex, myasthenia gravis, myelopathy, cranial neuropathy, polyneuropathy, vasculitis.

The term “immune related disease (or disorder or condition)” as used herein should be understood to encompass any disease disorder or condition selected from the group including but not limited to autoimmune diseases, inflammatory disorders and immune disorders associated with graft transplantation rejection, such as acute and chronic rejection of organ transplantation, allogenic stem cell transplantation, autologous stem cell transplantation, bone marrow tranplantation, and graft versus host disease.

As used herein the term “inflammatory disorders” and/or “inflammation”, used interchangeably, includes inflammatory abnormalities characterized by disregulated immune response to harmful stimuli, such as pathogens, damaged cells, or irritants. Inflammatory disorders underlie a vast variety of human diseases. Non-immune diseases with etiological origins in inflammatory processes include cancer, atherosclerosis, and ischaemic heart disease. Examples of disorders associated with inflammation include: Chronic prostatitis, Glomerulonephritis, Hypersensitivities, Pelvic inflammatory disease, Reperfusion injury, Sarcoidosis, Vasculitis, Interstitial cystitis, normocomplementemic urticarial vasculitis, pericarditis, myositis, anti-synthetase syndrome, scleritis, macrophage activation syndrome, Bechet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, TNF receptor-associated periodic syndrome (TRAPSP), gingivitis, periodontitis, hepatitis, cirrhosis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne.

### Use of Antibodies and Pharmaceutical Compositions for Treatment of Infectious Disease

According to at least some embodiments, antibodies and pharmaceutical compositions as described herein may optionally be used for treating infectious disease.

Chronic infections are often characterized by varying degrees of functional impairment of virus-specific T-cell responses, and this defect is a principal reason for the inability of the host to eliminate the persisting pathogen. Although functional effector T cells are initially generated during the early stages of infection, they gradually lose function during the course of the chronic infection as a result of persistant exposure to foreign antigen, giving rise to T cell exhaustion. Exhausted T cells express high levels of multiple co-inhibitory receptors such as CTLA-4, PD-1, and LAG3 (Crawford et al., Curr Opin Immunol. 2009; 21:179-186; Kaufmann et al., J Immunol 2009; 182:5891-5897, Sharpe et al., Nat Immunol 2007; 8:239-245). PD-1 overexpression by exhausted T cells was observed clinically in patients suffering from chronic viral infections including HIV, HCV and HBV (Crawford et al., Curr Opin Immunol 2009; 21:179-186; Kaufmann et al., J Immunol 2009; 182:5891-5897, Sharpe et al., Nat Immunol 2007; 8:239-245). There has been some investigation into this pathway in additional pathogens, including other viruses, bacteria, and parasites (Hofineyer et al., J Biomed Biotechnol. Vol 2011, Art. ID 451694, Bhadra et al., Proc Natl Acad. Sci. 2011; 108(22):9196-201). For example, the PD-1 pathway was shown to be involved in controlling bacterial infection using a sepsis model induced by the standard cecal ligation and puncture method. The absence of PD-1 in knockout mice protected from sepsis-induced death in this model (Huang et al., PNAS 2009: 106; 6303-6308).

T cell exhaustion can be reversed by blocking co-inhibitory pathways such as PD-1 or CTLA-4 (Rivas et al., J. Immunol. 2009; 183:4284-91; Golden-Mason et al., J. Virol. 2009; 83:9122-30; Hofineyer et al., J Biomed Biotechnol. Vol 2011, Art. ID 451694), thus allowing restoration of anti viral immune function. The therapeutic potential of co-inhibition blockade for treating viral infection was extensively studied by blocking the PD-1/PD-L1 pathway, which was shown to be efficacious in several animal models of infection including acute and chronic simian immunodeficiency virus (SIV) infection in rhesus macaques (Valu et al., Nature 2009; 458:206-210) and in mouse models of chronic viral infection, such as lymphocytic choriomeningitis virus (LCMV) (Barber et al., Nature. 2006; 439:682-7), and Theiler's murine encephalomyelitis virus (TMEV) model in SJL/J mice (Duncan and Miller PLoS One. 2011; 6:e18548). In these models PD-1/PD-L1 blockade improved anti viral responses and promoted clearance of the persisting viruses. In addition, PD-1/PD-L1 blockade increased the humoral immunity manifested as elevated production of specific anti-virus antibodies in the plasma, which in combination with the improved cellular responses leads to decrease in plasma viral loads and increased survival.

As used herein the term “infectious disorder and/or disease” and/or “infection”, used interchangeably, includes any disorder, disease and/or condition caused by presence and/or growth of pathogenic biological agent in an individual host organism. As used herein the term “infection” comprises the disorder, disease and/or condition as above, exhibiting clinically evident illness (i.e., characteristic medical signs and/or symptoms of disease) and/or which is asymtomatic for much or all of it course. As used herein the term “infection” also comprises disorder, disease and/or condition caused by persistence of foreign antigen that lead to exhaustion T cell phenotype characterized by impaired functionality which is manifested as reduced proliferation and cytokine production. As used herein the term “infectious disorder and/or disease” and/or “infection”, further includes any of the below listed infectious disorders, diseases and/or conditions, caused by a bacterial infection, viral infection, fungal infection and/or parasite infection. As used herein the term “viral infection” comprises any infection caused by a virus, optionally including but not limited to Retroviridae (e.g., human immunodeficiency viruses, such as HIV-1 or HIV-2, acquired immune deficiency (AIDS) also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever virus); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herperviridae (herpes simplex virus (HSV) 1 and 2, varicella zoster virus, cytomegalovirus (CMV), herpes viruses); Poxyiridae (variola virsues, vaccinia viruses, pox viruses); and Iridoviridae (e.g., African swine fever virus); and unclassified viruses (e.g., the etiological agents of Spongiform encephalopathies, the agent of delta hepatitides (thought to be a defective satellite of hepatitis B virus), the agents of non-A, non-B hepatitis (class 1-internally transmitted; class 2-parenterally transmitted (i.e., Hepatitis C); Norwalk and related viruses, and astroviruses) as well as Severe acute respiratory syndrome virus and respiratory syncytial virus (RSV).

As used herein the term “fungal infection” comprises any infection caused by a fungi, optionally including but not limited to Cryptococcus neoformans, Histoplasma capsulatum, Coccidioides immitis, Blastomyces dermatitidis, Chlamydia trachomatis, Candida albicans.

As used herein the term “parasite infection” comprises any infection caused by a parasite, optionally including but not limited to protozoa, such as Amebae, Flagellates, Plasmodium falciparum, Toxoplasma gondii, Ciliates, Coccidia, Microsporidia, Sporozoa; helminthes, Nematodes (Roundworms), Cestodes (Tapeworms), Trematodes (Flukes), Arthropods, and aberrant proteins known as prions.

An infectious disorder and/or disease caused by bacteria may optionally comprise one or more of Sepsis, septic shock, sinusitis, skin infections, pneumonia, bronchitis, meningitis, Bacterial vaginosis, Urinary tract infection (UCI), Bacterial gastroenteritis, Impetigo and erysipelas, Erysipelas, Cellulitis, anthrax, whooping cough, lyme disease, Brucellosis, enteritis, acute enteritis, Tetanus, diphtheria, Pseudomembranous colitis, Gas gangrene, Acute food poisoning, Anaerobic cellulitis, Nosocomial infections, Diarrhea, Meningitis in infants, Traveller's diarrhea, Hemorrhagic colitis, Hemolytic-uremic syndrome, Tularemia, Peptic ulcer, Gastric and Duodenal ulcers, Legionnaire's Disease, Pontiac fever, Leptospirosis, Listeriosis, Leprosy (Hansen's disease), Tuberculosis, Gonorrhea, Ophthalmia neonatorum, Septic arthritis, Meningococcal disease including meningitis, Waterhouse-Friderichsen syndrome, Pseudomonas infection, Rocky mountain spotted fever, Typhoid fever type salmonellosis, Salmonellosis with gastroenteritis and enterocolitis, Bacillary dysentery/Shigellosis, Coagulase-positive staphylococcal infections: Localized skin infections including Diffuse skin infection (Impetigo), Deep localized infections, Acute infective endocarditis, Septicemia, Necrotizing pneumonia, Toxinoses such as Toxic shock syndrome and Staphylococcal food poisoning, Cystitis, Endometritis, Otitis media, Streptococcal pharyngitis, Scarlet fever, Rheumatic fever, Puerperal fever, Necrotizing fasciitis, Cholera, Plague (including Bubonic plague and Pneumonic plague), as well as any infection caused by a bacteria selected from but not limited to Helicobacter pyloris, Boreliai burgdorferi, Legionella pneumophilia, Mycobacteria sps (e.g., M. tuberculosis, M. avium, M. Intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter erogenes, Klebsiella pneuomiae, Pasturella multicoda, Bacteroides sp., Fusobacterium nucleatum, Sreptobacillus moniliformis, Treponema pallidium, Treponema pertenue, Leptospira, and Actinomeyces israelli.

Non limiting examples of infectious disorder and/or disease caused by virus is selected from the group consisting of but not limited to acquired immune deficiency (AIDS), West Nile encephalitis, coronavirus infection, rhinovirus infection, influenza, dengue, hemorrhagic fever; an otological infection; severe acute respiratory syndrome (SARS), acute febrile pharyngitis, pharyngoconjunctival fever, epidemic keratoconjunctivitis, infantile gastroenteritis, infectious mononucleosis, Burkitt lymphoma, acute hepatitis, chronic hepatitis, hepatic cirrhosis, hepatocellular carcinoma, primary HSV-1 infection, (gingivostomatitis in children, tonsillitis & pharyngitis in adults, keratoconjunctivitis), latent HSV-1 infection (herpes labialis, cold sores), aseptic meningitis, Cytomegalovirus infection, Cytomegalic inclusion disease, Kaposi sarcoma, Castleman disease, primary effusion lymphoma, influenza, measles, encephalitis, postinfectious encephalomyelitis, Mumps, hyperplastic epithelial lesions (common, flat, plantar and anogenital warts, laryngeal papillomas, epidermodysplasia verruciformis), croup, pneumonia, bronchiolitis, Poliomyelitis, Rabies, bronchiolitis, pneumonia, German measles, congenital rubella, Hemorrhagic Fever, Chickenpox, Dengue, Ebola infection, Echovirus infection, EBV infection, Fifth Disease, Filovirus, Flavivirus, Hand, foot & mouth disease, Herpes Zoster Virus (Shingles), Human Papilloma Virus Associated Epidermal Lesions, Lassa Fever, Lymphocytic choriomeningitis, Parainfluenza Virus Infection, Paramyxovirus, Parvovirus B19 Infection, Picornavirus, Poxviruses infection, Rotavirus diarrhea, Rubella, Rubeola, Varicella, Variola infection.

An infectious disorder and/or disease caused by fungi optionally includes but is not limited to Allergic bronchopulmonary aspergillosis, Aspergilloma, Aspergillosis, Basidiobolomycosis, Blastomycosis, Candidiasis, Chronic pulmonary aspergillosis, Chytridiomycosis, Coccidioidomycosis, Conidiobolomycosis, Covered smut (barley), Cryptococcosis, Dermatophyte, Dermatophytid, Dermatophytosis, Endothrix, Entomopathogenic fungus, Epizootic lymphangitis, Epizootic ulcerative syndrome, Esophageal candidiasis, Exothrix, Fungemia, Histoplasmosis, Lobomycosis, Massospora cicadina, Mycosis, Mycosphaerella fragariae, Myringomycosis, Paracoccidioidomycosis, Pathogenic fungi, Penicilliosis, Thousand cankers disease, Tinea, Zeaspora, Zygomycosis.Non limiting examples of infectious disorder and/or disease caused by parasites is selected from the group consisting of but not limited to Acanthamoeba, Amoebiasis, Ascariasis, Ancylostomiasis, Anisakiasis, Babesiosis, Balantidiasis, Baylisascariasis, Blastocystosis, Candiru, Chagas disease, Clonorchiasis, Cochliomyia, Coccidia, Chinese Liver Fluke Cryptosporidiosis, Dientamoebiasis, Diphyllobothriasis, Dioctophyme renalis infection, Dracunculiasis, Echinococcosis, Elephantiasis, Enterobiasis, Fascioliasis, Fasciolopsiasis, Filariasis, Giardiasis, Gnathostomiasis, Hymenolepiasis, Halzoun Syndrome, Isosporiasis, Katayama fever, Leishmaniasis, lymphatic filariasis, Malaria, Metagonimiasis, Myiasis, Onchocerciasis, Pediculosis, Primary amoebic meningoencephalitis, Parasitic pneumonia, Paragonimiasis, Scabies, Schistosomiasis, Sleeping sickness, Strongyloidiasis, Sparganosis, Rhinosporidiosis, River blindness, Taeniasis (cause of Cysticercosis), Toxocariasis, Toxoplasmosis, Trichinosis, Trichomoniasis, Trichuriasis, Trypanosomiasis, Tapeworm infection.

A preferred example of infectious disease is a disease caused by any of hepatitis B, hepatitis C, infectious mononucleosis, EBV, cytomegalovirus, AIDS, HIV-1, HIV-2, tuberculosis, malaria and schistosomiasis.

According to at least some embodiments of the present invention, there is provided use of a combination of thetherapeutic agents and/or a pharmaceutical composition comprising same, as recited herein, and a known therapeutic agent effective for treating infection.

The therapeutic agents and/or a pharmaceutical composition comprising same, as recited herein, can be administered in combination with one or more additional therapeutic agents used for treatment of bacterial infections, including, but not limited to, antibiotics including Aminoglycosides, Carbapenems, Cephalosporins, Macrolides, Lincosamides, Nitrofurans, penicillins, Polypeptides, Quinolones, Sulfonamides, Tetracyclines, drugs against mycobacteria including but not limited to Clofazimine, Cycloserine, Cycloserine, Rifabutin, Rifapentine, Streptomycin and other antibacterial drugs such as Chloramphenicol, Fosfomycin, Metronidazole, Mupirocin, and Timidazole.

The therapeutic agents and/or a pharmaceutical composition comprising same, as recited herein, can be administered in combination with one or more additional therapeutic agents used for treatment of viral infections, including, but not limited to, antiviral drugs such as oseltamivir (brand name Tamiflu) and zanamivir (brand name Relenza) Arbidol—adamantane derivatives (Amantadine, Rimantadine)—neuraminidase inhibitors (Oseltamivir, Laninamivir, Peramivir, Zanamivir) nucleotide analog reverse transcriptase inhibitor including Purine analogue guanine (Aciclovir#/Valacyclovir, Ganciclovir/Valganciclovir, Penciclovir/Famciclovir) and adenine (Vidarabine), Pyrimidine analogue, uridine (Idoxuridine, Trifluridine, Edoxudine), thymine

(Brivudine), cytosine (Cytarabine); Foscarnet; Nucleoside analogues/NARTIs: Entecavir, Lamivudine, Telbivudine, Clevudine; Nucleotide analogues/NtRTIs: Adefovir, Tenofovir; Nucleic acid inhibitors such as Cidofovir; InterferonInterferon alfa-2b, Peginterferon alfa-2a; Ribavirin#/Taribavirin; antiretroviral drugs including zidovudine, lamivudine, abacavir, lopinavir, ritonavir, tenofovir/emtricitabine, efavirenz each of them alone or a various combinations, gp41 (Enfuvirtide), Raltegravir, protease inhibitors such as Fosamprenavir, Lopinavir and Atazanavir, Methisazone, Docosanol, Fomivirsen, Tromantadine.

The therapeutic agents and/or a pharmaceutical composition comprising same, as recited herein, can be administered in combination with one or more additional therapeutic agents used for treatment of fungal infections, including, but not limited to, antifungal drugs of the Polyene antifungals, Imidazole, triazole, and thiazole antifungals, Allylamines, Echinocandins or other anti fungal drugs.

### Pharmaceutical Compositions

In another aspect, the present invention provides a composition, e.g., a pharmaceutical composition, containing one or a combination of the therapeutic agent, according to at least some embodimants of the invention.

Thus, the present invention features a pharmaceutical composition comprising a therapeutically effective amount of a therapeutic agent according to at least some embodiments of the present invention.

The pharmaceutical composition according to at least some embodiments of the present invention is further preferably used for the treatment of cancer, as recited herein.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder. “Mammal” for purposes of treatment refers to any animal classified as a mammal, including humans, domestic and farm animals, and zoo, sports, or pet animals, such as dogs, horses, cats, cows, etc. Preferably, the mammal is human.

The term “therapeutically effective amount” refers to an amount of agent according to the present invention that is effective to treat a disease or disorder in a mammal.

The therapeutic agents of the present invention can be provided to the subject alone, or as part of a pharmaceutical composition where they are mixed with a pharmaceutically acceptable carrier.

A composition is said to be a “pharmaceutically acceptable carrier” if its administration can be tolerated by a receipient patient. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

Such compositions include sterile water, buffered saline (e.g., Tris-HCl, acetate, phosphate), pH and ionic strength and optionally additives such as detergents and solubulizing agents (e.g., Polysorbate 20, Polysorbate 80), antioxidants (e.g, ascorbic acid, sodium metabisulfite), preservatives (e.g, Thimersol, benzyl alcohol) and blulking substances (e.g., lactose, manitol). Non-aqueoes solvents or vehicles may also be used as detailed below.

Examples of suitable aqueous and nonaqueous carriers that may be employed in the pharmaceutical compositions according to at least some embodiments of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Depending on the route of administration, the active compound, i.e., monoclonal or polyclonal antibodies and antigen binding fragments and conjugates containing same, and/or alternative scaffolds, that specifically bind any one of LSR proteins, or bispecific molecule, may be coated in a material to protect the compound from the action of acids and other natural conditions that may inactivate the compound. The pharmaceutical compounds according to at least some embodiments of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the invention include intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intravenous and intradermal), transmucosal (e.g., sublingual administration), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In a specific embodiment, a protein, a therapeutic agent or a pharmaceutical composition according to at least some embodiments of the present invention can be administered intraperitoneally or intravenously.

Alternatively, an LSR specific antibody or can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition according to at least some embodiments of the invention can be administered with a needles hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-LSR antibodies can be formulated to ensure proper distribution in vivo. For example, the blood-brain bather (BBB) excludes many highly hydrophilic compounds. To ensure that the therapeutic compounds according to at least some embodiments of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The anti-LSR antibodies, according to at least some embodiments of the present invention, can be used as neutralizing antibodies. A Neutralizing antibody (Nabs), is an antibody that is capable of binding and neutralizing or inhibiting a specific antigen thereby inhibiting its biological effect, for example by blocking the receptors on the cell or the virus, inhibiting the binding of the virus to the host cell. NAbs will partially or completely abrogate the biological action of an agent by either blocking an important surface molecule needed for its activity or by interfering with the binding of the agent to its receptor on a target cell.

In yet another embodiment, immunoconjugates of the invention can be used to target compounds (e.g., therapeutic agents, labels, cytotoxins, radiotoxins immunosuppressants, etc.) to cells which have LSR cell surface receptors by linking such compounds to the antibody. Thus, the invention also provides methods for localizing ex vivo or in vivo cells expressing LSR (e.g., with a detectable label, such as a radioisotope, a fluorescent compound, an enzyme, or an enzyme co-factor). Alternatively, the immunoconjugates can be used to kill cells which have LSR cell surface receptors by targeting cytotoxins or radiotoxins to LSR antigen.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., soluble polypeptide conjugate containing the ectodomain of the LSR antigen, antibody, immunoconjugate, alternative scaffolds, and/or bispecific molecule, may be coated in a material to protect the compound from the action of acids and other natural conditions that may inactivate the compound. The pharmaceutical compounds according to at least some embodiments of the present invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the present invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions according to at least some embodiments of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the present invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an antibody according to at least some embodiments of the present invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 mug/ml and in some methods about 25-300 microgram/ml.

Alternatively, therapeutic agent can be administered as a sustained release formulation, in which case less frequent administration is required. Dosage and frequency vary depending on the half-life of the therapeutic agent in the patient. In general, human antibodies show the longest half life, followed by humanized antibodies, chimeric antibodies, and nonhuman antibodies. The half-life for fusion proteins may vary widely. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

### Formulations for Parenteral Administration

In a further embodiment, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., water soluble antioxidants such as ascorbic acid, sodium metabisulfite, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, alpha-tocopherol; and metal chelating agents, such as citric acid, ethylenediamine tetraacetic acid (EDTA), sorbitol, tartaric acid, phosphoric acid), and preservatives (e.g., Thimersol, benzyl alcohol) and bulking substances (e.g., lactose, mannitol). Examples of non-aqueous solvents or vehicles are ethanol, propylene glycol, polyethylene glycol, vegetable oils, such as olive oil and corn oil, gelatin, and injectable organic esters such as ethyl oleate. The formulations may be freeze dried (lyophilized) or vacuum dried and redissolved/resuspended immediately before use. The formulation may be sterilized by, for example, filtration through a bacteria retaining filter, by incorporating sterilizing agents into the compositions, by irradiating the compositions, or by heating thecompositions.

Formulations for Topical Administration

LSR polypeptides, fragments, fusion polypeptides, nucleic acids, and vectors disclosed herein can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns. A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges. Transdermal formulations may also be prepared. These will typically be ointments, lotions, sprays, or patches, all of which can be prepared using standard technology. Transdermal formulations will require the inclusion of penetration enhancers.

Controlled Delivery Polymeric Matrices

LSR polypeptides, fragments, fusion polypeptides, nucleic acids, and vectors disclosed herein may also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel. Either non-biodegradable or biodegradable matrices can be used for delivery of polypeptides or nucleic acids encoding the polypeptides, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, δ: 275-283 (1987); and Mathiowitz, et al., J. Appl Polymer ScL, 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

Diagnostic Uses of Anti-LSR Antibodies

According to at least some embodiments of the present invention, the antibodies (e.g., human monoclonal antibodies, multispecific and bispecific molecules and compositions) can be used to detect levels of LSR or levels of cells which contain LSR on their membrane surface, which levels can then be linked to certain disease symptoms. Alternatively, the antibodies can be used to inhibit or block LSR function which, in turn, can be linked to the prevention or amelioration of cancer. This can be achieved by contacting a sample and a control sample with the anti-LSR antibody under conditions that allow for the formation of a complex between the corresponding antibody and LSR. Any complexes formed between the antibody and LSR are detected and compared in the sample and the control.

According to at least some embodiments of the present invention, the antibodies (e.g., human antibodies, multispecific and bispecific molecules and compositions) can be initially tested for binding activity associated with therapeutic or diagnostic use in vitro. For example, compositions according to at least some embodiments of the present invention can be tested using low cytometric assays.

Also within the scope of the present invention are kits comprising the LSR specific antibody according to at least some embodiments of the present invention (e.g., human antibodies, alternative scaffolds, bispecific or multispecific molecules, or immunoconjugates) and instructions for use. The kit can further contain one or more additional reagents, such as an immunosuppressive reagent, a cytotoxic agent or a radiotoxic agent, or one or more additional human antibodies according to at least some embodiments of the present invention (e.g., a human antibody having a complementary activity which binds to an epitope in the antigen distinct from the first human antibody).

The antibodies according to at least some embodiments of the present invention can also be used to target cells expressing Fc gamma R or LSR for example for labeling such cells. For such use, the binding agent can be linked to a molecule that can be detected. Thus, the present invention provides methods for localizing ex vivo or in vitro cells expressing Fc receptors, such as Fc gamma R, or LSR antigen. The detectable label can be, e.g., a radioisotope, a fluorescent compound, an enzyme, or an enzyme co-factor.

In a particular embodiment, the present invention provides methods for detecting the presence and/or level of LSR antigen in a sample, or measuring the amount of LSR antigen, respectively, comprising contacting the sample, and a control sample, with an antibody, or an antigen binding portion thereof, which specifically binds to LSR, under conditions that allow for formation of a complex between the antibody or portion thereof and LSR. The formation of a complex is then detected, wherein a difference complex formation between the sample compared to the control sample is indicative the presence of LSR antigen in the sample. As noted the present invention in particular embraces assays for detecting LSR antigen in vitro and in vivo such as immunoassays, radioimmunoassays, radioassays, radioimaging assays, ELISAs, Western blot, FACS, slot blot, immunohistochemical assays, and other assays well known to those skilled in the art.

In yet another embodiment, immunoconjugates of the present invention can be used to target compounds (e.g., therapeutic agents, labels, cytotoxins, radiotoxins immunosuppressants, etc.) to cells which have LSR cell surface receptors by linking such compounds to the antibody. Thus, the present invention also provides methods for localizing ex vivo or in vivo cells expressing LSR (e.g., with a detectable label, such as a radioisotope, a fluorescent compound, an enzyme, or an enzyme co-factor). Alternatively, the immunoconjugates can be used to kill cells which have LSR cell surface receptors by targeting cytotoxins or radiotoxins to LSR antigen.

According to at least some embodiments, the present invention provides a method for imaging an organ or tissue, the method comprising: (a) administering to a subject in need of such imaging, a labeled polypeptide; and (b) detecting the labeled polypeptide to determine where the labeled polypeptide is concentrated in the subject. When used in imaging applications, the labeled polypeptides according to at least some embodiments of the present invention typically have an imaging agent covalently or noncovalently attached thereto. Suitable imaging agents include, but are not limited to, radionuclides, detectable tags, fluorophores, fluorescent proteins, enzymatic proteins, and the like. One of skill in the art will be familiar with other methods for attaching imaging agents to polypeptides. For example, the imaging agent can be attached via site-specific conjugation, e.g., covalent attachment of the imaging agent to a peptide linker such as a polyarginine moiety having five to seven arginines present at the carboxyl-terminus of and Fc fusion molecule. The imaging agent can also be directly attached via non-site specific conjugation, e.g., covalent attachment of the imaging agent to primary amine groups present in the polypeptide. One of skill in the art will appreciate that an imaging agent can also be bound to a protein via noncovalent interactions (e.g., ionic bonds, hydrophobic interactions, hydrogen bonds, Van der Waals forces, dipole-dipole bonds, etc.).

In certain instances, the polypeptide is radiolabeled with a radionuclide by directly attaching the radionuclide to the polypeptide. In certain other instances, the radionuclide is bound to a chelating agent or chelating agent-linker attached to the polypeptide. Suitable radionuclides for direct conjugation include, without limitation, 18 F, 124 I, 125 I, 131 I, and mixtures thereof. Suitable radionuclides for use with a chelating agent include, without limitation, 47 Sc, 64 Cu, 67 Cu, 89 Sr, 86 Y, 87 Y, 90 Y,105 Rh, 111 Ag, 111 In, 117m S n, 149 Pm, 153 Sm, 166 Ho, 177 Lu, 186 Re, 188 Re, 211 At, 212 Bi, and mixtures thereof. Preferably, the radionuclide bound to a chelating agent is 64 Cu, 90 Y, 111 In, or mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to polypeptides of the present invention. In particular, attachment can be conveniently accomplished using, for example, commercially available bifunctional linking groups (generally heterobifunctional linking groups) that can be attached to a functional group present in a non-interfering position on the polypeptide and then further linked to a radionuclide, chelating agent, or chelating agent-linker.

Non-limiting examples of fluorophores or fluorescent dyes suitable for use as imaging agents include Alexa Fluor® dyes (Invitrogen Corp.; Carlsbad, Calif.), fluorescein, fluorescein isothiocyanate (FITC), Oregon Green™; rhodamine, Texas red, tetrarhodamine isothiocynate (TRITC), CyDye™ fluors (e.g., Cy2, Cy3, Cy5), and the like.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al., Mol. Microbiol., 55:1767-1781 (2005), the GFP variant described in Crameri et al., Nat. Biotechnol., 14:315-319 (1996), the cerulean fluorescent proteins described in Rizzo et al., Nat. Biotechnol, 22:445 (2004) and Tsien,Annu. Rev. Biochem., 67:509 (1998), and the yellow fluorescent protein described in Nagai et al., Nat. Biotechnol., 20:87-90 (2002). DsRed variants are described in, e.g., Shaner et al., Nat. Biotechnol., 22:1567-1572 (2004), and include mStrawberry, mCherry, morange, mBanana, mHoneydew, and mTangerine. Additional DsRed variants are described in, e.g., Wang et al., Proc. Natl. Acad. Sci. U.S.A., 101:16745-16749 (2004) and include mRaspberry and mPlum. Further examples of DsRed variants include mRFPmars described in Fischer et al., FEBS Lett., 577:227-232 (2004) and mRFPruby described in Fischer et al., FEBS Lett., 580:2495-2502 (2006).

In other embodiments, the imaging agent that is bound to a polypeptide according to at least some embodiments of the present invention comprises a detectable tag such as, for example, biotin, avidin, streptavidin, or neutravidin. In further embodiments, the imaging agent comprises an enzymatic protein including, but not limited to, luciferase, chloramphenicol acetyltransferase, β-galactosidase, β-glucuronidase, horseradish peroxidase, xylanase, alkaline phosphatase, and the like.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled polypeptide of the present invention. Positron emission tomography (PET) is another suitable technique for detecting radiation in a subject. Miniature and flexible radiation detectors intended for medical use are produced by Intra-Medical LLC (Santa Monica, Calif.). Magnetic Resonance Imaging (MRI) or any other imaging technique known to one of skill in the art is also suitable for detecting the radioactive emissions of radionuclides. Regardless of the method or device used, such detection is aimed at determining where the labeled polypeptide is concentrated in a subject, with such concentration being an indicator of disease activity.

Non-invasive fluorescence imaging of animals and humans can also provide in vivo diagnostic information and be used in a wide variety of clinical specialties. For instance, techniques have been developed over the years for simple ocular observations following UV excitation to sophisticated spectroscopic imaging using advanced equipment (see, e.g., Andersson-Engels et al., Phys. Med. Biol., 42:815-824 (1997)). Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol., 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanuj am et al., IEEE Transactions on Biomedical Engineering, 48:1034-1041 (2001), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

According to some embodiments, the sample taken from a subject (patient) to perform the diagnostic assay according to at least some embodiments of the present invention is selected from the group consisting of a body fluid or secretion including but not limited to blood, serum, urine, plasma, prostatic fluid, seminal fluid, semen, the external secretions of the skin, respiratory, intestinal, and genitourinary tracts, tears, cerebrospinal fluid, synovial fluid, sputum, saliva, milk, peritoneal fluid, pleural fluid, cyst fluid, secretions of the breast ductal system (and/or lavage thereof), broncho alveolar lavage, lavage of the reproductive system and lavage of any other part of the body or system in the body; samples of any organ including isolated cells or tissues, wherein the cell or tissue can be obtained from an organ selected from, but not limited to lung, colon, ovarian and/or breast tissue; stool or a tissue sample, or any combination thereof. In some embodiments, the term encompasses samples of in vivo cell culture constituents. Prior to be subjected to the diagnostic assay, the sample can optionally be diluted with a suitable eluant.

In some embodiments, the phrase “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

In some embodiments, the phrase “differentially present” refers to differences in the quantity or quality of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

In some embodiments, the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the term “diagnosis” refers to the process of identifying a medical condition or disease by its signs, symptoms, and in particular from the results of various diagnostic procedures, including e.g. detecting the expression of the nucleic acids or polypeptides according to at least some embodiments of the invention in a biological sample (e.g. in cells, tissue or serum, as defined below) obtained from an individual. Furthermore, as used herein the term “diagnosis” encompasses screening for a disease, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. The diagnostic procedure can be performed in vivo or in vitro.

In some embodiments, the phrase “qualitative” when in reference to differences in expression levels of a polynucleotide or polypeptide as described herein, refers to the presence versus absence of expression, or in some embodiments, the temporal regulation of expression, or in some embodiments, the timing of expression, or in some embodiments, any post-translational modifications to the expressed molecule, and others, as will be appreciated by one skilled in the art. In some embodiments, the phrase “quantitative” when in reference to differences in expression levels of a polynucleotide or polypeptide as described herein, refers to absolute differences in quantity of expression, as determined by any means, known in the art, or in other embodiments, relative differences, which may be statistically significant, or in some embodiments, when viewed as a whole or over a prolonged period of time, etc., indicate a trend in terms of differences in expression.

In some embodiments, the term “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

In some embodiments, the term “level” refers to expression levels of RNA and/or protein or to DNA copy number of a marker of the present invention.

Typically the level of the marker in a biological sample obtained from the subject is different (i.e., increased or decreased) from the level of the same marker in a similar sample obtained from a healthy individual (examples of biological samples are described herein).

Numerous well known tissue or fluid collection methods can be utilized to collect the biological sample from the subject in order to determine the level of DNA, RNA and/or polypeptide of the marker of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the marker can be determined and a diagnosis can thus be made.

Determining the level of the same marker in normal tissues of the same origin is preferably effected along-side to detect an elevated expression and/or amplification and/or a decreased expression, of the marker as opposed to the normal tissues.

In some embodiments, the term “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

In some embodiments, the term “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

In some embodiments, the term “detect” refers to identifying the presence, absence or amount of the object to be detected.

In some embodiments, the term “label” includes any moiety or item detectable by spectroscopic, photo chemical, biochemical, immunochemical, or chemical means. For example, useful labels include 32P, 35S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, this invention provides a method for detecting the polypeptides of this invention in a biological sample, comprising: contacting a biological sample with an antibody specifically recognizing a polypeptide according to the present invention and detecting said interaction; wherein the presence of an interaction correlates with the presence of a polypeptide in the biological sample.

In some embodiments of the present invention, the polypeptides described herein are non-limiting examples of markers for diagnosing a disease and/or an indicative condition. Each marker of the present invention can be used alone or in combination, for various uses, including but not limited to, prognosis, prediction, screening, early diagnosis, determination of progression, therapy selection and treatment monitoring of a disease and/or an indicative condition.

Each polypeptide/polynucleotide of the present invention can be used alone or in combination, for various uses, including but not limited to, prognosis, prediction, screening, early diagnosis, determination of progression, therapy selection and treatment monitoring of disease and/or an indicative condition, as detailed above.

Such a combination may optionally comprise any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker.

In some embodiments of the present invention, there are provided of methods, uses, devices and assays for the diagnosis of a disease or condition. Optionally a plurality of markers may be used with the present invention. The plurality of markers may optionally include a markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purposes.

In one embodiment, the panels comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicators. In other embodiments, threshold levels of a diagnostic or prognostic indicators can be established, and the level of the indicators in a patient sample can simply be compared to the threshold levels. The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment.

The present invention also relates to kits based upon such diagnostic methods or assays. Also within the scope of the present invention are kits comprising LSR conjugates or antibody compositions of the invention (e.g., human antibodies, bispecific or multispecific molecules, or immunoconjugates) and instructions for use. The kit can further contain one or more additional reagents, such as an immunosuppressive reagent, a cytotoxic agent or a radiotoxic agent, or one or more additional human antibodies according to at least some embodiments of the invention (e.g., a human antibody having a complementary activity which binds to an epitope in the antigen distinct from the first human antibody).

Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Radio-immunoassay (RIA): In one version, this method involves precipitation of the desired substrate and in the methods detailed herein below, with a specific antibody and radiolabeled antibody binding protein (e.g., protein A labeled with 1125) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

As described herein, the term “theranostic” may optionally refer to first testing the subject, such as the patient, for a certain minimum level of LSR, for example optionally in the cancerous tissue and/or in the immune infiltrate, as described herein as a sufficient level of LSR expression. Testing may optionally be performed ex vivo, in which the sample is removed from the subject, or in vivo.

If the cancerous tissue and/or the immune infiltrate have been shown to have the minimum level of LSR, then an anti-LSR antibody, alone or optionally with other treatment modalities as described herein, may optionally be administered to the subject.

Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

The therapeutic compositions (e.g., human antibodies, multispecific and bispecific molecules and immunoconjugates) according to at least some embodiments of the invention which have complement binding sites, such as portions from IgG1, -2, or -3 or IgM which bind complement, can also be used in the presence of complement. In one embodiment, ex vivo treatment of a population of cells comprising target cells with a binding agent according to at least some embodiments of the invention and appropriate effector cells can be supplemented by the addition of complement or serum containing complement. Phagocytosis of target cells coated with a binding agent according to at least some embodiments of the invention can be improved by binding of complement proteins. In another embodiment target cells coated with the compositions (e.g., human antibodies, multispecific and bispecific molecules) according to at least some embodiments of the invention can also be lysed by complement. In yet another embodiment, the compositions according to at least some embodiments of the invention do not activate complement.

The therapeutic compositions (e.g., human antibodies, multispecific and bispecific molecules and immunoconjugates) according to at least some embodiments of the invention can also be administered together with complement. Thus, according to at least some embodiments of the invention there are compositions, comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules according to at least some embodiments of the invention and the complement or serum can be administered separately.

The present invention is further illustrated by the following examples. This information and examples is illustrative and should not be construed as further limiting. The contents of all figures and all references, patents and published patent applications cited throughout this application are expressly incorporated herein by reference.

### EXAMPLES

### Example 1

### Cloning of LSR Proteins

A. Cloning of LSR_T1_P5a ORF

Cloning of LSR_T1_P5a open reading frame (ORF) (SEQ ID NO: 154) was performed by PCR to generate LSR_P5a protein (SEQ ID NO: 11), as described below.

A PCR reaction was performed using PfuUltra II Fusion HS DNA Polymerase (Agilent, Catalog no. 600670) under the following conditions: 50 ng of pIRES_puro3_LSR_T1_P5a_Flag construct described above served as a template for a PCR reaction with 0.5 microliter of each of the primers 200_{—}369_LSR_Kozak_NheI (SEQ ID NO: 147) and 200-372_LSR_BamHI_Rev (SEQ ID NO: 152) in a total reaction volume of 25 μl. The reaction conditions were 5 minutes at 98° C.; 35 cycles of: 20 seconds at 98° C., 30 seconds at 55° C. and 1.5 minutes at 72° C.; then 10 minutes at 72° C. All of the primers that were used include gene specific sequences, restriction enzyme sites and Kozak sequence. The PCR product was separated on 1% agarose gel. After verification of the expected band size, the PCR product was purified using QIAquick™ Gel Extraction kit as described above.

The purified PCR product was digested with NheI and BamHI restriction enzymes (New England Biolabs, Beverly, Mass., U.S.A.). After digestion, the DNA was separated on a 1% agarose gel. The expected band size was excised and extracted from the gel as described above. The digested DNA was then ligated into pIRESpuro3 vector that was digested with NheI and BamHI as described above, incubated with Antarctic Phosphatase (New England Biolabs, Beverly, Mass., U.S.A., Catalog no. MO289L) for 30 minutes at 37° C. and purified from 1% agarose gel using QIAquick™ Gel Extraction kit as described above. The ligation reaction was performed with T4 DNA Ligase (Promega; Catalog no. M180A).

Sequence verification of both tagged and untagged constructs described above was performed (Hylabs, Rehovot, Israel). Two nucleotide mismatches were identified, as follows: G to A at nucleic acid position 119 of SEQ ID NO: 154, and A to G at nucleic acid position 626 from SEQ ID NO: 154, resulting in a nucleic sequence set forth in SEQ ID NO: 145 for the untagged construct, and SEQ ID NO: 146 for the tagged construct; yielding a polypeptide having an amino acid mismatch I to M in amino acid position 11, resulting in a protein having amino acid sequence set forth in SEQ ID NO: 143 for the untagged construct and SEQ ID NO: 144 for the tagged construct.

The above recombinant plasmids were processed for stable pool generation as described below.

2. Cloning of LSR_WT ORF

Cloning of LSR_WT open reading frame (ORF) was performed by substitution of Alanine at position 627 to Glycine by one-step site-directed mutagenesis PCR to generate LSR_WT protein (SEQ ID NO: 154), as described below.

A PCR reaction was performed using PfuUltra II Fusion HS DNA Polymerase (Agilent, Catalog no. 600670) under the following conditions: 20 ng of pIRES_puro3_LSR_T1_P5a_Flag_m construct described above (SEQ ID NO: 145), served as a template for a PCR reaction with 2.5 microliter of each of the primers 200_{—}398 (SEQ ID NO: 199) and 200-399 (SEQ ID NO: 200) in a total reaction volume of 50u1. The reaction conditions were 3 minutes at 95° C.; 12 cycles of: 1 minute at 95° C., 1 minute at 55° C. and 3 minutes at 72° C.; then 10 minutes at 72° C. 2u1 DpnI were added to the PCR reaction and incubate for 2 h at 37° C.

Sequence verification of tagged construct described above was performed (Hylabs, Rehovot, Israel).

The above recombinant plasmid was processed for stable pool generation as described below.

### 3. Cloning of LSR—SKIP4 ORF

Full length cDNA of human-LSR (SEQ ID NO: 201) variant skipping exon 4 was synthesized with a Flag tag at the C-terminus, and cloned in pUC57 vector by GenWiz (USA). This cDNA was subsequently cloned in the pRp3 mammalian expression vector, pcDNA3.1, to create an expression construct, as described below.

cDNA was digested with NheI and BamHI restriction enzymes and ligated to pIRESpuro3 (pRp3) mammalian expression vector (Clontech, Cat No: 631619) previously digested with the same enzymes. The resulting expression constructs were verified by sequence (SEQ ID No:201) and subsequently used for transfections and stable pool generation as described below.

### 4. Cloning of Mouse LSR-WT Flag Construct

Full length cDNA of mouse WT LSR (SEQ ID NO:202) was synthesized with a Flag tag at the C-terminus, cloned into pUC57 vector by GenScript, and subcloned into a mammalian expression vector, pcDNA3.1, to create an expression construct, as described below.

cDNA was digested with NheI and BamHI restriction enzymes and ligated to pcDNA3.1+ mammalian expression vector previously digested with the same enzymes. The resulting expression constructs were verified by sequence (SEQ ID No:202) and subsequently used for transfections and stable pool generation as described below.

5. Cloning of cyno LSR_WT ORF

Cloning of cyno LSR_WT open reading frame (ORF) (SEQ ID NO:203) was performed by PCR to generate cyno LSR_WT protein (SEQ ID NO: 203), as described below.

A PCR reaction was performed using G0 Taq DNA Polymerase (Promega, Catalog no. M3001) under the following conditions: Pool of Monkey cDNA (Biochain, Cat. No. C8534502-Cy, C8534501-Cy) served as a template for 2 different PCR reactions. The first with 1 microliter of each of the primers 200_{—}403_cLSR_Kozak_NheI (SEQ ID NO:204) and 200-407_cLSR_Rev (SEQ ID NO:205) and the second with 1 microliter of each of the primers 200_{—}404_cLSR_Flag_EcoRI (SEQ ID NO:206) and 200-406_cLSR_For (SEQ ID NO: 207), both in a total reaction volume of 50 μl.

The reaction conditions were 5 minutes at 95° C.; 40 cycles of: 30 seconds at 95° C., 30 seconds at 55° C. and 1 minute at 72° C.; then 5 minutes at 72° C. All of the primers that were used include gene specific sequences, restriction enzyme sites and Kozak sequence. The PCR product was separated on 1% agarose gel. After verification of the expected band size, the PCR products were purified using QIAquick™ Gel Extraction kit as described above. These purified PCR products used as a template for a PCR reaction under the following conditions: 5 minutes at 95° C.; 40 cycles of: 30 seconds at 95° C., 30 seconds at 55° C. and 1.5 minutes at 72° C.; then 5 minutes at 72° C., using G0 Taq DNA polymerase (Promega, Catalog no. M3001). The PCR product was loaded on 1% agarose gel and the product was purified the same way as described above.

The purified PCR product was digested with NheI and EcoRI restriction enzymes (New England Biolabs, Beverly, Mass., U.S.A.). After digestion, the DNA was separated on a 1% agarose gel. The expected band size was excised and extracted from the gel as described above. The digested DNA was then ligated into pIRESpuro3 vector that was digested with NheI and EcoRI as described above, incubated with Antarctic Phosphatase (New England Biolabs, Beverly, Mass., U.S.A., Catalog no. MO289L) for 30 minutes at 37° C. and purified from 1% agarose gel using QIAquick™ Gel Extraction kit as described above. The ligation reaction was performed with T4 DNA Ligase (Promega; Catalog no. M180A).

Sequence verification of tagged construct described above was performed (Hylabs, Rehovot, Israel).

The above recombinant plasmid was processed for stable pool generation as described below.

6. Cloning of cyno LSR_SKIP4 ORF

Cloning of cyno LSR_skip4 open reading frame (ORF) (SEQ ID NO:208) was performed by PCR to generate cyno LSR_skip4 protein (SEQ ID NO:208), as described below.

A PCR reaction was performed using G0 Taq DNA Polymerase (Promega, Catalog no. M3001) under the following conditions: Pool of Monkey cDNA (Biochain, Cat. No. C8534502-Cy, C8534501-Cy) served as a template for PCR reaction.

1 microliter of each of the primers 200_{—}403_cLSR_Kozak_NheI (SEQ ID NO:204) and 200-404_cLSR_Flag_EcoRI (SEQ ID NO: 206) in a total reaction volume of 50 μl.

The reaction conditions were 5 minutes at 95° C.; 40 cycles of: 30 seconds at 95° C., 30 seconds at 55° C. and 1.45 minutes at 72° C.; then 5 minutes at 72° C. All of the primers that were used include gene specific sequences, restriction enzyme sites and Kozak sequence. The PCR product was separated on 1% agarose gel. After verification of the expected band size, the PCR products were purified using QIAquick™ Gel Extraction kit as described above. The purified PCR product was digested with NheI and EcoRI restriction enzymes (New England Biolabs, Beverly, Mass., U.S.A.). After digestion, the DNA was separated on a 1% agarose gel. The expected band size was excised and extracted from the gel as described above. The digested DNA was then ligated into pIRESpuro3 vector that was digested with NheI and EcoRI as described above, incubated with Antarctic Phosphatase (New England Biolabs, Beverly, Mass., U.S.A., Catalog no. MO289L) for 30 minutes at 37° C. and purified from 1% agarose gel using QIAquick™ Gel Extraction kit as described above. The ligation reaction was performed with T4 DNA Ligase (Promega; Catalog no. M180A).

Sequence verification of tagged construct described above was performed (Hylabs, Rehovot, Israel).

The above recombinant plasmid was processed for stable pool generation as described below.

### Example 2

### Establishment of Stable Pools of Recombinant Cells Expressing LSR Proteins

1. Establishment of a Stable Pool of Recombinant Hek293T Cells Expressing LSR_P5a_FLAG_M Protein

HEK-293T cells were stably transfected with LSR_T1_P5a_Flag_m (SEQ ID NO: 146) and pIRESpuro3 empty vector plasmids as follows:

HEK-293T (ATCC, CRL-11268) cells were plated in a sterile 6 well plate suitable for tissue culture, containing 2 ml pre-warmed of complete media, DMEM [Dulbecco's modified Eagle's Media, Biological Industries (Beit Ha'Emek, Israel, catalog number: 01-055-1A)+10% FBS [Fetal Bovine Serum, Biological Industries (Beit Ha'Emek, Israel, catalog number: 04-001-1A)+4 mM L-Glutamine (Biological Industries (Beit Ha'Emek, Israel), catalog number: 03-020-1A). 500,000 cells per well were transfected with 2 μg of DNA construct using 6 μl FuGENE 6 reagent (Roche, catalog number: 11-814-443-001) diluted into 94u1 DMEM. The mixture was incubated at room temperature for 15 minutes. The complex mixture was added dropwise to the cells. The cells were placed in an incubator maintained at 37° C. with 5% CO2 content. 48 hours after the transfection, the cells were transferred to a 75 cm2 tissue culture flask containing 15 ml of selection medium: complete medium supplemented with 5 μg\ml puromycin (Sigma, catalog number P8833). Cells were placed in an incubator, and the medium was replaced every 3-4 days, until clone formation was observed.

2. Generation of Stable Transfectant Pools Expressing Human and cyno Wt and Skip4 LSR Proteins

HEK-293T (ATCC, CRL-11268) cells were transfected with the human and cyno LSR (SEQ ID NOs:154, 201, 203, 208)LSR pRp3 constructs described above or with the empty vector (pRp3) as negative control, using Fugene6 transfection reagent (Roche, Cat No: 111-988-387). Puromycin resistant colonies were selected for stable pool generation. For mouse LSR WT (SEQ ID NO:202), a different expression vector and other cell lines were used for generation of stable transfectants pools (see below).

3. Generation of Stable Transfectant Pools Expressing Mouse WT Protein

Stable transfectant cell pools expressing the WT-Mouse LSR-flag protein (SEQ ID NO: 202) were generated at GeneScript (USA Inc). The mouse WT LSR sequence (SEQ ID NO: 202) with the Flag tag at the C′-terminus was synthesized, cloned into pUC57 vector, and sub-cloned into a mammalian expression vector pcDNA3.1. The recombinant plasmid was transfected into CHO-K1 (ATCC, cat #CCL-61) and into HEK-293 (ATCC cat #CRL-1573™) cells. Cell pools of stable transfectants were screened using G418 and analyzed by western blot using anti-flag Ab.

### Example 3

### Expression Validation

A. Analysis of the Ectopic Expression of LSR_P5a_FLAG_M in Stably-Transfected HEK293T Cells

The expression of LSR_P5a_Flag_m (SEQ ID NO: 144) in stably-transfected HEK293T cells was determined by Western blot analysis of the cell lysates, using anti LSR Antibodies and anti flag antibody as indicated in Table 1.

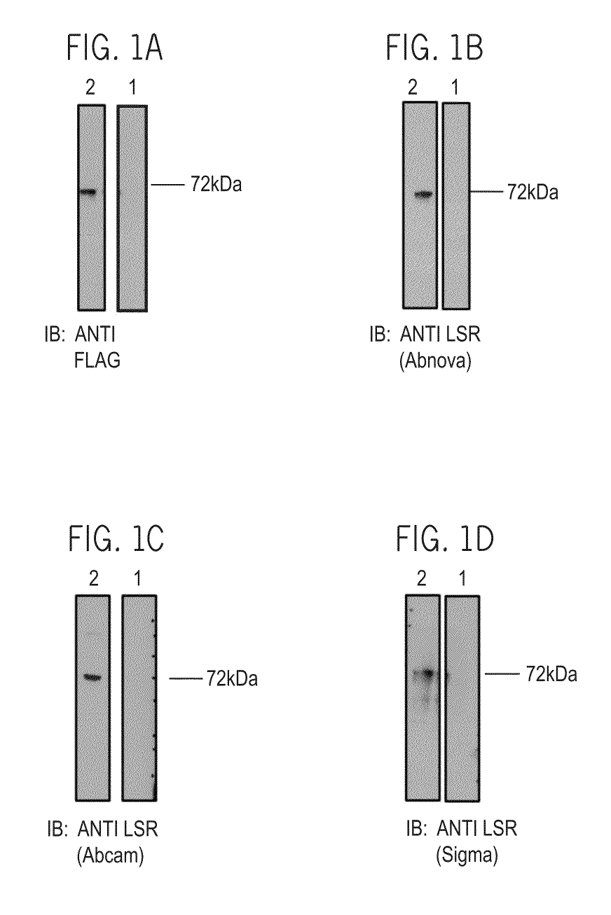

Cells were dissociated from the plate using Cell Dissociation Buffer Enzyme-Free PBS-Based (Gibco; 13151-014), washed in Dulbecco's Phosphate Buffered Saline (PBS) (Biological Industries, 02*023-1A) and centrifuged at 1200 g for 5 minutes. Whole cell extraction was performed by resuspending the cells in 50 mM Tris-HCl pH7.4, 150 mM NaCl, 1 mM EDTA, 1% Triton X-100, supplemented with 25× complete EDTA free protease inhibitor cocktail (Roche, 11 873 580 001) and vortexing for 20 seconds. Cell extracts were collected following centrifugation at 20000 g for 20 minutes at 4° C. and protein concentration was determined with Bradford Biorad Protein Assay (Biorad cat#500-0006). Equal protein amounts were analyzed by SDS-PAGE (Invitrogen NuPAGE 4-12% NuPAGE Bis Tris, Cat# NP0335, NP0322) and transferred to Nitrocellulose membrane (BA83, 0.2 μm, Schleicher & Schuell, Cat#401385). The membrane was blocked with TTBS (Biolab, Cat#: 20892323)/10% skim milk (Difco, Cat#232100) and incubated with the indicated primary antibodies (FIG. 1) diluted in TTBS/5% BSA (Sigma-Aldrich, A4503) at the indicated concentrations (Table 2), for 16 hours at 4° C. After 3 washes with TTBS, The membrane was further incubated for 1 hour at Room Temperature with the secondary-conjugated antibodies as indicated, diluted in TTBS. Chemiluminescence reaction was performed with ECL Western Blotting Detection Reagents (GE Healthcare, Cat # RPN2209) and the membrane was exposed to Super RX Fuji X-Ray film (Catalog no. 4741008389).

FIG. 1 demonstrates the expression of LSR_P5a_Flag_m protein (SEQ ID: 144) in recombinant HEK293T cells at the expected band size ˜70 kDa, as detected with anti Flag (Sigma cat#A8592) (FIG. 1A) and anti LSR antibodies as follow: Abnova, cat#H00051599-B01P (FIG. 1B) Abc am, cat ab59646 (FIG. 1C) and Sigma cat# HPA007270 (FIG. 1D).

B. Expression Validation of Human, Cyno and Mouse LSR_Wt and LSR SKIP4 in Stably-Transfected HEK293T Cells or in CHO-K1 Cells

The expression of human, cyno and mouse LSR_WT and LSR skip4 in stably-transfected HEK293T cells or in CHO-K1 cells was determined by Western blot analysis of the cell lysates, using anti LSR Antibodies and anti-flag antibody as indicated in Table 1.

Cells were dissociated from the plate using Cell Dissociation Buffer Enzyme-Free PBS-Based (Gibco; 13151-014), washed in Dulbecco's Phosphate Buffered Saline (PBS) (Biological Industries, 02*023-1A) and centrifuged at 1200 g for 5 minutes. Whole cell extraction was performed by resuspending the cells in 50 mM Tris-HCl pH7.4, 150 mM NaCl, 1 mM EDTA, 1% Triton X-100, supplemented with 25× complete EDTA free protease inhibitor cocktail (Roche, 11 873 580 001) and vortexing for 20 seconds. Cell extracts were collected following centrifugation at 20000 g for 20 minutes at 4° C. and protein concentration was determined with Bradford Biorad Protein Assay (Biorad cat#500-0006). Equal protein amounts were analyzed by SDS-PAGE (Invitrogen NuPAGE 4-12% NuPAGE Bis Tris, Cat# NP0335, NP0322) and transferred to Nitrocellulose membrane (BA83, 0.2 μm, Schleicher & Schuell, Cat#401385). The membrane was blocked with TTBS (Biolab, Cat#: 20892323)/5% skim milk (Difco, Cat#232100) and incubated with the indicated primary antibodies (FIG. 1) diluted in TTBS/5% BSA (Sigma-Aldrich, A4503) at the indicated concentrations (Table 1), for 1 hour at Room Temperature. After 3 washes with TTBS, The membrane was further incubated for 1 hour at Room Temperature with the secondary-conjugated antibodies Goat anti-Rabbit IgG-Peroxidase (Jackson, Cat. No. 111-035-003) diluted 1:20,000 in TTBS. Chemiluminescence reaction was performed with ECL Western Blotting Detection Reagents (GE Healthcare, Cat # RPN2209) and the membrane was exposed to Super RX Fuji X-Ray film (Catalog no. 4741008389).

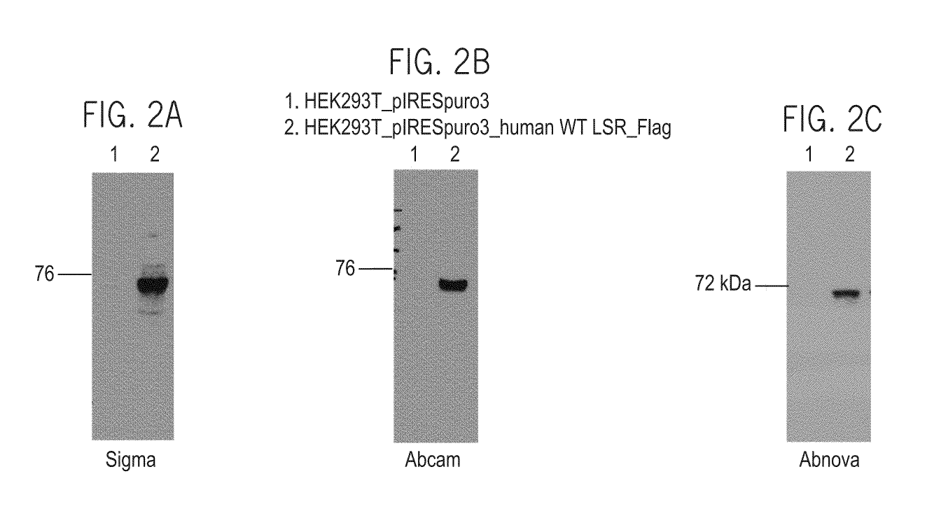

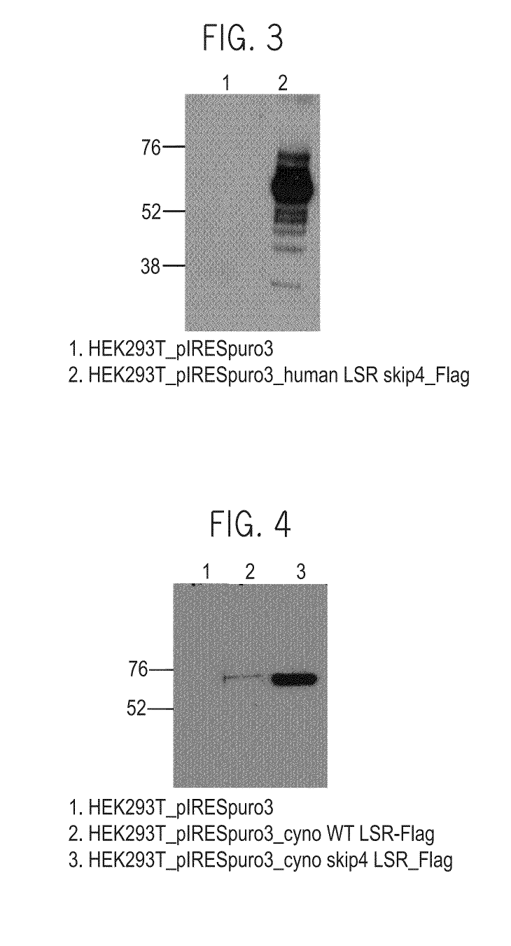

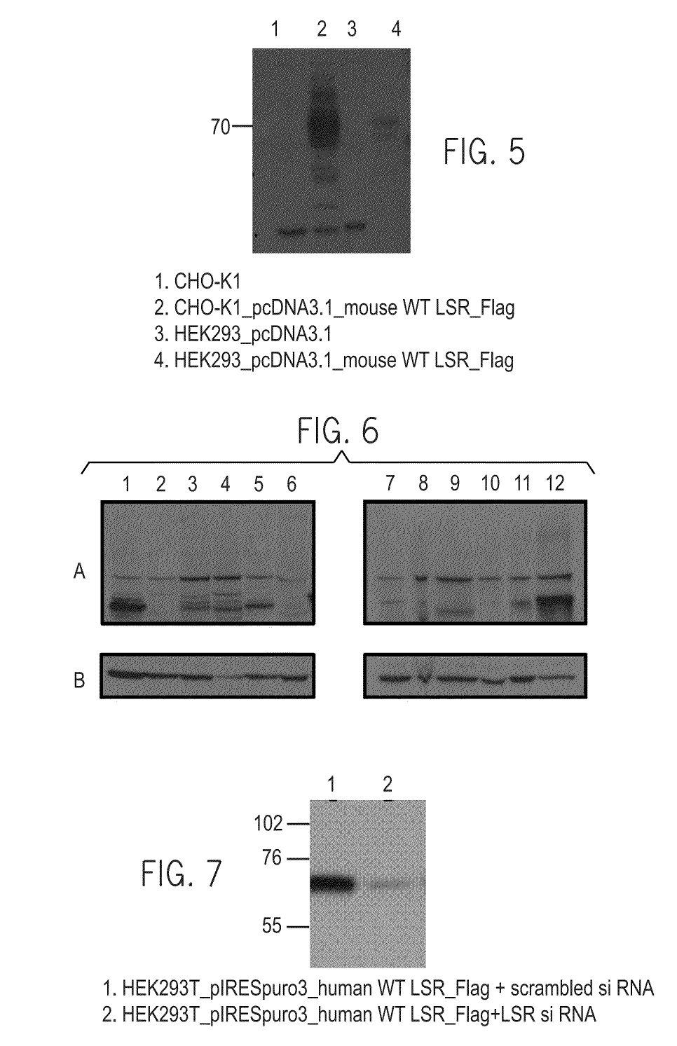

FIGS. 2, 3, 4, 5 present western blot analysis results demonstrating the expression of human (FIGS. 2, 3), cyno (FIG. 4) and mouse (FIG. 5) LSR_WT and LSR skip4 proteins (SEQ ID NOs:11, 13, 211, 212, 31) in recombinant HEK293T cells transfected with both LSR_WT and LSR skip4 constructs of human and cyno LSR) and in HEK293 and CHO-K1 cells transfected with WT construct of mouse LSR, at the expected band size ˜70 kDa, as detected with anti-Flag (Sigma cat#A8592) (FIG. 5) and anti LSR antibodies (Abcam, cat ab59646) (FIGS. 2, 3, 4).

FIG. 2 presents detection of human LSR_WT with 3 different commercial Abs against LSR: 2A refers to SIGMA Ab, whereas 2B to Abcam Ab and 2C to Abnova Ab (as detailed in Table 1). Lane #1 represents HEK293T_pIRESpuro3 empty vector transfected cells which were used as a negative control, whereas lane #2 represents HEK293T_pIRESpuro3_human WT LSR_Flag transfected cells.

FIG. 3 presents detection of the human LSR_skip4 protein using Abcam Ab. Lane #1 represents HEK293T_pIRESpuro3 empty vector transfected cells which were used as a negative control, whereas lane #2 represents HEK293T_pIRESpuro3_human LSR skip4_Flag transfected cells.

FIG. 4 presents detection of the cyno LSR_WT (lane 2) and LSR_skip4 (lane 3) proteins, both compared to the empty vector cells lysate (lane 1).

FIG. 5 presents detection by anti-flag Abs of mouse LSR_WT protein in CHO-K1 cells (lane 2) and in HEK293 cells (lane 4) compared to the relevant empty vector cells lysate (lanes 1 and 3 respectively).

TABLE 1
Primary and secondary antibodies

| Antibody | Application | Dilution |
|---|---|---|
| Mouse Anti FLAG-Cy3 (Sigma catalog number: | IF | 1:200 |
| A9594) | | |
| Mouse Anti FLAG-HRP (Sigma Catalog no. A8592) | WB | 1:2000 |
| Rabbit Anti LSR (Abcam catalog number: ab59646) | IF | 1:500 |
| | WB | 1:4000 |
| Rabbit Anti LSR (Sigma catalog number: | IF | 1:100 |
| HPA007270) | WB | 1:2500 |
| Mouse Anti LSR (Abnova catalog number: | IF | 1:500 |
| H00051599-B01P) | WB | 1:1000 |
| Mouse Anti GAPDH (Abcam catalog number: | WB | 1:1000 |
| ab9484) | | |
| Donkey Anti Rabbit Cy3 (Jackson ImmunoResearch | IF | 1:200 |
| Laboratories Inc. catalog no. 711-165-152) | | |
| Donkey Anti Mouse Dylight 549 (Jackson | IF | 1:100 |
| ImmunoResearch Laboratories Inc. catalog no. 715- | | |
| 506-150) | | |
| Peroxidase conjugated affinity purified Goat Anti | WB | 1:10000 |
| Rabbit IgG (Jackson ImmunoResearch Laboratories | | |
| Inc. catalog no. 111-035-003) | | |
| Peroxidase conjugated affinity purified Goat Anti- | WB | 1:10000 |
| Mouse IgG (Jackson ImmunoResearch Laboratories | | |
| Inc. catalog no. 115-035-146) | | |

C. Analysis of the Expression of Endogenous LSR Protein in Various Cell Lines

The expression of endogenous LSR protein in various cell lines was analyzed by Western Blotting as described below.

SK-OV3 (ATCC no. HTB-77) Caov3 (ATCC no. HTB-75), OVCAR3 (ATCC no. HTB-161), ES-2 (ATCC no. CRL-1978), OV-90 (ATCC no. CRL-11732), TOV112D (ATCC no. CRL-11731) and Hep G2 (ATCC no. HB-8065) cell extracts were prepared as described above.

HeLa (catalog no. sc-2200), MCF-7 (catalog no. sc-2206), CaCo2 (catalog no. sc-2262) and SkBR3 (catalog no. sc-2218) cell extracts were purchased from SantaCruz Biotechnology.

Equal protein amounts were analyzed by SDS-PAGE and transferred to Nitrocellulose membrane as described above. The membrane was blocked with TTBS (Biolab, Cat#: 20892323)/10% skim milk (Difco, Cat#232100) and incubated with anti LSR antibodies (Abcam,cat#ab59646) diluted in TTBS/5% BSA (Sigma-Aldrich, A4503) at the indicated concentrations (Table 2), for 16 hours at 4° C. After 3 washes with in TTBS, The membrane was further incubated for 1 hour at Room Temperature with the secondary-conjugated antibodies as indicated (Table 2), diluted in TBS. Chemiluminescence reaction was performed with ECL Western Blotting Detection Reagents (GE Healthcare, Cat # RPN2209) and the membrane was exposed to Super RX Fuji X-Ray film (Catalog no. 4741008389).

FIG. 6 demonstrates the endogenous expression of LSR in various cell lines. A band at 72 kDa corresponding to LSR was detected with anti LSR antibody in extracts of SK− OV3, Caov3, OVCAR3, OV-90, Hep G2, HeLa, CaCo2, and SkBR3 (FIG. 6A). Anti GAPDH (Abcam cat# ab9484) served as a loading control (FIG. 6B).

TABLE 2
Primary and secondary antibodies

| | Applica- | |
|---|---|---|
| Antibody | tion | Dilution |
| Mouse Anti FLAG-Cy3 (Sigma catalog number: A9594) | IF | 1:200 |
| Mouse Anti FLAG-HRP (Sigma Catalog no. A8592) | WB | 1:2000 |
| Rabbit Anti LSR (Abcam catalog number: ab59646) | IF | 1:500 |
| | WB | 1:4000 |
| Rabbit Anti LSR (Sigma catalog number: HPA007270) | IF | 1:100 |
| | WB | 1:2500 |
| Mouse Anti LSR (Abnova catalog number: H00051599- | IF | 1:500 |
| B01P) | WB | 1:1000 |
| Mouse Anti GAPDH (Abcam catalog number: ab9484) | WB | 1:1000 |
| Donkey Anti Rabbit Cy3 (Jackson ImmunoResearch | IF | 1:200 |
| Laboratories Inc. catalog no. 711-165-152) | | |
| Donkey Anti Mouse Dylight 549 (Jackson | IF | 1:100 |
| ImmunoResearch Laboratories Inc. catalog no. 715-506- | | |
| 150) | | |
| Peroxidase conjugated affinity purified Goat Anti Rabbit | WB | 1:10000 |
| IgG (Jackson ImmunoResearch Laboratories Inc. catalog | | |
| no. 111-035-003) | | |
| Peroxidase conjugated affinity purified Goat Anti-Mouse | WB | 1:10000 |
| IgG (Jackson ImmunoResearch Laboratories Inc. catalog | | |
| no. 115-035-146) | | |

### Example 4A

### Knock Down of LSR Protein Expression by siRNA

In order to verify the performance of siRNA (Thermo Scientific Cat#M-009672-00-0005) specific to the human LSR_WT (SEQ ID NO:11) protein, and to validate its specificity, knock down of LSR protein was performed on the HEK293T cells stably expressing the Human LSR_WT (SEQ ID NO:11).

Stably transfected recombinant HEK293T cells expressing human LSR_WT (SEQ ID NO:11) described above were plated in 6 wells plate in 2 ml Opti-MEMO I Reduced Serum Medium (Gibco, cat#31985-047) containing 10% FBS 24 hr prior the siRNA transfection. For each transfection sample, oligomer and lipofectamine 2000 transfection reagent (Invitrogen cat#11668019) complexes were prepared and added as follow: 100 μmol siRNA oligomer and 5u1 of the transfection reagent were mixed in a final volume of 250u1 optimum without serum. The above complexes were incubated at RT for 20 minutes and added to each well containing cells and medium. The plate was mixed gently and incubated for 48 hr at 37° C. in a CO₂ incubator, then the cells were collected from the plate using Cell Dissociation Buffer Enzyme-Free PBS-Based (Gibco; 13151-014), washed in Dulbecco's Phosphate Buffered Saline (PBS) (Biological Industries, 02*023-1A) and centrifuged at 1200 g for 5 minutes. Whole cell extraction and western analysis were performed as mentioned above.

The results shown in FIG. 7 demonstrate a dramatic decrease in the signal intensity of the ˜70 kDa band following transfection by the LSR specific siRNA (Thermo Scientific Cat#M-009672-00-0005) (lane 2), as compared to the scrambled siRNA (Thermo Scientific Cat#D-001810-01-05) (lane 1), indicating specific knock down of the ectopic expression of the Human WT LSR-flag protein.

### Example 4B

### Knock Down of LSR in Endogenous Cell Lines

Knockdown of the endogenous expression of LSR protein (SEQ ID NO:11) in HT29 cells or in HepG2/C3A cells was carried out by transient transfection of siRNA specific to LSR (SEQ ID NO:11). Cells were transfected with 30 μmol (10 nM) (for HT29 cells) and with 50 μmol (for HepG2/C3A) LSR specific siRNA (Thermo Scientific Cat#M-009672-00-0005) and scrambled siRNA (Thermo Scientific Cat#D-001810-01-05) as a negative control, using Lipofectamine® RNAiMAX Transfection Reagent (Invitrogen, Cat#13778-150). Following incubation of 72 hr (for HT29 cells) or 48 hr (for HepG2/C3A cells) cells were analyzed by FACS using hybridoma sup of the anti-LSR (SEQ ID NO:11) mAb 8C8 or by Western blot using commercial anti-LSR polyclonal antibodies, as described above in Table 1.

FIG. 16 demonstrates a specific knockdown of endogenous LSR (SEQ ID NO:11) protein expression analyzed by Western blot.

FIG. 17 demonstrates FACS analysis results of HT29 (FIG. 17A) and HepG2/C3A (FIG. 17B) cells following transient transfection with the LSR-specific siRNA (Thermo Scientific Cat#M-009672-00-0005).

Specific knockdown of endogenous LSR (SEQ ID NO:11) protein surface expression (arrow#1) is shown, as compared to cells transfected with scrambled-siRNA (Thermo Scientific Cat#D-001810-01-05) (arrow#2) as negative control.

### Example 5

### Determination of the Subcellular Localization of the Ectopic LSR Proteins in the Transfected Cells

A. Determination of the Sub Cellular Localization of the Ectopic LSR_P5a_FLAG_M in HEK293T Cells

The subcellular localization of the LSR_P5a_Flag_m protein (SEQ ID NO: 144) was determined in stably-transfected cells by confocal microscopy.

Stably transfected recombinant HEK293T cells expressing a LSR_P5a_Flag_m (SEQ ID NO: 144) described above were plated on coverslips pre-coated with Poly-L-Lysine (Sigma; Catalogue no. P4832). After 24 hrs the cells were processed for immunostaining and analyzed by confocal microscopy. The cover slip was washed in phosphate buffered saline (PBS), then fixed for 15 minutes in a solution of PBS/3.7% paraformaldehyde (PFA) (EMS, catalog number: 15710)/3% glucose (Sigma, catalog number: G5767). The PFA was Quenched with PBS/3 mM Glycine (Sigma, catalog number: G7126) for 5 minutes. After two 5-minute washes in PBS, the cells were permeabilized with PBS/0.1% Triton-X100 for 5 minutes at Room Temperature and washe twice in PBS. Then, blocking of non-specific regions was performed with PBS/5% Bovine Serum Albumin (BSA) (Sigma, catalog number: A4503) for 20 minutes. The coverslip was then incubated in a humid chamber for 1 hour with each of the primary antibodies antibodies diluted in PBS/5% BSA as indicated, followed by three 5-minute washes in PBS. The coverslips were then incubated for 30 minutes with the corresponding secondary antibody diluted in PBS/2.5% BSA at the indicated dilution. The antibodies and the dilutions that were used are specified in Table 2. After a prewash in Hank's Balanced Salt Solutions w/o phenol red (HBSS) (Biological Industries Catalog no. 02-016-1), the coverslip was incubated with WGA-Alexa 488 (Invitrogen, catalog number W11261) diluted 1:200 in HBSS for 10 min, washed in HBSS and incubated in BISBENZIMIDE H 33258 (Sigma, catalog number: 14530) diluted 1:1000 in HBSS. The coverslip was then mounted on a slide with Gel Mount Aqueous medium (Sigma, catalog number: G0918) and cells were observed for the presence of fluorescent product using confocal microscopy.

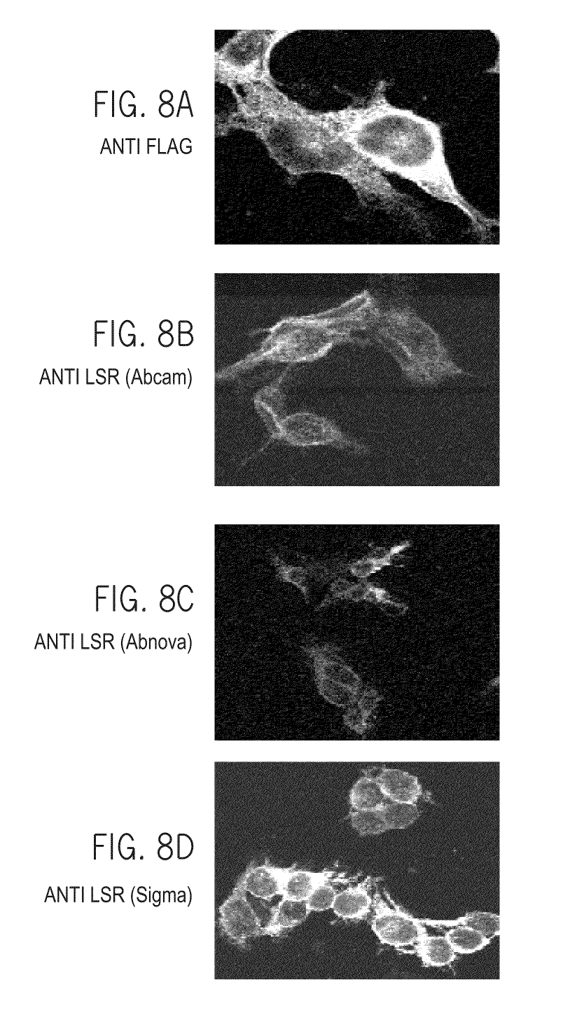

The subcellular localization of LSR_P5a_Flag_m is demonstrated in FIG. 8, LSR_P5a_Flag_m (SEQ ID NO: 144) is localized mainly to the cell cytoplasm, but can also be detected on the cell surface as detected with anti Flag (Sigma cat# A9594) (FIG. 8A) and anti LSR antibodies as follows: Abcam, cat ab59646 (FIG. 8B) Abnova, cat#H00051599-B01P (FIG. 8C) and Sigma cat# HPA007270 (FIG. 8D).

B. Determination of the Subcellular Localization of the Ectopic Human, Cyno and Mouse LSR_WT and LSR_SKIP4 in HEK293T and CHO-K1 Cells

The subcellular localization of the LSR_WT (SEQ ID NOs:11, 211, 31) and skip4 protein (SEQ ID NOs:13, 212) were determined in stably-transfected cells by confocal microscopy.

Stably transfected recombinant HEK293T cells expressing human and cyno LSR_WT (SEQ ID NOs:11, 211) and LSR_skip4 (SEQ ID NOs:13, 212) and CHO-Kt cells expressing mouse LSR_WT (SEQ ID NO:31), described above were plated on coverslips pre-coated with Poly-L-Lysine (Sigma; Catalogue no. P4832). After 24 hrs the cells were processed for immunostaining and analyzed by confocal microscopy. The cover slip was washed in phosphate buffered saline (PBS), then fixed for 15 minutes in a solution of PBS/3.7% paraformaldehyde (PFA) (EMS, catalog number: 15710)/3% glucose (Sigma, catalog number: G5767). The PFA was Quenched with PBS/3 mM Glycine (Sigma, catalog number: G7126) for 5 minutes. After two 5-minute washes in PBS, blocking of non-specific regions was performed with PBS/5% Bovine Serum Albumin (BSA) (Sigma, catalog number: A4503) for 20 minutes. The coverslip was then incubated in a humid chamber for 1 hour with each of the primary antibodies antibodies diluted in PBS/5% BSA as indicated, followed by three 5-minute washes in PBS. The coverslips were then incubated for 30 minutes with the corresponding secondary antibody diluted in PBS/2.5% BSA at the indicated dilution. The antibodies and the dilutions that were used are specified in Table 1. After a prewash in Hank's Balanced Salt Solutions w/o phenol red (HBSS) (Biological Industries Catalog no. 02-016-1), the coverslip was incubated with WGA-Alexa 488 (Invitrogen, catalog number W11261) diluted 1:200 in HBSS for 10 min, washed in HBSS and incubated in BISBENZIMIDE H 33258 (Sigma, catalog number: 14530) diluted 1:1000 in HBSS. The coverslip was then mounted on a slide with Gel Mount Aqueous medium (Sigma, catalog number G0918) and cells were observed for the presence of fluorescent product using confocal microscopy.

The subcellular localization of human, cyno and mouse LSR_WT (SEQ ID NOs:11, 211, 31, respectively) and cyno LSR_skip4 (SEQ ID NO:212) is demonstrated in FIGS. 9, 10, 11, 12. LSR protein is localized mainly to the cell cytoplasm, but can also be detected on the cell surface as detected with anti Flag (Sigma cat# A9594) (FIGS. 9A, 10A, 11A, 12A and 13A) and anti LSR antibodies as follows: Abcam, cat ab59646 (FIG. 9C) and Sigma cat# HPA007270 (FIGS. 9B, 10B, 11B, 12B and 13B). All recombinant cells expressing LSR proteins were compared to the empty vector cells which were used as a negative control (numbered as A-1, B-1 and C-1 in all figures). Arrows indicate membrane staining.

Human WT LSR (SEQ ID NO:11) was observed mainly in intracellular regions with both the anti-Flag (Sigma cat# A9594) (FIG. 9A-2) and anti-LSR antibodies (Sigma cat# HPA007270, Abcam, cat ab59646 FIGS. 9B-2 and 9C-2 respectively), with a low percentage of cells demonstrating membrane localization.

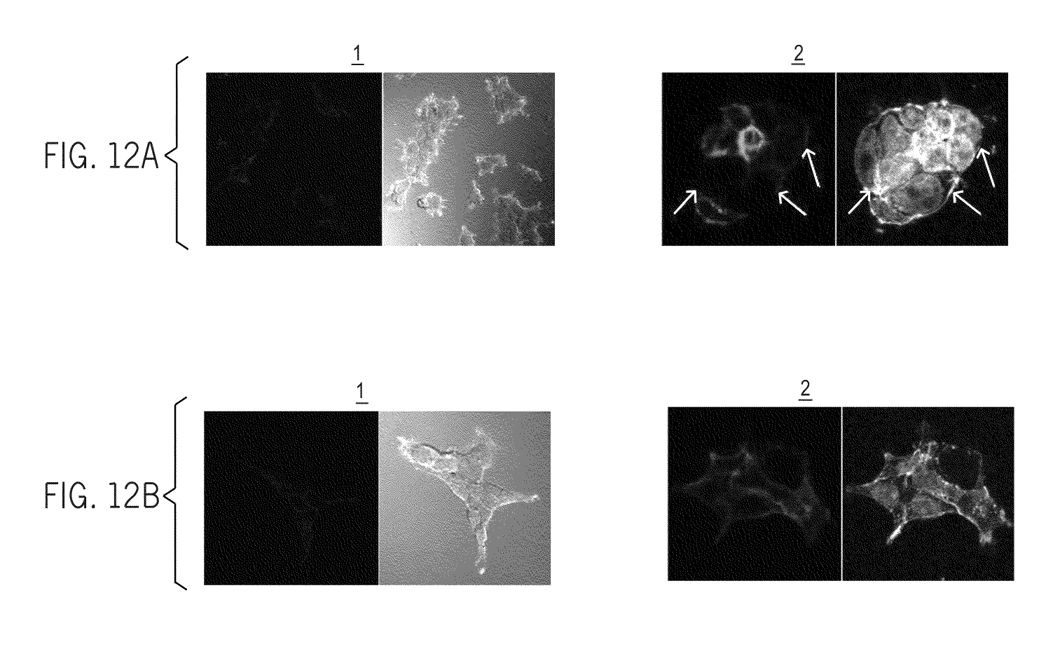

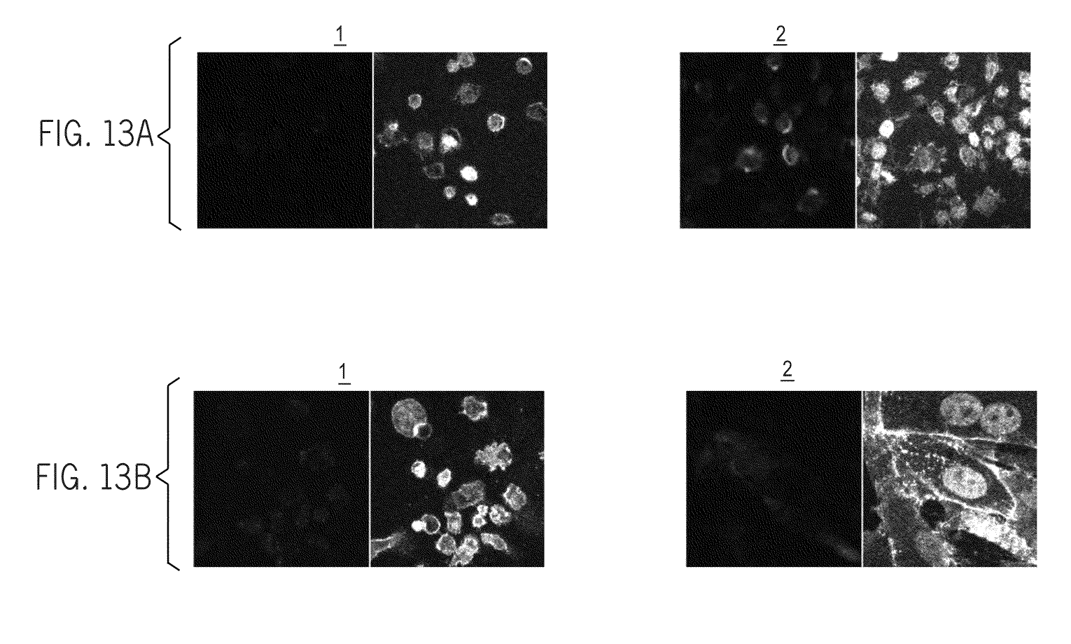

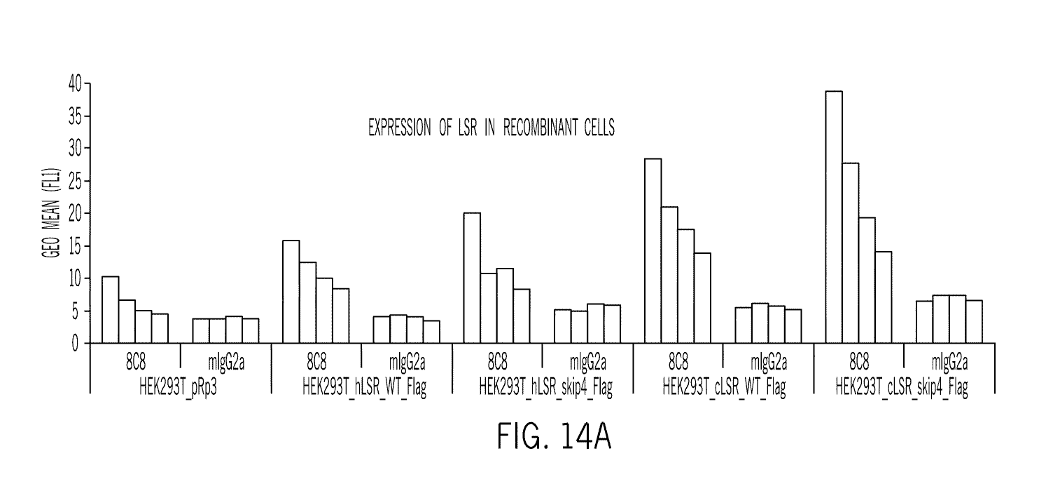

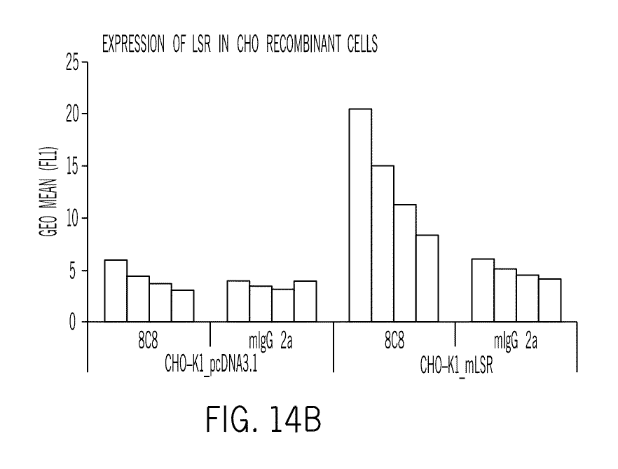

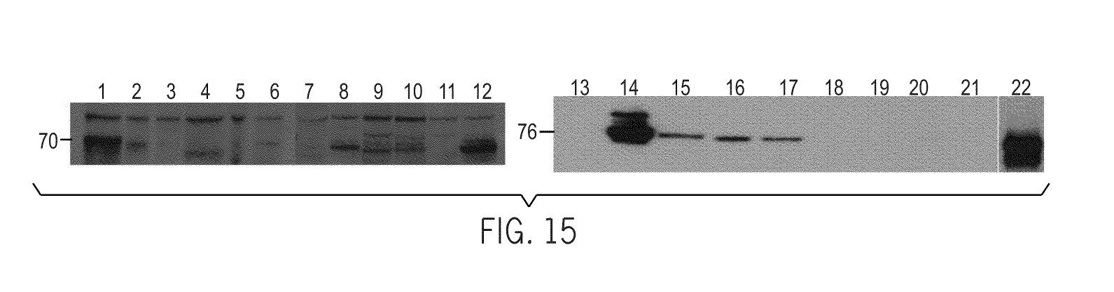

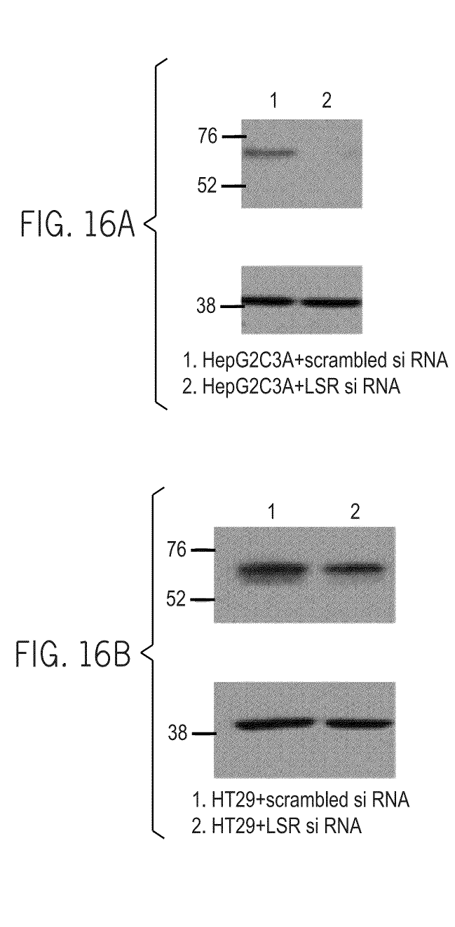

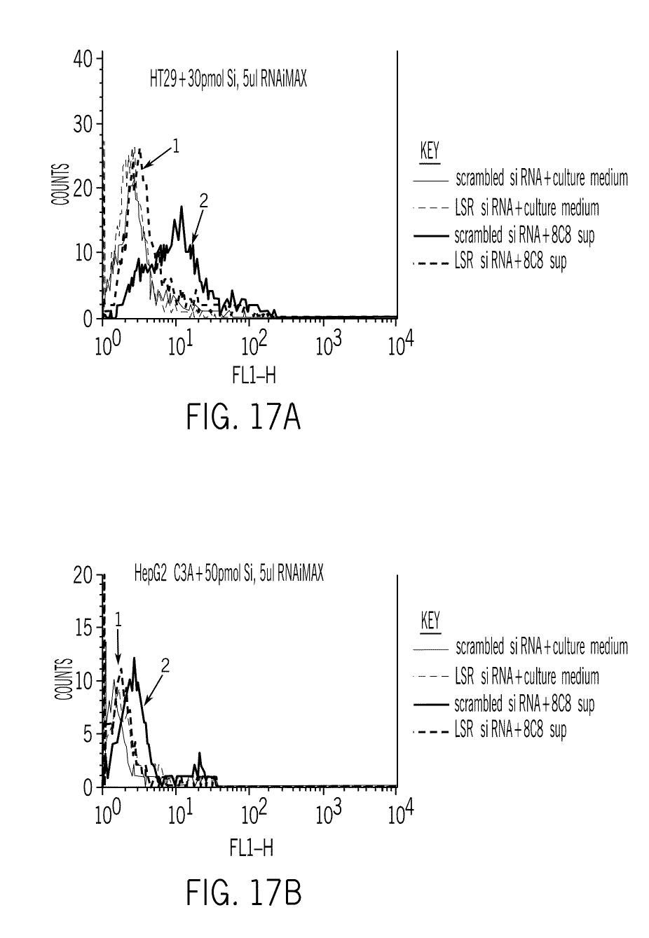

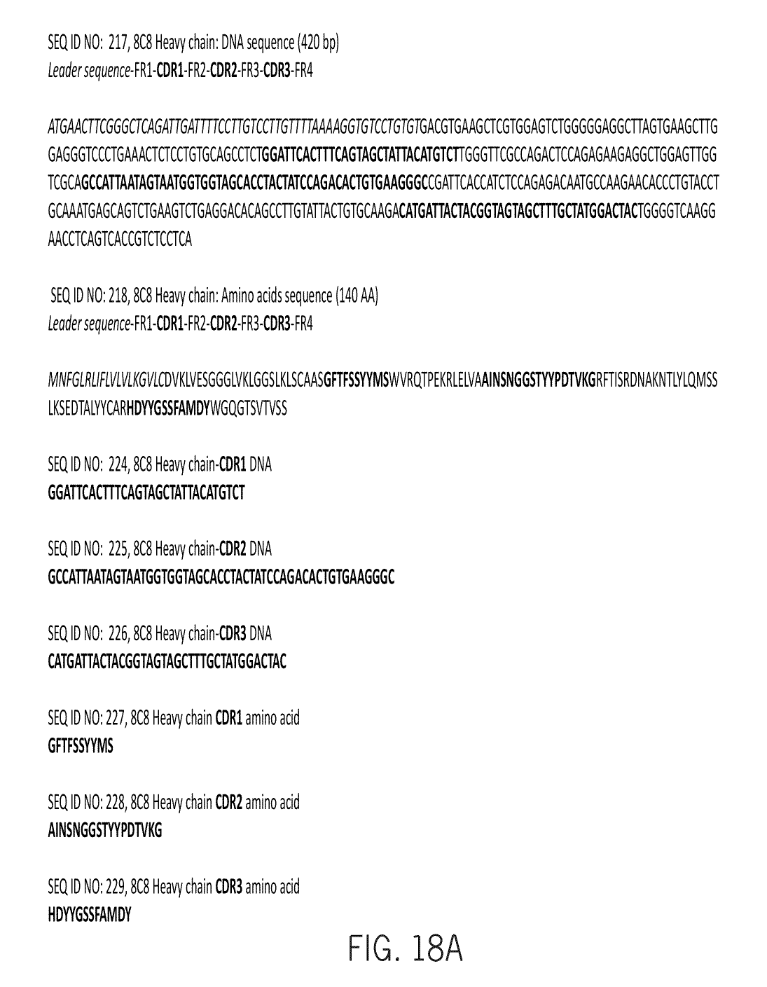

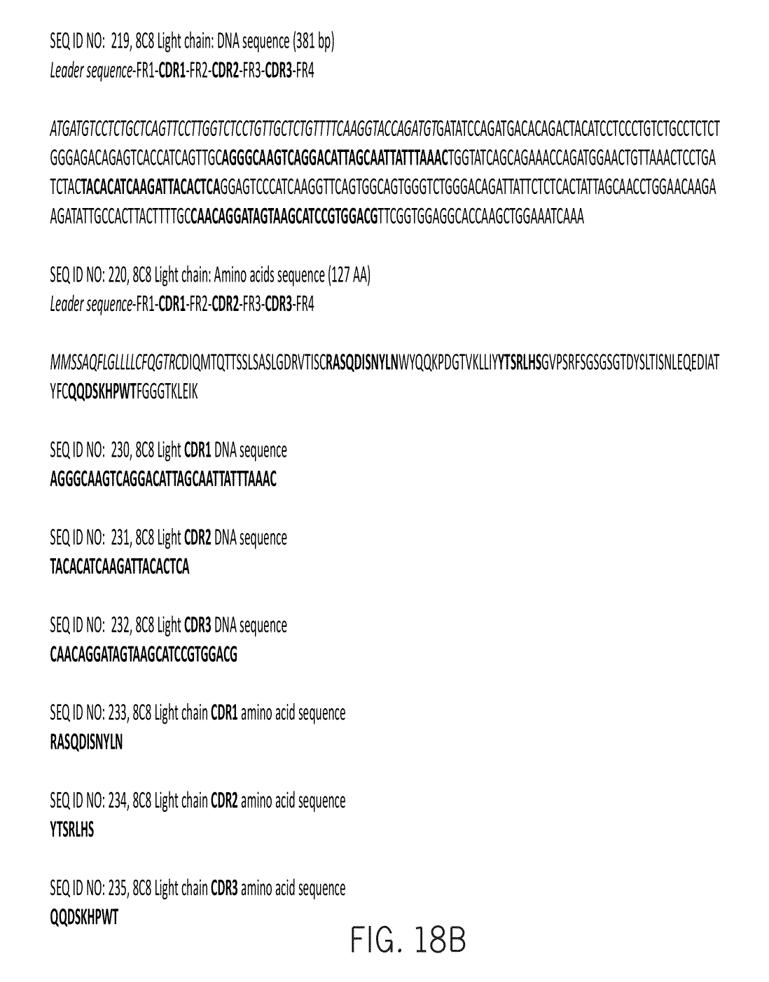

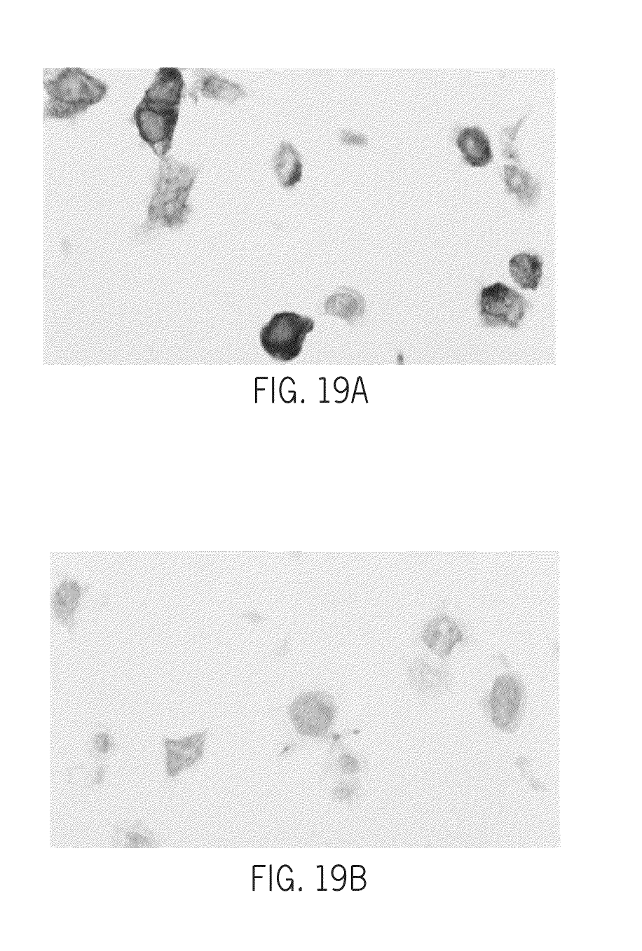

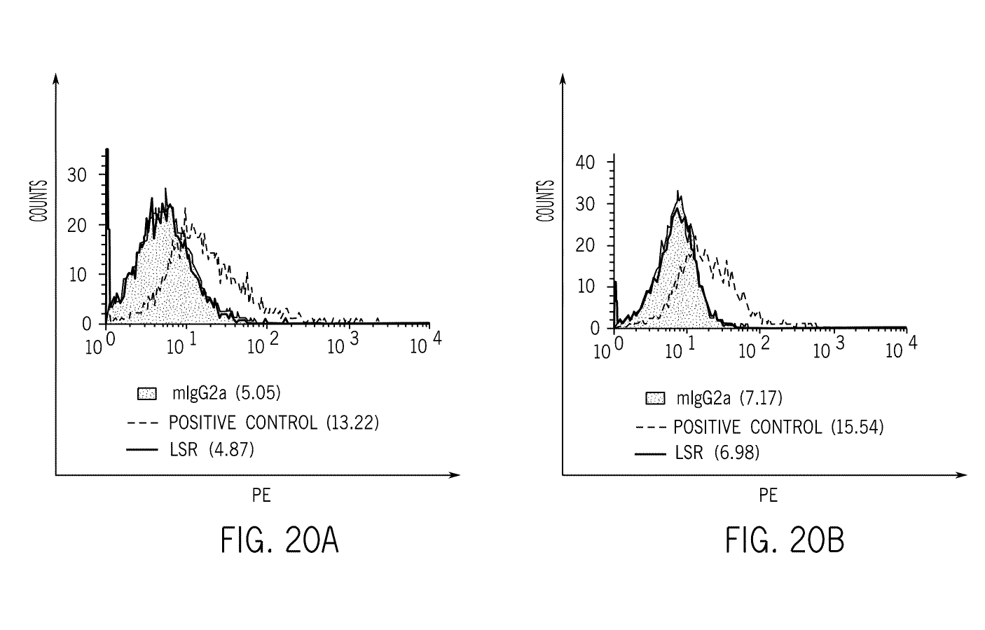

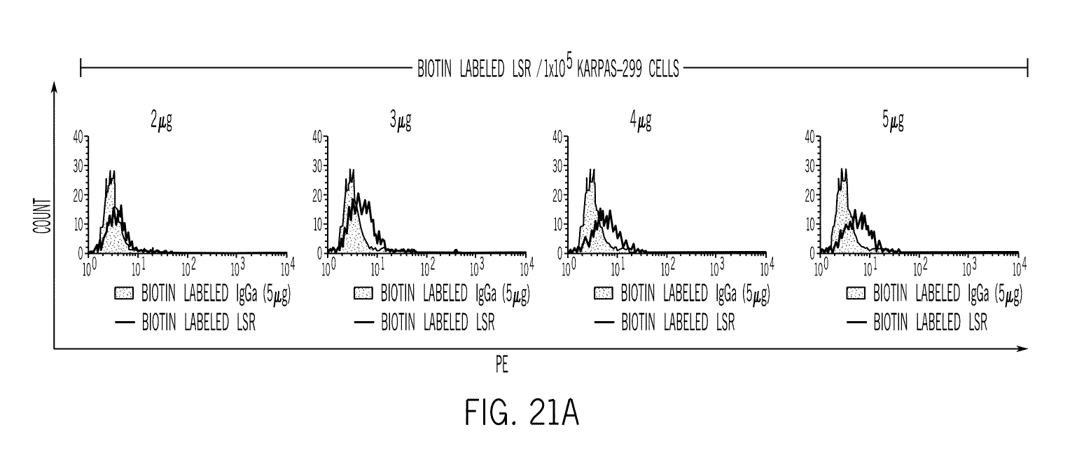

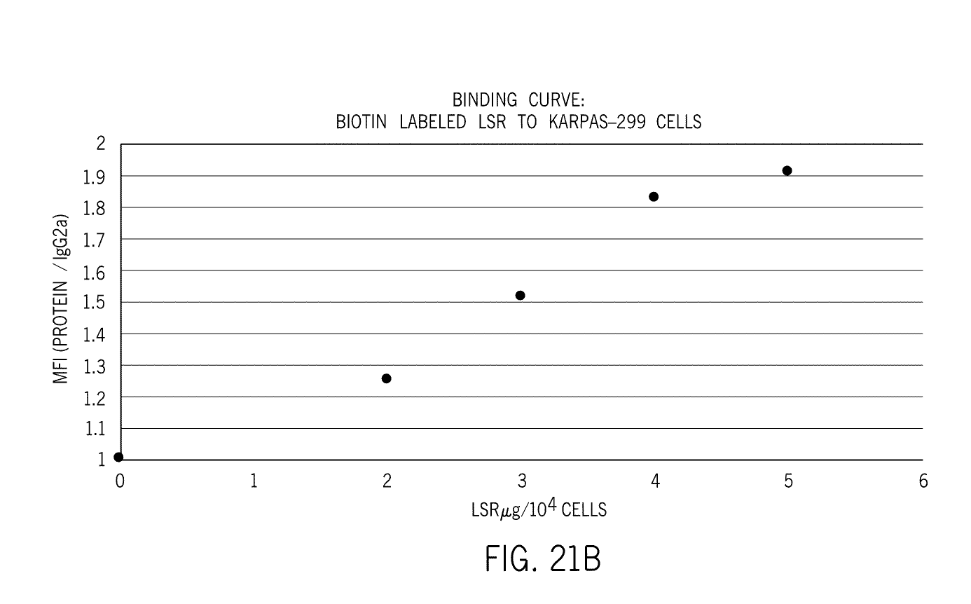

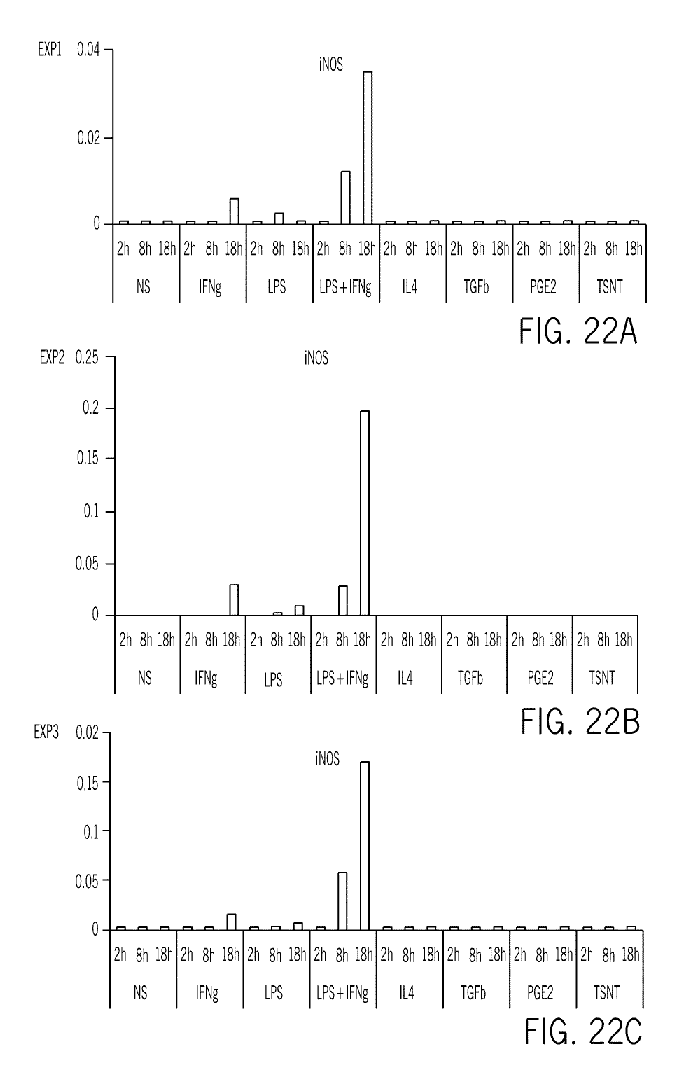

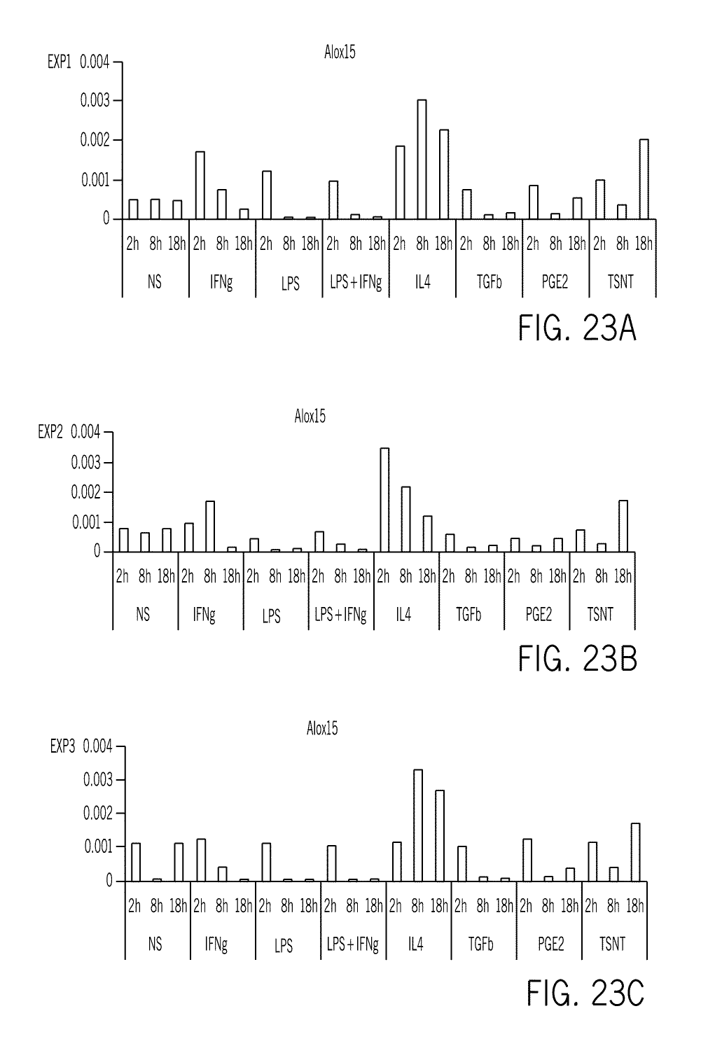

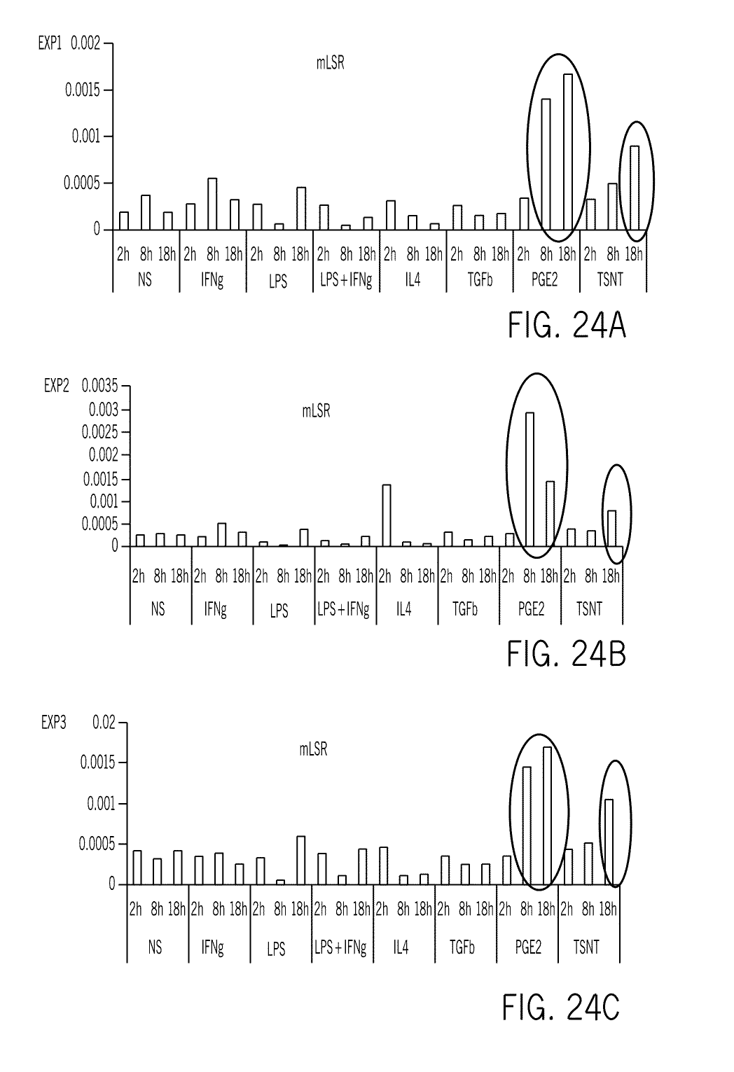

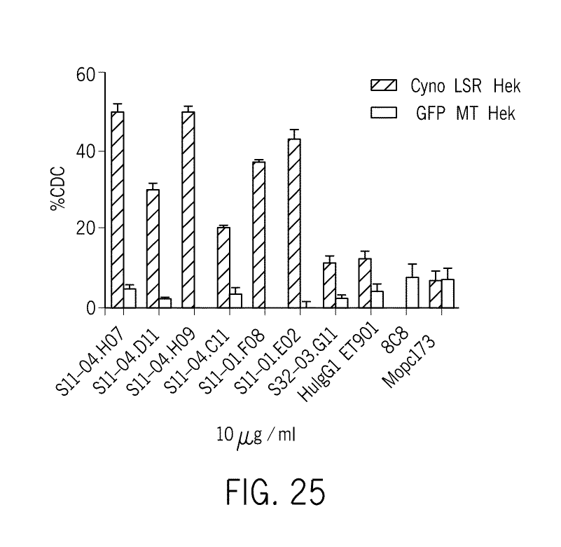

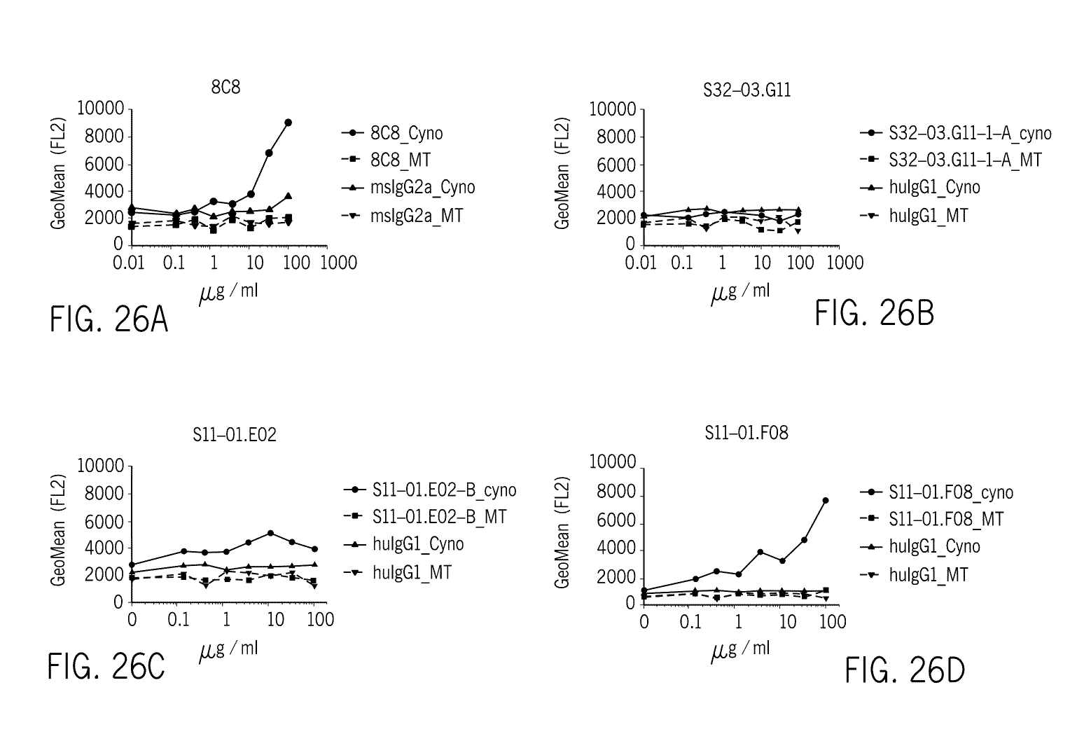

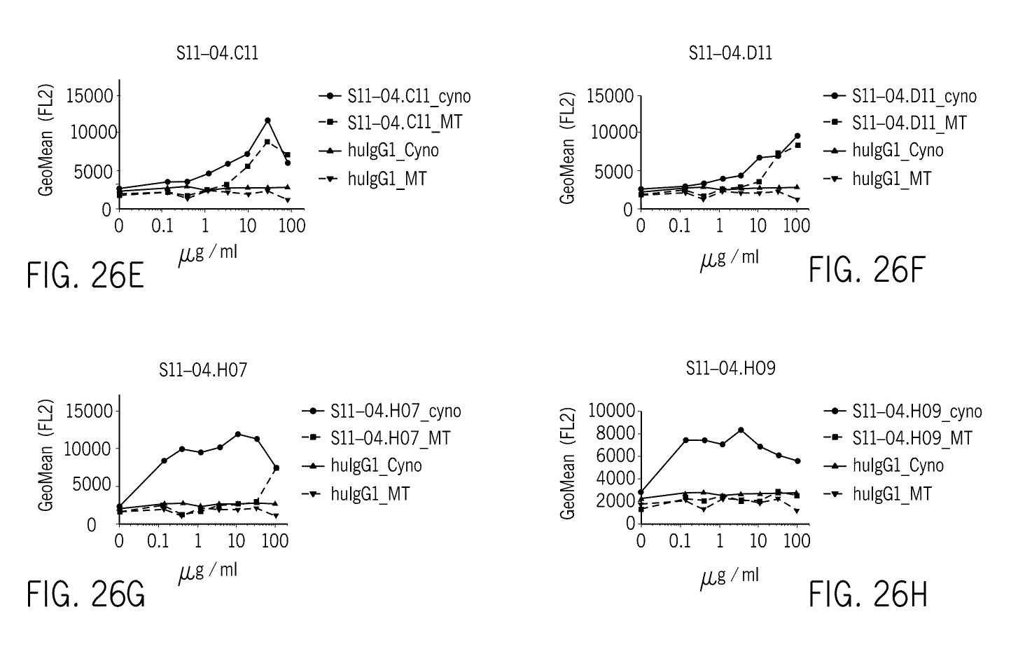

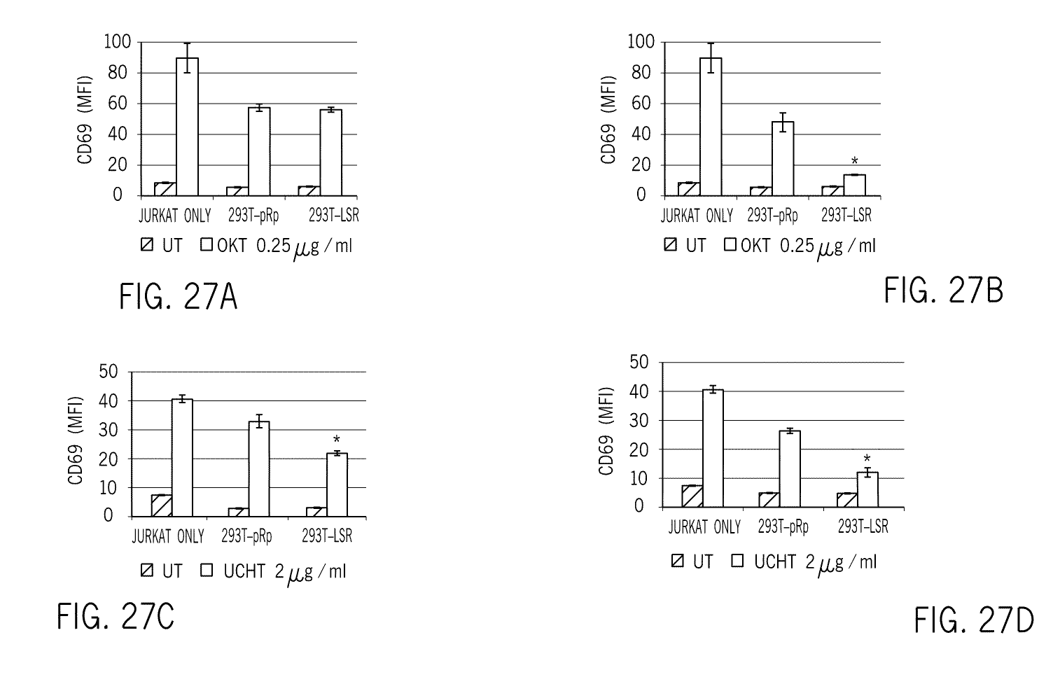

The cyno WT LSR (SEQ ID NO:211) protein was also observed mainly in intracellular regions, including in the ER and golgi, using an anti-flag antibody (FIG. 10A-2). However, the same cells expressing the cyno WT LSR (SEQ ID NO:211) protein showed a more pronounced membrane staining when using an anti-LSR antibody (Sigma cat# HPA007270) (FIG. 10B-2). Interestingly, using both antibodies, the expression on the cell surface was more pronounced in the recombinant cells expressing the cyno Skip4 LSR-flag variant (SEQ ID NO:212) as compared to recombinant cells expressing cyno WT LSR-flag protein (SEQ ID NO:211) (FIGS. 12A-2 and 12B-2).

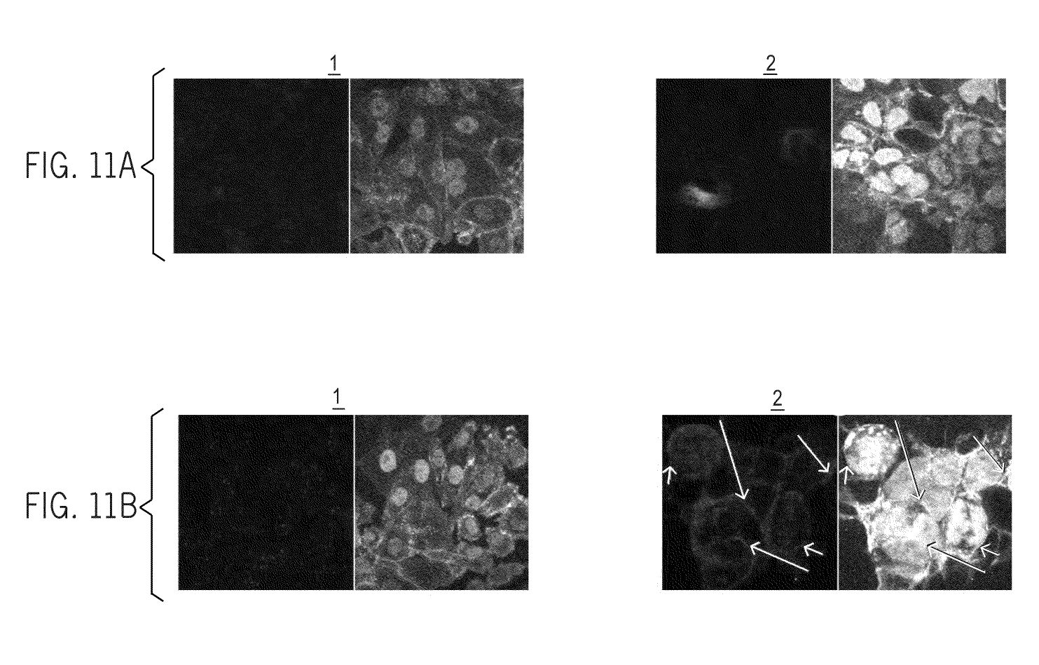

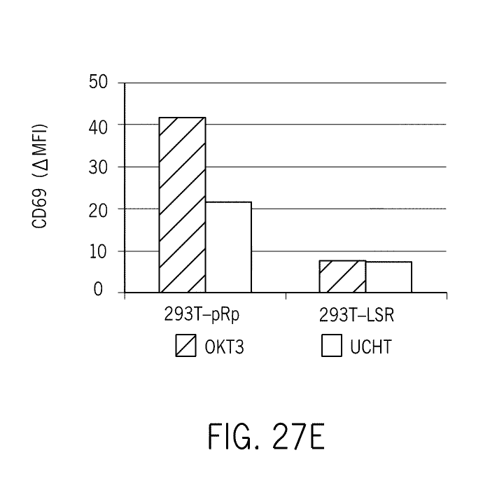

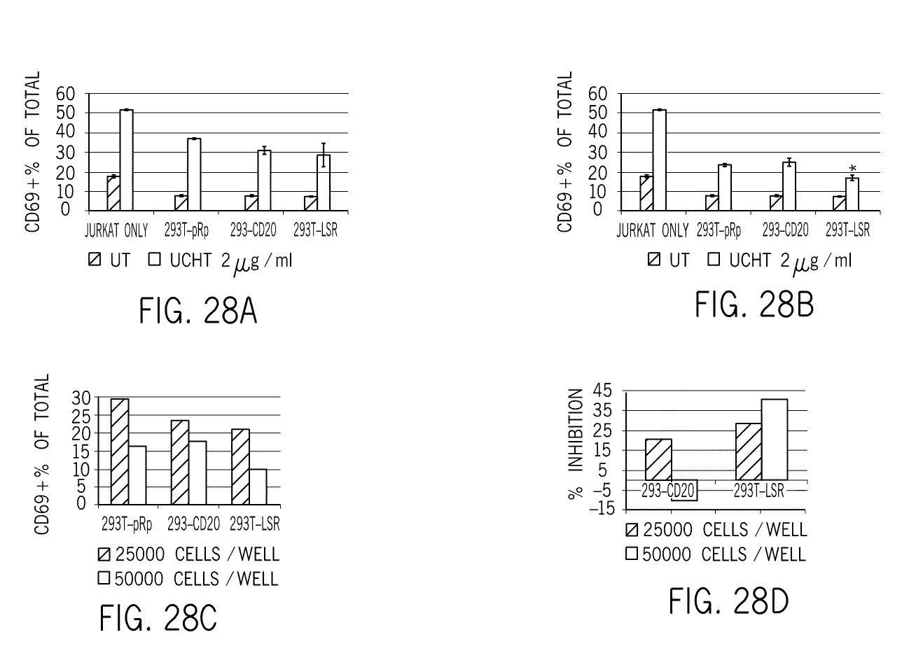

Recombinant HEK293 cells expressing the mouse WT LSR (SEQ ID NO:31) protein show a signal with the anti-flag antibody, albeit very weak (FIG. 11A), and a weak signal on the cell membrane using the specific anti-LSR antibody (Sigma cat# HPA007270) (FIG. 11B). Recombinant CHO-K1 cells expressing the mouse LSR-flag protein (SEQ ID NO:31) (FIGS. 13A and 13B) demonstrate ER localization with both antibodies.

### Example 6

A. Validation of Cell Surface Expression of Human and Cyno LSR Proteins by Facs Analysis of Stably Expressing Cells

HEK293T and CHO_K1 stably transfected cells over expressing the various LSR proteins (WT and skip4) (SEQ ID NOs:11, 13, 211, 212 and 31), were analyzed by flow cytometry (FACS) using the 8C8 hybridoma clone. As shown in FIG. 14, binding of the 8C8 mAb with different mAb concentrations to cells stably expressing the LSR proteins (human WT (SEQ ID NO:11), cyno WT (SEQ ID NO:211), mouse WT (SEQ ID NO:31), human and cyno Skip4 variants (SEQ ID NOs:13, 212 respectively) was considerably higher than that observed with cells transfected with the empty vector, or cells stained with the control culture medium, indicating cell membrane expression of the five LSR proteins.

B. Analysis of the Expression of Endogenous Lsr Protein in Various Cell Lines

The expression of endogenous LSR protein in various cancer lines (derived from ovary, liver, breast, cervix and colon, described in Table 3) was analyzed by Western Blotting as described below.

Whole cell extracts (50-75 ug for the cancer cell lines, and 30 ug for the ectopically expressing cell lines), were analyzed by SDS-PAGE and transferred to Nitrocellulose membrane as described above. The membrane was blocked with TTBS (Biolab, Cat#: 20892323)/5% skim milk (Difco, Cat#232100) and incubated with anti LSR antibodies (Abcam,cat#ab596460R Sigma, cat# HPA007270) diluted in TTBS/5% BSA (Sigma-Aldrich, A4503) at the indicated concentrations (Table 1), for 1 hour at Room Temperature. After 3 washes with in TTBS, the membrane was further incubated for 1 hour at Room Temperature with the secondary-conjugated antibodies as indicated (Table 1), diluted in TTBS. Chemiluminescence reaction was performed with ECL Western Blotting Detection Reagents (GE Healthcare, Cat # RPN2209) and the membrane was exposed to Super RX Fuji X-Ray film (Catalog no. 4741008389).

TABLE 3

| Lane | | | | | |
|---|---|---|---|---|---|
| No. in | | | | | |
| FIG. | | | | | |
| 15 | Cell line | ATCC No. | Morphology | Source | Disease |
| 2 | SK-BR-3 | HTB-30, | Epithelial | Breast | Adeno- |
| | | ATCC | | | carcinoma |
| 3 | MCF7 | HTB-22, | Epithelial | Breast | Adeno- |
| | | ATCC | | | carcinoma |
| 5 | HeLa | CCL-2, | Epithelial | Cervix | Adeno- |
| | | ATCC | | | carcinoma |
| 6 | Caco-2 | HTB-37, | Epithelial | Colon | colorectal |
| | | ATCC | | | adeno- |
| | | | | | carcinoma |
| 22 | HT-29 | HTB-38, | Epithelial | Colon | colorectal |
| | | ATCC | | | adeno- |
| | | | | | carcinoma |
| 7 | TOV- | CRL-11731, | Epithelial | Ovary | Primary |
| | 112D | ATCC | | | malignant |
| | | | | | adeno- |
| | | | | | carcinoma; |
| | | | | | endometrioid |
| | | | | | carcinoma |
| 8 | SK-OV-3 | HTB-77, | Epithelial | Ovary | Adeno- |
| | | ATCC | | | carcinoma |
| 9 | OVCAR3 | HTB-161, | Epithelial | Ovary | Adeno- |
| | | ATCC | | | carcinoma |
| 10 | OV-90 | CRL-11732, | Epithelial | Ovary | Malignant |
| | | ATCC | | | papillary serous |
| | | | | | adeno- |
| | | | | | carcinoma |
| 11 | ES2 | CRL-1978, | Epithelial | Ovary | clear cell |
| | | ATCC | | | carcinoma |
| 12 | Caov-3 | HTB-75, | Epithelial | Ovary | Adeno- |
| | | ATCC | | | carcinoma |
| 4, 15 | HepG2 | HB-8065, | Epithelial | Liver | Hepatocellular |
| | | ATCC | | | carcinoma |
| 16 | HepG2/ | CRL-10741, | Epithelial | Liver | Hepatocellular |
| | C3A | ATCC | | | carcinoma |
| 17 | Hep 3B | HB-8064, | Epithelial | Liver | Hepatocellular |
| | | ATCC | | | carcinoma |
| 18 | SNU182 | CRL-2235, | Epithelial | Liver | Hepatocellular |
| | | ATCC | | | carcinoma |
| 19 | SKHEP-1 | HTB-52, | Epithelial | Liver | Adeno- |
| | | ATCC | | | carcinoma |
| 20 | PLC/PRF5 | CRL-8024, | Epithelial | Liver | Hepatoma |
| | | ATCC | | | |
| 21 | Chang | 330139, CLS | Epithelial | Liver | Normal |
| | liver | | | | |

FIG. 15 demonstrates endogenous expression of LSR in various cell lines. A protein band corresponding to the expected ˜70 kDa LSR was observed in the positive control cells (lanes 1 & 14) and was also detected in several cell lines, pointing to endogenous expression of LSR in liver cancer cell lines HepB3, HepG2 and its derivative HepG2/C3A (lanes 4, 15, 16, 17), breast cancer cell line SK-BR-3 (lane#2) and ovarian cancer cell lines SKOV-3, OVCAR-3, ES-2, Caov-3 (lanes 8, 9, 10, 12).

### Example 7

### Generation of Mouse Monoclonal Antibodies Directed Against LSR

Production of murine monoclonal antibodies against the extra-cellular domain of human LSR protein (SEQ ID NO:10) was performed at BIOTEM (Parc d′ activite Bievre Dauphine, 885 rue Alphonse Gourju, 38140 APPRIEU, France), using a peptide immunization strategy. The peptides that were used for the immunization were taken from the extra cellular domain of the human LSR protein (SEQ ID NO:10), and are disclosed below.

The first phase of the project to raise anti-LSRmAbs included immunization of 3 BALB/c mice using two peptides derived from the ECD region of the LSR protein (SEQ ID NO:10) as follows: peptide 1: KSFCRDRIADAFSPASVD, corresponding to amino acid residues 81-98 of the SEQ ID NO:10, as set forth in SEQ ID NO:215, and peptide 2: CQDSVRTVRVVATKQGNA, corresponding to amino acid residues 118-135 of SEQ ID NO:10, as set forth in SEQ ID NO:216. The amino acid positions are counted from the second Met in the open reading frame.

The second phase of the protocol included fusion of the lymphocytes from the immunized mice with Sp2/O-Ag14 myeloma cells and plating out on 10 microtiter 96-well plates.

Mature clones were screened by ELISA using the human LSR fusion protein (SEQ ID NO:236), the peptides used for immunization (SEQ ID NOs: 215, 216), and the recombinant HEK293T cells expressing human WT LSR-flag protein. FACS analysis was subsequently carried out with purified monoclonal Ab, 8C8, using a goat anti mouse-Alexa Fluor 488 (Invitrogen cat# A10667) as secondary Ab for detection.

The third phase includes hybridoma cloning by limiting dilution and stabilization, and further processing for production and purification.

Culture supernatants of the hybridoma clones were analyzed by ELISA. As shown in Table 4, one positive clone (8C8) was identified that showed specific binding to the human LSR fusion protein (SEQ ID NO:236) and to HEK293T cells over expressing the human WT LSR protein, and not to non-relevant human IgG1 fusion protein or HEK293T cells transfected with empty pRp3 vector. This clone showed binding to peptide 1 (SEQ ID NO:215), but not to peptide 2 (SEQ ID NO:216).

The purified mAb, 8C8, of the positive clone was further analyzed by flow cytometry (FACS) using HEK293T transfected cells over expressing the various LSR proteins (SEQ ID NOs: 11, 13, 211, 212, 31).

TABLE 4

| | Absorbances against the following antigens: | | | | | | | | | | | | | |
|---|---|---|---|---|---|---|---|---|---|---|---|---|---|---|
| | | | | | CGEN | | | | | | Cells | | Cells | |
| | | | | | 15021-Fc | | Protein X-Fc | | | | HEK293T- | | HEK293T- | |
| Hybridoma | Peptide 1 | | Peptide 2 | | Human IgG1 | | Human IgG1 | | No antigen | | CGEN15022 | | pRp3 | |
| (Dilution of supernatant) | Pure | 1/10 | Pure | 1/10 | Pure | 1/10 | Pure | 1/10 | Pure | 1/10 | Pure | 1/10 | Pure | 1/10 |
| 8C8 | 1.824 | 2.085 | 0.041 | 0.041 | 1.048 | 1.187 | 0.047 | 0.041 | 0.043 | 0.039 | 0.310 | 0.281 | 0.100 | 0.134 |

The results presented in FIG. 14 point to the specificity of the 8C8 mAb and its suitability for FACS analysis, shown by the detection of the ectopic cell surface expression of LSR proteins using the mAb 8C8, as compared to the negative controls—mouse IgG2a (isotype control) and cells transfected with the empty vector. The specificity of the 8C8 hybridoma clone to LSR was further confirmed using siRNA-mediated specific knockdown of endogenous LSR cell surface expression on HT29 colon cancer cells endogenously, as described and shown above.

### Example 8

### Monoclonal Antibody Sequencing

Total RNA was extracted from frozen hybridoma cells following the technical manual of TRIzol® Plus RNA Purification System (Invitrogen, Cat. No.: 15596-026). The total RNA was analyzed by agarose gel electrophoresis. Total RNA was reverse transcribed into cDNA using isotype-specific anti-sense primers or universal primers following the technical manual of SuperScript™ III First-Strand Synthesis System (Invitrogen, Cat. No. 18080-051). RT-PCR was then performed to amplify the heavy and light chains of the antibody. The antibody fragments of VH and VL were amplified according to the standard operating procedure of RACE of GenScript.

Amplified antibody fragments were separately cloned into a standard cloning vector using standard molecular cloning procedures.

Colony PCR screening was performed to identify clones with inserts of correct sizes.

Ten single colonies with correct VH and VL insert sizes were sent for sequencing. The VH and VL genes of ten different clones were found nearly identical.

The consensus sequence, shown below is the sequence of the antibody produced by the hybridoma 8C8 antibody 8C8. The DNA and amino acid sequence of the heavy chain of the 8C8 antibody is shown in SEQ ID NOs: 217 and 218, respectively. The DNA and amino acid sequence of the light chain of the 8C8 antibody is shown in SEQ ID NOs: 219 and 220, respectively. The leader sequence is shown in Italic font; the sequences of CDR1, CDR2, CDR3 are shown in bold. The constant regions FR1, FR2, FR3 and FR4 are shown in a regular font. FIG. 18A presents the nucleic and amino acid sequences of the 8C8 antibody Heavy chain CDRs. The nucleic acid sequences of 8C8 antibody Heavy chain CDR1, CDR2, CDR3 are set forth in SEQ ID NOs: 224, 225, 226, respectively. The corresponding amino acid sequences of 8C8 antibody Heavy chain CDR1, CDR2, CDR3 are set forth in SEQ ID NOs: 227, 228, 229, respectively. FIG. 18B presents the nucleic and amino acid sequences of the 8C8 antibody Light chain CDRs. The nucleic acid sequences of 8C8 antibody Light chain CDR1, CDR2, CDR3 are set forth in SEQ ID NOs: 230, 231, 232, respectively. The corresponding amino acid sequences of 8C8 antibody Light chain CDR1, CDR2, CDR3 are set forth in SEQ ID NOs: 233, 234, 235, respectively.

SEQ ID NO: 217, 8C8 Heavy chain: DNA sequence (420 bp)
Leader sequence-FR1-CDR1-FR2-CDR2-FR3-CDR3-FR4

ATGAACTTCGGGCTCAGATTGATTTTCCTTGTCCTTGTTTTAAAAGG
TGTCCTGTGTGACGTGAAGCTCGTGGAGTCTGGGGGAGGCTTAGTGA
AGCTTGGAGGGTCCCTGAAACTCTCCTGTGCAGCCTCTGGATTCACT
TTCAGTAGCTATTACATGTCTTGGGTTCGCCAGACTCCAGAGAAGAG
GCTGGAGTTGGTCGCAGCCATTAATAGTAATGGTGGTAGCACCTACT
ATCCAGACACTGTGAAGGGCCGATTCACCATCTCCAGAGACAATGCC
AAGAACACCCTGTACCTGCAAATGAGCAGTCTGAAGTCTGAGGACAC
AGCCTTGTATTACTGTGCAAGACATGATTACTACGGTAGTAGCTTTG
CTATGGACTACTGGGGTCAAGGAACCTCAGTCACCGTCTCCTCA

SEQ ID NO: 218, 8C8 Heavy chain. Amino acids sequence (140 AA)
Leader sequence-FR1-CDR1-FR2-CDR2-FR3-CDR3-FR4

MNFGLRLIFLVLVLKGVLCDVKLVESGGGLVKLGGSLKLSCAASGFTFSS
YYMSWVRQTPEKRLELVAAINSNGGSTYYPDTVKGRFTISRDNAKNTLYL
QMSSLKSEDTALYYCARHDYYGSSFAMDYWGQGTSVTVSS

SEQ ID NO: 219, 8C8 Light chain: DNA sequence (381 bp)
Leader sequence-FR1-CDR1-FR2-CDR2-FR3-CDR3-1-R4

ATGATGTCCTCTGCTCAGTTCCTTGGTCTCCTGTTGCTCTGTTTTCAAGG
TACCAGATGTGATATCCAGATGACACAGACTACATCCTCCCTGTCTGCCT
CTCTGGGAGACAGAGTCACCATCAGTTGCAGGGCAAGTCAGGACATTAGC
AATTATTTAAACTGGTATCAGCAGAAACCAGATGGAACTGTTAAACTCCT
GATCTACTACACATCAAGATTACACTCAGGAGTCCCATCAAGGTTCAGTG
GCAGTGGGTCTGGGACAGATTATTCTCTCACTATTAGCAACCTGGAACAA
GAAGATATTGCCACTTACTTTTGCCAACAGGATAGTAAGCATCCGTGGAC
GTTCGGTGGAGGCACCAAGCTGGAAATCAAA

SEQ ID NO: 220, 8C8 Light chain: Amino acids sequence (127 AA)
Leader sequence-FR1-CDR1-1⁴R2-CDR2-FR3-CDR3-1-R4

MMSSAQFLGLLLLCFQGTRCDIQMTQTTSSLSASLGDRVTISCRASQDIS
NYLNWYQQKPDGTVKLLIYYTSRLHSGVPSRFSGSGSGTDYSLTISNLEQ
EDIATYFCQQDSKHPWTFGGGTKLEIK

SEQ ID NO: 224, 8C8 Heavy chain-CDR1DNA sequence

GGATTCACTTTCAGTAGCTATTACATGTCT

SEQ ID NO: 225, 8C8 Heavy chain-CDR2DNA sequence

GCCATTAATAGTAATGGTGGTAGCACCTACTATCCAGACACTGTGAA
GGGC

SEQ ID NO: 226, 8C8 Heavy chain-CDR3DNA sequence

CATGATTACTACGGTAGTAGCTTTGCTATGGACTAC

SEQ ID NO: 227, 8C8 Heavy chain CDR1 amino acid sequence

GFTFSSYYMS

SEQ ID NO: 228, 8C8 Heavy chain CDR2 amino acid sequence

AINSNGGSTYYPDTVKG

SEQ ID NO: 229, 8C8 Heavy chain CDR3 amino acid sequence

HDYYGSSFAMDY

SEQ ID NO: 230, 8C8 Light CDR1DNA sequence

AGGGCAAGTCAGGACATTAGCAATTATTTAAAC

SEQ ID NO: 231, 8C8 Light CDR2DNA sequence

TACACATCAAGATTACACTCA

SEQ ID NO: 232, 8C8 Light CDR3DNA sequence

CAACAGGATAGTAAGCATCCGTGGACG

SEQ ID NO: 233, 8C8 Light chain CDR1 amino acid sequence

RASQDISNYLN

SEQ ID NO: 234, 8C8 Light chain CDR2 amino acid sequence

YTSRLHS

SEQ ID NO: 235, 8C8 Light chain CDR3 amino acid sequence

QQDSKHPWT

### Example 9

### IHC Analysis of LSR Proteins

Calibration Study

In order to establish the correct antibody concentration and antigen retrieval for evaluation of LSR expression, a calibration study was carried out.

Formalin Fixed Paraffin Embedded (FFPE) sections (4 μm) of cell lines: HEK293T expressing LSR and, pRp ‘Empty Vector’ cells and full-face tissue sections of normal liver, tumour liver, breast tumour and ovarian tumour, described in Table 5 herein, were used.

A positive control of the detection of Von Willebrand's factor in sections of human colon, and a positive control cell line were included in the assay to validate the secondary antibody, LSR antibodies and detection reagents. A ‘no primary’ control was included.

The sections were deparaffinised, antigen retrieved and rehydrated using pH9.0 Flex+ 3-in-1 antigen retrieval buffer, in PT Link apparatus at 95° C. for 20 min Following antigen retrieval, sections were placed in Flex wash buffer for 10 min, and then loaded into a DAKO Autostainer Plus. The sections were then incubated for 10 min with Flex+ Peroxidase Blocking reagent, rinsed twice in 50 mM Tris. HCl, 150 mM NaCl, 0.1% Tween-20, pH 7.6 (TBST), followed by a 10 min incubation with Protein Block reagent (DAKO X0909).

The sections were incubated for 30 min with primary antibody diluted in DAKO Envision Flex antibody diluent (DAKO Cytomation, Cat # K8006). LSR pAb was tested at: 6, 4 and 2 μg/ml in the respective sections The negative control sections were incubated with non-immune rabbit IgG antibodies (Dako, CAT #0936) at 6, 4 and 2 μg/ml or in DAKO Envision Flex antibody diluent (‘no primary’ control).

Following incubation with primary antibodies, the sections were then rinsed twice in FLEX buffer, incubated with anti-mouse/rabbit Flex+ HRP for 20 min, rinsed twice in FLEX buffer and then incubated with diaminobenzidine (DAB) substrate for 10 min. The chromagenic reaction was stopped by rinsing the slides with distilled water. Following chromagenesis, the sections were counterstained with haematoxylin, dehydrated in an ascending series of ethanols (90-99-100%), cleared in three changes of xylene and coverslipped under DePeX. Stained sections were analyzed, and suitable digital images captured, using an Olympus BX51 microscope with a Leica DFC290 camera.

### IHC Tissue Microarray (TMA)

The aim of the study was to determine the LSR expression in various cancerous tissues using LSR specific antibodies according to at least some embodiments of the invention. The distribution of LSR in formalin-fixed/paraffin-embedded (FFPE) sections was examined using immunohistochemistry (IHC).

Using polyclonal Rabbit antibodies, as described above, in FFPE sections of tumour and normal tissue microarray (‘mutli-tumour TMA’) and in full face sections of normal lymph node, tonsil and spleen from three donors, as described in Table 6 herein. The TMA comprised 11 tissue types (Table 3): breast, colon, lymphoid and prostate (8 tumour and 2 normal samples of each), gastric, ovary, brain, kidney, liver and skin (4 tumour and 2 normal samples of each), and lung (8 non-small cell tumour and 4 small cell tumour samples, and 4 normal samples). Additional normal tissues, sections of lymph node (n=3), tonsil (n=3) and spleen (n=3) were sectioned and used in this study (Table 7). FFPE sections (4 μm) of cell line HEK293T expressing LSR, the ‘multi-tumour’ TMA and full-face sections of normal lymph node, tonsil and spleen were used. The sections were de-paraffinised, antigen retrieved and rehydrated using pH9.0 Flex+ 3-in-1 antigen retrieval buffers, in PT Link apparatus at 95° C. for 20 min with automatic heating and cooling. Following antigen retrieval, sections were washed in distilled water for 2×5 min then loaded into a DAKO Autostainer Plus. The sections were then incubated for 10 min with Flex+ Peroxidase Blocking reagent, rinsed twice in 50 mM Tris. HCl, 150 mM NaCl, 0.1% Tween-20, pH 7.6 (TBST), followed by a 10 min incubation with Protein Block reagent (DAKO X0909). The sections were incubated for 30 min with primary antibody diluted in DAKO Envision Flex antibody diluent (DAKO Cytomation, Cat # K8006). LSR poly clonal antibody was applied at 6 μg/ml. The negative control sections were incubated with non-immune rabbit IgG antibodies (Dako, CAT #0936) at 6 μg/ml or in DAKO Envision Flex antibody diluent (‘no primary’ control). Following incubation with primary antibodies, the sections were then rinsed twice in FLEX buffer, incubated with anti-mouse/rabbit Flex+ HRP for 20 min, rinsed twice in FLEX buffer and then incubated with diaminobenzidine (DAB) substrate for 10 min. The chromagenic reaction was stopped by rinsing the slides with distilled water. Following chromagenesis, the sections were counterstained with haematoxylin, dehydrated in an ascending series of ethanols (90-99-100%), and cleared in three changes of xylene and coverslipped under DePeX. Stained sections were analyzed, and suitable digital images captured, using an Olympus BX51 microscope with a Leica DFC290 camera. The sections were analysed for the intensity of the specific staining and a semi-quantitative scoring system was used. The core in the tissue array with the most intense LSR immunoreactivity was assigned a score of 3+and the intensities of the immunoreactivity in the other cores were scored relative to that of the 3+core. The percentage of LSR immunoreactive tumour was estimated and recorded using the following ranges: 0-25%, 25-50%, 50-75% and 75-100%.

### Results:

The following dataset represents the optimisation and detection of LSR Rabbit polyclonal antibody in specified positive control tissues and in an empty vector cell line. Assay positive controls demonstrating internal assay working conditions and antibody validation are shown in FIG. 19. FIG. 19 (X60) shows sections of LSR positive cells following pH9.0 AR. Panel A shows the slides incubated with LSR antibody applied at 2 μg/ml. Panel B shows the adjacent ‘no primary’ incubated sections. Positive LSR immunoreactivity is seen in Panel A, demonstrating valid antibody working conditions. The adjacent no primary section (Panel B) shows no apparent immunoreactivity. Specific immunoreactivity was detected in positive control tissues in both normal and tumour liver samples and in breast tumour samples. The LSR antibody demonstrated membrane and cytoplasmatic staining in the two normal liver donors and in one tumour donor.

Specific LSR immunoreactivity was detected in hepatocytes and the bile duct epithelium of the two normal liver samples at 6 and 4 μg/ml. In tumour liver, membrane and cytoplasmatic LSR immunoreactivity was seen, and was markedly intense in tumour cells compared to the normal liver samples. In ovarian tumour (DI 16974), specific membrane and cytoplasmatic LSR immunoreactivity was seen at the highest concentration (6 μg/ml). At 4 μg/ml, only cytoplasmic immunoreactivity was noted in the tumour cells. In sample DI 16976, specific cytoplasmic immunoreactivity was only observed in tumour cells at both 6 and 4 μg/ml. Non-specific immunoreactivity was detected, but only present at the highest concentration in both donor samples.

No apparent LSR immunoreactivity was detected in pRp3 empty vector cells. Non-specific immunoreactivity was detected.

LSR immunoreactive tissues have shown a varied in staining pattern and the level of intensity amongst tumour types. It was however noted that tumours of colon and liver stained the most intense, while other tumours showed moderate staining throughout the samples. In the majority of cores, immunoreactivity averaged 75-100%. Table 8 presents the full analysis of the TMA.

LSR Expression in Breast Tumors

Within the breast tumour set, the intensity of staining varied in the range of (1-2+) with two tumours scoring 2+. Only one core of breast tumour within the array was 0-25% immunoreactive, the majority were in the region of 75-100% immunoreactive. The 2+ scoring tumours were invasive ductal carcinomas (IDCs).

LSR Expression in Large Bowel Cancer

Within the large bowel cohort, eight samples were of moderately differentiated adenocarcinoma tumours. Six samples scored a (2+ or 3+), one scored the strongest level of immunoreactivity of (3+), and one sample scored (0-1+). All samples were 75-100% immunoreactive. In the one normal sample, specific cytoplasmic immunoreactivity was seen in the ascending portion of the mucosal epithelium, where the descending crypts exhibited a membrane-bound phenotype with a staining score of (2-3).

LSR Expression in Prostate Cancer

In the prostate tumours, the level of immunoreactivity varied between (1-2+). One tumour recorded a 2+ score (Gleason score 9) and two recorded a 1-2 score (Gleason scores 9 and 7) and three scored 1+ from a cohort of 8 adenocarcinomas. All prostate tumours appeared to be LSR immunoreactive. In these cores, immunoreactivity was cytoplasmic with exceptions where a prominent membrane phenotype was observed within the tumour epithelium. Within the normal prostate samples, staining was seen in the glandular epithelium and occasional smooth muscle regions with staining intensity of +1-+2.

LSR Expression in Lymphoma

Within lymphoma samples, a lower level of staining was observed—three samples of NHL scored (1+) and five of NHL and HLwith (0-1+), ranging in 75-100% immunoreactivity within cores (HL sample had an IHC score of 0-1). It was however noted that in one donor (15052), a discrete population of tumour cells demonstrated higher level of staining. In two samples of normal lymph node, cytoplasmic staining was seen in one sample within the germinal centres. A few discrete immune cells were observed to demonstrate immunoreactivity within the other sample.

LSR Expression in Lung Cancer:

In the lung tumour set, eight out of twelve tumour samples exhibited specific LSR immunereactivity. The intensity and immunoreactivity seen was varied within tumour types. In one sample of NSCL adenocarcinoma, the tumours were strongly immunoreactive with a score of 2+-3+, where in two samples of squamous carcinoma—an intensity score of (2+) and (1+-2+) was seen. Two samples of NSCLC had an intensity level of 1+-2+ and both were poorly to moderately differentiated. In three adenocarcinoma samples, two demonstrated a staining intensity score of 2+ where the other sample showed a staining score of only 1+, and was poorly differentiated. Most tumours were seen to be 75-100% immunoreactive. In the cohort of small cell carcinoma samples, no apparent immunoreactivity was seen in tumour cells; only occasional immunoreactivity was seen in putative macrophages. In samples of normal lung, positive cytoplasmic immunoreactivity was seen in the respiratory epithelium. Occasional pneumocytes were also seen to be immunoreactive with intensity of +1 to +2.

LSR Expression in Stomach Tumor

In stomach tumor, four adenocarcinoma samples (moderately differentiated) demonstrated apparently LSR expression within the tumours, of which, 75-100% were immunoreactive in each core. All samples were seen to vary in the level of scoring with one sample scoring 2+-3+, where the rest of the samples scored 1+ or 2+. It was noted in certain tissues—occasional prominent membrane-associated staining was seen, with infiltrating putative macrophages also demonstrating immunoreactivity. Within the normal stomach tissue, apparently specific cytoplasmic immunoreactivity was seen in the mucosal epithelium. In one particular donor (2874), cytoplasmic and nuclear staining was seen, with intensity of +1 to +2.

LSR Expression in Ovarian Cancer

The ovarian carcinoma cohort also demonstrated specific diffuse-cytoplasmic immunoreactivity in tumours with cores 75-100% immunoreactive. Staining intensity was to be variable within tumour types. Two samples of serous papillary carcinoma scored 0-1+ and 2+ respectively, with a few tumour cells showing a darker-intense staining pattern (Donor 13003). In the granulosa tumour, this was scored relatively weak (0-1+), however, the serous cystadenocarcinoma sample was seen to have an intense stain of 2+with associated-membrane immunoreactivity (Donor 9407). In normal ovary tissues, some specific immunoreactivity was noted in only one sample in the stromal region with staining intensity of +2.

LSR Expression in Melanoma

In skin melanoma, staining was seen weak (0-1+), with 25-50% of the tumour cells being immunoreactive. No apparent membrane-associated staining was observed within this tissue. In normal skin, no immunoreactivity was seen within the epidermis. Only occasional dermal lymphocytes were positively stained.

LSR Expression in Brain Tumor

In brain tumour, (grade 4-astrocytoma) two samples demonstrated immunoreactivity in 75-100% of the tumour cells. One donor (donor 9516) in particular, showed nuclear-membrane staining (2+), compared to donor 13845, with a less-intense stain (0-1+). One patient with grade 2 astrocytome had an IHC scone of 0. No correlation can be made between tumour type and level of immunoreactivity. In normal brain, one sample demonstrated apparently specific cytoplasmic immunoreactivity within the neuropil cells and other observed neuro-fibres.

LSR Expression in Renal Tumors

In the renal carcinomas, three clear-cell type tumours and one non-clear cell carcinoma demonstrated LSR immunoreactivity. The level of intensity varied between 1+-2+ with 75-100% of tumour cells being immunoreactive in each core. It was also noted that clear-cell carcinomas showed a cytoplasmic membrane-specific bound staining pattern compared to less differentiated cell types. In normal kidney samples, specific cytoplasmic staining was seen in the collecting tubules.

LSR Expression in Liver Tumors

In liver tumours, all three samples demonstrated prominent immunoreactivity in tumour cells, where 75-100% of tumour cells were stained in each core. Donor 19115 was assigned a score of 3+, (and was used to score other cores relative to its intensity level) as this was the most intensely stained core in the array. It was also noted that in a subset of cells an intense level of cytoplasmic staining was observed, compared to the surrounding tumours, with occasional membrane-associated immunoreactivity. In normal liver, specific membrane immunoreactivity was seen.

LSR Expression in Normal Lymphatic Tissue

In full-face sections of normal lymph node, tonsil and spleen, it was observed that the majority demonstrated positive cytoplasmic staining in the germinal centres from the three tissue sets, with a few putative macrophages also showing immunoreactivity. In lymph node, some cells were observed to show staining of the cytoplasmic-membrane within the lymphoid follicle and occasional smooth muscle was also stained.

TABLE 5
TMA samples characterisitcs (calibration)

| | Donor | | | | |
|---|---|---|---|---|---|
| Tissue | ID | Age | Sex | Clinical diagnosis | Pathology |
| Liver | 3323 | 77 | Female | Pulmonary | Sections of |
| parenchyma | | | | embolism(COD); | normal liver |
| | | | | Pulmonary | |
| | | | | infarction(COD); | |
| | | | | Myocardial | |
| | | | | infarction | |
| Liver | 14453 | 15 | Male | Cerebral | Sections of |
| parenchyma | | | | infarction(CoD); | normal liver |
| | | | | Cardiomyopathy; | with portal |
| | | | | Penicillin allergy | tracts |
| Tumour | 14826 | 66 | Female | Hepatocellular | Moderately |
| liver | | | | carcinoma; | differentiated |
| | | | | Alcoholic | hepatocellular |
| | | | | cirrhosis; | carcinoma |
| | | | | Cholecystitis, | |
| | | | | chronic | |
| Tumour | 16974 | 57 | Female | Cystadeno- | Cystadeno- |
| ovary | | | | carcinoma, ovary; | carcinoma |
| | | | | Heart disease; | |
| | | | | Diabetes | |
| Tumour | 16976 | 49 | Female | Cystadeno- | Cystadeno- |
| Ovary | | | | carcinoma, | carcinoma |
| | | | | ovary; Hyper- | |
| | | | | tension; Diabetes | |
| Breast | 13533 | 73 | Female | Breast carcinoma; | Poorly |
| tumour | | | | Intraductal breast | differentiated |
| | | | | carcinoma; Grade 3 | carcinoma |
| | | | | | consistent with |
| | | | | | grade 3 ductal |
| | | | | | carcinoma of |
| | | | | | breast. |
| Breast | 4957 | 86 | Female | Breast carcinoma; | Sections of |
| tumour | | | | Adenocarcinoma; | normal breast |
| | | | | Hypertension; | tissue |
| | | | | Leiomyoma; | |
| | | | | Arthritis; Migraine | |
| Hek293T- | | | | Positive | Positive |
| LSR | | | | | transfected cells |
| Hek 293T- | | | | Negative | Negative′ empty |
| Prp | | | | | vector′ cell lines |

TABLE 6
Cancer TMA samples characteristics

| | Donor | | | |
|---|---|---|---|---|
| Map ID | ID | Tissue | Age | Sex |
| 1 | 8589 | tumour: breast: ductal- | 46 | Female |
| | | adenocarcinoma | | |
| 2 | 8707 | tumour: breast: ductal- | 46 | Female |
| | | adenocarcinoma | | |
| 3 | 8723 | tumour: breast: ductal- | 74 | Female |
| | | adenocarcinoma | | |
| 4 | 15778 | tumour: breast: lobular carcinoma | 52 | Female |
| 5 | 3724 | tumour: breast: ductal- | 82 | Female |
| | | adenocarcinoma | | |
| 6 | 2953 | tumour: breast: ductal- | 67 | Female |
| | | adenocarcinoma | | |
| 7 | 9132 | tumour: breast: ductal- | 82 | Female |
| | | adenocarcinoma | | |
| 8 | 9298 | tumour: breast: ductal- | 73 | Female |
| | | adenocarcinoma | | |
| 9 | 5704 | Normal: breast | 46 | Female |
| 10 | 5347 | Normal: breast | 64 | Female |
| 11 | 3550 | tumour: colon: adenocarcinoma | 61 | Male |
| 12 | 15767 | Tumour: large | 58 | Female |
| | | intestine: adenocarcinoma | | |
| 13 | 4537 | intestine: adenocarcinoma Sigmoid | 44 | Female |
| | | colon carcinoma; | | |
| 14 | 2206 | tumour: colon: adenocarcinoma | 76 | Female |
| 15 | 9542 | tumour: colon: adenocarcinoma | 73 | Male |
| 16 | 2893 | tumour: colon: adenocarcinoma | 62 | Male |
| 17 | 15764 | Tumour: large | 75 | Female |
| | | intestine: adenocarcinoma | | |
| 18 | 15763 | Tumour: large | 69 | Female |
| | | intestine: adenocarcinoma | | |
| 19 | 2681 | Normal: colon | 54 | Female |
| 20 | 3121 | Normal: colon | 34 | Male |
| 21 | 15296 | tumour: prostate | 68 | Male |
| 22 | 15295 | tumour: prostate: adenocarcinoma | 71 | Male |
| 23 | 15301 | tumour: prostate: adenocarcinoma | 51 | Male |
| 24 | 15758 | tumour: prostate: adenocarcinoma | 74 | Male |
| 25 | 15745 | tumour: prostate: adenocarcinoma | 52 | Male |
| 26 | 15777 | tumour: prostate: adenocarcinoma | 68 | Male |
| 27 | 15755 | tumour: prostate: adenocarcinoma | 55 | Male |
| 28 | 15756 | tumour: prostate: adenocarcinoma | 68 | Male |
| 29 | 5678 | Normal: Prostate Gland | 48 | Male |
| 30 | 13951 | Normal: Prostate Gland | 37 | Male |
| 31 | 15052 | Lymphoma | 45 | Female |
| 32 | 15760 | Tumour: lymphoma | 72 | Female |
| 33 | 17549 | Tumour: lymphoma | 47 | Male |
| 34 | 15039 | Lymphoma | 47 | Male |
| 35 | 15034 | Lymphoma | 71 | Male |
| 36 | 15037 | Lymphoma | 53 | Female |
| 37 | 17547 | Lymphoma | | |
| 38 | 15775 | Tumour: lymphoma | 75 | Female |
| 39 | 4655 | Normal: lymph-node | 1 | Female |
| 40 | 10789 | Normal: lymph-node | 58 | Male |
| 41 | 12053 | tumour: lung | 72 | Male |
| 42 | 15772 | Tumour: lung: non-small cell | 44 | Male |
| | | carcinoma | | |
| 43 | 13586 | tumour: lung | 67 | Female |
| 44 | 2760 | tumour: lung: squamous-cell- | 64 | Male |
| | | carcinoma | | |
| 45 | 9354 | tumour: lung: adenocarcinoma | 63 | Male |
| 46 | 3473 | tumour: lung: adenocarcinoma | 72 | Male |
| 47 | 2765 | tumour: lung: adenocarcinoma | 64 | Female |
| 48 | 4852 | tumour: lung: adenocarcinoma | 56 | Female |
| 49 | 10414 | small cell | 74 | Male |
| 50 | 15055 | tumour: lung: small cell | 52 | Male |
| 51 | 15054 | tumour: lung: small cell | 65 | Male |
| 52 | 15053 | tumour: lung: small cell | 52 | Male |
| 53 | 1311 | lung: parenchyma | 36 | Female |
| 54 | 141 | ung: parenchyma | 39 | Female |
| 55 | 5767 | Normal: lung: parenchyma | 45 | Male |
| 56 | 2649 | Normal: lung: parenchyma | 37 | Male |
| 57 | 14551 | tumour: stomach | 83 | Female |
| 58 | 10656 | tumour: stomach | 74 | Male |
| 59 | 13200 | tumour: stomach | 85 | Male |
| 60 | 2295 | tumour: stomach | 66 | Female |
| 61 | 13665 | stomach: body | 57 | Female |
| 62 | 2874 | stomach: body | 53 | Male |
| 63 | 12998 | tumour: ovary | 78 | Female |
| 64 | 13003 | tumour: ovary | 74 | Female |
| 65 | 5739 | tumour: ovary | 48 | Female |
| 66 | 9407 | tumour: ovary | 75 | Female |
| 67 | 4739 | Normal: ovary | 42 | Female |
| 68 | 4781 | Normal: ovary | 34 | Female |
| 69 | 15759 | Tumour: skin melanoma | 65 | Male |
| 70 | 15753 | Tumour: skin melanoma | 46 | Female |
| 71 | 15038 | Tumour: skin melanoma | 41 | Male |
| 72 | 15343 | Tumour: skin melanoma | 24 | Male |
| 73 | 13110 | Normal: skin | 22 | Female |
| 74 | 15415 | Normal: skin | 45 | Female |
| 75 | 15342 | Tumour: brain: glioblastoma | 56 | Male |
| | | multiforme | | |
| 76 | 9514 | tumour: brain | 17 | Male |
| 77 | 13845 | tumour: brain | 58 | Male |
| 78 | 9516 | tumour: brain | 25 | Female |
| 79 | 2007 | Normal: brain: cortex: frontal | 40 | Male |
| 80 | 4585 | Normal: brain: cortex: frontal | 85 | Male |
| 81 | 13266 | tumour: kidney | 71 | Male |
| 82 | 14125 | tumour: kidney | 41 | Male |
| 83 | 4764 | tumour: kidney | 66 | Male |
| 84 | 9043 | tumour: kidney | 45 | Male |
| 85 | 2874 | Normal: kidney: cortex | 53 | Male |
| 86 | 4818 | Normal: kidney: cortex | 52 | Female |
| 87 | 19115 | Tumour: liver | | |
| 88 | 15757 | Tumour: liver | 25 | Male |
| 89 | 14826 | tumour: liver | 66 | Female |
| 90 | 19114 | Tumour: liver | | |
| 91 | 1991 | Normal: liver: parenchyma | 79 | Female |
| 92 | 3123 | Normal: liver: parenchyma | 31 | Male |

TABLE 7
Lympatic organ TMA samples charateristics

| | | | | Clinical | |
|---|---|---|---|---|---|
| Tissue | ID | Age | Sex | diagnosis | Pathology Report |
| Lymph- | 53524 | 73 | F | Subarachnoid | Sections of lymph node |
| node | | | | haemorrhage | showing normal histological |
| | | | | (COD); | features with lymphoid |
| | | | | Hypertension; | aggregates and well defined |
| | | | | Non-insulin | sinuses. DIAGNOSIS: normal |
| | | | | dependent | lymph node. |
| | | | | diabetes | |
| | | | | mellitus | |
| Lymph- | 3217 | 36 | M | Intracranial | Sections of lymph node |
| node | | | | haemorrhage | showing normal histological |
| | | | | (COD); Organ | features with lymphoid |
| | | | | donor | aggregates and well defined |
| | | | | | sinuses. DIAGNOSIS: normal |
| | | | | | lymph node. |
| Lymph- | 9191 | 32 | M | Pulmonary | A lymph node containing |
| node | | | | arterial | many macrophages which |
| | | | | hypertension | are filled with anthracotic |
| | | | | and heart | pigment. No significant |
| | | | | defect | pathological abnormality. |
| Tonsil | 10821 | 17 | F | Tonsillitis, | Non dysplastic squamous |
| | | | | chronic | epithelium overlying normal |
| | | | | | tonsillar lymphoid tissue. |
| Tonsil | 10045 | 25 | F | Tonsillitis | Normal tonsillar tissue |
| | | | | | including epithelium and |
| | | | | | lymphoid follicles. |
| Tonsil | 11024 | 6 | M | Tonsillitis, | Tonsil with few neutrophils |
| | | | | chronic; | in the epithelium. |
| | | | | Dyspnoea | |
| Spleen | 14345 | 60 | M | Tonsillitis | Normal spleen. White and |
| | | | | | red pulp present. |
| Spleen | 13851 | 18 | F | Intracerebral | Normal spleen with normal |
| | | | | haemorrhage | red and white pulp |
| | | | | (CoD); | identified. Moderate |
| | | | | Hypertension; | preservation |
| | | | | Hyper- | |
| | | | | lipidaemia; | |
| | | | | Non-insulin | |
| | | | | dependent | |
| | | | | diabetes | |
| | | | | mellitus; | |
| | | | | Arthritis | |
| Spleen | 15947 | 53 | M | Intracranial | Normal spleen. |
| | | | | haemorrhage | |
| | | | | (CoD); | |
| | | | | Endometriosis | |

TABLE 8
cancer TMA samples IHC analysis

| | | | | % | |
|---|---|---|---|---|---|
| | | | Tumour | tumour | |
| Map | | | immuno | immuno | |
| ID | Tissue | Diagnosis | reactivity | reactivity | coments |
| 1 | tumour: breast: ductal- | Intra duct and | 1-2+ | 0-25 | |
| | adenocarcinoma | invasive ductal | | | |
| | | carcinoma | | | |
| 2 | tumour: breast: ductal- | Invasive ductal | 0 | 0 | |
| | adenocarcinoma | carcinoma | | | |
| 3 | tumour: breast: ductal- | Primary | 2+ | 75 | |
| | adenocarcinoma | (invasive ductal | | | |
| | | pattern) | | | |
| 4 | tumour: breast: lobular | | — | — | Core loss |
| | carcinoma | | | | |
| 5 | tumour: breast: ductal- | | 2+ | 75-100 | |
| | adenocarcinoma | | | | |
| 6 | tumour: breast: ductal- | DCIS (ductal | | | |
| | adenocarcinoma | carcinoma in | — | — | Core loss |
| | | situ) | | | |
| 7 | tumour: breast: ductal- | sarcomatoid | — | — | Core loss |
| | adenocarcinoma | ductal | | | |
| 8 | tumour: breast: ductal- | Invasive ductal | — | — | Core loss |
| | adenocarcinoma | | | | |
| 9 | breast | Normal breast | — | — | Core loss |
| | | tissue | | | |
| 10 | breast | Normal breast | — | — | Core loss |
| | | tissue | | | |
| 11 | tumour: colon: | Differentiated | 2-3+ | 75-100 | tumour |
| | adenocarcinoma | adenocarcinoma | | | cells. |
| | | | | | Putative |
| | | | | | macrophages |
| 12 | Tumour: large | Moderately | — | — | Core loss |
| | intestine: | differentiated | | | |
| | adenocarcinoma | | | | |
| 13 | Sigmoid colon | adenocarcinoma. | 3+ | 75-100 | |
| | carcinoma; | Moderately | | | |
| | | differentiated | | | |
| 14 | tumour: colon: | invasive | 2-3+ | 75-100 | |
| | adenocarcinoma | | | | |
| 15 | tumour: colon: | Moderately | 2-3+ | 75-100 | |
| | adenocarcinoma | differentiated | | | |
| | | adenocarcinoma. | | | |
| 16 | tumour: colon: | Well | 2-3+ | 75-100 | |
| | adenocarcinoma | differentiated | | | |
| | | adenocarcinoma | | | |
| 17 | Tumour: large | Moderately | 0-1 | 75-100 | Core is |
| | intestine: | differentiated | | | 75% |
| | adenocarcinoma | adenocarcinoma. | | | necrotic |
| 18 | Tumour: large | Moderately | 2-3+ | 75-100 | |
| | intestine: | differentiated | | | |
| | adenocarcinoma | adenocarcinoma. | | | |
| 19 | colon | Normal colon: | | | 2-3 |
| | | full thickness. | | | Mucosal |
| | | | | | staining - |
| | | | | | Mucosal |
| | | | | | staining - |
| 20 | colon | Full thickness | — | — | Core loss |
| | | normal colon | | | |
| 21 | tumour: prostate | Adenocarcinoma | — | — | Core loss |
| 22 | tumour: prostate: | Adenocarcinoma | 1+ | 50-75 | |
| | adenocarcinoma | Gleason Score | | | |
| | | 3 + 3 = 6 | | | |
| 23 | tumour: prostate: | Gleason Score | 1+ | 0-25 | |
| | adenocarcinoma | 3 + 4 = 7 | | | |
| 26 | tumour: prostate: | Gleason Score | 1+ | 75-100 | |
| | adenocarcinoma | 4 + 4 = 8 | | | |
| 27 | tumour: prostate: | Gleason Score | 1-2+ | 0-25 | |
| | adenocarcinoma | 3 + 4 = 7 | | | |
| 28 | tumour: prostate: | Gleason Score | 2+ | 75-100 | |
| | adenocarcinoma | 4 + 5 = 9 | | | |
| 29 | Prostate Gland | Normal | | | 1+ |
| | | prostatic tissue | | | cytoplasmic |
| | | | | | staining |
| | | | | | in |
| | | | | | glandular |
| | | | | | epithelium. |
| | | | | | Occasional |
| | | | | | membrane |
| | | | | | stain, 1+ |
| | | | | | staining in |
| | | | | | smooth |
| | | | | | muscle |
| 30 | Prostate Gland | Normal prostate | | | 1+ |
| | | | | | prominent |
| | | | | | membrane |
| | | | | | staining |
| | | | | | in luminal |
| | | | | | surface. |
| | | | | | Core 75% |
| | | | | | loss |
| 31 | Lymphoma | Lymph node | 1+ | 0-25 | Discrete |
| | | infiltrated by | | | population |
| | | large cell | | | of |
| | | lymphoma | | | tumour |
| | | | | | cells - |
| | | | | | darker |
| | | | | | staining |
| | | | | | (2+). |
| 32 | Tumour: lymphoma | Low Grade Non- | 1+ | 50-75 | |
| | | Hodgkin's | | | |
| | | Lymphoma | | | |
| 33 | Tumour: lymphoma | | 0-1 | 75-100 | |
| 34 | Lymphoma | High grade Non- | 0-1 | 75-100 | |
| | | Hodgkin's | | | |
| | | Lymphoma | | | |
| 35 | Lymphoma | Non-Hodgkin's | 1+ | 75-100 | |
| | | Lymphoma | | | |
| 36 | Lymphoma | Non-Hodgkin's | 1+ | 75-100 | |
| | | Lymphoma | | | |
| 37 | Lymphoma | | 0-1 | 75 | |
| 38 | Tumour: lymphoma | Hodgkin's | 0-1 | 75 | |
| | | Lymphoma | | | |
| 39 | lymph-node | | — | — | 0-1 |
| | | | | | cytoplasmic |
| | | | | | staining. |
| | | | | | In sub-set |
| | | | | | of immune |
| | | | | | cells - macs. |
| | | | | | Staining |
| | | | | | only in |
| | | | | | these |
| 40 | lymph-node | Normal lymph | — | — | 1-2+ |
| | | node. | | | cytoplasmic |
| | | | | | staining |
| | | | | | in germinal |
| | | | | | centres - |
| | | | | | diffuse |
| | | | | | staining. |
| 41 | tumour: lung | Poorly | 1-2+ | 75-100 | |
| | | differentiated | | | |
| | | non-small cell | | | |
| | | carcinoma with | | | |
| | | some squamoid | | | |
| | | features | | | |
| 42 | Tumour: lung: non- | Poorly | 1-2+ | 0-25 | |
| | small cell carcinoma | Differentiated | | | |
| | | non-small Cell | | | |
| | | Carcinoma | | | |
| 43 | tumour: lung | Moderately to | 1-2+ | 75-100 | |
| | | poorly | | | |
| | | differentiated | | | |
| | | squamous | | | |
| | | carcinoma. | | | |
| 44 | tumour: lung: | Moderately well | 2+ | 75-100 | |
| | squamous-cell-carcinoma | differentiated | | | |
| | | keratinising | | | |
| | | squamous cell | | | |
| | | carcinoma | | | |
| 45 | tumour: lung: | large cell type | 2-3+ | 75-100 | |
| | adenocarcinoma | showing | | | |
| | | features of an | | | |
| | | adenocarcinoma | | | |
| 46 | tumour: lung: | Poorly | 1+ | 75-100 | |
| | adenocarcinoma | differentiated | | | |
| | | adenocarcinoma | | | |
| 49 | tumour: lung | small cell | -ve | — | Negative |
| | | | | | staining |
| 50 | tumour: lung | small cell | -ve | — | Negative |
| | | | | | staining |
| 51 | tumour: lung | small cell | -ve | — | Negative |
| | | | | | staining |
| 52 | tumour: lung | small cell | -ve | — | Negative |
| | | | | | staining |
| 53 | lung: parenchyma | Normal lung | — | — | 1+ |
| | | | | | Cytoplasmic |
| | | | | | staining |
| | | | | | in |
| | | | | | respiratory |
| | | | | | epithelium. |
| 54 | lung: parenchyma | Normal lung | — | — | terminal |
| | | | | | bronchiole |
| | | | | | columnar |
| | | | | | epithelium |
| | | | | | staining |
| | | | | | (1-2+) |
| 55 | lung: parenchyma | Normal lung | — | — | Terminal |
| | | | | | bronchiole |
| | | | | | columnar |
| | | | | | epithelium |
| | | | | | staining |
| | | | | | (1-2+). |
| | | | | | Macrophages |
| | | | | | 1+ staining. |
| 56 | lung: parenchyma | Normal lung | — | — | No |
| | | | | | alveolar |
| | | | | | staining. |
| | | | | | Macrophages |
| | | | | | positive |
| | | | | | staining |
| | | | | | (0-1+) |
| 57 | tumour: stomach | Moderately | 2-3+ | 75-100 | |
| | | differentiated | | | |
| | | adenocarcinoma | | | |
| 58 | tumour: stomach | Moderately | 1+ | 75-100 | |
| | | differentiated | | | |
| | | adenocarcinoma | | | |
| 59 | tumour: stomach | Moderately | 1+ | 75-100 | Diffuse |
| | | differentiated | | | cytoplasmic |
| | | adenocarcinoma | | | staining. |
| | | | | | Macrophages |
| | | | | | (1+) staining. |
| 60 | tumour: stomach | Moderately | 2+ | 75-100 | |
| | | differentiated | | | |
| | | adenocarcinoma | | | |
| 61 | stomach: body | Normal stomach | — | — | 1+ |
| | | | | | Cytoplasmic |
| | | | | | and |
| | | | | | membrane |
| | | | | | staining |
| | | | | | in mucosal |
| | | | | | epithelium |
| 62 | stomach: body | Normal stomach | — | — | 1-2+ |
| | | | | | Cytoplasmic |
| | | | | | and |
| | | | | | membrane |
| | | | | | staining |
| | | | | | in mucosal |
| | | | | | epithelium. |
| | | | | | Occasional |
| | | | | | nuclear |
| | | | | | staining. |
| 63 | tumour: ovary | A serous | 2+ | 75-100 | |
| | | papillary cystic | | | |
| | | carcinoma. | | | |
| 64 | tumour: ovary | Invasive serous | 0-1+ | 75-100 | |
| | | papillary | | | |
| | | carcinoma. | | | |
| 65 | tumour: ovary | Granulosa cell | 0-1 | 75-100 | |
| | | tumour. | | | |
| 66 | tumour: ovary | Serous | 2+ | 75-100 | |
| | | cystadenocarcinoma | | | |
| 67 | ovary | Normal ovary | — | — | Granular |
| | | | | | epithelium |
| | | | | | occasional |
| | | | | | discreet |
| | | | | | cells (2+) |
| | | | | | staining. |
| | | | | | Stromal |
| | | | | | region - |
| | | | | | negative |
| | | | | | staining, |
| 68 | ovary | Normal ovary | — | — | No |
| | | | | | staining |
| | | | | | observed. |
| 69 | Tumour: skin | Malignant | 0-1 | N/A | PD |
| | | Melanoma | | | |
| 70 | Tumour: skin | High grade | — | — | Core loss |
| | | malignant | | | |
| | | Melanoma | | | |
| 71 | melanoma | Malignant | 0-1 | 25-50 | |
| | | melanoma | | | |
| 72 | Tumour: skin | Malignant | 0 | 0 | |
| | | Melanoma | | | |
| 73 | skin | Normal skin. | — | — | Epidermis |
| | | | | | negative |
| | | | | | staining |
| 74 | skin | Normal skin. | — | — | Epidermis |
| | | | | | negative |
| | | | | | staining. |
| | | | | | Occasional |
| | | | | | dermal |
| | | | | | lymphocytes |
| | | | | | positive |
| | | | | | (1+) |
| | | | | | staining. |
| 75 | Tumour: brain | Glioblastoma | — | — | Core loss |
| | | multiforme | | | |
| 76 | tumour: brain | Astrocytoma; | 0 | 0 | No |
| | | grade 2. | | | staining |
| | | | | | observed |
| 77 | tumour: brain | Glioblastoma | 0-1 | 75-100 | |
| | | multiforme; | | | |
| | | synonym grade | | | |
| | | 4 Astrocytoma | | | |
| 78 | tumour: brain | Astrocytoma; | 2+ | 75-100 | |
| | | grade 4. | | | |
| 79 | brain: cortex: frontal | Normal brain | — | — | 0-1 |
| | | | | | Cytoplasmic |
| | | | | | staining |
| | | | | | in |
| | | | | | neuropil. |
| 80 | brain: cortex: frontal | Normal brain | — | — | Negative |
| | | cortex | | | staining |
| | | | | | observed. |
| 81 | tumour: kidney | Well | 1+ | 75-100 | |
| | | differentiated | | | |
| | | renal clear cell | | | |
| | | carcinoma | | | |
| 82 | tumour: kidney | Histology | 1+ | 75-100 | |
| | | consistent with | | | |
| | | renal cell | | | |
| | | carcinoma. | | | |
| 83 | tumour: kidney | Renal cell (clear | 1-2+ | 50-75 | |
| | | cell) carcinoma | | | |
| 84 | tumour: kidney | Clear cell renal | 1-2+ | 75-100 | |
| | | cell carcinoma | | | |
| | | of kidney. | | | |
| 85 | kidney: cortex | Normal renal | — | — | 0-1+ |
| | | cortex | | | Cytoplasmic |
| | | | | | staining |
| | | | | | in |
| | | | | | collecting |
| | | | | | tubules |
| 86 | kidney: cortex | Normal renal | — | — | 2+ |
| | | cortex. | | | Cytoplasmic |
| | | | | | staining |
| | | | | | in |
| | | | | | collecting |
| | | | | | tubules |
| 87 | Tumour: liver | Hepatocellular | 3+ | 75-100 | |
| | | carcinoma | | | |
| 88 | Tumour: liver | Fibrolamellar | 1+ | 0-25 | |
| | | Hepatocellular | | | |
| | | Carcinoma | | | |
| 89 | tumour: liver | Low Grade | 2+ | 75-100 | |
| | | hepatocellular | | | |
| | | carcinoma | | | |
| 90 | Tumour: liver | Hepatocellular | — | — | Core loss |
| | | carcinoma | | | |
| 91 | liver: parenchyma | Normal liver | — | — | 0-1 |
| | | | | | Cytoplasmic/ |
| | | | | | membrane |
| | | | | | staining |
| 92 | liver: parenchyma | Liver - normal | — | — | 0-1 |
| | | limits. | | | Membrane |
| | | | | | staining |

### Top 4Tm Tissue Microarray Study Using LSR Specific Pab;

The ‘Top 4’ TMA consisted of cores from 4 tissue types: breast (4 normal and 26 tumors), large intestine (4 normal and 26 tumors), lung (4 normal and 26 tumors) and prostate (4 normal and 26 tumors). The TMA layout is shown in Table 9. FFPE sections (4 μm) of cell line HEK293T LSR, the ‘Top4’ multi-tumor TMA were used. Unless otherwise indicated, all incubations were carried out at room temperature. The sections were de-paraffinized, antigen retrieved and rehydrated using pH9.0 Flex+ 3-in-1 antigen retrieval buffers, in PT Link apparatus at 95° C. for 20 min with automatic heating and cooling. Following antigen retrieval, sections were washed in Flex (TBST) buffer for 2×5 min then loaded into a DAKO Autostainer Plus. The sections were then incubated for 10 min with Flex+ Peroxidase Blocking reagent, rinsed twice in 50 mM Tris. HCl, 150 mM NaCl, 0.1% Tween-20, pH 7.6 (TBST), followed by a 10 min incubation with Protein Block reagent (DAKO X0909). The sections were incubated for 30 min with primary antibody diluted in DAKO Envision Flex antibody diluent (DAKO Cytomation, Cat # K8006). Anti LSR pAb (Abcam ab169583) was applied at 6 μg/ml. Anti Von Willebrand factor (VWf) antibody was applied at 1 μg/ml. The negative control sections were incubated with non-immune rabbit IgG antibodies (Dako, CAT #0936) at 6 and 1 μg/ml or in DAKO Envision Flex antibody diluent (‘no primary’ control). Following incubation with primary antibodies, the sections were then rinsed twice in FLEX buffer, incubated with anti-mouse/rabbit Flex+HRP for 20 min, rinsed twice in FLEX buffer and then incubated with diaminobenzidine (DAB) substrate for 10 min The chromogenic reaction was stopped by rinsing the slides with distilled water. Following chromagenesis, the sections were counterstained with haematoxylin, dehydrated in an ascending series of ethanols (90-99-100%), and cleared in three changes of xylene and coverslipped under DePeX. Stained sections were analyzed, and suitable digital images captured, using an Olympus BX51 microscope with a Leica DFC290 camera.

Within the breast tumor set, the intensity of staining was heterogeneous in the majority of cases; the immunoreactivity seen was weak to moderate. In this cohort, two samples scored a maximum intensity of 3-3+within 50-100% of reactive tumours. Within fourteen of the samples, the staining intensity scored a maximum of 2+, within 25-100% of tumours (grades 2/3). Ten other samples scored a lower intensity of 1+, of which most tumor samples were 25-50% reactive. Few other reactive tumor samples were also seen to be 0-25% to 75-100% weakly stained. Within the stromal regions, infiltrating immune cells were also positively stained. The majority of tumours were mainly infiltrating ductal and lobular carcinomas—mixed grades. There was no observed pattern of immunoreactivity that could be specifically attributed to tumor type or grade. In normal breast, specific cytoplasmic immunoreactivity was seen within the glandular acini.

Within the large bowel cohort, the adenocarcinoma samples were all immunoreactive, with the exception of one sample in this study. Within the cohort, the majority of tumours were poor to well differentiated types. In eight samples, an assigned score of 3+ staining was seen within 25-100% of tumours of grades 2/3. Most of these tumours had a distinct feature exhibiting a prominent cytoplasmic-membrane phenotype. In ten samples, a score of 2+ was seen in 25-100% of reactive tumor grades 2/3. In six samples, a lower score of 1+ was assigned to tumours, with the majority demonstrating 0-25%-50-75% reactivity. An apparently consistent pattern of staining, relative to the assigned intensity scores was seen in these tumor sets. Higher scores demonstrated a prominent membrane phenotype. In normal tissue samples, specific immunoreactivity was detected in mucosal epithelium and micro-vascular elements.

In the lung tumor set, specific immunoreactivity was seen in the majority of tumours investigated, where a weak to moderate staining intensity was noted. The majority of tumours were non-small cell carcinomas (NSCLC)—of adenocarcinoma origin, of well to poorly differentiated cell types. In these tumours, six samples were assigned a maximum intensity score of 2+ staining, of which 50-100% of tumours were immunoreactive in most respective cores. Eighteen samples were assigned a weaker score of 1/1+ staining, within 25-100% of tumours. In one sample of small-cell carcinoma, specific cytoplasmic immunoreactivity was seen to be weakly stained (1+), within 25-100% of tumours over the three cores analyzed. Other notable staining features were seen in vascular elements, and intensely-stained infiltrating immune cells. In the normal lung tissue, specific immunoreactivity was detected in a sample of bronchiole epithelium, and free macrophages of the alveolar spaces. Negative staining was seen in the alveolar septa.

In the set of prostate tumours, specific staining was seen in most samples, where intensity of staining was weak to moderate in the tumor epithelium. In one sample, a maximum assigned score of (3) staining was seen in 75-100% of tumours, (Gleason score 4+5), with a membranous pattern of immunoreactivity. Seven other samples had a score of 2+ within 25-100% of tumours. Most of these resided in tumor islands, with Gleason scores ranging from (3+4) and (4+3) respectively. Lastly, seventeen tumor samples were scored a weaker 1/1+ staining in 25-100% of tumours. In the normal prostate tissues, a few samples demonstrated weak-cytoplasmic and occasional cytoplasmic-membrane staining in the glandular epithelium. In general, immunoreactivity was either blush staining or negative in epithelium. Other notable staining was seen in putative infiltrating immune cells.

TABLE 9
Top4 TMA tissue annotation

| | Case | | | |
|---|---|---|---|---|
| Tissue | ID | AGE | SEX | Pathology |
| Breast | 51168 | 40 | F | Normal |
| Breast | 14784 | 38 | F | Normal |
| Breast | 11292 | 53 | F | Normal |
| Breast | 33349 | 61 | F | Normal |
| Breast | 8998 | 45 | F | Infiltrating Ductal Carcinoma. Grade 2/3 |
| Breast | 9100 | 36 | F | Infiltrating Ductal Carcinoma. Grade 2/3 |
| Breast | 9553 | 53 | F | Infiltrating Ductal Carcinoma. Grade 2 |
| Breast | 26375 | 87 | F | Infiltrating Ductal Carcinoma. Moderately |
| | | | | Differentiated |
| Breast | 36386 | 67 | F | Infiltrating Ductal Carcinoma. Grade 3 |
| Breast | 41690 | 70 | F | Medullary Carcinoma. Grade 2 |
| Breast | 8980 | 45 | F | Infiltrating Ductal Carcinoma. Grade 1/3 |
| Breast | 9133 | 42 | F | Infiltrating Ductal Carcinoma. Grade 2 |
| Breast | 9536 | 44 | F | Infiltrating Ductal Carcinoma |
| Breast | 12281 | 33 | F | Infiltrating Ductal Carcinoma 2 |
| Breast | 33139 | 54 | F | Infiltrating Ductal Carcinoma Grade III |
| Breast | 16996 | 74 | F | Infiltrating Ductal Carcinoma. Grade 1 |
| Breast | 33162 | 75 | F | Infiltrating Ductal Carcinoma Grade III |
| Breast | 33119 | 43 | F | Infiltrating Ductal Carcinoma Ductal carcinoma in situ |
| | | | | Grade III |
| Breast | 12759 | 49 | F | Infiltrating ductal carcinoma Ductal carcinoma in situ |
| | | | | Grade II |
| Breast | 15537 | 61 | F | Infiltrating Ductal Carcinoma Poorly Differentiated |
| Breast | 16957 | 63 | F | Infiltrating Ductal Carcinoma Fibrocystic change. |
| | | | | Grade 3/3 |
| Breast | 17916 | 75 | F | Infiltrating Ductal Carcinoma |
| Breast | 18381 | 37 | F | Infiltrating Ductal Carcinoma. Grade 3/3 |
| Breast | 27813 | 53 | F | Infiltrating Ductal Carcinoma of the breast. Grade 3 |
| Breast | 59919 | 53 | F | Infiltrating ductal carcinoma of the breast. Grade II |
| Breast | 35405 | 53 | F | Infiltrating Ductal Carcinoma, Lobular carcinoma of |
| | | | | the breast. Grade 3 |
| Breast | 54741 | 43 | F | Infiltrating ductal mixed with other types of |
| | | | | carcinoma of the breast. Grade II |
| Breast | 54876 | 51 | F | Infiltrating ductal carcinoma of the breast. Grade II |
| Breast | 55082 | 62 | F | Infiltrating ductal carcinoma of the breast. Grade II |
| Breast | 55238 | 70 | F | Infiltrating ductal and lobular carcinoma of the |
| | | | | breast. Grade II |
| Rectum | 9533 | 66 | M | Normal |
| Rectum | 9608 | 53 | M | Normal |
| Rectum | 16049 | 45 | M | Normal |
| Colon | 17038 | 49 | F | Normal |
| Colon | 36317 | 73 | F | Moderately differentiated: Adenocarcinoma of the |
| | | | | large intestine |
| Colon | 19746 | 89 | M | Moderately Differentiated: Adenocarcinoma of the |
| | | | | sigmoid colon |
| Colon | 22182 | 63 | M | Moderate to Poorly Differentiated: Adenocarcinoma |
| | | | | of the cecum |
| Colon | 25846 | 66 | F | Moderate to Poorly Differentiated: Adenocarcinoma |
| | | | | of the colon |
| Colon | 38189 | 41 | F | Well to Moderately Differentiated: Adenocarcinoma |
| | | | | of the colon |
| Colon | 41243 | 73 | M | Moderately differentiated: Adenocarcinoma of the |
| | | | | colon |
| Rectum | 52940 | 74 | F | Moderately differentiated: Adenocarcinoma of the |
| | | | | rectum |
| Colon | 53615 | 48 | F | Moderately differentiated: Adenocarcinoma of the |
| | | | | colon descending |
| Colon | 54804 | 43 | M | Moderate to poorly differentiated: Adenocarcinoma |
| | | | | of the large intestine |
| Rectum | 55076 | 70 | M | Poorly differentiated: Adenocarcinoma of the rectum |
| Colon | 55827 | 57 | M | Moderately Differentiated: Adenocarcinoma of the |
| | | | | large intestine |
| Colon | 9566 | 66 | F | Moderately Differentiated Adenocarcinoma of the |
| | | | | colon |
| Colon | 11960 | 62 | F | Moderately Differentiated: Adenocarcinoma |
| Rectum | 44270 | 66 | M | Grade 3: Adenocarcinoma of the rectum |
| Rectum | 18671 | 65 | M | Moderately Differentiated: Adenocarcinoma of the |
| | | | | rectum |
| Rectum | 19950 | 64 | F | Moderately Differentiated: Adenocarcinoma of the |
| | | | | rectum |
| Rectum | 20022 | 53 | M | Moderately Differentiated: Adenocarcinoma of the |
| | | | | rectum |
| Rectum | 20061 | 62 | M | Moderately Differentiated: Mucinous |
| | | | | adenocarcinoma of the rectum |
| Rectum | 20560 | 58 | M | Moderately Differentiated: Adenocarcinoma of |
| | | | | rectum |
| Rectum | 20921 | 75 | F | Moderately Differentiated: Adenocarcinoma of the |
| | | | | rectum |
| Rectum | 20990 | 84 | F | Moderately Differentiated: Adenocarcinoma of |
| | | | | rectum |
| Colon | 19948 | 71 | M | Grade2: Tubular adenocarcinoma of the ascending |
| | | | | colon |
| Rectum | 20895 | 25 | F | Moderately Differentiated Adenocarcinoma of the |
| | | | | rectum |
| Colon | 22072 | 67 | M | Well Differentiated Adenocarcinoma of the colon |
| Colon | 36274 | 61 | F | Moderate to poorly differentiated Adenocarcinoma |
| | | | | of the large intestine |
| Colon | 42362 | 58 | F | Moderately differentiated Adenocarcinoma of the |
| | | | | colon |
| Lung | 6692 | 37 | M | Normal |
| Lung | 7892 | 48 | M | Normal |
| Lung | 7893 | 75 | F | Normal |
| Lung | 7900 | 75 | F | Normal |
| Lung | 61215 | 59 | M | Non-small cell carcinoma: Moderately differentiated |
| | | | | Adenocarcinoma of the lung |
| Lung | 62743 | 75 | M | Non-small cell carcinoma: Moderately differentiated |
| | | | | Adenocarcinoma of the lung |
| Lung | 30426 | 63 | M | Non-Small Cell Carcinoma: Squamous Cell, Poorly |
| | | | | differentiated |
| Lung | 40222 | 52 | M | Non-small Cell Carcinoma: Grade 3 |
| Lung | 45409 | 52 | M | Non-Small Cell Carcinoma: Grade 3 |
| Lung | 45925 | 62 | M | Non-Small Cell Carcinoma: Squamous cell, Moderate |
| | | | | to poorly differentiated |
| Lung | 46417 | 70 | M | Non-Small Cell Carcinoma: Squamous Cell, |
| | | | | Moderately differentiated |
| Lung | 47183 | 68 | F | Non-Small Cell Carcinoma: Squamous Cell, |
| | | | | Moderately differentiated |
| Lung | 47184 | 57 | M | Non-Small Cell Carcinoma: Squamous Cell, Poorly |
| | | | | differentiated |
| Lung | 47231 | 78 | M | Non-Small Cell Carcinoma: Squamous Cell, Moderate |
| | | | | to poorly differentiated |
| Lung | 62745 | 47 | M | Non-small cell carcinoma: Adenocarcinoma, |
| | | | | Moderate to poorly differentiated |
| Lung | 50538 | 56 | M | Non-Small Cell Carcinoma: Squamous Cell, |
| | | | | Keratinizing. Moderate to poorly differentiated |
| Lung | 52835 | 59 | M | Non-small cell carcinoma: Squamous cell carcinoma, |
| | | | | keratinizing Well to moderately differentiated |
| Lung | 59389 | 65 | M | Non-small cell carcinoma: Papillary adenocarcinoma, |
| | | | | Moderate to poorly differentiated |
| Lung | 55383 | 58 | M | Non-small cell carcinoma: Large cell carcinoma, |
| | | | | Poorly differentiated |
| Lung | 57766 | 62 | M | Non-small cell carcinoma: Squamous cell carcinoma, |
| | | | | large cell, non-keratinizing, Moderate to poorly |
| | | | | differentiated |
| Lung | 57771 | 61 | M | Non-small cell carcinoma: Squamous cell carcinoma, |
| | | | | large cell, non-keratinizing, Poorly differentiated |
| Lung | 57772 | 59 | M | Non-small cell carcinoma: Squamous cell carcinoma, |
| | | | | keratinizing, Moderately differentiated |
| Lung | 59370 | 51 | M | Non-small cell carcinoma: Large cell carcinoma, |
| | | | | Undifferentiated |
| Lung | 4457 | 73 | M | Squamous Cell Carcinoma: Moderately Differentiated |
| Lung | 30167 | 57 | M | Non-small cell carcinoma: Adenocarcinoma, Poorly |
| | | | | differentiated |
| Lung | 10891 | 56 | M | Non-small Cell Carcinoma: Poorly differentiated |
| Lung | 12002 | 74 | F | Non-small Cell Carcinoma: Poorly differentiated |
| Lung | 12047 | 60 | F | Adenocarcinoma: Moderate to Poorly Differentiated |
| Lung | 61076 | 69 | M | Non-small cell carcinoma: Poorly differentiated |
| | | | | Adenocarcinoma of the lung |
| Lung | 42315 | 67 | M | Small Cell Carcinoma: Undifferentiated Small cell |
| | | | | carcinoma of the lung |
| Prostate | 11657 | 62 | M | Normal |
| gland | | | | |
| Prostate | 35710 | 63 | M | Normal |
| gland | | | | |
| Prostate | 35711 | 59 | M | Normal |
| gland | | | | |
| Prostate | 35707 | 63 | M | Normal |
| gland | | | | |
| Prostate | 11813 | 62 | M | Adenocarcinoma of the prostate: 3 + 4 |
| gland | | | | |
| Prostate | 69088 | 84 | M | Adenocarcinoma of the prostate gland: 4 + 5 = 9 |
| gland | | | | |
| Prostate | 68933 | 68 | M | Adenocarcinoma of the prostate gland: 3 + 4 = 7 |
| gland | | | | |
| Prostate | 23502 | 56 | M | Adenocarcinoma of prostate gland: Gleason Score |
| gland | | | | 2 + 3 = 5 |
| Prostate | 28142 | 52 | M | Adenocarcinoma of the prostate: Gleason Score |
| gland | | | | 3 + 3 = 6 |
| Prostate | 29979 | 64 | M | Adenocarcinoma of the prostate: Gleason Score |
| gland | | | | 4 + 3 = 7 |
| Prostate | 70295 | 67 | M | Adenocarcinoma of the prostate gland: 4 + 5 = 9 |
| gland | | | | |
| Prostate | 21336 | 58 | M | Infiltrating adenocarcinoma of the prostate. |
| gland | | | | |
| Prostate | 68925 | 63 | M | Adenocarcinoma of the prostate gland 4 + 3 = 7 |
| gland | | | | |
| Prostate | 23480 | 60 | M | Adenocarcinoma of the prostate Gleason Score |
| gland | | | | 3 + 3 = 6 |
| Prostate | 23504 | 55 | M | Adenocarcinoma of the prostate Gleason Score |
| gland | | | | 4 + 4 = 8 |
| Prostate | 24029 | 67 | M | Adenocarcinoma of the prostate Gleason Score |
| gland | | | | 3 + 4 = 7 |
| Prostate | 26159 | 61 | M | Adenocarcinoma of the prostate Gleason Score |
| Gland | | | | 3 + 4 = 7 |
| Prostate | 34589 | 59 | M | Adenocarcinoma of the prostate Gleason Score |
| Gland | | | | 4 + 3 = 7 |
| Prostate | 34876 | 74 | M | Adenocarcinoma of the prostate gland 4 + 3 = 7 |
| Gland | | | | |
| Prostate | 69073 | 71 | M | Adenocarcinoma of the prostate gland 4 + 3 = 7 |
| Gland | | | | |
| Prostate | 38248 | 56 | M | Adenocarcinoma of the prostate. High grade |
| Gland | | | | prostatic intraepithelial neoplasia, Gleason Score 3 + 4 = 7 |
| Prostate | 39553 | 55 | M | Adenocarcinoma of the prostate 3 + 4 = 7 |
| Gland | | | | |
| Prostate | 40334 | 67 | M | Adenocarcinoma of the prostate 3 + 4 = 7 |
| Gland | | | | |
| Prostate | 68934 | 65 | M | Adenocarcinoma of the prostate gland 4 + 3 = 7 |
| Gland | | | | |
| Prostate | 53065 | 62 | M | Adenocarcinoma of the prostate gland 3 + 4 = 7 |
| Gland | | | | |
| Prostate | 54842 | 52 | M | Carcinoma, undifferentiated of the prostate gland |
| Gland | | | | |
| Prostate | 59769 | 65 | M | Adenocarcinoma of the prostate gland 4 + 3 = 7 |
| Gland | | | | |
| Prostate | 69085 | 74 | M | Adenocarcinoma of the prostate gland 4 + 3 = 7 |
| Gland | | | | |
| Prostate | 58979 | 59 | M | Adenocarcinoma of the prostate gland 3 + 3 = 6 |
| Gland | | | | |
| Prostate | 58946 | 63 | M | Adenocarcinoma of the prostate gland 3 + 3 = 6 |
| Gland | | | | |

### Example 10

### Mouse CD4⁺T Cell Stimulation for Binding Assay

Since the expression of some of the B7/CD28 family receptors (e.g. CTLA4, PD-1 and the un-known receptor for B7-H4) is activation dependent, in this experiment it was tested whether the counter-receptor of LSR is expressed on activated T cells. Towards this, murine primary CD4 T cells were activated using plate bound anti-CD3 antibodies in the presence of soluble anti-CD28 antibodies, or by general activation using Concanavalin A (Con A).
1. Concanavalin A (Con A) stimulation—Purified CD4+T cells were seeded in flat bottom 96-well plates at 2×10⁵ cells per well. Con A (3 μg/ml, Sigma, Cat. C5275) was added and cells were incubated at 37° C. for 48 hr.
2. Anti-CD3+anti-CD28 stimulation—for anti-CD3 immobilization, 75 μl of 5 μg/ml anti-CD3 (0.5 mg/ml, cat.553058 BD) were added per well of flat bottom 96-well plate and incubated at room temperature for at least 2 hr. Wells were washed 3 times with 200 μl of PBS. Purified CD4+T cells were seeded at 2×10⁵ per well with 2 ug/ml of soluble anti CD-28 (eBioscience, cat.16-0281-85), and incubated at 37° c. for 48 hr.

### Binding Assay

1×10⁵ CD4+T cells, stimulated with Con A or anti-CD3/CD28, were used for binding. ECD of mouse LSR fused to mouse IgG2a Fc M:M (batch #35) (SEQ ID NO:221) was used in this binding assay. As positive control, a non-glycosylated derivative of ECD of human ILDR2 fused to mouse IgG2a Fc H:M (with the N297A mutation in the Fc, batch #23) (SEQ ID NO:223), was tested in parallel. Detection of protein binding was carried out as described above, for binding to T cell lines.

FIG. 20 demonstrates binding assay results with stimulated CD4+T cells. According the results, no binding of mouse ECD of LSR fused to mouse IgG2a Fc M:M (batch #35) (SEQ ID NO:221) (at 2 μg/50 μl/well) to anti CD3/CD28 or Con A activated murine T cells was shown.

### Example 11

### Binding of Increasing Concentrations of Biotin Labeled Mouse ECD of LSR Fused to Mouse IgG2a fc to karpas-299 Cells

KARPAS-299 cells (ACC31, DSMZ) were used to test whether mouse LSR-ECD fused to mouse IgG2a Fc (SEQ ID NO:221) binding can be detected with higher protein concentrations. Staining of KARPAS-299 cells was performed at increasing concentrations of biotin-labeled mouse LSR-ECD fused to mouse IgG2a Fc (SEQ ID NO:221).

Cells were incubated with various concentrations (2 ug, 3 ug, 4 ug and 5 ug) of biotin labeled-mouse LSR-ECD fused to mouse IgG2a Fc protein (SEQ ID NO:221), or 5p.g biotin-labeled control mIgG2a. Following cell washing, Streptavidin-PE was added and binding was evaluated by FACS analysis. FIG. 23A presents histograms showing Median Fluorescence Intensity (MFI). FIG. 23B presents the dose-dependent curve showings the binding (MFI) ratio of mouse LSR-ECD fused to mouse IgG2a Fc (SEQ ID NO:221) vs. control-mIgG2a relative to various protein concentrations of mouse LSR-ECD fused to mouse IgG2a Fc.

As shown in FIG. 21, binding of biotinylated mouse LSR-ECD fused to mouse IgG2a Fc (SEQ ID NO:221) was detected at concentrations higher than 3 μg/50 μl/well in a dose-dependent manner.

### Example 12

### Evaluation of mrna expression levels in Macrophages Under M1 and M2 Polarizing Conditions

The aim of this study was to evaluate the effect of polarizing signals (for M1: IFNg, LPS, IFNg+LPS; for M2: IL-4, TGFb, glococorticoids, tumor supernatants) on the expression of LSR mRNA using a mouse LSR TaqMan probe, (Applied Biosystems; CAT#: Mm00660290_ml) on macrophage differentiation at the mRNA level.

Macrophages are highly plastic cells, able to undergo distinct polarized activation in response to signals derived from the microenvironment, and in particular signals from the adaptive immune system are major determinant of macrophage polarization. In particular, microbial products and inflammatory cytokines induce macrophages to undergo the classic activation profile (also known as M1), which is characterized by the potentiation of their ability to kill intracellular microorganisms and an abundant production of inflammatory cytokines (TNF, IL-12, IL-23) and proinflammatory mediators (nitric oxide and reactive oxygen intermediates). On the other hand, alternatively activated M2 macrophages are promoted by various signals, including IL-4/IL-13, TGF, glucocorticoids, IL-10, and sustain angiogenesis and tissue remodelling during inflammation resolution. Macrophage polarized activation has significant impact in inflammatory and autoimmune disorders and in tumor biology. In particular, whereas M1 macrophages sustain pathogen killing and predispose to tumor initiation in tissues undergoing chronic inflammation, alternatively activated macrophages have immunoregulatory functions, participate to wound healing and in established tumors promote tissue remodelling and angiogenesis supporting tumor growth and metastatization Importantly, differential polarization of macrophages has profound impact on lymphocyte activation. M1 macrophages have a strong propensity to present antigen to T cells and activate a protective immune response, while M2 macrophages are more prone to an immunoregulatory/immunosuppressive phenotype.

In this example, the effect of polarizing signals (for M1: IFNg (Peprotech

315-05), LPS (ALEXIS, ALX-581-009-L002), IFNg+LPS; for M2: IL-4 (Peprotech 214-14), TGFb (Peprotech 100-21), prostaglandin E2 (Cayman Chemical 14010) tumor supernatants) on the expression of LSR on macrophage differentiation at the mRNA level was evaluated. Expression levels of four prototypical M1 (iNOS and CD80) and M2 (human ALOX15 or mouse 12/15, IL-10) highly polarized markers, plus 1 housekeeping gene were also evaluated as control. As the kinetic of expression of M1 and M2 genes is often different, cells were treated with polarizing signals for different periods, including 2, 8 and 18 hours. Three biological replicates were investigated.

The experiment was performed with murine macrophages. Murine peritoneal macrophages were obtained from mice that had received injections of 500 microL 3% (wt/vol) thioglycollate medium (Difco, Detroit, Mich.) 4 days prior to isolation. After purification (adherence), cells were rested for 1 hour in standard culture conditions and subsequently used for experiments

After stimulation, total RNA was extracted using TRIzol (Invitrogen Life Technologies), retrotranscribed, and prepared for a custom-designed 384 wells TaqMan Low Density Arrays (LDA) LSR (Applied Biosystems; CAT#: Mm00660290_ml) markers on M1 and M2 polarized activation. Samples were analyzed using a 7900HT system with a TaqMan LDA Upgrade (Applied Biosystems) and SDS2.1 software. Experiments were conducted on three independent macrophage preparations and in duplicate.

### Results:

In order to validate the experimental system, expression levels of prototypic M1 and M2 markers were evaluated under the different stimulations as described above. As shown in FIG. 22, the expression of INOS which is a prototypic M1 marker was elevated upon LPS+INFγ (M1 driving) stimulation. As shown in FIG. 23, the expression of ALOX15 which is a prototypic M2 marker was elevated upon IL4 and tumor supernatant (M2 driving) stimulation.

Next, the mRNA expression levels of LSR were evaluated under the different stimulations and different time points as described above. As shown in FIG. 24, the expression of LSR was elevated upon prostaglandin E and tumor supernatant (M2 driving) stimulation. The effect of prostaglandin E2 is evident after 8 hours of activation and the effect of the tumor supernatant is evident after 18 hours of activation. The absolute expression levels of LSR under the designated stimulations are comparable to the expression levels of ALOX15 (an M2 prototypic marker) under M2 driving conditions.

### CONCLUSIONS

The results of this study demonstrate up regulation of LSR mRNA expression on the alternatively activated M2 macrophages.

Alternatively activated M2 macrophages have immunoregulatory functions, participate in wound healing and in established tumors, promote tissue remodelling and angiogenesis supporting tumor growth and metastatization. Thus, without wishing to be bound by a single hypothesis, the expression of LSR on M2 polarized macrophages has implication for the use of anti-LSR therapeutic antibodies for the treatment of cancer either by depletion of the deleterious tumor associated M2 macrophages or by inhibition of their immunosuppressive function and thus enhancing immune surveillance.

### Example 13

### Role of LSR Proteins as Modulators of Cancer Immune Surveillance

1) In Vivo Proof of Concept

a) Mouse Cancer Syngeneic Model:

(i) Tumor cells, over expressing LSR proteins or a non-relevant control protein are transplanted to genetically matched mice. Tumor volume (and tumor weight after sacrificing the animals) are then examined to demonstrate delay in the tumor growth (i.e. tumor over expressing LSR grow faster than tumors over expressing the non-relevant control protein). Ex vivo analysis of immune cells from tumor draining lymph nodes is carried out to evaluate the ratio of regulatory T cells and effector T cells.

(ii) Treatment of syngeneic tumor with neutralizing antibodies directed against LSR protein as mono-therapy. Tumor cells are transplanted to genetically identical mice. Tumor bearing mice are injected with different doses of neutralizing antibodies aimed against LSR protein. As a result of treatment with neutralizing antibodies specific for LSR protein the rejection of the tumor is increased (i.e. in mice treated with neutralizing antibodies against LSR protein tumors grow slower than tumors in mice treated with non-relevant antibody). Ex vivo analysis of immune cells from tumor draining lymph nodes is carried out to determine of the ratio of regulatory T cells and effector T cells.

The tumor cells lines tested are from various origins including colon, breast, and ovary carcinomas, melanoma, sarcomas and hematological cancers. Syngeneic models are performed in several mouse strains including BALB/c, C57b1/6 and C3H/Hej. In the first set of experiments the syngeneic transplantable models used are primarily those proved as predictive for cancer immunotherapy. These include: B16-F10 melanoma (according to the method described in Tihui Fu et al Cancer Res 2011; 71: 5445-5454), MC38 colon cancer (according to the method described in Ngiow S F et al. Cancer Res. 2011 May 15; 71(10):3540-51), ID8 ovarian cancer (according to the method described in Krempski et al. J Immunol 2011; 186:6905-6913), MCA105 sarcoma (according to the method described in Wang et al. J. Exp. Med. Vol. 208 No. 3 577-592), CT26 colon carcinoma (according to the method described in Ngiow S F et al. Cancer Res. 2011 May 15; 71(10):3540-51) and 4T1 mammary carcinoma (according to the method described in Takeda K et al. J. Immunol. 2010 May 15; 184(10):5493-501) of BALB/c background.

(iii) Establishment of a syngeneic tumor and treatment with neutralizing antibodies directed against LSR protein in combination with additional lines of treatment. Tumor cells are transplanted to genetically identical mice. After the establishment of tumors, mice are injected IP with different doses of neutralizing antibodies aimed against LSR protein in combination with conventional chemotherapy (e.g. cyclophosphamide, according to the method described in Mkrtichyan et al. Eur. J. immunol. 2011; 41, 2977-2986), in combination with other immune checkpoint blockers (e.g. PD1 and CTLA4, according to the method described in Curran et al.; Proc Natl Acad Sci USA. 2010 Mar. 2; 107(9):4275-80), in combination with other immune-modulators (e.g. anti-IL18, according to the method described in Terme et al.; cancer res. 2011; 71: 5393-5399), in combination with cancer vaccine (according to the method described in Hurwitz et al. Cancer Research 60, 2444-2448, May 1, 2000) or in combination with radio-therapy (according to the method described in Verbrugge et al. Cancer Res 2012; 72:3163-3174).

(iv) Human cancer Xenograft model: Human cancer cell lines, endogenously expressing LSR are transplanted into immune-deficient mice. Tumor volume in mice treated with anti-LSR antibody vs. mice treated with non-relevant isotype matched antibody will be assessed. In one arm of the study anti-LSR antibodies are conjugated to a toxin (according to the method described in Luther N et al. Mol Cancer Ther. 2010 April; 9(4):1039-46) to assess antibody drug conjugate (ADC) activity. In another arm of the experiment, mice are treated with human IgG1 or mouse IgG2a isotype antibodies against LSR (according to the method described in Holbrook E. Kohrt et al. J Clin Invest. 2012 Mar. 1; 122(3): 1066-1075).These antibody isotypes are used to assess antibody-dependent cellular cytotoxicity (ADCC) mediated tumor elimination.

2) In Vitro Validation of Natural Killer (NK) Cell Activity

a) Binding Assay:

(i) Binding assay with human LSR ECD-FC proteins on activated primary-culture NK cells is performed as described in J Immunol 2005; 174; 6692-6701.1f the counter receptor of LSR is expressed on NK cells, binding of LSR ECD-Fc is observed.

(ii) Binding assay with a specific antibody directed against the any one of LSR proteins on activated primary-culture NK cells is performed as described in PNAS, 2009, vol. 109; 17858-17863. If any one LSR is expressed on NK cells, binding of LSR specific antibody, respectively, is observed.

(iii) Binding assay with human LSR ECD-FC proteins on various human cancer cell lines that may serve as target cells for NK killing is performed as described in J Immunol 2006; 176; 6762-6769. If the counter receptor of any one of LSR is expressed on the cancer target cells, binding of LSR ECD-Fc, respectively is observed.

b) Functional Killing Assay:

(i) Killing assays are performed using an over expression system (either NK cells or cancer target cells, over expressing any one of LSR proteins). The NK cells (effector; e) are co-incubated with radioactive (S35) labeled cancer target cells (target; t) in various e: t ratios, as described in PNAS, 2009, vol. 109; 17858-17863. Lysis of target cells by NK killing activity is then evaluated by measurement of radioactive emission. Over expression of any one of LSR proteins on the target cancer cells and/or the NK cell lines lead to down regulation of the NK mediated killing activity.

(ii) Killing assays are performed in the presence of the human LSR ECD-FC proteins, as described in PLoS ONE; 2010; Vol. 5; p. 1-10. Treatment with the ECD-Fc of any of LSR interfere with the interaction of LSR with their counter receptors and thus decrease their inhibitory activity, giving rise to enhanced killing activity.

(iii) Killing assays are performed in the presence of a neutralizing antibody directed against any one of LSR proteins, as described in PNAS, 2009, vol. 109; 17858-17863. Treatment with neutralizing antibodies directed towards any of LSR, give rise to enhanced NK killing activity.

(iv) “Re-directed killing assay” is performed as follows: cancer target cells expressing high density Fc receptors are coated with activating antibodies directed against any one of LSR proteins and exposed to NK cells (expressing the designated LSR protein), as described in PNAS, 2009, vol. 109; 17858-17863. Cross linking of any one of LSR with activating antibodies give rise to reduced NK mediated killing activity.

3) In Vitro Validation of Cytotoxic T Lymphocyte (CTL) Activity

a) Expression of LSR Proteins in Melanoma Cell Lines.

Flow cytometric assessment of the LSR expression is carried out on various melanoma cell lines.

b) Functional Assay.

To perform functional assays, a primary human lymphocytes expressing the F4 TCR, which is a MART-1-specific (melanoma specific antigen) is used. TCR recognizes HLA-A2+/MART1+melanoma cells as described in Morgan et al, Science, 2006. OKT3-stimulated primary human lymphocytes (obtained from the local Blood Bank) are transduced with a retroviral vector encoding the F4-TCR. Then, OKT3 (anti CD3)-stimulated primary human lymphocytes are cultured in lymphocyte medium (containing IL-2). These pre-stimulated lymphocytes are incubated with melanoma cell lines over expressing full length LSR. The read outs include cytokine secretion, activation markers and cytotoxicity) at 3 different time points.

4) Expression Analysis

a) Expression of LSR Proteins on Tumor and Immune Cells Isolated from Human Tumor Biopsies

(i) Expression validation of LSR proteins using specific antibodies directed against the LSR proteins is carried out on separated cell populations from the tumor. Various cell populations are freshly isolated from tumor biopsies (e.g. Tumor cells, endothelia, tumor associated macrophages (TAMS) and DCs, B cells and different T cell sub-sets (CD4, CD8 and Tregs) as described in Kryczek I. et al., J. Exp. Med.; 2006; Vol. 203; p. 871-881 and Cancer res. 2007; 67; 8900-8905, to demonstrate expression of LSR in tumor cells and on tumor stroma and immune infiltrate.

(ii) Binding assay is performed with the human LSR ECD-FC proteins on separated cell populations from the tumor. Various cell populations from tumor biopsies (e.g. Tumor cells, endothelia, tumor associated macrophages (TAMS) and DCs, B cells and different T cells (CD4, CD8 and Tregs) are freshly isolated from tumors as described in J. Exp. Med.; 2006; Vol. 203; p. 871-881 and Cancer res. 2007; 67; 8900-8905, to show expression of the counter receptor for LSR in tumor cells and on tumor stroma and immune cells.

b) Expression of LSR Proteins on Cells Isolated from Draining Lymph Nodes and Spleens of Tumor Bearing Mice

(i) Expression validation of LSR proteins using specific antibodies directed against LSR proteins is done on epithelial cancer cells as well as on immune cells from tumor draining lymph nodes vs. spleen of tumor bearing C57 mice, as described in M Rocha et al., Clinical Cancer Research 1996 Vol. 2, 811-820. Three different cancer types are tested: B16 (melanoma), ID8 (ovarian) and MC38 (colon)), in order to evaluate expression of LSR in tumor cells and in immune cells within the tumor draining lymph node.

(ii) Binding assay with mouse LSR ECD-FC proteins on cells isolated from epithelial cancer as well as on immune cells from tumor draining lymph nodes versus spleen of tumor bearing C57 mice is carried out as described above, to show expression of the counter receptor for LSR in tumor cells and in immune cells in the tumor draining lymph node.

c) Expression of LSR Proteins on M2 Polarized Macrophages

(i) Expression validation of LSR proteins using specific antibodies directed against LSR proteins, is done on primary monocytes isolated from peripheral blood, differentiated into macrophages and exposed to “M2 driving stimuli” (e.g. IL4, IL10, Glucocorticoids, TGF beta), as described in Biswas S K, Nat. Immunol. 2010; Vol. 11; p. 889-896, to show expression of LSR in M2 differentiated Macrophages.

ii) Binding assay with LSR human ECD-FC proteins on primary monocytes isolated from peripheral blood, differentiated into macrophages and exposed to “M2 driving stimuli” (e.g. IL4, IL10, glucocorticoids, TGF beta) is carried out as described above, to evaluate expression of the counter receptor for LSR in M2 differentiated Macrophages.

d. Expression of LSR Proteins on Myeloid Derived Supressor Cells (MDSCs)

(i) Expression validation of LSR proteins using specific antibodies directed against LSR proteins, respectively, is done on primary MDSCs isolated from Tumor bearing mice, as described in Int Immunopharmacol. 2009 July; 9(7-8):937-48. Epub 2009 Apr. 9.

ii) Binding assays are carried out with LSR human ECD-Fc proteins described in PCT/IB2012/051868, owned in common with the present application, on primary MDSCs isolated from tumor bearing mice.

### Example 14

### Anti-Tumor Effect of Blocking Antibody Against the LSR Protein in Combination with Blockade of Known Immune Checkpoints

Inhibitory receptors on immune cells are pivotal regulators of immune escape in cancer. Among these are known immune checkpoints such as CTLA4, PD-1 and LAG-3. Blockade of a single immune checkpoint often leads to enhanced effector T cell infiltration of tumors, but may also lead to compensatory upregulation in these T cells of the other unblocked negative receptors. However, blockade of more than one inhibitory pathway allows T cells to carry out a more efficient tumor response, and increases the ratio of effector T cells (Teffs) to regulatory T cells (Tregs). Specifically, dual blockade of such inhibitory receptors has been shown to exert synergistic therapeutic effect in animal tumor models (Curran et al 2010 PNAS107: 4275-4280; Woo et al 2011 Cancer Res. 72: 917-927). Based on these findings, the combination of anti-CTLA-4 and anti-PD-1 blocking antibodies is being tested in clinical trials in patients with metastatic melanoma.

The combination of blocking antibodies against LSR and against PD-1 is tested in the syngeneic cancer MC38 model in the C57B1/6 background (as described in Woo et al 2011 Cancer Res. 72: 917-927). Briefly, MC38 cells (2×10⁶) are implanted s.c. C57B1/6 mice. Mice with palpable tumors are injected i.p. at a dosage of 10 mg/kg anti-LSR mAb and/or anti-PD-1 mAb (4H2). Isotype Control Ab is dosed at 20 mg/kg or added to individual anti-PD-1 or anti-LSR antibody treatments at 10 mg/kg. Tumor volumes are measured with an electronic caliper, and effect on tumor growth is calculated. The therapeutic effect, manifested as inhibition of tumor growth, is enhanced upon combination of the blocking antibodies against the two targets, PD-1 or LSR. The frequency of effector T cells=Teffs (CD8+IFNg+) cells and the ratio of Teffs and Tregs are determined in tumor draining lymph nodes and non-draining lymph nodes.

### Example 15

### Anti-Tumor Effect of Blocking Antibody Against the LSR Protein in Combination with Metronomic Therapy with Cyclophosphamide

Cyclophosphamide has been used as a standard alkylating chemotherapeutic agent against certain solid tumors and lymphomas because of its direct cytotoxic effect and its inhibitory activity against actively dividing cells. While high doses of cyclophosphamide may lead to depletion of immune cells, low doses have been shown to enhance immune responses and induce anti-tumor immune-mediated effects, primarily by reducing the number and function of immunosuppressive Treg cells (Brode and Cooke 2008 Crit. Rev. Immunol. 28: 109-126). Metronomic therapy using classical chemotherapies other than cyclophosphamide has also been shown to have immunostimulatory effects, including gemcitabine; platinum based compounds such as oxaliplatin, cisplatin and carboplatin; anthracyclines such as doxorubicin; taxanes such as paclitaxel and docetaxel; microtubule inhibitors such as vincristine.

Combination therapy of cyclophosphamide with other immunotherapies, such as anti-4-1BB activating Ab or anti-PD1 blocking Ab, resulted in synergistic anticancer effects (Kim et al. 2009 Mol Cancer Ther 8:469-478; Mkrtichyan et al. 2011 Eur. J. Immunol. 41:2977-2986).

Anti-LSR blocking mAb is tested in combination with cyclophosphamide in the syngeneic B16 melanoma model in the C57BL/6 background (as described in Kim et al. 2009 Mol Cancer Ther 8:469-478). Briefly, C57BL/6 mice are injected s.c. with 4×10⁵ B16-F10 melanoma cells. A single i.p. injection of cyclophosphamide (150 mg/kg) is administered on the day of tumor implantation, and five injections of 100 μg of the neutralizing antibody against LSR, 5 d apart beginning on the day of tumor implantation. To examine the antitumor effects of combination therapy on established tumors, the combination therapy is given beginning either at day 5 or day 10 after tumor cells injection. Tumor volumes are measured with an electronic caliper, and effect on tumor growth is calculated. The therapeutic effect, manifested as inhibition of tumor growth, is enhanced upon combination of cyclophosphamide with the blocking antibodies against LSR. The frequency of effector T cells=Teffs (CD8+IFNg+) cells and the ratio of Teffs and Tregs are determined in tumor draining lymph nodes and non-draining lymph nodes.

### Example 16

### Anti-Tumor Effect of Blocking Antibody Against the LSR Protein in Combination with Cellular Tumor Vacclnes

Therapeutic cancer vaccines enable improved priming of T cells and improved antigen presentation as agents potentiating anti-tumor responses. Among these, are cellular tumor vaccines that use whole cells or cell lysates either as the source of antigens or as the platform in which to deliver the antigens. Dendritic cell (DC)-based vaccines focus on ex vivo antigen delivery to DCs. Other therapeutic cancer vaccines consist of tumor cells genetically modified to secrete immune stimulatory cytokines or growth factors, such as GM-CSF (granulocyte-macrophage colony-stimulating factor) or Flt3-ligand, aim to deliver tumor antigens in vivo in an immune stimulatory context to endogenous DCs.

Several in vivo studies have shown a potent therapeutic effect of immunecheckpoint blockade, such as anti-CTLA-4 antibodies, in poorly immunogenic tumors only when combined with GM-CSF or Flt3-ligand-transduced tumor vaccines, termed Gvax and Fvax, respectively (van Elsas et al 1999 J. Exp. Med. 190: 355-366; Curran and Allison 2009 Cancer Res. 69: 7747-7755), and that the antibody alone was effective only in the most immunogenic tumor models in mice. Furthermore, combination of two immunotherapeutic agents, such as anti-CTLA4 and anti-PD-1 blocking antibodies, is more effective in conjuction with therapeutic cancer vaccine, such as Gvax or Fvax (Curran et al 2010 PNAS107: 4275-4280)

The effect of LSR neutralizing antibody in combination with tumor cell vaccine, is tested using irradiated melanoma cells engineered to secrete GMCSF or Flt3-ligand (GVAX or FVAX respectively) in the presence or absence of anti-PD-1 blocking antibody (as described in Curran et al 2010 PNAS107: 4275-4280). Briefly, mice are injected in the flank i.d. at day 0 with 5×10⁴ B16-BL6 cells and treated on days 3, 6, and 9 with 10⁶ irradiated (150 Gy) gene-modified B16 cells (expressing GMCSF or Flt3-ligand) on the contralateral flank in combination with intraperitoneal administration of 100 ug of anti-LSR blocking antibody, with or without 100 ug of anti-PD-1 blocking antibody (clone RMP1-14) or anti-PDL-1 blocking antibody (9G2). Isotype Ig is used as negative control. Tumor volumes are measured with an electronic caliper, and effect on tumor growth is calculated. The therapeutic effect, manifested as inhibition of tumor growth, is enhanced upon combination of the blocking antibodies against LSR with the gene modified tumor cell vaccine. Anti-PD-1 or anti-PDL-1 blocking antibodies further enhance this effect. The frequency of effector T cells=Teffs (CD8+IFNg+) cells and the ratio of Teffs and Tregs are determined in tumor draining lymph nodes and non-draining lymph nodes.

### Example 17

### Anti-Tumor Effect of Blocking Antibody Against the LSR Protein in Combination with Radiotherapy

Radiotherapy has long been used as anti-cancer therapy because of its powerful anti-proliferative and death-inducing capacities. However, recent preclinical and clinical data indicate that immunogenic cell death may also be an important consequence of ionizing radiation, and that localized radiotherapy can evoke and/or modulate anti-tumor immune responses (Reits et al 2006 J. Exp. Med. 203:1259-1271). Preclinical studies have shown enhanced therapeutic effects in combined treatment of radiotherapy and immunotherapy, including blocking antibodies to immune checkpoints such as CTLA4 and PD-1, in the absence or presence of an additional immunotherapy such as activating anti-4-1BB Abs (Demaria et al 2005 Clin. Can. Res. 11:728-734; Verbruge et al 2012 Can. Res. 72:3163-3174).

The combination of blocking anti-LSR antibodies and radiotherapy will be assessed using a syngeneic 4T1 mammary carcinoma cell line in the BALB/c background (as described in Demaria et al 2005 Clin. Can. Res. 11:728-734). Briefly, 5×10⁴ 4T1 cells are injected s.c. in the flank of BALB/c mice. Treatment begins when tumors reach an average diameter of 5 mm (65 mm³ in volume) Animal groups include treatment with each modality alone (anti-LSR or radiotherapy) and with the isotype Ig Control, and combination of anti-LSR with radiotherapy, or of Ig Control with radiotherapy. Radiotherapy is delivered to the primary tumor by one or two fractions (48 hrs interval) of 12Gy. Anti-LSR Ab or Ig control are given i.p. at 200 ug, on days 1, 4 and 7 after radiotherapy. In an additional set of experiments, blocking anti-PD-1 mAb (RMP1-14) and activating anti-4-1BB mAb (3E1). Tumor volumes are measured with an electronic caliper, and effect on tumor growth is calculated. The therapeutic effect, manifested as inhibition of tumor growth, is enhanced upon combination of the blocking antibodies against LSR with radiotherapy. Anti-PD-1 blocking antibodies or anti-4-1BB activating Abs, further enhance this effect. The frequency of effector T cells=Teffs (CD8+IFNg+) cells and the ratio of Teffs and Tregs are determined in tumor draining lymph nodes and non-draining lymph nodes.

### Example 18

### LSR-ECD-IgG Fusion Protein Upregulates Differentiation of Inducible Regulatory T Cells (iTregs) In Vitro

Tregs play an essential role in the immunosuppressive networks that contribute to tumor-immune evasion. To test the ability of an anti LSR antibody to block Treg differentiation, it is tested whether the interaction of LSR fusion protein with naive T cells affects their differentiation to iTregs. To this aim, mLSR-ECD-mouse IgG2a (SEQ ID NOs:221) and hLSR-ECD-human IgG1 fusion proteins (SEQ ID NOs:222; 236) are used, and their effect on differentiation of regulatory T cells is evaluated by testing the expression of the regulatory T cell marker, FoxP3, by CD4+CD25+ purified T cells when incubated in the presence of iTreg driving conditions.

T cell activation is either antigen-specific or polyclonal via anti-CD3. Naive CD4+T cells are isolated from mouse spleen or human PBMCs via automax sort (CD4-negative sort and CD25 positive isolation, followed by CD62L-positive sort).

The cells are activated in the presence of iTreg driving conditions (i.e. IL-2, TGF-beta and either Control Ig or LSR-ECD-IgG fusion proteins. On day 4 of culture, cells are harvested and stained for viability, CD4, CD25, and FoxP3 expression.

In additional studies, iTregs are sorted and tested for their functionality (i.e. their ability to inhibit T cell activation in co-culture assays).

Without wishing to be limited by a single hypothesis, the LSR-Ig fusion proteins enhance the differentiation of CD4 T cells to iTregs as the LSR pathway is involved in iTregs induction and differentiation, which implies that targeting LSR with blocking monoclonal antibodies inhibits iTregs accumulation and immunosuppressive function. Furthermore, by inhibiting LSR immune checkpoint activity, such blocking antibodies enhance effector T cell activity. Thus the enhancement of effector T cell activity and inhibition of iTreg immunosuppressive activity by LSR blocking antibodies lead to enhanced beneficial effects in cancer therapy using such antibodies, alone, or in combination with a potentiating agent.

### Example 19

### The Effectof LSR-IG Fusion Protein on Th Differentiation

The effect of LSR-Ig fusion protein on Th differentiation using mouse and human CD4+T cells upon activation under specific Th driving conditions is tested. Murine T cell activation is either antigen-specific or polyclonal. Without wishing to be limited by a single hypothesis, the results of these experimental settings, using mouse or human cells, point to an immunomodulatory effect of LSR on T cells, whereby Th1 and Th17 driven responses (secretion of proinflammatory cytokines and cell proliferation under Th1 and Th17 driving conditions) are inhibited, while secretion of anti-inflammatory cytokines (Th2 derived, and IL-10) are promoted.

It is known that one of the mechanisms by which tumors evade immune surveillance is promotion of a Th2/M2 oriented immune response (Biswas S K, et al., 2010 October; 11(10):889-96). Thus, without wishing to be limited by a single hypothesis, a neutralizing antibody which suppresses the above demonstrated immunomodulatory effect of LSR (i.e. promotion of Th2 response and inhibition of Th1 response) is beneficial for treatment of cancer.

### Example 20

### Development of Fully Human Anti-LSR Antibodies

Generation of Human Monoclonal Antibodies Against LSR Antigen

Fusion proteins composed of the extracellular domain of the LSR linked to a mouse IgG2 Fc polypeptide are generated by standard recombinant methods and used as antigen for immunization.

Transgenic HuMab Mouse.

Fully human monoclonal antibodies to LSR are prepared using mice from the HCo7 strain of the transgenic HuMab Mouse. RTM., which expresses human antibody genes. In this mouse strain, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al. (1996) Nature Biotechnology 14:845-851, and a human heavy chain transgene, HCo7, as described in U.S. Pat. Nos. 5,545,806; 5,625,825; and 5,545,807.

HuMab Immunizations:

To generate fully human monoclonal antibodies to LSR, mice of the HCo7 HuMab Mouse strain can be immunized with purified recombinant LSR fusion protein derived from mammalian cells that are transfected with an expression vector containing the gene encoding the fusion protein. General immunization schemes for the HuMab Mouse are described in Lonberg, N. et al (1994) Nature 368(6474): 856-859; Fishwild, D. et al. (1996) Nature Biotechnology 14: 845-851 and PCT Publication WO 98/24884. The mice are 6-16 weeks of age upon the first infusion of antigen. A purified recombinant LSR antigen preparation (5-50.mu.g, purified from transfected mammalian cells expressing LSR fusion protein) is used to immunize the HuMab mice intraperitoneally.

Transgenic mice are immunized twice with antigen in complete Freund's adjuvant or Ribi adjuvant IP, followed by 3-21 days IP (up to a total of 11 immunizations) with the antigen in incomplete Freund's or Ribi adjuvant. The immune response is monitored by retroorbital bleeds. The plasma is screened by ELISA (as described below), and mice with sufficient titers of anti-LSR human immunoglobulin are used for fusions. Mice are boosted intravenously with antigen 3 days before sacrifice and removal of the spleen.

Selection of HuMab Mice Producing Anti-LSR Antibodies:

To select HuMab mice producing antibodies that bind LSR sera from immunized mice is tested by a modified ELISA as originally described by Fishwild, D. et al. (1996). Briefly, microtiter plates are coated with purified recombinant LSR fusion protein at 1-2.mu.g/ml in PBS, 50.mu.1/wells incubated 4 degrees C. overnight then blocked with 200.mu.1/well of 5% BSA in PBS. Dilutions of plasma from LSR-immunized mice are added to each well and incubated for 1-2 hours at ambient temperature. The plates are washed with PBS/Tween and then incubated with a goat-anti-human kappa light chain polyclonal antibody conjugated with alkaline phosphatase for 1 hour at room temperature. After washing, the plates are developed with pNPP substrate and analyzed by spectrophotometer at OD 415-650. Mice that developed the highest titers of anti-LSR antibodies are used for fusions. Fusions are performed as described below and hybridoma supernatants are tested for anti-LSR activity by ELISA.

Generation of Hybridomas Producing Human Monoclonal Antibodies to LSR.

The mouse splenocytes, isolated from the HuMab mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the number of P3X63 Ag8.6.53 (ATCC CRL 1580) nonsecreting mouse myeloma cells with 50% PEG (Sigma). Cells are plated at approximately 1×10⁻⁵/well in flat bottom microtiter plate, followed by about two week incubation in selective medium containing 10% fetal calf serum, supplemented with origen (IGEN) in RPMI, L-glutamine, sodium pyruvate, HEPES, penicillin, streptamycin, gentamycin, 1×HAT, and beta-mercaptoethanol. After 1-2 weeks, cells are cultured in medium in which the HAT is replaced with HT. Individual wells are then screened by ELISA (described above) for human anti-LSR monoclonal IgG antibodies. Once extensive hybridoma growth occurred, medium is monitored usually after 10-14 days. The antibody secreting hybridomas are replated, screened again and, if still positive for human IgG, anti-LSR monoclonal antibodies are subcloned at least twice by limiting dilution. The stable subclones are then cultured in vitro to generate small amounts of antibody in tissue culture medium for further characterization.

Hybridoma clones are selected for further analysis.

### Structural Characterization of Desired Anti-LSR Human Monoclonal Antibodies

The cDNA sequences encoding the heavy and light chain variable regions of the obtained anti-LSR monoclonal antibodies are obtained from the resultant hybridomas, respectively, using standard PCR techniques and are sequenced using standard DNA sequencing techniques.

The nucleotide and amino acid sequences of the heavy chain variable region and of the light chain variable region are identified. These sequences may be compared to known human germline immunoglobulin light and heavy chain sequences and the CDRs of each heavy and light of the obtained anti-LSR sequences identified.

Characterization of Binding Specificity and Binding Kinetics of Anti-LSR Human Monoclonal Antibodies

The binding affinity, binding kinetics, binding specificity, and cross-competition of anti-LSR antibodies are examined by Biacore analysis. Also, binding specificity is examined by flow cytometry.

Binding Affinity and Kinetics

Anti-LSR antibodies produced according to the invention are characterized for affinities and binding kinetics by Biacore analysis (Biacore AB, Uppsala, Sweden). Purified recombinant human LSR fusion protein is covalently linked to a CM5 chip (carboxy methyl dextran coated chip) via primary amines, using standard amine coupling chemistry and kit provided by Biacore. Binding is measured by flowing the antibodies in HBS EP buffer (provided by BIAcore AB) at a concentration of 267 nM at a flow rate of 50.mu.1/min. The antigen-association antibodies association kinetics is followed for 3 minutes and the dissociation kinetics is followed for 7 minutes. The association and dissociation curves are fit to a 1:1 Langmuir binding model using BlAevaluation software (Biacore AB). To minimize the effects of avidity in the estimation of the binding constants, only the initial segment of data corresponding to association and dissociation phases are used for fitting.

Epitope Mapping of Obtained Anti-LSR Antibodies

Biacore is used to determine epitope grouping of anti-LSR HuMAbs. Obtained anti-LSR antibodies are used to map their epitopes on the LSR antigen. These different antibodies are coated on three different surfaces of the same chip to 8000 RUs each. Dilutions of each of the mAbs are made, starting at 10 mu.g/mL and is incubated with Fc fused LSR (50 nM) for one hour. The incubated complex is injected over all the three surfaces (and a blank surface) at the same time for 1.5 minutes at a flow rate of 20.mu.L/min. Signal from each surface at end of 1.5 minutes, after subtraction of appropriate blanks, has been plotted against concentration of mAb in the complex. Upon analysis of the data, the anti-LSR antibodies are categorized into different epitope groups depending on the epitope mapping results. The functional properties thereof are also compared.

Chinese hamster ovary (CHO) cell lines that express LSR protein at the cell surface are developed and used to determine the specificity of the LSR HuMAbs by flow cytometry. CHO cells are transfected with expression plasmids containing full length cDNA encoding a transmembrane forms of LSR antigen or a variant thereof. The transfected proteins contained an epitope tag at the N-terminus are used for detection by an antibody specific for the epitope. Binding of a anti-LSR MAb is assessed by incubating the transfected cells with each of the r LSR Abs at a concentration of 10 mu.g/ml. The cells are washed and binding is detected with a FITC-labeled anti-human IgG Ab. A murine anti-epitope tag Ab, followed by labeled anti-murine IgG, is used as the positive control. Non-specific human and murine Abs are used as negative controls. The obtained data is used to assess the specificity of the HuMAbs for the LSR antigen target.

These antibodies and other antibodies specific to LSR may be used in the afore-described anti-LSR related therapies such as treatment of cancers wherein LSR antigen is differentially expressed and/or for modulating (enhancing or inhibiting) B7 immune co-stimulation involving the LSR antigen such as in the treatment of cancers and autoimmune diseases wherein such antibodies will e.g., prevent negative stimulation of T cell activity against desired target cancer cells or prevent the positive stimulation of T cell activity thereby eliciting a desired anti-autoimmune effect.

Generation of monoclonal antibodies to LSR using antibody libraries.

Those skilled in the art will also appreciate that DNA encoding antibodies or antibody fragments (e.g., antigen binding sites) may also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H.R. and Chames, Immunol. Today 27:371 (2000); Nagy et al Nat. Med. 5:801 (2002); Huie et al, Proc. Natl. Acad. ScL USA 95:2682 (2001); Lui et al, J. MoI. Biol. 5/5:1063 (2002), each of which is incorporated herein by reference. Several publications (e.g., Marks et al, Bio/Technology 70:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al, Nat. Biotechnol. 75:1287 (2000); Wilson et al, Proc. Natl. Acad. ScL USA 98:3750 (2001); or Irving et al, J. Immunol. Methods 248:3\ (2001)). In yet another embodiment, cell surface libraries can be screened for antibodies (Boder et al, Proc. Natl. Acad. ScL USA 97:10701 (2000); Daugherty et al, J. Immunol. Methods 243:21 1 (2000)).

Such procedures provide alternatives to traditional hybridoma techniques for the isolation and subsequent cloning of monoclonal antibodies. In phage display methods, functional antibody domains are displayed on the surface of phage particles, which carry the polynucleotide sequences encoding them. For example, DNA sequences encoding VH and VL regions are amplified or otherwise isolated from animal cDNA libraries (e.g., human or murine cDNA libraries of lymphoid tissues) or synthetic cDNA libraries.

In certain embodiments, the DNA encoding the VH and VL regions are joined together by an scFv linker by PCR and cloned into a phagemid vector (e.g., p CANTAB 6 or pComb 3 HSS).

The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., LSR polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al, J. Immunol. Methods 752:41-50 (1995); Ames et al, J. Immunol. Methods/£4:177-186 (1995); Kettleborough et al, Eur. J. Immunol. 24:952-95% (1994); Persic et al, Gene 757:9-18 (1997); Burton et al, Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety. As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria.

### Example 21

### Effector Function Activity Through Complement Dependent Cytotoxicity (CDC) of Anti LSR Human IgG1 Antibodies on LSR-Ectopic Expressing Cell Lines

Human IgG1 antibodies against LSR, 511-01.E02, S11-01.F08, 511-04. C11, 511-04.D11, 511-04.H07, 511-04.H09, 532-03.G11 were generated via phage display screening of the XOMA 031 human Fab library.

### Phage Panning Details:

### Preparation of Biotinylated LSR:

The human LSR antigen (LSR extracellular domain fused to a mouse IgG2a Fc region) and negative control protein (‘control antigen’, also comprised of a mIgG2a Fc fusion) were biotinylated to facilitate solution-based phage panning. All proteins were diluted to 1 mg/mL in 500 μL PBS, then labeled with a Sulfo-NHS-LC-Biotin kit at a 3:1 biotin:protein ratio, as per manufacturer's instructions (Pierce, Rockford, Ill.). Free biotin was removed by dialyzing samples overnight against PBS pH 7.4 using 0.5 mL 3500 MWCO Slide-A-Lyzer cassettes (Pierce). Dialyzed proteins were stored at −80° C.

### Phage Panning of Human Antibody Library:

Panning reactions were carried out in solution using streptavidin-coated magnetic beads to capture the biotinylated antigens. All washing and elution steps were conducted using a magnetic rack to capture the beads (Promega, Madison, Wis.). All incubation steps were conducted at room temperature with gentle mixing on a tube rotator (BioExpress, Kaysville, Utah). The panning experiment was conducted as outlined in Table 1:

TABLE 1
antigen density and washing stringency used
for phage panning against LSR.

| Panning conditions | Round | Antigen (pmols) | Washes |
|---|---|---|---|
| Panning experiment A | 1 | 100 | 3× PBS-T; 3× PBS |
| Constant Ag density | 2 | 100 | 6× PBS-T; 6× PBS |
| | 3 | 100 | 6× PBS-T; 6× PBS |

### Preparation of Phage Library for Panning:

All phage panning experiments used the X0MA031 human fab antibody phage display library (XOMA Corporation, Berkeley, Calif.). Sufficient phage for a 50-fold over-representation of the library were blocked by mixing 1:1 with 10% skim milk powder in PBS (final skim milk concentration 5%) and incubating for 1 hr.

### Antigen Coupling to Streptavidin Beads:

For each panning reaction, three 100 μL aliquots of Dynal streptavidin-coated magnetic beads (Life Technologies) were blocked by suspension in 1 mL of blocking buffer (5% skim milk powder in PBS) and incubated alongside the blocking phage library. One blocked bead aliquot was mixed with an amount of biotinylated LSR dependent on the panning round and reaction conditions (Table 1). The other two aliquots were mixed with 100 pmols of the ‘depletion’ control antigen. Biotin-labeled antigens were coupled to the beads for 1 hr at RT. Beads were washed twice with PBS to remove free antigen and re-suspended in 100 μL blocking buffer.

Depletion of Mouse IgG2a Fc Binders from the Phage Library:

It was necessary to remove unwanted binders to the Fc region of LSR before phage panning could commence. To achieve this, blocked phage was mixed with beads coupled to the control antigen and incubated for 1 hr. The beads (and presumably unwanted mIgG2a Fc-binders) were then discarded. The ‘depleted’ phage library supernatant was used for three rounds of panning on the LSR antigen.

Fab PPE Production and Screening:

Eluted phage pools from panning round 3 were diluted and infected into TG1 E. coli cells (Lucigen, Middleton, Wis.) so that single colonies were generated when spread on a 2YTCG agar plate.

Fab proteins secreted into the E. coli periplasm were extracted for analysis. Cells were harvested by centrifugation at 2500×G, the supernatants were discarded and pellets were re-suspended in 75 μL ice-cold PPB buffer (Teknova). Extracts were incubated for 10 mins at 4° C. with 1000 rpm shaking, and then 225 μL ice-cold ddH₂O was then added and incubated for a further 1 hr. The resulting periplasmic extract (PPE) was cleared by centrifugation at 2500×G and transferred to separate plates or tubes for ELISA and FACS analysis. Note that all extraction buffers contained Halt (Pierce) protease inhibitors Periplasmic extracts containing expressed Fabs were tested by ELISA and FACS analysis for binding to LSR. Twelve sequence-unique Fabs were identified that bind to LSR by ELISA. Of these, seven were FACS positive for binding. Six of the FACS binders were reformatted to full-length human IgG1 antibodies, and purified for functional testing. The sequences of the purified antibodies further used for functional testing are described below.

The aim of the functional analysis was to establish a functional assay addressing the complement fixing ability of LSR monoclonal antibodies on a cell line that express LSR and to use this assay to screen for potential therapeutic antibodies for CDC effector function.

Materials and Methods

LSR Expressing Cell Lines:

HEK293T cells expressing cynomolgus LSR or GFP expressing vector or empty vector alone were generated as described herein above. HEK293T transfected cells were cultured in complete media (CM): 5 ug/ml puromycin in DMEM supplemented with 10% FBS, Glutamine and Penstrep.

Antibodies:

Human IgG1 antibodies against LSR, S11-01.E02 (SEQ ID NOs: 244 and 238, for VL and VH, respectively), S11-01.F08 (SEQ ID NOs: 260 and 250, for VL and VH, respectively), S11-04.C11 (SEQ ID NOs: 273 and 263, for VL and VH, respectively), S11-04.D11 (SEQ ID NOs: 281 and 279, for VL and VH, respectively), S11-04.H07 (SEQ ID NOs: 308 and 283, for VL and VH, respectively), S11-04.H09 (SEQ ID NOs: 289 and 286, for VL and VH, respectively), 532-03.G11 (SEQ ID NOs: 308 and 293, for VL and VH, respectively) were generated as described herein. Human IgG1 isotype control (cat# ET-901) was purchased from Eureka Therapeutics, USA, Mouse IgG2a monoclonal antibody against human LSR, 8C8, was generated at Biotem, France. Mouse IgG2a Isotype control, clone MOPC-173 (cat#400224), was purchased from Biolegend, USA.

A standardized numbering scheme for antibodies was first introduced by Kabat et al. (1983). This numbering scheme was derived on the basis of sequence alignments when no structural information for antibodies was available. Chothia and Lesk (1987) examined the variable domains of antibody structures and showed that the sites of insertions and deletions (indels) in CDR-L1 and CDR-H1 suggested by Kabat on the basis of sequence were not structurally correct. This led to the introduction of the Chothia numbering scheme. In both Kabat and Chothia schemes, the numbering is based on the most common sequence lengths and insertions are accommodated with insertion letters (e.g. 30a).

Abhinandan and Martin (2008) have examined the annotations with standard numbering that are present in the Kabat databank and have found that approximately 10% of sequences have an error in the (manually applied) numbering. To perform this analysis they have created a software tool (AbNum) that applies the Kabat or Chothia numbering in an automatic and reliable manner. The numbering generated by the AbNum program was compared with the numbering appearing in Kabat. Where differences were identified, the numbering was examined manually to determine whether the error was in AbNum or in Kabat. After several rounds of refinement of the software, they determined that all errors appear to be in Kabat.

When numbering is being done, CDRs can be defined. There are three popular ways to define CDRs: (1) The Kabat definition, which is based on sequence variability and is the most commonly used; (2) The Chothia definition, which is based on the location of the structural loop regions; (3) The AbM definition, which is a compromise between the previous two, used by Oxford Molecular's AbM antibody modelling software. Detailed description of the three ways is described at http://www.bioinf.org.uk/abs/. There are also few other proprietary methods to define CDRs (such as (4) the SegAgent™ software program, developed by XOMA (US) LLC, Berkeley Calif., the principles of which are described in http://www.imgt.org/IMGTScientificChart/Nomenclature/IMGT-FRCDRdefinition.html). A summary of the three most popular ways is presented in the Table below.

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32 . . . 34 | H30-H35B |
| | (Kabat Numbering) | | | |
| H1 | H31-H35 | H26-H35 | H26-H32 | H30-H35 |
| | (Chothia Numbering) | | | |
| H2 | H50-H65 | H50-H58 | H52-H56 | H47-H58 |
| H3 | H95-H102 | H95-H102 | H95-H102 | H93-H101 |

Based on the various CDR definitions, the following CDRs were defined for these LSR antibodies (the number in the parenthesis near each SEQ ID NO corresponds to the method of CDR detection used for the corresponding CDR definition, as described above):

S11-01.E02, light chain CDR1 depicted in SEQ ID NOs:245 (1-3), 246 (4), light chain CDR2 depicted in SEQ ID NOs:247 (1-3), 248 (4), light chain CDR3 depicted in SEQ ID NO:249 (1-4), heavy chain CDR1 depicted in SEQ ID NOs:239 (3), 240 (4), 241 (1), 252 (2), heavy chain CDR2 depicted in SEQ ID NOs:256 (4), 287 (2-3), 288 (1), heavy chain CDR3 depicted in SEQ ID NOs:242 (1-3), 243 (4).
S11-01.F08, light chain CDR1 depicted in SEQ ID NOs:303 (1-3), 304 (4), light chain CDR2 depicted in SEQ ID NOs:261 (1-3), 306 (4), light chain CDR3 depicted in SEQ ID NO:262 (1-4), heavy chain CDR1 depicted in SEQ ID NOs:251 (3), 252 (2), 253 (4), 254 (1), heavy chain CDR2 depicted in SEQ ID NOs:255 (2-3), 256 (4), 257 (1), heavy chain CDR3 depicted in SEQ ID NOs:258 (1-3), 259 (4).
S11-04.C11, light chain CDR1 depicted in SEQ ID NOs:274 (1-3), 275 (4), light chain CDR2 depicted in SEQ ID NOs:276 (1-3), 277 (4), light chain CDR3 depicted in SEQ ID NO:278 (1-4), heavy chain CDR1 depicted in SEQ ID NOs:264 (3), 265 (2), 266 (4), 267 (1), heavy chain CDR2 depicted in SEQ ID NOs:268 (2-3), 269 (4), 270 (1), heavy chain CDR3 depicted in SEQ ID NOs:271 (1-3), 272 (4).
S11-04.D11, light chain CDR1 depicted in SEQ ID NOs:274 (1-3), 275 (4), light chain CDR2 depicted in SEQ ID NOs:276 (1-3), 277 (4), light chain CDR3 depicted in SEQ ID NO:282 (1-4), heavy chain CDR1 depicted in SEQ ID NOs:264 3), 265 (2), 266 (4), 267 (1), heavy chain CDR2 depicted in SEQ ID NOs:268 (2-3), 269 (4), 280 (1), heavy chain CDR3 depicted in SEQ ID NOs:271 (1-3), 272 (4).
S11-04.H07, light chain CDR1 depicted in SEQ ID NOs:303 (1-3), 304 (4), light chain CDR2 depicted in SEQ ID NOs:305 (1-3), 306 (4), light chain CDR3 depicted in SEQ ID NO:307 (1-4), heavy chain CDR1 depicted in SEQ ID NOs:239 (3), 240 (4), 241 (1), 252 (2), heavy chain CDR2 depicted in SEQ ID NOs:256 (4), 287 (2-3), 288 (1), heavy chain CDR3 depicted in SEQ ID NOs:284 (1-3), 285 (4).
S11-04.H09, light chain CDR1 depicted in SEQ ID NOs:290 (1-3), 291 (4), light chain CDR2 depicted in SEQ ID NOs:305 (1-3), 306 (4), light chain CDR3 depicted in SEQ ID NO:292 (1-4), heavy chain CDR1 depicted in SEQ ID NOs:251 (3), 252 (2), 253 (4), 254 (1), heavy chain CDR2 depicted in SEQ ID NOs:256 (4), 287 (2-3), 288 (1), heavy chain CDR3 depicted in SEQ ID NOs:258 (1-3), 259 (4).
S32-03.G11, light chain CDR1 depicted in SEQ ID NOs:303 (1-3), 304 (4), light chain CDR2 depicted in SEQ ID NOs:305 (1-3), 306 (4), light chain CDR3 depicted in SEQ ID NO:307 (1-4), heavy chain CDR1 depicted in SEQ ID NOs:294 (3), 295 (2), 296 (4), 297 (1), heavy chain CDR2 depicted in SEQ ID NOs:298 (2-3), 299 (4), 300 (1), heavy chain CDR3 depicted in SEQ ID NOs:301 (1-3), 302 (4).

Reagents: Purified rabbit complement (cat# CL-3441) was purchased from Cedarlane laboratories, Canada. Cell Titer Glo reagent was purchased from Promega, USA.

Cytotoxic Assay: The CDC activity of LSR antibodies against HEK293 ectopically expressing cyno LSR or control GFP was evaluated using cell titer glo reagent. Cells were plated at a density of 2.5×10⁴ cells per well in a 96 well white tissue culture plate in 50u1 of CM. After allowing cells to settle for 2 hours, serial dilutions of 2× antibody, isotype or media alone were added in equal volume to respective wells. After 10 minutes at room temperature, 4u1 of freshly reconstituted complement was added to each testing well except control wells and plates incubated at 37 degrees for one hour. Plates were then equilibrated to room temperature for 10 minutes, and 100u1 of cell titer glo reagent added per well and incubated at RT for 10 minutes. Luminescence was measured on Victor2 plate reader (Perkin Elmer) as RLU (relative luminescence units). Data was analyzed in Excel and plotted in GraphPad

Prism. Percent CDC was calculated as follows: 100-[(RLU experimental well/RLU control well) X 100]. Conditions were run in triplicate and data shown here is representative of one experiment.

FACS Analysis: HEK293 transfected with empty vector or cyno LSR expressing HEK293 cells were washed and stained in 50u1 of different concentrations of LSR antibodies or isotype control in FACS buffer (PBS (Life Technologies), 0.5% BSA (Sigma Aldrich)) at 4 degrees C. for 60 minutes. Cells were washed once in FACS buffer, re-suspended in 50u1 of anti-mouse IgG biotinylated (GE cat#RPN1001V) or anti-human Fc biotinylated (Jackson cat#109-065-097) at 10 ug/ml for 30 minutes at 4 degrees C. Cells were washed in FACS buffer, re-suspended in 50u1 of 1:100 dilution of SA-PE (Jackson cat#016-110-084) made in FACS buffer for 30 minutes. Cells were washed twice and re suspended in a final volume of 100u1 of FACS buffer. Samples were read on the Intellicyt HTFC. Data was analyzed by FCS Express (DeNovo), exported to Excel and plotted in GraphPad Prism.

### Results

As shown in FIG. 25, LSR human IgG1 antibodies were shown to demonstrate CDC activity on HEK293 ectopically expressing LSR.

In this experiment the activity of 7 human IgG1 monoclonal antibodies against LSR protein ectopically expressed on HEK293T cells was evaluated at a single concentration of 10 ug/ml. As shown in FIG. 25, 6 of 7 antibodies displayed CDC activity on HEK293T expressing LSR cells whilst showing no activity on the GFP control cell line. 8C8, a mouse IgG2a monoclonal against human LSR, showed no activity in this assay, similarly to the negative controls hIgG1 (ET901) and mIgG2a (Mopc173), and to one of the anti-LSR antibodies which showed no activity (532-03.G11).

All six antibodies showing CDC activity bound the LSR expressing cell line with none or low binding to empty vector control cell line by FACS staining, as shown in FIGS. 26 C, D, E, F, G and H. The antibody 532-03.G11, which showed no specific CDC activity, did not bind LSR expressing HEK293 cells, as shown in FIG. 26B. Isotype control of human IgG1 did not bind to either LSR expressing cells or to cells transfected with empty vector. The mouse 8C8 mAb showed binding to the LSR expressing HEK293 cells (FIG. 26A), but was devoid of CDC activity (FIG. 25)

Several human anti-LSR antibodies showed CDC activity on HEK293T cells expressing LSR protein. This assay could be used to characterize functional Abs of LSR. The results raise the possibility that LSR therapeutic antibodies of the human IgG1 sub-class, known for complement fixing activity, could potentially act through multiple mechanisms of action, including CDC mediated effector function on LSR expressing cancer cells.

### Example 22

### In-Vitro Testing of the Effect of LSR, Expressed on HEK 293T Cells, on Activation of Jurkat Cells and Decreasing Inhibition of Anti CD3-Mediated Activation of Jurkat T Cells by Reformatted ABS as Measured by CD69 Expression

In order to evaluate the effect of the native cell surface expressed LSR protein on T cell activation, a co-culture assay of HEK-293T cells over expressing LSR and Jurkat cells (derived from a human T cell leukemia) activated by plate-bound anti-CD3 antibodies were used.

### Materials and Methods

### Anti-CD3 Mediated Activation of Jurkat T Cells as Measured by CD69 Expression.

Day 1:
1. Anti-CD3 (Clone OKT3, eBioscience; cat#16-0037-85 or clone UCHT1, BD Bioscience; cat#555329) diluted in 1×PBS was immobilized on a flat-bottom 96-well plate in 75 μL/well at the indicated concentrations
2. Plates were wrapped with parafilm and incubated at 4° C. O.N.
3. HEK-293T cell pools stably transfected with expression constructs of the pRp3 plasmid, encoding LSR or CD20 (as negative control), or with the empty vector pRp3, were seeded at a concentration of 12×10⁶ cells per T75 plate and cultured in DMEM medium supplemented with 10% FBS, L-glutamine, penicillin, and streptomycin in a humidified incubator O.N.

Day 2:
1. Wells coated with anti-CD3 were washed ×3 with 200 μl of X1 PBS. Fluid was decanted in a sterile environment. After the last wash, the plate was blotted on a sterile absorbent paper to remove any residual fluid.
2. HEK-293T cells, seeded the day before, were treated with mitomycin C (Sigma, M4287): 900 μl of a 0.5 mg/ml solution freshly prepared in H2O were added directly to 8.1 ml of growth medium, to obtain a final concentration of 50 μg/ml. Cells were incubated with mitomycin C for 1 hour at 37° C.
3. Mitomycin C treated HEK-293T cells were washed ×3 with 10 ml of 1×PBS and removed by addition of 2 ml of cell dissociation buffer (Gibco; Cat. 13151-014).
4. Detached HEK-293T cells were re-suspended in 8 ml of RPMI supplemented with 10% FBS, L-glutamine, penicillin, and streptomycin (Jurkat cells' growth medium).
5. Cells were counted using a Beckman coulter counter and diluted to 0.5×10⁶ cells per ml.
6. Cells were serially diluted and seeded at the indicated concentrations in 100 μl per well of Jurkat cells' growth medium (described above).
7. HEK 293T cells were incubated for 2 hours to allow attachment.
8. 50,000 Jurkat cells (ATCC, clone E6-1, TIB-152) were added to each well at a volume of 100 μl per well in Jurkat cells' growth medium (described above).
9. Cells were co-cultured O.N. at 37° C. in a humidified incubator.

Day 3:
1. Cells were transferred to U-shape plates, centrifuged 5 minutes at 1500 rpm, 4° C., and the supernatant was decanted.
2. Anti-CD69 Ab (Biolegend, PE-anti human CD69, clone FN50, cat#310906, 10 μg/ml, 2 μl/well) and Fc-blocker (Miltenyi Biotec, human FcR blocking reagent, cat#120000-442, 1 μl/well) were diluted in ice-cold FACS buffer (1×PBS+0.5% BSA+2 mM EDTA+0.05% azide) and added in a final volume of 50 μl per well.
3. The wells content was mixed gently by pipetting (without making air bubbles).
4. Plates were incubated on ice for 30 minutes.
5. Cells were washed once with 200 μl of FACS buffer and the plates were centrifuged 5 mM at 1500 rpm, 4° C. Sup was discarded by decanting.
6. Cells were resuspended in 200 μl of FACS buffer and transferred to FACS tubes filled with additional 100 μl FACS buffer.
7. Jurkat cells were analyzed by flow cytometry for cell surface expression of CD69 (Mean Fluorescence Intensity (MFI) or the percentage of cells expressing CD69 out of total T cells). Jurkat cells were gated according to Forward Scatter (FSC) vs. Side Scatter (SSC). Gating procedure was validated by staining the cells with anti-CD2 antibody (Biolegend; clone RPA-2.10, Cat. 300206) in order to identify the Jurkat T cells.

### Results

### Inhibition of Anti CD3-Mediated Activation of Jurkat T Cells as Measured by CD69 Expression.

HEK-293T transfectants expressing the full length LSR protein were co-cultured with Jurkat T cells activated by plate-bound anti-CD3 antibodies, as described in Mat & Meth. HEK-293T cells transfected with the vector only (pRp3) were used as a negative control. Representative results shown in FIG. 27 indicate that Jurkat T cells stimulated with two different clones of anti-human CD3 antibodies (OKT3 or UCHT1) exhibit reduced activation in the presence of LSR-expressing HEK-293T cells, as manifested by reduced upregulation of CD69, an early marker of T cell activation, in comparison to the effect of HEK-293T cells transfected with the vector only (pRp3). The inhibitory effect of LSR is best detected when using 50,000 HEK-293T transfected cells per well. In FIG. 28, results from a similar experiment are shown. In this experiment, UCHT1 anti-CD3 Ab clone was used, and results are shown as % of Jurkat cells expressing CD69. The findings were compared to those obtained with HEK-293T cells expressing the CD20 protein, which did not show an effect.

FIG. 27: LSR expressed on HEK-293T cells inhibits Jurkat cells activation. HEK-293T cells expressing LSR or the pRp3 empty vector were seeded at 25,000 (A and C) or 50,000 (B and D) cells per well, in wells pre-coated with 0.25 μg/ml of anti-CD3 (OKT3 clone) (A and B) or 2 μg/ml of anti-CD3 (UCHT1 clone) (C and D). Jurkat cells were added 2 hours later at 50,000 cells per well, and the co-cultures were incubated O.N. Cells were analyzed for the expression of CD69 by flow cytometry. As reference, CD69 values of untreated Jurkat cells (UT), i.e. not treated with anti-CD3, are shown. AMFI values of CD69 between untreated and anti-CD3 treated Jurkat cells in the presence of 50,000 HEK-293 transfected cells are presented in (E)* indicates value significantly different from that of the empty vector (p<0.05, Student's t-test).

FIG. 28: HEK-293T cells expressing LSR inhibit Jurkat cells activated with anti-CD3, as opposed to HEK-293T cells expressing CD20. HEK-293T cells expressing LSR, CD20 or the pRp3 empty vector were seeded at 25,000 (A) or 50,000 (B) cells per well in wells pre-coated with 2 μg/ml of anti-CD3 (UCHT1 clone). Jurkat cells were added 2 hours later at 50,000 cells per well and the co-cultures were incubated O.N. Cells were analyzed for the expression of CD69 by flow cytometry. The percentage of Jurkat cells expressing CD69 is shown in (A) and (B). The difference in the % CD69 between the activated cells to non-activated ones (ACD69%) is shown in (C). The percentage of inhibition of CD69 upregulation is shown in (D)* indicates value significantly different from that of the empty vector (p<0.05, Student's t-test).

Based on these results, the cell surface expressed form of LSR expressed on the cell surface of HEK-293T cells inhibits Jurkat T cell activation. These results further support previous findings obtained with the LSR fusion protein, composed of the ectodomain of LSR fused to mIgG2a Fc, which demonstrated an inhibitory effect on T cell activation in various experimental settings.

### Decreasing Inhibition of Anti CD3-Mediated Activation of Jurkat T Cells by Reformatted Abs as Measured by CD69 Expression.

In order to evaluate the functional effect of LSR specific Abs on T cell activation, a co-culture assay, as described above, was performed in the presence of the different LSR hIgG1 Abs described herein.

HEK-293T cells expressing the full length LSR wild type protein were co-cultured with Jurkat T cells activated by plate-bound anti-CD3 antibodies in the presence or absence of LSR specific Abs. LSR specific Abs were added to each well to a final concentration of 20 ug/ml in a total volume of 50u1 of Jurkat cells growth medium. HEK-293T cells transfected with the vector only (pRp3) were used as a negative control. Co-culture of HEK-293T cells expressing LSR with Jurkat T cells, in the presence of a negative control (hIgG1) leads to inhibition of CD69 expression, as demonstrated in Example 22 herein. Preliminary results indicate that addition of at least one LSR hIgG1 Ab, S11-04.H07, increase the intensity of CD69 expression, although this trend did not reach statistical significance, thus reducing the inhibitory effect mediated by the cell surface expressed LSR protein. This observation suggests that this Ab (which was previously shown to specifically bind to cell surface expressed LSR) might possess a functional effect by neutralizing the T cell inhibition mediated by LSR.

Despite demonstrating neutralizing activity in the co-culture assay, the tested antibodies had no effect on LSR inhibition of T-cell activation in an assay format involving plate bound fusion protein. Without wishing to be limited in any way, this disparity between the assays may be due to a number of factors, including differences in target density, conformational anomalies introduced by protein binding to the plates, or unexpected effects of the Fc region of the fusion protein on T-cell activation.

The invention has been described and various embodiments provided relating to manufacture and selection of desired anti-LSR antibodies for use as therapeutics and diagnostic methods for various diseases. Different embodiments may optionally be combined herein in any suitable manner, beyond those explicit combinations and subcombinations shown herein. The invention is now further described by the claims which follow.

1-89. (canceled)

90. A method of treating a subject with cancer, the method comprising administering an antibody or a fragment specifically binding to SEQ ID NO: 10 for treating the subject, wherein the cancer is selected from the group consisting of ductal-adenocarcinoma, infiltrating ductal carcinoma, lobular carcinoma, mucinous adenocarcinoma, intra duct and invasive ductal carcinoma, Scirrhous adenocarcinoma, Moderate to Poorly Differentiated Adenocarcinoma of the cecum, Well, Moderate and Poorly Differentiated Adenocarcinoma of the colon, Tubular adenocarcinoma, Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum, Well to Poorly differentiated Non-small cell carcinoma, Squamous Cell Carcinoma, preferably Moderately Differentiated Squamous Cell Carcinoma, Moderately to poorly differentiated squamous carcinoma, Moderately well differentiated keratinising squamous cell carcinoma, large cell adenocarcinoma, Small cell lung cancer, Adenocarcinoma Gleason Grade 5 to 9, Infiltrating adenocarcinoma, High grade prostatic intraepithelial neoplasia, undifferentiated carcinoma, moderately differentiated gastric adenocarcinoma, serous papillary cystic carcinoma, Serous cystadenocarcinoma, Invasive serous papillary carcinoma, Glioblastoma multiforme, Astrocytoma, Astrocytoma grade 4, Clear cell renal cell carcinoma, Hepatocellular carcinoma, Low Grade hepatocellular carcinoma, Fibrolamellar Hepatocellular Carcinoma, large cell lymphoma, and High and low grade Non-Hodgkin's Lymphoma; with the proviso that if the cancer is ovarian cancer, it is not Granulosa cell tumor of the ovary and with the proviso that if the cancer is brain cancer, it is not Astrocytoma grade 2.

91. The method of claim 90, wherein said antibody has an antigen-binding region that binds specifically to amino acids 30-110 of SEQ ID NO 10 and that does not specifically bind to any other portion of SEQ ID NO 10, wherein said other portion of SEQ ID NO:10 comprises amino acids 1-29 or amino acids 111 to 234 of SEQ ID NO: 10.

92. The method of claim 91, wherein said antibody has an antigen-binding region that binds specifically to SEQ ID NO 215 or to SEQ ID NO 216 and that does not specifically bind to any other portion of SEQ ID NO 10, wherein said other portion of SEQ ID NO:10 comprises amino acids 1-80 or amino acids 99 to 234 of SEQ ID NO: 10 for SEQ ID NO 215, or wherein said other portion of SEQ ID NO:10 comprises amino acids 1-117 or amino acids 136 to 234 of SEQ ID NO: 10 for SEQ ID NO 216.

93. The method of claim 90, wherein said administering said antibody or fragment comprises administering said antibody or fragment in a pharmaceutical composition comprising a pharmaceutical carrier.

94. The method of claim 90, wherein said administering further comprises administering another therapeutic agent or therapy useful for treating cancer.

95. The method of claim 94, wherein therapeutic agent or therapy is administered to a subject simultaneously with the antibody or fragment.

96. The method of claim 94, wherein therapeutic agent or therapy is administered to a subject sequentially with the antibody or fragment.

97. The method of claim 94, wherein the therapy comprises one or more of radiotherapy, cryotherapy, photodynamic therapy, adoptive cell transfer or surgery.

98. The method of claim 94, wherein the therapeutic agent is selected from the group consisting of cytotoxic drugs, a therapeutic cancer vaccine, an additional antibody, peptides, pepti-bodies, small molecules, chemotherapeutic agents, cytotoxic and cytostatic agents, immunological modifiers, interferons, interleukins, immunostimulatory growth hormones, cytokine therapy, vitamins, minerals, aromatase inhibitors, RNAi, Histone Deacetylase Inhibitors and proteasome inhibitors.

99. The method of claim 98, wherein the chemotherapeutic agent is selected from the group consisting of platinum based compounds, antibiotics with anti-cancer activity, Anthracyclines, Anthracenediones, alkylating agents, antimetabolites, Antimitotic agents, Taxanes, Taxoids, microtubule inhibitors, Vinca alkaloids, Folate antagonists, Topoisomerase inhibitors, Antiestrogens, Antiandrogens, Aromatase inhibitors, GnRh analogs, inhibitors of 5α-reductase and biphosphonates.

100. The method of claim 98, wherein the therapeutic agent is selected from the group consisting of histone deacetylase (HDAC) inhibitors, proteasome inhibitors, mTOR pathway inhibitors, JAK2 inhibitors, tyrosine kinase inhibitors (TKIs), PI3K inhibitors, Protein kinase inhibitors, Inhibitors of serine/threonine kinases, inhibitors of intracellular signaling, inhibitors of Ras/Raf signaling, MEK inhibitors, AKT inhibitors, inhibitors of survival signaling proteins, cyclin dependent kinase inhibitors, therapeutic monoclonal antibodies, TRAIL pathway agonists, anti-angiogenic agents, metalloproteinase inhibitors, cathepsin inhibitors, inhibitors of urokinase plasminogen activator receptor function, immunoconjugates, antibody drug conjugates, antibody fragments, bispecfic antibodies and bispecific T cell engagers (BiTEs).

101. The method of claim 98, wherein the additional antibody is selected from the group consisting of cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab and aflibercept.

102. The method of claim 98, wherein the therapeutic agent is selected from the group consisting of antimitotic drugs, cyclophosphamide, gemcitabine, mitoxantrone, fludarabine, thalidomide, thalidomide derivatives, COX-2 inhibitors, depleting or killing antibodies that directly target Tregs through recognition of Treg cell surface receptors, anti-CD25 daclizumab, basiliximab, ligand-directed toxins, denileukin diftitox (Ontak)—a fusion protein of human IL-2 and diphtheria toxin, or LMB-2—a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin.

103. The method of claim 98, wherein the additional antibody is selected from antagonistic antibodies targeting one or more of CTLA4, PD-1, PDL-1, LAG-3, TIM-3, BTLA, B7-H4, B7-H3, VISTA, or Agonistic antibodies targeting one or more of CD40, CD137, OX40, GITR, CD27, CD28 or ICOS, or a combination thereof.

104. The method of claim 98, wherein the therapeutic cancer vaccine is selected from exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cell-based vaccines, whole tumor cell vaccines, gene modified tumor cells expressing GM-CSF, ICOS and/or Flt3-ligand, oncolytic virus vaccines.

105. The method of claim 98, wherein the cytokine therapy is selected from one or more of the following cytokines such as IL-2, IL-7, IL-12, IL-15, IL-17, IL-18 and IL-21, IL23, IL-27, GM-CSF, IFNα (interferon alpha), IFNα-2b, IFNβ, IFNγ, and their different strategies for delivery.

106. The method of claim 97, wherein the adoptive cell transfer therapy is carried out following ex vivo treatment selected from expansion of the patient autologous naturally occurring tumor specific T cells or genetic modification of T cells to confer specificity for tumor antigens.

107. The method of claim 90, wherein the cancer is non-metastatic.

108. The method of claim 90, wherein the cancer is invasive.

109. The method of claim 90, wherein the cancer is metastatic.

110. A diagnostic method for determining whether to administer an antibody or fragment to a subject, wherein the antibody or a fragment specifically binds to SEQ ID NO: 10, wherein the diagnostic method is performed ex vivo, comprising contacting a tissue sample from the subject with the antibody or a fragment specifically binding to SEQ ID NO: 10 ex vivo and detecting specific binding thereto, wherein specific binding to the sample indicates the presence of cancer and wherein the cancer is selected from the group consisting of ductal-adenocarcinoma, infiltrating ductal carcinoma, lobular carcinoma, mucinous adenocarcinoma, intra duct and invasive ductal carcinoma, Scirrhous adenocarcinoma, Moderate to Poorly Differentiated Adenocarcinoma of the cecum, Well, Moderate and Poorly Differentiated Adenocarcinoma of the colon, Tubular adenocarcinoma, Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum, Well to Poorly differentiated Non-small cell carcinoma, Squamous Cell Carcinoma, preferably Moderately Differentiated Squamous Cell Carcinoma, Moderately to poorly differentiated squamous carcinoma, Moderately well differentiated keratinising squamous cell carcinoma, large cell adenocarcinoma, Small cell lung cancer, Adenocarcinoma Gleason Grade 5 to 9, Infiltrating adenocarcinoma, High grade prostatic intraepithelial neoplasia, undifferentiated carcinoma, moderately differentiated gastric adenocarcinoma, serous papillary cystic carcinoma, Serous cystadenocarcinoma, Invasive serous papillary carcinoma, Glioblastoma multiforme, Astrocytoma, Astrocytoma grade 4, Clear cell renal cell carcinoma, Hepatocellular carcinoma, Low Grade hepatocellular carcinoma, Fibrolamellar Hepatocellular Carcinoma, large cell lymphoma, and High and low grade Non-Hodgkin's Lymphoma; with the proviso that if the cancer is ovarian cancer, it is not Granulosa cell tumor of the ovary and with the proviso that if the cancer is brain cancer, it is not Astrocytoma grade 2.


SEQUENCE LISTING (308 sequences)

SEQ ID NO: 1 - protein, 297 aa, Artificial sequence
  Synthetic polypeptide
MAVLFLLLFLCGTPQAADNMQAIYVALGEAVELPCPSPPTLHGDEHLSWFCSPAAGSFTTLVAQVQVGRPAPDPGKPGRESRLRLLGNYSLWLEGSKEEDAGRYWCAVLGQHHNYQNWRVYDVLVLKGSQLSARAADGSPCNVLLCSVVPSRRMDSVTWQEGKGPVRGRVQSFWGSEAALLLVCPGEGLSEPRSRRPRIIRCLMTHNKGVSFSLAASIDASPALCAPSTGWDMPWILMLLLTMGQGVVILALSIVLWRQRVRGAPGRDASIPQFKPEIQVYENIHLARLGPPAHKPR

SEQ ID NO: 2 - protein, 218 aa, Artificial sequence
  Synthetic polypeptide
ADNMQAIYVALGEAVELPCPSPPTLHGDEHLSWFCSPAAGSFTTLVAQVQVGRPAPDPGKPGRESRLRLLGNYSLWLEGSKEEDAGRYWCAVLGQHHNYQNWRVYDVLVLKGSQLSARAADGSPCNVLLCSVVPSRRMDSVTWQEGKGPVRGRVQSFWGSEAALLLVCPGEGLSEPRSRRPRIIRCLMTHNKGVSFSLAASIDASPALCAPSTGWDMP

SEQ ID NO: 3 - protein, 540 aa, Artificial sequence
  Synthetic polypeptide
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGIVGTIVSLLLLGLAIISGLLLHYSPVFCWKVGNTSRGQNMDDVMVLVDSEEEEEEEEEEEEDAAVGEQEGAREREELPKEIPKQDHIHRVTALVNGNIEQMGNGFQDLQDDSSEEQSDIVQEEDRPV

SEQ ID NO: 4 - protein, 383 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 5 - protein, 439 aa, Artificial sequence
  Synthetic polypeptide
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGIVGTIVSLLLLGLAIISGLLLHYSPVFCWKVGNTSRGQNMDDVMVLVDSEEEEEEEEEEEEDAAVGEQEGAREREELPKEIPKQDHIHRVTALVNGNIEQMGNGFQDLQDDSSEEQSDIVQEEDRPV

SEQ ID NO: 6 - protein, 282 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 7 - protein, 366 aa, Artificial sequence
  Synthetic polypeptide
MALPPGPAALRHTLLLLPALLSSGWGELEPQIDGQTWAERALRENERHAFTCRVAGGPGTPRLAWYLDGQLQEASTSRLLSVGGEAFSGGTSTFTVTAHRAQHELNCSLQDPRSGRSANASVILNVQFKPEIAQVGAKYQEAQGPGLLVVLFALVRANPPANVTWIDQDGPVTVNTSDFLVLDAQNYPWLTNHTVQLQLRSLAHNLSVVATNDVGVTSASLPAPGLLATRVEVPLLGIVVAAGLALGTLVGFSTLVACLVCRKEKKTKGPSRHPSLISSDSNNLKLNNVRLPRENMSLPSNLQLNDLTPDSRAVKPADRQMAQNNSRPELLDPEPGGLLTSQGFIRLPVLGYIYRVSSVSSDEIWL

SEQ ID NO: 8 - protein, 206 aa, Artificial sequence
  Synthetic polypeptide
ELEPQIDGQTWAERALRENERHAFTCRVAGGPGTPRLAWYLDGQLQEASTSRLLSVGGEAFSGGTSTFTVTAHRAQHELNCSLQDPRSGRSANASVILNVQFKPEIAQVGAKYQEAQGPGLLVVLFALVRANPPANVTWIDQDGPVTVNTSDFLVLDAQNYPWLTNHTVQLQLRSLAHNLSVVATNDVGVTSASLPAPGLLATRVE

SEQ ID NO: 9 - protein, 232 aa, Artificial sequence
  Synthetic polypeptide
MELPLSQATLRHTLLLLPALLSSGQGELAPQIDGQTWAERALRENEHHAFTCRVAGGSATPRLAWYLDGQLQEATTSRLLSVGGDAFSGGTSTFTVTAQRSQHELNCSLQDPGSGRPANASVILNVQFKPEIAQVGAKYQEAQGPGLLVVLFALVRANPPANVTWIDQDGPVTVNASDFLVLDAQNYPWLTNHTVQLQLRSLAHNLSVVATNDVGVTSASLPAPGLLATRIE

SEQ ID NO: 10 - protein, 211 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIED

SEQ ID NO: 11 - protein, 601 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 12 - protein, 170 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIED

SEQ ID NO: 13 - protein, 582 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 14 - protein, 151 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLD

SEQ ID NO: 15 - protein, 533 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 16 - protein, 532 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 17 - protein, 493 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGMYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 18 - protein, 552 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 19 - protein, 429 aa, Artificial sequence
  Synthetic polypeptide
MAGLRVLLCLGALLARQGSAGLQLLLNPSRANLSVRPNSEVLPGIHPDLEAVAIGEVHDNVTLRCGSASGSRGLVTWYRNDSEPAFLVSFNSSLPPAAPRFSLEDAGALRIEALRLEDDGNYTCQEVLNETHWFPVRLRVASGPAYVEVNISATGTLPNGTLYAARGSQVDFNCCSAAQPPPEVEWWIQTHSIPEFLGKNLSANSFTLMLMSQNLQGNYTCSATNVLSGRQRKVTTELLVYWPPPSAPQCSVEVSSESTTLELACNWDGGYPDPTFLWTEEPGGTIMGNSKLQTLSPAQLLEGKKFKCVGNHILGPESGASCVVKLSSPLLPSQPMRTCFVGGNVTLTCEVSGANPPARIQWLRNLTQPAIQPSSHYIITQQGQSSSLTIHNCSQDLDEGFYYCQAENLVGVRATNIWLSVKEPLNIGG

SEQ ID NO: 20 - protein, 236 aa, Artificial sequence
  Synthetic polypeptide
MAVVVFLLFLCGHSQAVADSIQTIYVASGESVEMPCPSPPSLLGGQLLTWFRSPVAGSSTILVAQVQVDKPVSDLRKPEPDSRYKLFGNYSLWLEGSRDEDAGRYWCTVMDQNHKYQNWRVYDVSVLKGSQFSVKSPDGPSCAALLCSVVPARRLDSVTWLEGRNTVRGHAQYFWGEGAALLLVCPTEGLPETRARRPRNIRCLLPQNKRFSFSLAAASAEPSPTVCATLPSWDVP

SEQ ID NO: 21 - protein, 206 aa, Artificial sequence
  Synthetic polypeptide
MAPAASACAGAPGSHPATTIFVCLFLIIYCPDRASAIQVTVPDPYHVVILFQPVTLHCTYQMSNTLTAPIVIWKYKSFCRDRVADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLGRTSEAPELLPGFRAGPLED

SEQ ID NO: 22 - protein, 192 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLD

SEQ ID NO: 23 - protein, 451 aa, Artificial sequence
  Synthetic polypeptide
DSIQTIYVASGESVEMPCPSPPSLLGGQLLTWFRSPVAGSSTILVAQVQVDKPVSDLRKPEPDSRYKLFGNYSLWLEGSRDEDAGRYWCTVMDQNHKYQNWRVYDVSVLKGSQFSVKSPDGPSCAALLCSVVPARRLDSVTWLEGRNTVRGHAQYFWGEGAALLLVCPTEGLPETRARRPRNIRCLLPQNKRFSFSLAAASAEPSPTVCATLPSWDVPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - protein, 641 aa, Artificial sequence
  Synthetic polypeptide
LQLLLNPSRANLSVRPNSEVLPGIHPDLEAVAIGEVHDNVTLRCGSASGSRGLVTWYRNDSEPAFLVSFNSSLPPAAPRFSLEDAGALRIEALRLEDDGNYTCQEVLNETHWFPVRLRVASGPAYVEVNISATGTLPNGTLYAARGSQVDFNCCSAAQPPPEVEWWIQTHSIPEFLGKNLSANSFTLMLMSQNLQGNYTCSATNVLSGRQRKVTTELLVYWPPPSAPQCSVEVSSESTTLELACNWDGGYPDPTFLWTEEPGGTIMGNSKLQTLSPAQLLEGKKFKCVGNHILGPESGASCVVKLSSPLLPSQPMRTCFVGGNVTLTCEVSGANPPARIQWLRNLTQPAIQPSSHYIITQQGQSSSLTIHNCSQDLDEGFYYCQAENLVGVRATNIWLSVKEPLNIGGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 25 - protein, 439 aa, Artificial sequence
  Synthetic polypeptide
ELAPQIDGQTWAERALRENEHHAFTCRVAGGSATPRLAWYLDGQLQEATTSRLLSVGGDAFSGGTSTFTVTAQRSQHELNCSLQDPGSGRPANASVILNVQFKPEIAQVGAKYQEAQGPGLLVVLFALVRANPPANVTWIDQDGPVTVNASDFLVLDAQNYPWLTNHTVQLQLRSLAHNLSVVATNDVGVTSASLPAPGLLATRIEEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - protein, 403 aa, Artificial sequence
  Synthetic polypeptide
IQVTVPDPYHVVILFQPVTLHCTYQMSNTLTAPIVIWKYKSFCRDRVADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLGRTSEAPELLPGFRAGPLEDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - protein, 233 aa, Artificial sequence
  Synthetic polypeptide
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 28 - protein, 365 aa, Artificial sequence
  Synthetic polypeptide
MELPLSQATLRHTLLLLPALLSSGQGELAPQIDGQTWAERALRENEHHAFTCRVAGGSATPRLAWYLDGQLQEATTSRLLSVGGDAFSGGTSTFTVTAQRSQHELNCSLQDPGSGRPANASVILNVQFKPEIAQVGAKYQEAQGPGLLVVLFALVRANPPANVTWIDQDGPVTVNASDFLVLDAQNYPWLTNHTVQLQLRSLAHNLSVVATNDVGVTSASLPAPGLLATRIEVPLLGIVVAGGLALGTLVGFSTLVACLVCRKEKKTKGPSRRPSLISSDSNNLKLNNVRLPRENMSLPSNLQLNDLTPDLRGKATERPMAQHSSRPELLEAEPGGLLTSRGFIRLPMLGYIYRVSSVSSDEIWL

SEQ ID NO: 29 - protein, 300 aa, Artificial sequence
  Synthetic polypeptide
MAVVVFLLFLCGHSQAVADSIQTIYVASGESVEMPCPSPPSLLGGQLLTWFRSPVAGSSTILVAQVQVDKPVSDLRKPEPDSRYKLFGNYSLWLEGSRDEDAGRYWCTVMDQNHKYQNWRVYDVSVLKGSQFSVKSPDGPSCAALLCSVVPARRLDSVTWLEGRNTVRGHAQYFWGEGAALLLVCPTEGLPETRARRPRNIRCLLPQNKRFSFSLAAASAEPSPTVCATLPSWDVPWILVLLFTAGQGVTIIALSIVLWRRRRAQGSRDREPSVPHFKPEVQVYENIHLARLSPPNHKTR

SEQ ID NO: 30 - protein, 558 aa, Artificial sequence
  Synthetic polypeptide
MAGLRVLLCLGALLARQGSAGLQLLLNPSRANLSVRPNSEVLPGIHPDLEAVAIGEVHDNVTLRCGSASGSRGLVTWYRNDSEPAFLVSFNSSLPPAAPRFSLEDAGALRIEALRLEDDGNYTCQEVLNETHWFPVRLRVASGPAYVEVNISATGTLPNGTLYAARGSQVDFNCCSAAQPPPEVEWWIQTHSIPEFLGKNLSANSFTLMLMSQNLQGNYTCSATNVLSGRQRKVTTELLVYWPPPSAPQCSVEVSSESTTLELACNWDGGYPDPTFLWTEEPGGTIMGNSKLQTLSPAQLLEGKKFKCVGNHILGPESGASCVVKLSSPLLPSQPMRTCFVGGNVTLTCEVSGANPPARIQWLRNLTQPAIQPSSHYIITQQGQSSSLTIHNCSQDLDEGFYYCQAENLVGVRATNIWLSVKEPLNIGGIVGTVVSLLLLGLAVVSGLTLYYSPAFWWKGGSTFRGQDMGDVMVLVDSEEEEEEEEEEEEKEDVAEEVEQETNETEELPKGISKHGHIHRVTALVNGNLDRMGNGFQEFQDDSDGQQSGIVQEDGKPV

SEQ ID NO: 31 - protein, 594 aa, Artificial sequence
  Synthetic polypeptide
MAPAASACAGAPGSHPATTIFVCLFLIIYCPDRASAIQVTVPDPYHVVILFQPVTLHCTYQMSNTLTAPIVIWKYKSFCRDRVADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLGRTSEAPELLPGFRAGPLEDWLFVVVVCLASLLFFLLLGICWCQCCPHTCCCYVRCPCCPDKCCCPEALYAAGKAATSGVPSIYAPSIYTHLSPAKTPPPPPAMIPMRPPYGYPGDFDRTSSVGGHSSQVPLLREVDGSVSSEVRSGYRIQANQQDDSMRVLYYMEKELANFDPSRPGPPNGRVERAMSEVTSLHEDDWRSRPSRAPALTPIRDEEWNRHSPRSPRTWEQEPLQEQPRGGWGSGRPRARSVDALDDINRPGSTESGRSSPPSSGRRGRAYAPPRSRSRDDLYDPDDPRDLPHSRDPHYYDDLRSRDPRADPRSRQRSHDPRDAGFRSRDPQYDGRLLEEALKKKGAGERRRVYREEEEEEEEGHYPPAPPPYSETDSQASRERRMKKNLALSRESLVV

SEQ ID NO: 32 - protein, 575 aa, Artificial sequence
  Synthetic polypeptide
MAPAASACAGAPGSHPATTIFVCLFLIIYCPDRASAIQVTVPDPYHVVILFQPVTLHCTYQMSNTLTAPIVIWKYKSFCRDRVADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLDWLFVVVVCLASLLFFLLLGICWCQCCPHTCCCYVRCPCCPDKCCCPEALYAAGKAATSGVPSIYAPSIYTHLSPAKTPPPPPAMIPMRPPYGYPGDFDRTSSVGGHSSQVPLLREVDGSVSSEVRSGYRIQANQQDDSMRVLYYMEKELANFDPSRPGPPNGRVERAMSEVTSLHEDDWRSRPSRAPALTPIRDEEWNRHSPRSPRTWEQEPLQEQPRGGWGSGRPRARSVDALDDINRPGSTESGRSSPPSSGRRGRAYAPPRSRSRDDLYDPDDPRDLPHSRDPHYYDDLRSRDPRADPRSRQRSHDPRDAGFRSRDPQYDGRLLEEALKKKGAGERRRVYREEEEEEEEGHYPPAPPPYSETDSQASRERRMKKNLALSRESLVV

SEQ ID NO: 33 - DNA, 654 bp, Artificial sequence
  Synthetic polynucleotide
gcagacaacatgcaggccatctatgtggccttgggggaggcagtagagctgccatgtccctcaccacctactctacatggggacgaacacctgtcatggttctgcagccctgcagcaggctccttcaccaccctggtagcccaagtccaagtgggcaggccagccccagaccctggaaaaccaggaagggaatccaggctcagactgctggggaactattctttgtggttggagggatccaaagaggaagatgccgggcggtactggtgcgctgtgctaggtcagcaccacaactaccagaactggagggtgtacgacgtcttggtgctcaaaggatcccagttatctgcaagggctgcagatggatccccctgcaatgtcctcctgtgctctgtggtccccagcagacgcatggactctgtgacctggcaggaagggaagggtcccgtgaggggccgtgttcagtccttctggggcagtgaggctgccctgctcttggtgtgtcctggggaggggctttctgagcccaggagccgaagaccaagaatcatccgctgcctcatgactcacaacaaaggggtcagctttagcctggcagcctccatcgatgcttctcctgccctctgtgccccttccacgggctgggacatgcct

SEQ ID NO: 34 - DNA, 1149 bp, Artificial sequence
  Synthetic polynucleotide
gttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccagcggcccctatcagattgaggtccacatcgtggccaccggcacactccccaacggcaccctctatgcagccaggggctcccaggtggacttcagctgcaacagcagctccaggccaccacccgtggttgaatggtggttccaggccctgaattccagcagcgagtcctttggccacaacctgacagtcaactttttctcactgttactgatatcgccaaacctccaagggaactacacctgtttagccttgaatcagctcagcaagagacatcgaaaggtgaccaccgagctcctggtctactatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcgggggg

SEQ ID NO: 35 - DNA, 1320 bp, Artificial sequence
  Synthetic polynucleotide
atggccgcaggcggcagtgcgcccgagccccgcgtcctcgtctgcctcggggcgctcctggccggctgggtcgccgtaggattggaggctgttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccaatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcggggggattgtgggaaccattgtgagcctccttctgctgggactggccattatctcagggcttctgttgcattatagccctgtgttctgctggaaagtaggaaacacttccaggggacaaaacatggatgatgtcatggttttggtggattcagaagaggaagaggaggaggaggaggaggaggaggaagatgctgcagtaggggaacaggagggagcacgtgagagagaggagttgccaaaagaaatacctaagcaggaccacattcacagagtgaccgccttggtgaatgggaacatagaacagatgggaaatggattccaggatcttcaagatgacagcagtgaggagcaaagtgacattgttcaagaagaagacaggccagtctga

SEQ ID NO: 36 - DNA, 846 bp, Artificial sequence
  Synthetic polynucleotide
gttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccaatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcgggggg

SEQ ID NO: 37 - DNA, 612 bp, Artificial sequence
  Synthetic polynucleotide
gagttggagccacaaatagatggtcagacctgggctgagcgggcacttcgggagaatgaacgccacgccttcacctgccgggtggcaggggggcctggcacccccagattggcctggtatctggatggacagctgcaggaggccagcacctcaagactgctgagcgtgggaggggaggccttctctggaggcaccagcaccttcactgtcactgcccatcgggcccagcatgagctcaactgctctctgcaggaccccagaagtggccgatcagccaacgcctctgtcatccttaatgtgcaattcaagccagagattgcccaagtcggcgccaagtaccaggaagctcagggcccaggcctcctggttgtcctgtttgccctggtgcgtgccaacccgccggccaatgtcacctggatcgaccaggatgggccagtgactgtcaacacctctgacttcctggtgctggatgcgcagaactacccctggctcaccaaccacacggtgcagctgcagctccgcagcctggcacacaacctctcggtggtggccaccaatgacgtgggtgtcaccagtgcgtcgcttccagccccagggcttctggctacccgg

SEQ ID NO: 38 - DNA, 1101 bp, Artificial sequence
  Synthetic polynucleotide
atggcgctgcctccaggcccagccgccctccggcacacactgctgctcctgccagcccttctgagctcaggttggggggagttggagccacaaatagatggtcagacctgggctgagcgggcacttcgggagaatgaacgccacgccttcacctgccgggtggcaggggggcctggcacccccagattggcctggtatctggatggacagctgcaggaggccagcacctcaagactgctgagcgtgggaggggaggccttctctggaggcaccagcaccttcactgtcactgcccatcgggcccagcatgagctcaactgctctctgcaggaccccagaagtggccgatcagccaacgcctctgtcatccttaatgtgcaattcaagccagagattgcccaagtcggcgccaagtaccaggaagctcagggcccaggcctcctggttgtcctgtttgccctggtgcgtgccaacccgccggccaatgtcacctggatcgaccaggatgggccagtgactgtcaacacctctgacttcctggtgctggatgcgcagaactacccctggctcaccaaccacacggtgcagctgcagctccgcagcctggcacacaacctctcggtggtggccaccaatgacgtgggtgtcaccagtgcgtcgcttccagccccagggcttctggctacccgggtggaagtgccactgctgggcattgttgtggctgctgggcttgcactgggcaccctcgtggggttcagcaccttggtggcctgcctggtctgcagaaaagagaagaaaaccaaaggcccctcccggcacccatctctgatatcaagtgactccaacaacctaaaactcaacaacgtgcgcctgccacgggagaacatgtccctcccgtccaaccttcagctcaatgacctcactccagattccagagcagtgaaaccagcagaccggcagatggctcagaacaacagccggccagagcttctggacccggagcccggcggcctcctcaccagccaaggtttcatccgcctcccagtgctgggctatatctatcgagtgtccagcgtgagcagtgatgagatctggctctga

SEQ ID NO: 39 - protein, 232 aa, Artificial sequence
  Synthetic polypeptide
MALPPGPAALRHTLLLLPALLSSGWGELEPQIDGQTWAERALRENERHAFTCRVAGGPGTPRLAWYLDGQLQEASTSRLLSVGGEAFSGGTSTFTVTAHRAQHELNCSLQDPRSGRSANASVILNVQFKPEIAQVGAKYQEAQGPGLLVVLFALVRANPPANVTWIDQDGPVTVNTSDFLVLDAQNYPWLTNHTVQLQLRSLAHNLSVVATNDVGVTSASLPAPGLLATRVE

SEQ ID NO: 40 - DNA, 510 bp, Artificial sequence
  Synthetic polynucleotide
atccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagac

SEQ ID NO: 41 - DNA, 453 bp, Artificial sequence
  Synthetic polynucleotide
atccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgac

SEQ ID NO: 42 - DNA, 1599 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 43 - DNA, 1596 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 44 - DNA, 1473 bp, Artificial sequence
  Synthetic polynucleotide
atccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 45 - DNA, 1479 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaatgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 46 - DNA, 1656 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 47 - protein, 492 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 48 - protein, 491 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 49 - protein, 452 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGMYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 50 - protein, 511 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 51 - DNA, 20 bp, Artificial sequence
  Synthetic polynucleotide
gagaacttggcaggctctcc

SEQ ID NO: 52 - DNA, 20 bp, Artificial sequence
  Synthetic polynucleotide
cacacttcccagcagatgtc

SEQ ID NO: 53 - DNA, 34 bp, Artificial sequence
  Synthetic polynucleotide
ctagctagccaccatggcagtcttattcctcctc

SEQ ID NO: 54 - DNA, 30 bp, Artificial sequence
  Synthetic polynucleotide
cgcgaattcgcctgggcttgtgggcaggtg

SEQ ID NO: 55 - DNA, 1662 bp, Artificial sequence
  Synthetic polynucleotide
atggcagtcttattcctcctcctgttcctatgtggaactccccaggctgcagacaacatgcaggccatctatgtggccttgggggaggcagtagagctgccatgtccctcaccacctactctacatggggacgaacacctgtcatggttctgcagccctgcagcaggctccttcaccaccctggtagcccaagtccaagtgggcaggccagccccagaccctggaaaaccaggaagggaatccaggctcagactgctggggaactattctttgtggttggagggatccaaagaggaagatgccgggcggtactggtgcgctgtgctaggtcagcaccacaactaccagaactggagggtgtacgacgtcttggtgctcaaaggatcccagttatctgcaagggctgcagatggatccccctgcaatgtcctcctgtgctctgtggtccccagcagacgcatggactctgtgacctggcaggaagggaagggtcccgtgaggggccgtgttcagtccttctggggcagtgaggctgccctgctcttggtgtgtcctggggaggggctttctgagcccaggagccgaagaccaagaatcatccgctgcctcatgactcacaacaaaggggtcagctttagcctggcagcctccatcgatgcttctcctgccctctgtgccccttccacgggctgggacatgccttggattctgatgctgctgctcacaatgggccagggagttgtcatcctggccctcagcatcgtgctctggaggcagagggtccgtggggctccaggcagagatgcctcgattcctcagttcaaacccgaaatccaggtctatgagaacatccatttggcccgtcttggcccacctgcccacaagcccaggcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 56 - protein, 553 aa, Artificial sequence [sequence omitted]

SEQ ID NO: 57 - DNA, 891 bp, Artificial sequence
  Synthetic polynucleotide
atggcagtcttattcctcctcctgttcctatgtggaactccccaggctgcagacaacatgcaggccatctatgtggccttgggggaggcagtagagctgccatgtccctcaccacctactctacatggggacgaacacctgtcatggttctgcagccctgcagcaggctccttcaccaccctggtagcccaagtccaagtgggcaggccagccccagaccctggaaaaccaggaagggaatccaggctcagactgctggggaactattctttgtggttggagggatccaaagaggaagatgccgggcggtactggtgcgctgtgctaggtcagcaccacaactaccagaactggagggtgtacgacgtcttggtgctcaaaggatcccagttatctgcaagggctgcagatggatccccctgcaatgtcctcctgtgctctgtggtccccagcagacgcatggactctgtgacctggcaggaagggaagggtcccgtgaggggccgtgttcagtccttctggggcagtgaggctgccctgctcttggtgtgtcctggggaggggctttctgagcccaggagccgaagaccaagaatcatccgctgcctcatgactcacaacaaaggggtcagctttagcctggcagcctccatcgatgcttctcctgccctctgtgccccttccacgggctgggacatgccttggattctgatgctgctgctcacaatgggccagggagttgtcatcctggccctcagcatcgtgctctggaggcagagggtccgtggggctccaggcagagatgcctcgattcctcagttcaaacccgaaatccaggtctatgagaacatccatttggcccgtcttggcccacctgcccacaagcccagg

SEQ ID NO: 58 - protein, 297 aa, Artificial sequence [sequence omitted]

SEQ ID NO: 59 - protein, 234 aa, Artificial sequence
  Synthetic polypeptide
MAVLFLLLFLCGTPQAADNMQAIYVALGEAVELPCPSPPTLHGDEHLSWFCSPAAGSFTTLVAQVQVGRPAPDPGKPGRESRLRLLGNYSLWLEGSKEEDAGRYWCAVLGQHHNYQNWRVYDVLVLKGSQLSARAADGSPCNVLLCSVVPSRRMDSVTWQEGKGPVRGRVQSFWGSEAALLLVCPGEGLSEPRSRRPRIIRCLMTHNKGVSFSLAASIDASPALCAPSTGWDMP

SEQ ID NO: 60 - protein, 413 aa, Artificial sequence
  Synthetic polypeptide
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 61 - protein, 312 aa, Artificial sequence
  Synthetic polypeptide
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 62 - protein, 649 aa, Artificial sequence
  Synthetic polypeptide
MQQDGLGVGTRNGSGKGRSVHPSWPWCAPRPLRYFGRDARARRAQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 63 - protein, 630 aa, Artificial sequence
  Synthetic polypeptide
MQQDGLGVGTRNGSGKGRSVHPSWPWCAPRPLRYFGRDARARRAQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 64 - protein, 588 aa, Artificial sequence
  Synthetic polypeptide
AQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 65 - protein, 583 aa, Artificial sequence [sequence omitted]

SEQ ID NO: 66 - protein, 581 aa, Artificial sequence
  Synthetic polypeptide
MQQDGLGVGTRNGSGKGRSVHPSWPWCAPRPLRYFGRDARARRAQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 67 - protein, 493 aa, Artificial sequence; identical to SEQ ID NO: 17
  Synthetic polypeptide

SEQ ID NO: 68 - protein, 602 aa, Artificial sequence
  Synthetic polypeptide
MALLAGGLSRGLGSHPAAPGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPMEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPNGRVERAMSEVTSLHEDDWRSRPSRGPALTPIRDEEWGGHSPRSPRGWDQEPPREQAGGGWRARRPRARSVDALDDLTPPSTAESGSRSPTSSGGRRGRAYMPPRSRSRDDLYDQDDSRDFPRSRDSHYDDFRSRERPPADPRSHHHRTRDPRDHGSRSGDLLYDGRLLEEAVRKKGSEERRRPHKEEEEEAYYPPAPPPYSETDSQASRERRLKKNLALSRESLVV

SEQ ID NO: 69 - protein, 534 aa, Artificial sequence
  Synthetic polypeptide
MALLAGGLSRGLGSHPAAPGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPNGRVERAMSEVTSLHEDDWRSRPSRGPALTPIRDEEWGGHSPRSPRGWDQEPPREQAGGGWRARRPRARSVDALDDLTPPSTAESGSRSPTSSGGRRGRAYMPPRSRSRDDLYDQDDSRDFPRSRDSHYDDFRSRERPPADPRSHHHRTRDPRDHGSRSGDLLYDGRLLEEAVRKKGSEERRRPHKEEEEEAYYPPAPPPYSETDSQASRERRLKKNLALSRESLVV

SEQ ID NO: 70 - protein, 232 aa, Artificial sequence
  Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 466 aa, Artificial sequence [sequence omitted]

SEQ ID NO: 72 - protein, 645 aa, Artificial sequence [sequence omitted]

SEQ ID NO: 73 - protein, 544 aa, Artificial sequence
  Synthetic polypeptide
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 464 aa, Artificial sequence [sequence omitted]

SEQ ID NO: 75 - protein, 443 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 424 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 765 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 764 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 725 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGMYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 784 aa, Artificial sequence [sequence omitted]

SEQ ID NO: 81 - protein, 106 aa, Artificial sequence
  Synthetic polypeptide
ADNMQAIYVALGEAVELPCPSPPTLHGDEHLSWFCSPAAGSFTTLVAQVQVGRPAPDPGKPGRESRLRLLGNYSLWLEGSKEEDAGRYWCAVLGQHHNYQNWRVYD

SEQ ID NO: 82 - protein, 89 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQV

SEQ ID NO: 83 - protein, 93 aa, Artificial sequence
  Synthetic polypeptide
PYQIEVHIVATGTLPNGTLYAARGSQVDFSCNSSSRPPPVVEWWFQALNSSSESFGHNLTVNFFSLLLISPNLQGNYTCLALNQLSKRHRKVT

SEQ ID NO: 84 - protein, 87 aa, Artificial sequence
  Synthetic polypeptide
PPPSAPQCWAQMASGSFMLQLTCRWDGGYPDPDFLWIEEPGGVIVGKSKLGVEMLSESQLSDGKKFKCVTSHIVGPESGASCMVQIR

SEQ ID NO: 85 - protein, 94 aa, Artificial sequence
  Synthetic polypeptide
PSLLSEPMKTCFTGGNVTLTCQVSGAYPPAKILWLRNLTQPEVIIQPSSRHLITQDGQNSTLTIHNCSQDLDEGYYICRADSPVGVREMEIWLS

SEQ ID NO: 86 - protein, 185 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVT

SEQ ID NO: 87 - protein, 279 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIR

SEQ ID NO: 88 - protein, 374 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLS

SEQ ID NO: 89 - protein, 187 aa, Artificial sequence
  Synthetic polypeptide
PYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIR

SEQ ID NO: 90 - protein, 282 aa, Artificial sequence
  Synthetic polypeptide
PYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLS

SEQ ID NO: 91 - protein, 182 aa, Artificial sequence
  Synthetic polypeptide
PPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLS

SEQ ID NO: 92 - protein, 178 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIR

SEQ ID NO: 93 - protein, 273 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLS

SEQ ID NO: 94 - protein, 94 aa, Artificial sequence
  Synthetic polypeptide
PQIDGQTWAERALRENERHAFTCRVAGGPGTPRLAWYLDGQLQEASTSRLLSVGGEAFSGGTSTFTVTAHRAQHELNCSLQDPRSGRSANASVI

SEQ ID NO: 95 - protein, 145 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYA

SEQ ID NO: 96 - protein, 72 aa, Artificial sequence
  Synthetic polypeptide
CPSPPTLHGDEHLSWFCSPAAGSFTTLVAQVQVGRPAPDPGKPGRESRLRLLGNYSLWLEGSKEEDAGRYWC

SEQ ID NO: 97 - protein, 60 aa, Artificial sequence
  Synthetic polypeptide
CGNISGLRGQVTWYRNNSEPVFLLSSNSSLRPAEPRFSLVDATSLHIESLSLGDEGIYTC

SEQ ID NO: 98 - protein, 49 aa, Artificial sequence
  Synthetic polypeptide
CNSSSRPPPVVEWWFQALNSSSESFGHNLTVNFFSLLLISPNLQGNYTC

SEQ ID NO: 99 - protein, 46 aa, Artificial sequence
  Synthetic polypeptide
CRWDGGYPDPDFLWIEEPGGVIVGKSKLGVEMLSESQLSDGKKFKC

SEQ ID NO: 100 - protein, 58 aa, Artificial sequence
  Synthetic polypeptide
CQVSGAYPPAKILWLRNLTQPEVIIQPSSRHLITQDGQNSTLTIHNCSQDLDEGYYIC

SEQ ID NO: 101 - protein, 56 aa, Artificial sequence
  Synthetic polypeptide
CRVAGGPGTPRLAWYLDGQLQEASTSRLLSVGGEAFSGGTSTFTVTAHRAQHELNC

SEQ ID NO: 102 - protein, 108 aa, Artificial sequence
  Synthetic polypeptide
CTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYC

SEQ ID NO: 103 - DNA, 2405 bp, Artificial sequence
  Synthetic polynucleotide
tccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaaaaaaaaaaaaaaaaa

SEQ ID NO: 104 - DNA, 20 bp, Artificial sequence
  Synthetic polynucleotide
tgggaacaagagggcatctg

SEQ ID NO: 105 - DNA, 22 bp, Artificial sequence
  Synthetic polynucleotide
ccaccactgcatcaaattcatg

SEQ ID NO: 106 - DNA, 86 bp, Artificial sequence
  Synthetic polynucleotide
tgggaacaagagggcatctgctaaagtttcagattccatttctgctcagtatccagtagtggatcatgaatttgatgcagtggtgg

SEQ ID NO: 107 - DNA, 1435 bp, Artificial sequence
  Synthetic polynucleotide
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 108 - DNA, 21 bp, Artificial sequence
  Synthetic polynucleotide
tgacactggcaaaacaatgca

SEQ ID NO: 109 - DNA, 21 bp, Artificial sequence
  Synthetic polynucleotide
ggtccttttcaccagcaagct

SEQ ID NO: 110 - DNA, 94 bp, Artificial sequence
  Synthetic polynucleotide
tgacactggcaaaacaatgcagactttgctttccttggtcaggcagtataatccaaagatggtcaaggtcgcaagcttgctggtgaaaaggacc

SEQ ID NO: 111 - DNA, 2395 bp, Artificial sequence
  Synthetic polynucleotide
agaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtacacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagcc

SEQ ID NO: 112 - DNA, 20 bp, Artificial sequence
  Synthetic polynucleotide
gaggccgtcaccaagaacat

SEQ ID NO: 113 - DNA, 19 bp, Artificial sequence
  Synthetic polynucleotide
ggacagccggtcagagctc

SEQ ID NO: 114 - DNA, 111 bp, Artificial sequence
  Synthetic polynucleotide
gaggccgtcaccaagaacattcacgagtcctgcatgagccagataggctggaaccgcatcatcgtggagaagcccttcgggagggacctgcagagctctgaccggctgtcc

SEQ ID NO: 115 - DNA, 1536 bp, Artificial sequence
  Synthetic polynucleotide
gtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtaaaaaaaaaaaaaaaaaaaaaaaaaaaaaaaaaaa

SEQ ID NO: 116 - DNA, 19 bp, Artificial sequence
  Synthetic polynucleotide
tgagagtgattcgcgtggg

SEQ ID NO: 117 - DNA, 21 bp, Artificial sequence
  Synthetic polynucleotide
ccagggtacgaggctttcaat

SEQ ID NO: 118 - DNA, 91 bp, Artificial sequence
  Synthetic polynucleotide
tgagagtgattcgcgtgggtacccgcaagagccagcttgctcgcatacagacggacagtgtggtggcaacattgaaagcctcgtaccctgg

SEQ ID NO: 119 - DNA, 748 bp, Artificial sequence
  Synthetic polynucleotide
gcagataatgggaggagccgggcccgagcgagctctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgcaaaaaaaaaaaaaaaa

SEQ ID NO: 120 - DNA, 23 bp, Artificial sequence
  Synthetic polynucleotide
tggcaagaagaaggtctggttag

SEQ ID NO: 121 - DNA, 22 bp, Artificial sequence
  Synthetic polynucleotide
tgatcagcccatctttgatgag

SEQ ID NO: 122 - DNA, 101 bp, Artificial sequence
  Synthetic polynucleotide
tggcaagaagaaggtctggttagaccccaatgagaccaatgaaatcgccaatgccaactcccgtcagcagatccggaagctcatcaaagatgggctgatca

SEQ ID NO: 123 - DNA, 186 bp, Artificial sequence
  Synthetic polynucleotide
ttcactgtcactgcccatcgggcccagcatgagctcaactgctctctgcaggaccccagaagtggccgatcagccaacgcctctgtcatccttaatgtgcaattcaagccagagattgcccaagtcggcgccaagtaccaggaagctcagggcccaggcctcctggttgtcctgtttgccctggtg

SEQ ID NO: 124 - DNA, 21 bp, Artificial sequence
  Synthetic polynucleotide
ttcactgtcactgcccatcgg

SEQ ID NO: 125 - DNA, 21 bp, Artificial sequence
  Synthetic polynucleotide
caccagggcaaacaggacaac

SEQ ID NO: 126 - DNA, 1125 bp, Artificial sequence
  Synthetic polynucleotide
atggcgctgcctccaggcccagccgccctccggcacacactgctgctcctgccagcccttctgagctcaggttggggggagttggagccacaaatagatggtcagacctgggctgagcgggcacttcgggagaatgaacgccacgccttcacctgccgggtggcaggggggcctggcacccccagattggcctggtatctggatggacagctgcaggaggccagcacctcaagactgctgagcgtgggaggggaggccttctctggaggcaccagcaccttcactgtcactgcccatcgggcccagcatgagctcaactgctctctgcaggaccccagaagtggccgatcagccaacgcctctgtcatccttaatgtgcaattcaagccagagattgcccaagtcggcgccaagtaccaggaagctcagggcccaggcctcctggttgtcctgtttgccctggtgcgtgccaacccgccggccaatgtcacctggatcgaccaggatgggccagtgactgtcaacacctctgacttcctggtgctggatgcgcagaactacccctggctcaccaaccacacggtgcagctgcagctccgcagcctggcacacaacctctcggtggtggccaccaatgacgtgggtgtcaccagtgcgtcgcttccagccccagggcttctggctacccgggtggaagtgccactgctgggcattgttgtggctgctgggcttgcactgggcaccctcgtggggttcagcaccttggtggcctgcctggtctgcagaaaagagaagaaaaccaaaggcccctcccggcacccatctctgatatcaagtgactccaacaacctaaaactcaacaacgtgcgcctgccacgggagaacatgtccctcccgtccaaccttcagctcaatgacctcactccagattccagagcagtgaaaccagcagaccggcagatggctcagaacaacagccggccagagcttctggacccggagcccggcggcctcctcaccagccaaggtttcatccgcctcccagtgctgggctatatctatcgagtgtccagcgtgagcagtgatgagatctggctcgactacaaagacgatgacgacaagtga

SEQ ID NO: 127 - DNA, 37 bp, Artificial sequence
  Synthetic polynucleotide
aaagctagcgccaccatggcgctgcctccaggcccag

SEQ ID NO: 128 - DNA, 53 bp, Artificial sequence
  Synthetic polynucleotide
aaagaattctcacttgtcgtcatcgtctttgtagtcgagccagatctcatcac

SEQ ID NO: 129 - protein, 374 aa, Artificial sequence
  Synthetic polypeptide
MALPPGPAALRHTLLLLPALLSSGWGELEPQIDGQTWAERALRENERHAFTCRVAGGPGTPRLAWYLDGQLQEASTSRLLSVGGEAFSGGTSTFTVTAHRAQHELNCSLQDPRSGRSANASVILNVQFKPEIAQVGAKYQEAQGPGLLVVLFALVRANPPANVTWIDQDGPVTVNTSDFLVLDAQNYPWLTNHTVQLQLRSLAHNLSVVATNDVGVTSASLPAPGLLATRVEVPLLGIVVAAGLALGTLVGFSTLVACLVCRKEKKTKGPSRHPSLISSDSNNLKLNNVRLPRENMSLPSNLQLNDLTPDSRAVKPADRQMAQNNSRPELLDPEPGGLLTSQGFIRLPVLGYIYRVSSVSSDEIWLDYKDDDDK

SEQ ID NO: 130 - DNA, 1100 bp, Artificial sequence
  Synthetic polynucleotide
atggcgctgcctccaggcccagccgccctccggcacacactgctgctcctgccagcccttctgagctcaggttggggggagttggagccacaaatagatggtcagacctgggctgagcgggcacttcgggagaatgaacgccacgccttcacctgccgggtggcaggggggcctggcacccccagattggcctggtatctggatggacagctgcaggaggccagcacctcaagactgctgagcgtgggaggggaggccttctctggaggcaccagcaccttcactgtcactgcccatcgggcccagcatgagctcaactgctctctgcaggaccccagaagtggccgatcagccaacgcctctgtcatccttaatgtgcaattcaagccagagattgcccaagtcggcgccaagtaccaggaagctcagggcccaggcctcctggttgtcctgtttgccctggtgcgtgccaacccgccggccaatgtcacctggatcgaccaggatgggccagtgactgtcaacacctctgacttcctggtgctggatgcgcagaactacccctggctcaccaaccacacggtgcagctgcagctccgcagcctggcacacaacctctcggtggtggccaccaatgacgtgggtgtcaccagtgcgtcgcttccagccccagggcttctggctacccgggtggaagtgccactgctgggcattgttgtggctgctgggcttgcactgggcaccctcgtggggttcagcaccttggtggcctgcctggtctgcagaaaagagaagaaaaccaaaggcccctcccggcacccatctctgatatcaagtgactccaacaacctaaaactcaacaacgtgcgcctgccacgggagaacatgtccctcccgtccaaccttcagctcaatgacctcactccagattccagagcagtgaaaccagcagaccggcagatggctcagaacaacagccggccagagcttctggacccggagcccggcggcctcctcaccagccaaggtttcatccgcctcccagtgctgggctatatctatcgagtgtccagcgtgagcagtgatgagatctggctctg

SEQ ID NO: 131 - DNA, 29 bp, Artificial sequence
  Synthetic polynucleotide
aaagaattctcagagccagatctcatcac

SEQ ID NO: 132 - DNA, 1476 bp, Artificial sequence
  Synthetic polynucleotide
atccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 133 - DNA, 2201 bp, Artificial sequence
  Synthetic polynucleotide
cgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 134 - DNA, 19 bp, Artificial sequence
  Synthetic polynucleotide
atttgggtcgcggttcttg

SEQ ID NO: 135 - DNA, 21 bp, Artificial sequence
  Synthetic polynucleotide
tgccttgacattctcgatggt

SEQ ID NO: 136 - DNA, 133 bp, Artificial sequence
  Synthetic polynucleotide
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 137 - DNA, 182 bp, Artificial sequence
  Synthetic polynucleotide
gtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctt

SEQ ID NO: 138 - DNA, 20 bp, Artificial sequence
  Synthetic polynucleotide
gtcgacaaccagctcaatgc

SEQ ID NO: 139 - DNA, 20 bp, Artificial sequence
  Synthetic polynucleotide
aaggtcaggtcagcatttcc

SEQ ID NO: 140 - DNA, 154 bp, Artificial sequence
  Synthetic polynucleotide
atgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctgg

SEQ ID NO: 141 - DNA, 20 bp, Artificial sequence
  Synthetic polynucleotide
atgctgacctgacctttgac

SEQ ID NO: 142 - DNA, 20 bp, Artificial sequence
  Synthetic polynucleotide
ccaggtaagagctcagccac

SEQ ID NO: 143 - protein, 601 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPMEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 144 - protein, 609 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPMEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVDYKDDDDK

SEQ ID NO: 145 - DNA, 1806 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagagccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatggaagactggctcttcgtggttgtggtatgcctggctgccttcctcatcttcctcctcctgggcatctgctggtgccagtgctgcccgcacacttgctgctgctacgtcaggtgcccctgctgcccagacaagtgctgctgccccgaggccctgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtctga

SEQ ID NO: 146 - DNA, 1830 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagagccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatggaagactggctcttcgtggttgtggtatgcctggctgccttcctcatcttcctcctcctgggcatctgctggtgccagtgctgcccgcacacttgctgctgctacgtcaggtgcccctgctgcccagacaagtgctgctgccccgaggccctgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtcgactacaaagacgatgacgacaagtga

SEQ ID NO: 147 - DNA, 32 bp, Artificial sequence
  Synthetic polynucleotide
tatgctagcgccaccatggcgctgttggccgg

SEQ ID NO: 148 - DNA, 25 bp, Artificial sequence
  Synthetic polynucleotide
tcagacgactaaactttcccgactc

SEQ ID NO: 149 - DNA, 20 bp, Artificial sequence
  Synthetic polynucleotide
caggtaagagctcagccacc

SEQ ID NO: 150 - DNA, 20 bp, Artificial sequence
  Synthetic polynucleotide
ggtggctgagctcttacctg

SEQ ID NO: 151 - DNA, 58 bp, Artificial sequence
  Synthetic polynucleotide
gatggatcctcacttgtcgtcatcgtctttgtagtcgacgactaaactttcccgactc

SEQ ID NO: 152 - DNA, 34 bp, Artificial sequence
  Synthetic polynucleotide
gatggatcctcagacgactaaactttcccgactc

SEQ ID NO: 153 - protein, 8 aa, Artificial sequence
  Synthetic polypeptide
DYKDDDDK

SEQ ID NO: 154 - DNA, 1950 bp, Artificial sequence
  Synthetic polynucleotide
atgcaacaggacggacttggagtagggacaaggaacggaagtgggaaggggaggagcgtgcacccctcctggccttggtgcgcgccgcgccccctaaggtactttggaagggacgcgcgggccagacgcgcccagacggccgcgatggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagactggctcttcgtggttgtggtatgcctggctgccttcctcatcttcctcctcctgggcatctgctggtgccagtgctgcccgcacacttgctgctgctacgtcaggtgcccctgctgcccagacaagtgctgctgccccgaggccctgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtctga

SEQ ID NO: 155 - DNA, 1356 bp, Artificial sequence
  Synthetic polynucleotide
atccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaatgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 156 - protein, 232 aa, Artificial sequence
  Synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 217 aa, Artificial sequence
  Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 217 aa, Artificial sequence
  Synthetic polypeptide
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 159 - protein, 2 aa, Artificial sequence
  Synthetic polypeptide
GS

SEQ ID NO: 160 - protein, 4 aa, Artificial sequence
  Synthetic polypeptide
GSGS

SEQ ID NO: 161 - protein, 2 aa, Artificial sequence
  Synthetic polypeptide
AS

SEQ ID NO: 162 - protein, 4 aa, Artificial sequence
  Synthetic polypeptide
GGGS

SEQ ID NO: 163 - protein, 5 aa, Artificial sequence
  Synthetic polypeptide
GGGGS

SEQ ID NO: 164 - protein, 10 aa, Artificial sequence
  Synthetic polypeptide
GGGGSGGGGS

SEQ ID NO: 165 - protein, 15 aa, Artificial sequence
  Synthetic polypeptide
GGGGSGGGGSGGGGS

SEQ ID NO: 166 - protein, 20 aa, Artificial sequence
  Synthetic polypeptide
GGGGSGGGGSGGGGSGGGGS

SEQ ID NO: 167 - DNA, 7 bp, Artificial sequence
  Synthetic polynucleotide
agaaaka

SEQ ID NO: 168 - protein, 12 aa, Artificial sequence
  Synthetic polypeptide
AGAAAKGAAAKA

SEQ ID NO: 169 - protein, 17 aa, Artificial sequence
  Synthetic polypeptide
AGAAAKGAAAKGAAAKA

SEQ ID NO: 170 - protein, 22 aa, Artificial sequence
  Synthetic polypeptide
AGAAAKGAAAKGAAAKGAAAKA

SEQ ID NO: 171 - protein, 27 aa, Artificial sequence
  Synthetic polypeptide
AGAAAKGAAAKGAAAKGAAAKGAAAKA

SEQ ID NO: 172 - protein, 450 aa, Artificial sequence
  Synthetic polypeptide
ADNMQAIYVALGEAVELPCPSPPTLHGDEHLSWFCSPAAGSFTTLVAQVQVGRPAPDPGKPGRESRLRLLGNYSLWLEGSKEEDAGRYWCAVLGQHHNYQNWRVYDVLVLKGSQLSARAADGSPCNVLLCSVVPSRRMDSVTWQEGKGPVRGRVQSFWGSEAALLLVCPGEGLSEPRSRRPRIIRCLMTHNKGVSFSLAASIDASPALCAPSTGWDMPEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 615 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 514 aa, Artificial sequence
  Synthetic polypeptide
VVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 438 aa, Artificial sequence
  Synthetic polypeptide
ELEPQIDGQTWAERALRENERHAFTCRVAGGPGTPRLAWYLDGQLQEASTSRLLSVGGEAFSGGTSTFTVTAHRAQHELNCSLQDPRSGRSANASVILNVQFKPEIAQVGAKYQEAQGPGLLVVLFALVRANPPANVTWIDQDGPVTVNTSDFLVLDAQNYPWLTNHTVQLQLRSLAHNLSVVATNDVGVTSASLPAPGLLATRVEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 402 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 383 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 724 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 723 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 684 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGMYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 743 aa, Artificial sequence
  Synthetic polypeptide
IQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - DNA, 702 bp, Artificial sequence
  Synthetic polynucleotide
atggcagtcttattcctcctcctgttcctatgtggaactccccaggctgcagacaacatgcaggccatctatgtggccttgggggaggcagtagagctgccatgtccctcaccacctactctacatggggacgaacacctgtcatggttctgcagccctgcagcaggctccttcaccaccctggtagcccaagtccaagtgggcaggccagccccagaccctggaaaaccaggaagggaatccaggctcagactgctggggaactattctttgtggttggagggatccaaagaggaagatgccgggcggtactggtgcgctgtgctaggtcagcaccacaactaccagaactggagggtgtacgacgtcttggtgctcaaaggatcccagttatctgcaagggctgcagatggatccccctgcaatgtcctcctgtgctctgtggtccccagcagacgcatggactctgtgacctggcaggaagggaagggtcccgtgaggggccgtgttcagtccttctggggcagtgaggctgccctgctcttggtgtgtcctggggaggggctttctgagcccaggagccgaagaccaagaatcatccgctgcctcatgactcacaacaaaggggtcagctttagcctggcagcctccatcgatgcttctcctgccctctgtgccccttccacgggctgggacatgcct

SEQ ID NO: 183 - DNA, 1239 bp, Artificial sequence
  Synthetic polynucleotide
atggccgcaggcggcagtgcgcccgagccccgcgtcctcgtctgcctcggggcgctcctggccggctgggtcgccgtaggattggaggctgttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccagcggcccctatcagattgaggtccacatcgtggccaccggcacactccccaacggcaccctctatgcagccaggggctcccaggtggacttcagctgcaacagcagctccaggccaccacccgtggttgaatggtggttccaggccctgaattccagcagcgagtcctttggccacaacctgacagtcaactttttctcactgttactgatatcgccaaacctccaagggaactacacctgtttagccttgaatcagctcagcaagagacatcgaaaggtgaccaccgagctcctggtctactatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcgggggg

SEQ ID NO: 184 - DNA, 936 bp, Artificial sequence
  Synthetic polynucleotide
atggccgcaggcggcagtgcgcccgagccccgcgtcctcgtctgcctcggggcgctcctggccggctgggtcgccgtaggattggaggctgttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccaatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcgggggg

SEQ ID NO: 185 - DNA, 690 bp, Artificial sequence
  Synthetic polynucleotide
atggcgctgcctccaggcccagccgccctccggcacacactgctgctcctgccagcccttctgagctcaggttggggggagttggagccacaaatagatggtcagacctgggctgagcgggcacttcgggagaatgaacgccacgccttcacctgccgggtggcaggggggcctggcacccccagattggcctggtatctggatggacagctgcaggaggccagcacctcaagactgctgagcgtgggaggggaggccttctctggaggcaccagcaccttcactgtcactgcccatcgggcccagcatgagctcaactgctctctgcaggaccccagaagtggccgatcagccaacgcctctgtcatccttaatgtgcaattcaagccagagattgcccaagtcggcgccaagtaccaggaagctcagggcccaggcctcctggttgtcctgtttgccctggtgcgtgccaacccgccggccaatgtcacctggatcgaccaggatgggccagtgactgtcaacacctctgacttcctggtgctggatgcgcagaactacccctggctcaccaaccacacggtgcagctgcagctccgcagcctggcacacaacctctcggtggtggccaccaatgacgtgggtgtcaccagtgcgtcgcttccagccccagggcttctggctacccgg

SEQ ID NO: 186 - DNA, 633 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagac

SEQ ID NO: 187 - DNA, 576 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgac

SEQ ID NO: 188 - DNA, 1533 bp, Artificial sequence
  Synthetic polynucleotide
atccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtc

SEQ ID NO: 189 - DNA, 1398 bp, Artificial sequence
  Synthetic polynucleotide
atggcagtcttattcctcctcctgttcctatgtggaactccccaggctgcagacaacatgcaggccatctatgtggccttgggggaggcagtagagctgccatgtccctcaccacctactctacatggggacgaacacctgtcatggttctgcagccctgcagcaggctccttcaccaccctggtagcccaagtccaagtgggcaggccagccccagaccctggaaaaccaggaagggaatccaggctcagactgctggggaactattctttgtggttggagggatccaaagaggaagatgccgggcggtactggtgcgctgtgctaggtcagcaccacaactaccagaactggagggtgtacgacgtcttggtgctcaaaggatcccagttatctgcaagggctgcagatggatccccctgcaatgtcctcctgtgctctgtggtccccagcagacgcatggactctgtgacctggcaggaagggaagggtcccgtgaggggccgtgttcagtccttctggggcagtgaggctgccctgctcttggtgtgtcctggggaggggctttctgagcccaggagccgaagaccaagaatcatccgctgcctcatgactcacaacaaaggggtcagctttagcctggcagcctccatcgatgcttctcctgccctctgtgccccttccacgggctgggacatgcctgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 190 - DNA, 1935 bp, Artificial sequence
  Synthetic polynucleotide
atggccgcaggcggcagtgcgcccgagccccgcgtcctcgtctgcctcggggcgctcctggccggctgggtcgccgtaggattggaggctgttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccagcggcccctatcagattgaggtccacatcgtggccaccggcacactccccaacggcaccctctatgcagccaggggctcccaggtggacttcagctgcaacagcagctccaggccaccacccgtggttgaatggtggttccaggccctgaattccagcagcgagtcctttggccacaacctgacagtcaactttttctcactgttactgatatcgccaaacctccaagggaactacacctgtttagccttgaatcagctcagcaagagacatcgaaaggtgaccaccgagctcctggtctactatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcgggggggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 191 - DNA, 1632 bp, Artificial sequence
  Synthetic polynucleotide
atggccgcaggcggcagtgcgcccgagccccgcgtcctcgtctgcctcggggcgctcctggccggctgggtcgccgtaggattggaggctgttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccaatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcgggggggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 192 - DNA, 1386 bp, Artificial sequence
  Synthetic polynucleotide
atggcgctgcctccaggcccagccgccctccggcacacactgctgctcctgccagcccttctgagctcaggttggggggagttggagccacaaatagatggtcagacctgggctgagcgggcacttcgggagaatgaacgccacgccttcacctgccgggtggcaggggggcctggcacccccagattggcctggtatctggatggacagctgcaggaggccagcacctcaagactgctgagcgtgggaggggaggccttctctggaggcaccagcaccttcactgtcactgcccatcgggcccagcatgagctcaactgctctctgcaggaccccagaagtggccgatcagccaacgcctctgtcatccttaatgtgcaattcaagccagagattgcccaagtcggcgccaagtaccaggaagctcagggcccaggcctcctggttgtcctgtttgccctggtgcgtgccaacccgccggccaatgtcacctggatcgaccaggatgggccagtgactgtcaacacctctgacttcctggtgctggatgcgcagaactacccctggctcaccaaccacacggtgcagctgcagctccgcagcctggcacacaacctctcggtggtggccaccaatgacgtgggtgtcaccagtgcgtcgcttccagccccagggcttctggctacccgggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 193 - DNA, 1329 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagacgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 194 - DNA, 1272 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgacgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 195 - DNA, 2295 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtcgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 196 - DNA, 2292 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtcgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 197 - DNA, 2175 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaatgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtcgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 198 - DNA, 2352 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagtgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtcgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 199 - DNA, 30 bp, Artificial sequence
  Synthetic polynucleotide
tcaggcggggcccatagaagactggctctt

SEQ ID NO: 200 - DNA, 30 bp, Artificial sequence
  Synthetic polynucleotide
aagagccagtcttctatgggccccgcctga

SEQ ID NO: 201 - DNA, 1749 bp, Artificial sequence
  Synthetic polynucleotide
atggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgtcgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgactggctcttcgtggttgtggtatgcctggctgccttcctcatcttcctcctcctgggcatctgctggtgccagtgctgcccgcacacttgctgctgctacgtcaggtgcccctgctgcccagacaagtgctgctgccccgaggccctgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtctga

SEQ ID NO: 202 - DNA, 1785 bp, Artificial sequence
  Synthetic polynucleotide
atggcccctgccgcatccgcctgtgctggagcccctggaagtcaccccgcaaccactattttcgtctgtctgtttctgattatctattgccctgatcgagcaagcgccatccaggtgaccgtcccagacccttaccacgtggtcattctgttccagcctgtcacactgcattgcacttatcagatgagtaacaccctgacagccccaatcgtgatttggaaatacaagagcttctgtcgagacagggtcgcagatgccttttctccagctagtgtggacaaccagctgaatgcacagctggccgctgggaaccctggctacaatccatatgtggagtgccaggatagcgtgagaactgtccgggtggtcgccaccaaacagggcaacgctgtgacactgggggactactatcagggcaggagaatcactattaccggcaatgccgatctgacctttgagcagacagcttggggagactccggggtgtactattgttcagtggtcagcgcccaggacctggatggcaacaatgaggcttacgcagaactgatcgtgctgggacggacttctgaggctcccgaactgctgcctggattccgcgcagggcctctggaggattggctgtttgtggtcgtggtctgcctggccagcctgctgttctttctgctgctgggcatttgctggtgtcagtgctgtccccacacttgctgttgctatgtgaggtgtccatgttgccccgacaaatgttgctgtcctgaggccctgtacgcagccggcaaggctgcaacctcaggagtgcctagcatctacgctccaagcatttatacacacctgtccccagcaaaaactccccctccaccccctgccatgatccctatgaggccaccctacggctatccaggagacttcgatagaactagctccgtcggcggacattctagtcaggtgcccctgctgagagaggtggatgggtctgtctcaagcgaagtgcggagtggctaccgcattcaggccaatcagcaggacgatagcatgcgggtgctgtactatatggagaaggaactggccaactttgacccttcccgaccaggacctccaaatggacgagtcgagcgagccatgtccgaagtgacctctctgcacgaggacgattggagatcccgaccatctcgagcacccgctctgacccctatccgagatgaggaatggaacaggcatagtcccaggtcacctagaacatgggagcaggaacccctgcaggagcagcctcgaggaggatggggatcagggcgcccaagagctaggagcgtggacgcactggacgatatcaatcgccccggaagcacagaatccgggcgatcctctccacctagttcaggcaggaggggacgagcatacgcaccaccaagaagccggtcccgagacgatctgtatgacccagacgatcccagagatctgccacactcccgggacccccattactatgacgatctgcgatctagggacccaagggcagatcccagaagtcggcagaggagccacgaccccagagatgctggattccgatctagggaccctcagtatgatgggaggctgctggaggaagcactgaagaaaaagggcgccggagagcgaaggagagtgtacagagaggaggaagaagaggaagaggaaggccattaccctccagccccccctccatatagcgagacagactctcaggctagtcgcgaacggcgcatgaagaagaatctggcactgtcacgggagagcctggtggtgtga

SEQ ID NO: 203 - DNA, 1803 bp, Artificial sequence
  Synthetic polynucleotide
atggcgctggtggccggcgggctctgtagagggctgggctcccacccggccgccccaggccgggacgcggtagtcttcgtgtggctcctgcttagcacctggttcacagcccctgccagggccatccaggtgaccgtgtccgacccctaccacgtggtgatcctcttccagcccgtgaccctaccctgtacctaccagatgacctcgacccccacgcaacccatcgtcatctggaagtacaagtctttctgccgggaccgcatcgctgatgccttctccccagccagcgtcgacaaccagctcaatgcccagctggtagccgggaacccaggctacaacccctacgtcgagtgccaggacagcgtgcgcaccgtcagggtcgtggccaccaagcagggcaacgctgtgaccctgggagattactaccagggtcggaggattaccatcaccggaaatgctgacctgacctttgacgagacggcgtggggggacagcggtgtgtattactgctctgtggtctcagcccaggacctcgaggggaacaatgaggcctacgcagagctcatcgtccttgggaggacctcaggggtggctgagctcttacctggttttcaggcggggcccatggaagactggctcttcgtggttgtggtgtgcctggctgccttcctcgtcttcctcctcctgggcatctgctggtgccagtgctgcccacacacttgctgctgctacgtcaggtgcccctgctgcccagacaagtgctgctgccctgaggccctgtatgccgccggcaaagcagccacctcaggtgttcccagcatctatgcccccagcacctatgcccacctgtctcccgccaagaccccgcccctaccaactgtgattcccatgggccctgcctacaacgggtaccctggagggtaccctggggacctcgacaggagtagctcagctggtggccaaggttcctacgtacccctgcttcgggacacagacagcagtgtggcctctgaagtccgcagtggctacaggattcaggccagccagcaagacgactccatgcgggtcctgtactacatggagaaggagctggccaatttcgacccttctcgacctggcccccccaatggccgtgtggagcgggccatgagtgaagtcacctccctccatgaggacgactggcgatctcggcctcctcggggccctgccctcaccccgatccgggatgaggagtggggtggccactccccccggagtcccaggggatgggacccagagacccccagggagcaggcaggcgggggctggcgggccaggcggccccgggcccgctccgtggacgccctggacgacctcaccccgccaagctccgccgagtcagggagcagatctcccccgagtagtggtgggaggagaggccgggcctacatgcccccgcggagccgcagccgggacgacctctatgaccaggaagactcgagagacttcccacgctcccgggaccctcactacgacgacatcaggtctcgggagcgcctccctgccgaccccaggtcccactaccaccgtacccgggaccctcgggacaacggctccaggtccggggaccccccctatgatgggcggctactggaggaggccgtgaagaagaaggggtcgggggagagacggagaccctacaaggaggaagaggcctactatccgcccgcgccgcccccgtactcggagaccgactcgcaggcgtcccgcgagcgcaggctcaagaagaacctggccctgagtcgggaaagtttagtcgtctga

SEQ ID NO: 204 - DNA, 31 bp, Artificial sequence
  Synthetic polynucleotide
aaagctagcgccaccatggcgctggtggccg

SEQ ID NO: 205 - DNA, 19 bp, Artificial sequence
  Synthetic polynucleotide
caggtaagagctcagccac

SEQ ID NO: 206 - DNA, 56 bp, Artificial sequence
  Synthetic polynucleotide
aaagaattctcacttgtcgtcatcgtctttgtagtcgacgactaaactttcccgac

SEQ ID NO: 207 - DNA, 19 bp, Artificial sequence
  Synthetic polynucleotide
gtggctgagctcttacctg

SEQ ID NO: 208 - DNA, 1747 bp, Artificial sequence
  Synthetic polynucleotide
atggcgctggtggccggcgggctctgtagagggctgggctcccacccggccgccccaggccgggacgcggtagtcttcgtgtggctcctgcttagcacctggttcacagcccctgccagggccatccaggtgaccgtgtccgacccctaccacgtggtgatcctcttccagcccgtgaccctaccctgtacctaccagatgacctcgacccccacgcaacccatcgtcatctggaagtacaagtctttctgccgggaccgcatcgctgatgccttctccccagccagcgtcgacaaccagctcaatgcccagctggtagccgggaacccaggctacaacccctacgtcgagtgccaggacagcgtgcgcaccgtcagggtcgtggccaccaagcagggcaacgctgtgaccctgggagattactaccagggtcggaggattaccatcaccggaaatgctgacctgacctttgacgagacggcgtggggggacagcggtgtgtattactgctctgtggtctcagcccaggacctcgaggggaacaatgaggcctacgcagagctcatcgtccttgactggctcttcgtggttgtggtgtgcctggctgccttcctcgtcttcctcctcctgggcatctgctggtgccagtgctgcccacacacttgctgctgctacgtcaggtgcccctgctgcccagacaagtgctgctgccctgaggccctgtatgccgccggcaaagcagccacctcaggtgttcccagcatctatgcccccagcacctatgcccacctgtctcccgccaagaccccgcccctaccaactgtgattcccatgggccctgcctacaacgggtaccctggagggtaccctggggacctcgacaggagtagctcagctggtggccaaggttcctacgtacccctgcttcgggacacagacagcagtgtggcctctgaagtccgcagtggctacaggattcaggccagccagcaagacgactccatgcgggtcctgtactacatggagaaggagctggccaatttcgacccttctcgacctggcccccccaatggccgtgtggagcgggccatgagtgaagtcacctccctccatgaggacgactggcgatctcggcctcctcggggccctgccctcaccccgatccgggatgaggagtggggtggccactccccccggagtcccaggggatgggacccagagacccccagggagcaggcaggcgggggctggcgggccaggcggccccgggcccgctccgtggacgccctggacgacctcaccccgccaagctccgccgagtctgggagcagatctcccccgagtagtggtgggaggagaggccgggcctacatgcccccgcggagccgcagccgggacgacctctatgaccaagaagactcgagagacttcccacgctcccgggaccctcactacgacgacatcaggtctcgggagcgcctccctgccgaccccaggtcccactaccaccgtacccgggaccctcgggacaacggctccaggtccggggaccccccctatgatgggcggctactggaggaggccgtgaagaagaaggggtcgggggagagacggagaccctacaaggaggaagaggcctactatccgcccgcgccgcccccgtactcggagaccgactcgcaggcgtcccgcgagcgcaggctcaagaagaacctggccctgagtcgggaaagtttagtcgtcgtga

SEQ ID NO: 209 - protein, 601 aa, Artificial sequence; identical to SEQ ID NO: 11
  Synthetic polypeptide

SEQ ID NO: 210 - protein, 582 aa, Artificial sequence; identical to SEQ ID NO: 13
  Synthetic polypeptide

SEQ ID NO: 211 - protein, 600 aa, Artificial sequence
  Synthetic polypeptide
MALVAGGLCRGLGSHPAAPGRDAVVFVWLLLSTWFTAPARAIQVTVSDPYHVVILFQPVTLPCTYQMTSTPTQPIVIWKYKSFCRDRIADAFSPASVDNQLNAQLVAGNPGYNPYVECQDSVRTVRVVATKQGNAVTLGDYYQGRRITITGNADLTFDETAWGDSGVYYCSVVSAQDLEGNNEAYAELIVLGRTSGVAELLPGFQAGPMEDWLFVVVVCLAAFLVFLLLGICWCQCCPHTCCCYVRCPCCPDKCCCPEALYAAGKAATSGVPSIYAPSTYAHLSPAKTPPLPTVIPMGPAYNGYPGGYPGDLDRSSSAGGQGSYVPLLRDTDSSVASEVRSGYRVQASQQDDSMRVLYYMEKELANFDPSRPGPPNGRVEWAMSEVTSLHEDDWRSRPPWGPALTPIRDEEWGGHSPRSPRGWDPETPREQAGGGWRARRPRARSVDALDDLTPPSSAESGSRSPPSSGGRRGRAYMPPRSRSRDDLYDQEDSRDFPRSRDPHYDDIRSRERLPADPRSHYHRTRDPRDNGSRSGDPPYDGRLLEEAVKKKGSGERRRPYKEEEAYYPPAPPPYSETDSQASRERRLKKNLALSRESLVV

SEQ ID NO: 212 - protein, 581 aa, Artificial sequence
  Synthetic polypeptide
MALVAGGLCRGLGSHPAAPGRDAVVFVWLLLSTWFTAPARAIQVTVSDPYHVVILFQPVTLPCTYQMTSTPTQPIVIWKYKSFCRDRIADAFSPASVDNQLNAQLVAGNPGYNPYVECQDSVRTVRVVATKQGNAVTLGDYYQGRRITITGNADLTFDETAWGDSGVYYCSVVSAQDLEGNNEAYAELIVLDWLFVVVVCLAAFLVFLLLGICWCQCCPHTCCCYVRCPCCPDKCCCPEALYAAGKAATSGVPSIYAPSTYAHLSPAKTPPLPTVIPMGPAYNGYPGGYPGDLDRSSSAGGQGSYVPLLRDTDSSVASEVRSGYRIQASQQDDSMRVLYYMEKELANFDPSRPGPPNGRVERAMSEVTSLHEDDWRSRPPRGPALTPIRDEEWGGHSPRSPRGWDPETPREQAGGGWRARRPRARSVDALDDLTPPSSAESGSRSPPSSGGRRGRAYMPPRSRSRDDLYDQEDSRDFPRSRDPHYDDIRSRERLPADPRSHYHRTRDPRDNGSRSGDPPYDGRLLEEAVKKKGSGERRRPYKEEEAYYPPAPPPYSETDSQASRERRLKKNLALSRESLVV

SEQ ID NO: 213 - protein, 594 aa, Artificial sequence; identical to SEQ ID NO: 31
  Synthetic polypeptide

SEQ ID NO: 214 - protein, 211 aa, Artificial sequence; identical to SEQ ID NO: 10
  Synthetic polypeptide

SEQ ID NO: 215 - protein, 18 aa, Artificial sequence
  Synthetic polypeptide
KSFCRDRIADAFSPASVD

SEQ ID NO: 216 - protein, 18 aa, Artificial sequence
  Synthetic polypeptide
CQDSVRTVRVVATKQGNA

SEQ ID NO: 217 - DNA, 420 bp, Artificial sequence
  Synthetic polynucleotide
atgaacttcgggctcagattgattttccttgtccttgttttaaaaggtgtcctgtgtgacgtgaagctcgtggagtctgggggaggcttagtgaagcttggagggtccctgaaactctcctgtgcagcctctggattcactttcagtagctattacatgtcttgggttcgccagactccagagaagaggctggagttggtcgcagccattaatagtaatggtggtagcacctactatccagacactgtgaagggccgattcaccatctccagagacaatgccaagaacaccctgtacctgcaaatgagcagtctgaagtctgaggacacagccttgtattactgtgcaagacatgattactacggtagtagctttgctatggactactggggtcaaggaacctcagtcaccgtctcctca

SEQ ID NO: 218 - protein, 140 aa, Artificial sequence
  Synthetic polypeptide
MNFGLRLIFLVLVLKGVLCDVKLVESGGGLVKLGGSLKLSCAASGFTFSSYYMSWVRQTPEKRLELVAAINSNGGSTYYPDTVKGRFTISRDNAKNTLYLQMSSLKSEDTALYYCARHDYYGSSFAMDYWGQGTSVTVSS

SEQ ID NO: 219 - DNA, 381 bp, Artificial sequence
  Synthetic polynucleotide
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctgggacagattattctctcactattagcaacctggaacaagaagatattgccacttacttttgccaacaggatagtaagcatccgtggacgttcggtggaggcaccaagctggaaatcaaa

SEQ ID NO: 220 - protein, 127 aa, Artificial sequence
  Synthetic polypeptide
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQDSKHPWTFGGGTKLEIK

SEQ ID NO: 221 - protein, 439 aa, Artificial sequence
  Synthetic polypeptide
MAPAASACAGAPGSHPATTIFVCLFLIIYCPDRASAIQVTVPDPYHVVILFQPVTLHCTYQMSNTLTAPIVIWKYKSFCRDRVADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLGRTSEAPELLPGFRAGPLEDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 222 - protein, 444 aa, Artificial sequence
  Synthetic polypeptide
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 223 - protein, 428 aa, Artificial sequence
  Synthetic polypeptide
MDRVLLRWISLFWLTAMVEGLQVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMGSENLYFQGSGEPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 224 - DNA, 30 bp, Artificial sequence
  Synthetic polynucleotide
ggattcactttcagtagctattacatgtct

SEQ ID NO: 225 - DNA, 51 bp, Artificial sequence
  Synthetic polynucleotide
gccattaatagtaatggtggtagcacctactatccagacactgtgaagggc

SEQ ID NO: 226 - DNA, 36 bp, Artificial sequence
  Synthetic polynucleotide
catgattactacggtagtagctttgctatggactac

SEQ ID NO: 227 - protein, 10 aa, Artificial sequence
  Synthetic polypeptide
GFTFSSYYMS

SEQ ID NO: 228 - protein, 17 aa, Artificial sequence
  Synthetic polypeptide
AINSNGGSTYYPDTVKG

SEQ ID NO: 229 - protein, 12 aa, Artificial sequence
  Synthetic polypeptide
HDYYGSSFAMDY

SEQ ID NO: 230 - DNA, 33 bp, Artificial sequence
  Synthetic polynucleotide
agggcaagtcaggacattagcaattatttaaac

SEQ ID NO: 231 - DNA, 21 bp, Artificial sequence
  Synthetic polynucleotide
tacacatcaagattacactca

SEQ ID NO: 232 - DNA, 27 bp, Artificial sequence
  Synthetic polynucleotide
caacaggatagtaagcatccgtggacg

SEQ ID NO: 233 - protein, 11 aa, Artificial sequence
  Synthetic polypeptide
RASQDISNYLN

SEQ ID NO: 234 - protein, 7 aa, Artificial sequence
  Synthetic polypeptide
YTSRLHS

SEQ ID NO: 235 - protein, 9 aa, Artificial sequence
  Synthetic polypeptide
QQDSKHPWT

SEQ ID NO: 236 - protein, 443 aa, Artificial sequence; identical to SEQ ID NO: 75
  Synthetic polypeptide

SEQ ID NO: 237 - protein, 81 aa, Homo sapiens
LLSTWCTAPARAIQVTVSNPYHVVILFQPVTLPCTYQMTSTPTQPIVIWKYKSFCRDRIADAFSPASVDNQLNAQLAAGNP

SEQ ID NO: 238 - protein, 115 aa, Artificial sequence
  Synthetic polypeptide
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLNMDYWGQGTLVTVSS

SEQ ID NO: 239 - protein, 10 aa, Artificial sequence
  Synthetic polypeptide
GFTFSSYGMH

SEQ ID NO: 240 - protein, 8 aa, Artificial sequence
  Synthetic polypeptide
GFTFSSYG

SEQ ID NO: 241 - protein, 9 aa, Artificial sequence
  Synthetic polypeptide
FTFSSYGMH

SEQ ID NO: 242 - protein, 6 aa, Artificial sequence
  Synthetic polypeptide
DLNMDY

SEQ ID NO: 243 - protein, 8 aa, Artificial sequence
  Synthetic polypeptide
AKDLNMDY

SEQ ID NO: 244 - protein, 110 aa, Artificial sequence
  Synthetic polypeptide
QTVVTQEPSLTVSPGGTVTLTCASSTGAVTSGYYPTWIQQKPGQAPRALIHTTSYKHSWTPARFSGSLVGDKAALTLSGVQPEDEAEYYCLLQYGGAWVFGGGTKVTVLG

SEQ ID NO: 245 - protein, 14 aa, Artificial sequence
  Synthetic polypeptide
ASSTGAVTSGYYPT

SEQ ID NO: 246 - protein, 9 aa, Artificial sequence
  Synthetic polypeptide
TGAVTSGYY

SEQ ID NO: 247 - protein, 7 aa, Artificial sequence
  Synthetic polypeptide
TTSYKHS

SEQ ID NO: 248 - protein, 3 aa, Artificial sequence
  Synthetic polypeptide
TTS

SEQ ID NO: 249 - protein, 9 aa, Artificial sequence
  Synthetic polypeptide
LLQYGGAWV

SEQ ID NO: 250 - protein, 118 aa, Artificial sequence
  Synthetic polypeptide
QVQLVESGGGLVKPGRSLRLSCAASGFTFSSYAMHWVRQAPGKGLEWVAVISYDGSNKHYADSVKGRFTISRDNSKNTLYLQMNSLRAEDTAVYYCARDSPGNAFDIWGQGTMVTVSS

SEQ ID NO: 251 - protein, 10 aa, Artificial sequence
  Synthetic polypeptide
GFTFSSYAMH

SEQ ID NO: 252 - protein, 6 aa, Artificial sequence
  Synthetic polypeptide
GFTFSS

SEQ ID NO: 253 - protein, 8 aa, Artificial sequence
  Synthetic polypeptide
GFTFSSYA

SEQ ID NO: 254 - protein, 9 aa, Artificial sequence
  Synthetic polypeptide
FTFSSYAMH

SEQ ID NO: 255 - protein, 10 aa, Artificial sequence
  Synthetic polypeptide
VISYDGSNKH

SEQ ID NO: 256 - protein, 8 aa, Artificial sequence
  Synthetic polypeptide
ISYDGSNK

SEQ ID NO: 257 - protein, 17 aa, Artificial sequence
  Synthetic polypeptide
VISYDGSNKHYADSVKG

SEQ ID NO: 258 - protein, 9 aa, Artificial sequence
  Synthetic polypeptide
DSPGNAFDI

SEQ ID NO: 259 - protein, 11 aa, Artificial sequence
  Synthetic polypeptide
ARDSPGNAFDI

SEQ ID NO: 260 - protein, 112 aa, Artificial sequence
  Synthetic polypeptide
DVVMTQSPLSLPVTPGEPASISCRSSQSLLHSNGYNYLDWYLQKPGQSPQLLIYLGSTRASGVPDRFSGSGSGTDFTLKISRVEAEDVGVYYCMQALQTWTFGQGTKVEIKR

SEQ ID NO: 261 - protein, 7 aa, Artificial sequence
  Synthetic polypeptide
LGSTRAS

SEQ ID NO: 262 - protein, 8 aa, Artificial sequence
  Synthetic polypeptide
MQALQTWT

SEQ ID NO: 263 - protein, 120 aa, Artificial sequence
  Synthetic polypeptide
QVQLVQSGAEVKKPGASVKVSCKVSGYSLTELSMNWVRQAPGKGLEWMGGLDPEDRESIYAQKFQGRVTMTEDTSTDTAYLELSSLSSEDTAVYYCATNYFDISGYFDLWGQGTLVTVSS

SEQ ID NO: 264 - protein, 10 aa, Artificial sequence
  Synthetic polypeptide
GYSLTELSMN

SEQ ID NO: 265 - protein, 6 aa, Artificial sequence
  Synthetic polypeptide
GYSLTE

SEQ ID NO: 266 - protein, 8 aa, Artificial sequence
  Synthetic polypeptide
GYSLTELS

SEQ ID NO: 267 - protein, 9 aa, Artificial sequence
  Synthetic polypeptide
YSLTELSMN

SEQ ID NO: 268 - protein, 10 aa, Artificial sequence
  Synthetic polypeptide
GLDPEDRESI

SEQ ID NO: 269 - protein, 8 aa, Artificial sequence
  Synthetic polypeptide
LDPEDRES

SEQ ID NO: 270 - protein, 17 aa, Artificial sequence
  Synthetic polypeptide
GLDPEDRESIYAQKFQG

SEQ ID NO: 271 - protein, 11 aa, Artificial sequence
  Synthetic polypeptide
NYFDISGYFDL

SEQ ID NO: 272 - protein, 13 aa, Artificial sequence
  Synthetic polypeptide
ATNYFDISGYFDL

SEQ ID NO: 273 - protein, 111 aa, Artificial sequence
  Synthetic polypeptide
QAGLTQPPSVSKGLRQTATLTCTGNSNNVGYQGAAWLQQQQGHPPKFLSYRSNNRPSGISERVSASRSGNTASLTIAGLQPEDEADYYCSAWDTSLSAWVFGGGTKVTVLG

SEQ ID NO: 274 - protein, 13 aa, Artificial sequence
  Synthetic polypeptide
TGNSNNVGYQGAA

SEQ ID NO: 275 - protein, 8 aa, Artificial sequence
  Synthetic polypeptide
SNNVGYQG

SEQ ID NO: 276 - protein, 7 aa, Artificial sequence
  Synthetic polypeptide
RSNNRPS

SEQ ID NO: 277 - protein, 3 aa, Artificial sequence
  Synthetic polypeptide
RSN

SEQ ID NO: 278 - protein, 11 aa, Artificial sequence
  Synthetic polypeptide
SAWDTSLSAWV

SEQ ID NO: 279 - protein, 120 aa, Artificial sequence
  Synthetic polypeptide
QVQLVQSGAEVKKPGASVKVSCKVSGYSLTELSMNWVRQAPGKGLEWMGGLDPEDRESIYAQKFQGRVTMTEDTSTDTAYLELSSLSSEDTAVYYCATNYFDISGYFDLWGQGTMVTVSS

SEQ ID NO: 280 - protein, 17 aa, Artificial sequence
  Synthetic polypeptide
GLDPEDRESIIAQKFQG

SEQ ID NO: 281 - protein, 111 aa, Artificial sequence
  Synthetic polypeptide
QAGLTQPPSVSKGLRQTATLTCTGNSNNVGYQGAAWLQQQQGHPPKFLSYRSNNRPSGISERVSASRSGNTASLTITGLQPEDEADYYCSAWDSSLSTWVFGGGTKVTVLG

SEQ ID NO: 282 - protein, 11 aa, Artificial sequence
  Synthetic polypeptide
SAWDSSLSTWV

SEQ ID NO: 283 - protein, 116 aa, Artificial sequence
  Synthetic polypeptide
EVQLVESGGGVVRPGRSLRLSCAASGFTFSSYGMHWVRQAPGKGLEWVAVISYDGSNKYYADSVKGRFTISRDNSKNTLYLQMNSLRAEDTAVYYCARGINWFDPWGQGTLVTVSS

SEQ ID NO: 284 - protein, 7 aa, Artificial sequence
  Synthetic polypeptide
GINWFDP

SEQ ID NO: 285 - protein, 9 aa, Artificial sequence
  Synthetic polypeptide
ARGINWFDP

SEQ ID NO: 286 - protein, 118 aa, Artificial sequence
  Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDSPGNAFDIWGQGTMVTVSS

SEQ ID NO: 287 - protein, 10 aa, Artificial sequence
  Synthetic polypeptide
VISYDGSNKY

SEQ ID NO: 288 - protein, 17 aa, Artificial sequence
  Synthetic polypeptide
VISYDGSNKYYADSVKG

SEQ ID NO: 289 - protein, 112 aa, Artificial sequence
  Synthetic polypeptide
DVVMTQSPLSLPVTPGEPASVSCKSSQSLLRSNGYNYLDWYLQKPGQSPQLLIYLGSNRASGVPDRFSGSGSGTDFTLKISRVEAEDVGVYYCMQALESWTFGQGTKVDIKR

SEQ ID NO: 290 - protein, 16 aa, Artificial sequence
  Synthetic polypeptide
KSSQSLLRSNGYNYLD

SEQ ID NO: 291 - protein, 11 aa, Artificial sequence
  Synthetic polypeptide
QSLLRSNGYNY

SEQ ID NO: 292 - protein, 8 aa, Artificial sequence
  Synthetic polypeptide
MQALESWT

SEQ ID NO: 293 - protein, 130 aa, Artificial sequence
  Synthetic polypeptide
EVQLVQSGAEVKKPGASVKVSCKVSGYTLTELSMHWVRQAPGKGLEWMGGFDPEDGETIYSQKFQGRVTMTEDTSTDTAYMELSSLRSEDTAVYYCATYDYDSSGFSRGVPEENNAFDIWGQGTMVTVSS

SEQ ID NO: 294 - protein, 10 aa, Artificial sequence
  Synthetic polypeptide
GYTLTELSMH

SEQ ID NO: 295 - protein, 6 aa, Artificial sequence
  Synthetic polypeptide
GYTLTE

SEQ ID NO: 296 - protein, 8 aa, Artificial sequence
  Synthetic polypeptide
GYTLTELS

SEQ ID NO: 297 - protein, 9 aa, Artificial sequence
  Synthetic polypeptide
YTLTELSMH

SEQ ID NO: 298 - protein, 10 aa, Artificial sequence
  Synthetic polypeptide
GFDPEDGETI

SEQ ID NO: 299 - protein, 8 aa, Artificial sequence
  Synthetic polypeptide
FDPEDGET

SEQ ID NO: 300 - protein, 17 aa, Artificial sequence
  Synthetic polypeptide
GFDPEDGETIYSQKFQG

SEQ ID NO: 301 - protein, 21 aa, Artificial sequence
  Synthetic polypeptide
YDYDSSGFSRGVPEENNAFDI

SEQ ID NO: 302 - protein, 23 aa, Artificial sequence
  Synthetic polypeptide
ATYDYDSSGFSRGVPEENNAFDI

SEQ ID NO: 303 - protein, 16 aa, Artificial sequence
  Synthetic polypeptide
RSSQSLLHSNGYNYLD

SEQ ID NO: 304 - protein, 11 aa, Artificial sequence
  Synthetic polypeptide
QSLLHSNGYNY

SEQ ID NO: 305 - protein, 7 aa, Artificial sequence
  Synthetic polypeptide
LGSNRAS

SEQ ID NO: 306 - protein, 3 aa, Artificial sequence
  Synthetic polypeptide
LGS

SEQ ID NO: 307 - protein, 8 aa, Artificial sequence
  Synthetic polypeptide
MQALQGWT

SEQ ID NO: 308 - protein, 112 aa, Artificial sequence
  Synthetic polypeptide
DVMMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQGWTFGQGTKVEIKR